(19) United States
(12) Patent Application Publication
(10) Pub. No.: US 2025/0333539 A1
(43) Pub. Date: Oct. 30, 2025

(54) ACTIVATABLE ANTIGEN-BINDING PROTEIN CONSTRUCTS AND USES OF THE SAME

(71) Applicant: CytomX Therapeutics, Inc., South San Francisco, CA (US)

(72) Inventors: Laurie WONG, San Mateo, CA (US); Jason Gary SAGERT, South San Francisco, CA (US); Shuoyen Jack LIN, San Bruno, CA (US); Ellaine Anne Mariano FOX, San Carlos, CA (US); Madan M. PAIDHUNGAT, San Francisco, CA (US)

(21) Appl. No.: 18/849,502

(22) PCT Filed: Mar. 23, 2023
(86) PCT No.: PCT/US2023/064881

§ 371 Date: Sep. 22, 2024

Related U.S. Application Data
(60) Provisional application No. 63/322,711, filed on Mar. 23, 2022.

(51) Int. Cl. C07K 16/32; A61K 39/00; A61P 35/00; C07K 16/28

(52) U.S. Cl. CPC C07K 16/32; A61P 35/00; C07K 16/2809; A61K 2039/505; C07K 2317/31; C07K 2317/35; C07K 2317/522; C07K 2317/565; C07K 2317/622; C07K 2317/92; C07K 2317/94; C07K 2319/50

(57) ABSTRACT

Provided herein are protease-activatable antigen-binding protein constructs (ABPC) that when activated, generate an ABPC that is capable of specifically binding a biological target. Also provided are activatable bispecific ABPCs that, when activated, are capable of specifically binding two different biological targets. Activatable ABPCs that, when activated, are capable of binding human epidermal growth factor receptor 2 (HER2) are provided, along with anti-HER2 masking moieties.

### CROSS-REFERENCE TO RELATED APPLICATION

This application is a 35 U.S.C. 371 National Phase Entry Application of PCT/US2023/064881, filed Mar. 23, 2023, which claims the benefit of provisional application U.S. Ser. No. 63/322,711, filed Mar. 23, 2022, pursuant 35 U.S.C. § 119(e), which is incorporated herein by reference in its entirety.

### TECHNICAL FIELD

The present disclosure relates to the field of biotechnology, and more specifically, to activatable antigen-binding molecules.

### REFERENCE TO SEQUENCE LISTING

The “Sequence Listing” submitted electronically concurrently herewith pursuant 37 C.F.R. § 1.821 in computer readable form (CRF) as file name CYTX-055-PCT_SL.xml is incorporated herein by reference. The electronic copy of the Sequence Listing was created on Mar. 20, 2023, and the size on disk is 687 kilobytes.

### BACKGROUND

Monoclonal antibodies have emerged as a promising class of drugs for the treatment of a variety of disorders. In some cases, however, their effectiveness is limited due to toxicities that arise due to broad target expression. In addition, antibody-based therapies have exhibited other limitations, such as rapid clearance from the circulation following administration.

Accordingly, a need exists for therapeutics that have the effectiveness of monoclonal antibody therapy, without the toxicities that are often observed with such therapies.

### SUMMARY OF THE INVENTION

In one aspect, the present disclosure provides masking moieties that are capable of masking the binding activity of a HER2 binding domain, wherein the masking moiety comprises an amino acid sequence that differs in from 0-2 amino acid positions from an amino acid sequence selected from the group consisting of SEQ ID NO:11, SEQ ID NO:12, SEQ ID NO:13, SEQ ID NO:14. SEQ ID NO:15, SEQ ID NO:16. SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015)

In another aspect, the present disclosure provides an activatable antigen-binding protein construct (ABPC) comprising:
(A) an antigen binding polypeptide complex (ABPC) that comprises a first anti-HER2 heavy chain variable domain (HVD1) and a first anti-HER2 light chain variable domain (LVD1) that together form a HER-2 binding domain.
wherein the HVD1 comprises:
(i) an HVD1 CDR1 comprising the amino acid sequence DTYIH (SEQ ID NO:1).
(ii) an HVD2 CDR2 comprising the amino acid sequence RIYPTNGYTRYADSVKG (SEQ ID NO: 2), and
(iii) an HVD3 CDR3 comprising the amino acid sequence WGGDGFYAMDY (SEQ ID NO:3), and
wherein the LVD1 comprises
(i) an LVD1 CDR1 comprising the amino acid sequence RASQDVNTAVA (SEQ ID NO:4),
(ii) an LVD1 CDR2 comprising the amino acid sequence SASFLYS (SEQ ID NO:5), and
(iii) an LVD1 CDR3 comprising the amino acid sequence QQHYTTPPT (SEQ ID NO:6); and
(B) a first cleavable moiety (CM) that comprises a substrate for a protease, and
(C) a first anti-HER2 masking moiety (MM1) comprising an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO:14, SEQ ID NO: 15, SEQ ID NO:16, SEQ ID NO: 17. SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:4 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015),
wherein the MM1, first CM, first HVD1, and first LVD1 are disposed within one or more polypeptides.

In a still further aspect, the ABPC further comprises a first binding domain that specifically binds a second biological target (BD2), wherein the first BD2 comprises a first light chain variable domain (LVD2) and a first heavy chain variable domain (HVD2), and a first masking moiety that attenuates the binding of the BD2 to the second biological target (MM2), wherein the activatable ABPC is an activatable bispecific ABPC. In some of these aspects, the second biological target is an immune cell surface antigen.

In another aspect, the present disclosure provides an activatable monovalent, bispecific ABPC comprising a first polypeptide, a second polypeptide, and a third polypeptide wherein:
(1) the first polypeptide comprises a second masking moiety (MM2), a first cleavable moiety (CM1), a second heavy chain variable domain (HVD2), a second light chain variable domain (LVD2), and a first Fc domain (Fc1),
(2) the second polypeptide comprises a first masking moiety (MM1), a second cleavable moiety (CM2), a first light chain variable domain (LVD1) and a constant light chain domain (CL), and
(3) the third polypeptide comprises a first heavy chain variable domain (HVD1) and a second Fc domain (Fc2),
wherein:
the LVD1 and the HVD1 together form a first binding domain (BD1) that specifically binds a first biological target,
the LVD2 and the HVD2 together form a second binding domain (BD2) that specifically binds a second biological target,
MM1 comprises a peptide that attenuates binding of the BD1 to the first biological target,
MM2 comprises a peptide that attenuates binding of the BD2 to the second biological target,
the first CM and the second CM each independently comprises a substrate for a protease and
wherein the Fc and the Fc2 bind each other.

### BRIEF DESCRIPTION OF THE DRAWINGS

FIG. 1 is a schematic of an illustrative activatable ABPC structure.

FIG. 2 is a schematic of an illustrative activatable divalent, bispecific ABPC.

FIG. 3 is a schematic of an illustrative activatable monovalent, bispecific ABPC.

FIG. 4 is a plot depicting binding versus concentration for several activatable anti-HER2 ABPCs with different anti-HER2 masking moieties (MM1) as measured in a HER2-based ELISA, along with a trastuzumab control (“Her2”). The experiments were performed as described in Example 3.

FIG. 5 is a plot depicting binding versus concentration for several activatable divalent bispecific anti-HER2, anti-CD3 ABPCs with different MM1s in a BT474 cell-based HER2-binding assay, along with controls (trastuzumab (“Her2”), and an unmasked anti-HER2, anti-CD3 bispecific antibody (“Her2/CD3”)). The experiments were performed as described in Example 4.

FIG. 6 is a plot depicting binding versus concentration for several activatable divalent, bispecific anti-HER2, anti-CD3 ABPCs with different MM1s as measured in an MDA-MB-231-Luc2 cell-based HER2-binding assay, along with controls (unmasked Trastuzumab (“Her2”) and an unmasked anti-HER2, anti-CD3 bispecific antibody (“Her2/CD3”)). The experiments were performed as described in Example 4.

FIG. 7 is a plot depicting binding versus concentration for several activatable divalent, bispecific anti-HER2, anti-CD3 ABPCs with different MM1s as measured in a SKBR3 cell-based HER2-binding assay along with controls (trastuzumab (“Her2”) and a mask-free anti-HER2, anti-CD3 bispecific antibody (“Her2/CD3”)). The experiments were performed as described in Example 4.

FIG. 8 is a plot depicting binding versus concentration for several activatable divalent, bispecific anti-HER2, anti-CD3 ABPCs with different MM1s as measured in a SKOV3 cell-based HER2-binding assay, along with controls (trastuzumab (“Her2”) and a mask-free anti-HER2, anti-CD3 bispecific antibody (“Her2/CD3”)). The experiments were performed as described in Example 4.

FIG. 9 is a plot depicting binding versus concentration for several activatable divalent, bispecific anti-HER2, anti-CD3 ABPCs with different MM1s as measured in a Jurkat cell-based CD3-binding assay, along with controls (trastuzumab (“Her2”) and a mask-free anti-HER2, anti-CD3 bispecific antibody (“Her2/CD3”). The experiments were performed as described in Example 4.

FIG. 10 is a plot of % cytotoxicity versus concentration for several activatable divalent, bispecific anti-HER2, anti-CD3 ABPCs with different MM1s as measured in an SKOV3 cell-based cytotoxicity assay, along with controls (trastuzumab (“Her2”), a mask-free anti-HER2, anti-CD3 bispecific antibody (“Her2/CD3”), and untreated cells). The experiments were performed as described in Example 4.

FIG. 11 is a plot of % cytotoxicity versus concentration for several activatable divalent, bispecific anti-HER2, anti-CD3 ABPCs with different MM1s as measured in an SKBR3 cell-based cytotoxicity assay, along with controls (trastuzumab (“Her2”), a mask-free anti-HER2, anti-CD3 bispecific antibody (“Her2/CD3”), and untreated cells (negative control)). The experiments were performed as described in Example 4.

FIGS. 12A and 12B are plots showing tumor volume over time in a NCl-N87, NSG mouse model following treatment with activatable divalent bispecific anti-HER2, anti-CD3 ABPC CI101 (5 mpk), along with controls (PBS and activated CI101 (“Act-C101”, 1 mpk) (FIG. 12A), and activatable divalent bispecific anti-HER2, anti-CD3 ABPCs CI124 (0.3 mpk and 1 mpk), CI156 (0.3 mpk and 1 mpk), along with controls (PBS and activated CI124 (0.3 mpk)) (FIG. 12B). The experiments were performed as described in Example 6.

FIG. 13 is a plot depicting binding versus concentration for several activatable divalent, bispecific anti-HER2, anti-CD3 ABPCs with different MM1s as measured in an OE33-Luc2 cell-based HER2-binding assay, with control (activated CI124 (“Act-CI124”)). The experiments were performed as described in Example 5.

FIG. 14 is a plot depicting binding versus concentration for several activatable divalent, bispecific anti-HER2, anti-CD3 ABPCs with different MM1s as measured in a Jurkat cell-based CD3-binding assay, along with a control (activated CI124 (“Act-CI124”)). The experiments were performed as described in Example 5.

FIG. 15 is a plot of % cytotoxicity versus concentration for several activatable divalent, bispecific anti-HER2, anti-CD3 ABPCs with different MM1s as measured in an OE33-Luc2 cell-based toxicity assay, along with a control (activated CI124 (“Act-CI124”)). The experiment was performed as described in Example 5.

FIG. 16 depicts a 4-12% SDS-PAGE gel loaded with 3 μg of different activatable Fc-heterodimeric, monovalent, bispecific anti-HER2, anti-CD3 ABPCs. 1a: CI238 heterodimeric-IgG1 monovalent HER2 after a single protein-A purification: 1b: sample 1a after size-exclusion chromatography (SEC) purification to enrich heterodimer; 2: CI239 heterodimeric-IgG4 monovalent HER2 after a single protein-A purification. NR=non-reduced, R=reduced prior to gel run. The light-chain and short heavy-chain (HCB) run as a doublet in the 28 kDa range. The experiment was performed as described in Example 7.

FIG. 17 is a plot of % cytotoxicity versus concentration for several activatable Fc-heterodimeric, monovalent, bispecific anti-HER2, anti-CD3 ABPCs with different MM1s, as measured in a OE33-Luc2 cell-based assay, along with a control (activated CI124 (“Act-CI124”). The experiment was performed as described in Example 8.

### DETAILED DESCRIPTION

Provided herein are protease-activatable antigen-binding protein constructs (ABPC) that when activated, generate an ABPC that is capable of specifically binding human epidermal growth factor receptor 2 (HER2). HER2 is a member of the epidermal growth factor (EGF) receptor family of receptor tyrosine kinases that can form a heterodimer with other ligand bound EGF receptor family members. Heterodimer formation can stabilize ligand binding and enhance kinase-mediated activation of downstream signaling pathways. Aberrant expression and/or activity of HER2 and HER2-related signaling has been implicated in the pathogenesis of many diseases and disorders, such as cancer. The HER2 polypeptide is encoded by the erb-b2 receptor tyrosine kinase 2 gene (erbb2). Anti-HER2 monoclonal antibodies have been shown to be effective in the treatment of HER2 positive cancers.

### Activatable Antigen-Binding Polypeptide Complexes (ABPCs).

The present disclosure provides activatable antigen-binding polypeptide complexes (ABPCs) that are designed to mitigate the potential for side effects of monoclonal antibody therapeutics. In specific embodiments, the present disclosure provides activatable anti-HER2 ABPCs that are designed to mitigate the potential for side effects due to off-target binding of an anti-HER2 antibody. As used herein, the term, “anti-HER2 ABPC” refers to an antigen binding protein complex that binds HER2 More specifically, in one embodiment, the present disclosure provides an activatable antigen-binding polypeptide construct (ABPC) comprising:
(A) an antigen binding polypeptide complex (ABPC) that comprises a first anti-HER2 heavy chain variable domain (HVD1) and a first anti-HER2 light chain variable domain (LVD1) that together form a HER-2 binding domain,
   wherein the HVD1 comprises:
   (i) an HCDR1 comprising the amino acid sequence DTYIH (SEQ ID NO:1),
   (ii) an HCDR2 comprising the amino acid sequence RIYPTNGYTRYADSVKG (SEQ ID NO.2), and
   (iii) an HCDR3 comprising the amino acid sequence WGGDGFYAMDY (SEQ ID NO 3), and
   wherein the LVD1 comprises
   (i) an LCDR1 comprising the amino acid sequence RASQDVNTAVA (SEQ ID NO:4).
   (ii) an LCDR2 comprising the amino acid sequence SASFLYS (SEQ ID NO:5), and
   (iii) an LCDR3 comprising the amino acid sequence QQHYTTPPT (SEQ ID NO:6); and
(B) a first cleavable moiety (CM) that comprises a substrate for a protease, and
(C) a first anti-HER2 masking moiety (MM1) comprising an amino acid sequence that differs in from 0-2 amino acid positions from an amino acid sequence selected from the group consisting of SEQ ID NO: 11 (F3.11), SEQ ID NO:12 (F3.17), SEQ ID NO:13 (F3.18), SEQ ID NO:14 (F3.19), SEQ ID NO:15 (F3.23), SEQ ID NO: 16 (F3.42), SEQ ID NO:17 (F3.43), SEQ ID NO.18 (F3.45), SEQ ID NO:19 (F4.2), SEQ ID NO:20 (F4.3), SEQ ID NO:21 (F4.10), SEQ ID NO 22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO.47 (mLW009), SEQ ID NO:48 (mLW010), SEQ ID NO:49 (mLW011), SEQ ID NO:50 (mLW012), SEQ ID NO:51 (mLW01), SEQ ID NO:52 (mLW014), and SEQ ID NO:53 (mLW015),
   wherein the MM1, first CM, first HVD1, and first LVD1 are disposed within one or more polypeptides.

In some embodiments, MM1 comprises an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO. 18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

In certain embodiments, the MM1 comprises an amino acid sequence that differs in from 0-2 amino acid positions from an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

In some embodiments, the MM1 comprises an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

The anti-HER2 MM1s (anti-HER2 binding domain masking moieties) described herein have been identified as being very effective at masking the binding ability of HER2 binding domains. The terms “masking moiety” and “MM”, are used interchangeably herein to refer to a peptide that, when positioned proximal to the ABPC, attenuates binding of the ABPC to its target(s).

The terms “cleavable moiety” and “CM” are used interchangeably herein to refer to a peptide that includes a substrate for at least one protease. Hence, the CM is thus susceptible to cleavage upon exposure to that protease. The CM is positioned relative to the MM and ABPC, such that cleavage results in the untethering of the MM from its position proximal to the ABPC resulting in the generation of an “activated” ABPC (also referred to herein as “unmasking”). Unmasking of the activatable ABPC results in an activated ABPC having greater binding affinity for its biological target (e.g., HER2) as compared to the corresponding activatable ABPC.

The ABPC component of the activatable ABPCs described herein may have the structure of any of a variety of known antigen binding structures, including, for example, an antibody, a fragment antigen binding (Fab), a F(ab′)₂ fragment, a single chain Fv (scFv), a bispecific antibody (such as, for example, a BiTE, a DART, and the like), and the like. As such, the activatable ABPCs described herein may comprise one, two, three, or four or more polypeptides. As described in more detail hereinbelow, the activatable ABPCs may further comprise one or more additional components, including, for example, a spacer, one or more linkers, a constant light chain domain (CL), a CH1 domain, a hinge region (HR), a first Fc domain (Fc1), a second Fc domain (Fc2), and the like. The terms “hinge region”, “hinge”, “hinge domain” and “HD” are used interchangeably herein.

The CDR sequences specified herein are determined in accordance with the Kabat numbering system (i.e., the “Kabat CDRs”) as described in Abhinandan, K. R, and Martin, A. C. R. (2008) “Analysis and improvements to Kabat and structurally correct numbering of antibody variable domains”, Molecular Immunology, 45, 3832-3839, which is incorporated herein by reference in its entirety. The Kabat CDRs are defined as CDR-L1: residues L24-L34; CDR-L2: residues L50-L56; CDR-L3 residues L89-L97; CDR-H1: residues H31-H35; CDR-H2: residues H50-H65; and CDR-H3: residues H95-102, wherein “L” refers to the light chain variable domain and “H” refers to the heavy chain variable domain.

In some of embodiments, the activatable anti-HER2 ABPC of the present disclosure comprises:
(A) an HVD1 having
   (i) an HCDR1 comprising the amino acid sequence of SEQ ID NO:1,
   (ii) an HCDR2 comprising the amino acid sequence of SEQ ID NO:2,
   (iii) an HCDR3 comprising the amino acid sequence of SEQ ID NO:3, and
   (iv) an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7 and
(B) an LVD1 (anti-HER2 LVD) having
   (i) an LCDR1 comprising the amino acid sequence of SEQ ID NO:4,
   (ii) an LCDR2 comprising the amino acid sequence of SEQ ID NO:5,
   (iii) an LCDR3 comprising the amino acid sequence of SEQ ID NO:6, and
   (iv) an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8

In certain of the embodiments described herein, the LVD1 (anti-HER2 HVD) comprises the amino acid sequence of SEQ ID NO:7 and the LVD1 (anti-HER2 LVD) comprises the amino acid sequence of SEQ ID NO:8. In some of these embodiments, the activatable anti-HER2 ABPC comprises an anti-HER2 antibody heave chain comprising the amino acid sequence of SEQ ID NO:9. In certain embodiments, the activatable anti-HER2 ABPC comprises an anti-HER2 antibody light chain comprising the amino acid sequence of SEQ ID NO. 10. In a specific embodiment, the activatable ABPC comprises an anti-HER antibody heavy chain comprising the amino acid sequence of SEQ ID NO:9 and an anti-HER2 antibody light chain comprising the amino acid sequence of SEQ ID NO:10.

In certain embodiments, MM1, CM1, HVD1, and LVD1 are deployed within the same polypeptide. In some of these aspects, the structural arrangement of these components, from N-terminus to C-terminus, is selected from the group consisting of:

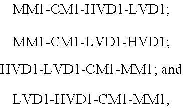

wherein each “-” is a direct or indirect linkage (e.g., via a linker). In some aspects, the LVD1 and the HVD1 components are situated within the structure of an scFv cassette, i.e., LVD1-linker-HVD1 or HVD1-linker-LVD1 Linker moieties that are suitable for use in the activatable anti-HER2 ABPCs of the present disclosure are described in more detail hereinbelow.

In some aspects, the activatable anti-HER2 ABPC of the present disclosure comprises at least (i) a first polypeptide comprising the HVD1, and (ii) at least a second polypeptide comprising the MM1, the CM1, and the LVD1. In other aspects, the activatable ABPC comprises at least (i) a first polypeptide comprising the MM1, the CM1, and the HVD1, and (ii) at least a second polypeptide comprising the LVD1.

In a specific aspect, the present disclosure provides an activatable divalent, monospecific anti-HER2 ABPC that comprises
(1) a first polypeptide comprising a first anti-HER2 heavy chain variable domain (HVD1) and a first Fc1, wherein the first HVD1 comprises (i) an HCDR1 having the amino acid sequence of SEQ ID NO:1, (ii) an HCDR2 having the amino acid sequence of SEQ ID NO:2, and (iii) an HCDR3 having the amino acid sequence of SEQ ID NO:3;
(2) a second polypeptide comprising a first MM1, a first cleavable moiety (CM), a first anti-HER2 light chain variable domain (LVD1) and a first constant light chain domain (CL), wherein the first anti-HER2 LVD1 comprises (i) an LCDR1 comprising the amino acid sequence of SEQ ID NO:4, (ii) an LCDR2 comprising the amino acid sequence of SEQ ID NO:5, and (iii) an LCDR3 comprising the amino acid sequence of SEQ ID NO:6,
(3) a third polypeptide comprising a second anti-HER2 HVD1 and a second Fc domain (Fc2), wherein the second anti-HER2 HVD1 comprises (i) an HCDR1 having the amino acid sequence of SEQ ID NO:1, (ii) an HCDR2 having the amino acid sequence of SEQ ID NO:2, and (iii) an HCDR3 having the amino acid sequence of SEQ ID NO:3; and
(4) a fourth polypeptide comprising a second MM1, a second CM, a second anti-HER2 light chain variable domain (LVD1) and a second constant light chain domain (CL), wherein the second anti-HER2 LVD1 comprises (i) an LCDR1 comprising the amino acid sequence of SEQ ID NO:4. (ii) an LCDR2 comprising the amino acid sequence of SEQ ID NO:5, and (iii) an LCDR3 comprising the amino acid sequence of SEQ ID NO:6,
wherein the first MM1 and the second MM1 each independently comprise an amino acid sequence that differs in from 0-2 amino acid positions from an amino acid sequence selected from the group consisting of SEQ ID NO:11 (F3.11), SEQ ID NO: 12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.0c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015); and
wherein the Fc1 and the Fc2 bind each other.

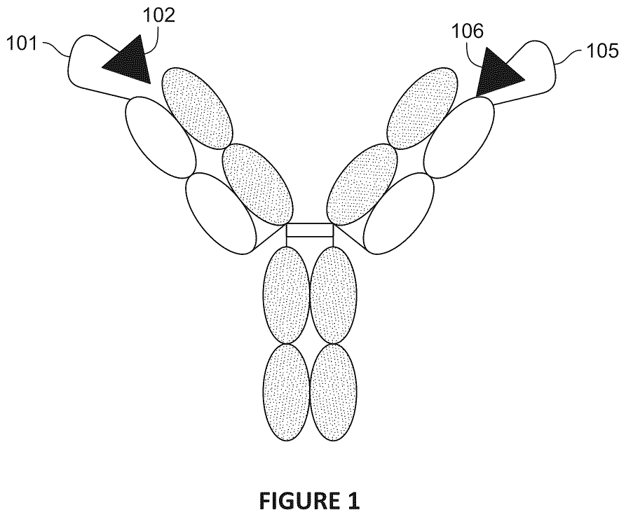

A schematic of an illustrative activatable ABPC having the above-described format is shown in FIG. 1, which depicts a first polypeptide and a third polypeptide (dark grey) each comprising, from N-terminus to C-terminus, a heavy chain variable domain (HVD1), a CH1 domain, a hinge domain, and an Fc domain; a second polypeptide (light grey, left) comprising, from N-terminus to C-terminus, a first MM1 102, a first CM 101, a first light chain variable domain (LVD1) and a first constant light chain domain (CL), and a fourth polypeptide (light grey, right) comprising, from N-terminus to C-terminus, a second MM1 106, a second CM 105, a second LVD1 and a second CL.

In some embodiments, first MM1 and the second MM1 each independently comprises an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015);

In certain embodiments, the first MM1 and the second MM1 each independently comprises an amino acid sequence that differs in from 0-2 amino acid positions from an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

In some embodiments, the first MM1 and the second MM1 each independently comprises an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

In some aspects of the above-described activatable anti-HER2 ABPCs, the Fc1 and the Fc2 comprise the same amino acid sequence. In other aspects, the Fc1 and the Fc2 comprise different amino acid sequences. Likewise, in some embodiments, the CM1 and the CM2 comprise the same amino acid sequence. In other embodiments, the CM1 and the CM2 comprise different amino acid sequences. In certain embodiments, the first CL domain and the second CL domain comprise the same amino acid sequence. In some embodiments, the first polypeptide further comprises a first CH1 domain and/or a first hinge domain and the third pol peptide further comprises a second CH1 domain and/or a second hinge domain. In certain embodiments, the first polypeptide further comprises a first CH1 domain and a first hinge domain and the third polypeptide further comprises a second CH1 domain and a second hinge domain. In some aspects, the first CH1 domain and the second CH1 comprise the same amino acid sequence. In other aspects, the first CH1 domain and the second CH1 domain comprise different amino acid sequences. In some embodiments, the first hinge domain and the second hinge domain comprise the same amino acid sequence. In other embodiments, the first hinge domain (i.e., hinge region) and the second hinge domain (i.e., hinge region) comprise different amino acid sequences. Fc, CL, CH1 and hinge domains that are suitable for use in the activatable anti-HER2 ABPCs of the present disclosure are described in more detail hereinbelow.

In certain aspects, the first polypeptide and the third polypeptide comprise the same amino acid sequences, and the second polypeptide and the fourth polypeptide comprise the same amino acid sequences. Illustrative activatable anti-HER2 ABPCs comprise a heavy chain polypeptide (e.g., first and third polypeptide) that comprises the amino acid sequence of SEQ ID NO:601 and a light chain polypeptide (e.g., second and fourth polypeptide) that comprises the amino acid sequence selected from the group consisting of SEQ ID NOs 603, 605, 608, 610, 612, 614, 616, 618, 620, 622, 624, 626, 628, 630, 632, 634, and 638.

Activatable anti-HER2 ABPCs of the present disclosure include activatable multispecific ABPCs, such as, for example, an activatable bispecific ABPC. As used herein, the term “activatable multispecific ABPC” refers to an activatable ABPC that can bind to at least two different biological targets when activated by protease cleavage. The terms “activatable bispecific ABPC” refers to an activatable ABPC that can bind to two different biological targets when activated by protease cleavage. The term “activatable bispecific anti-HER2 ABPC” refers to an activatable bispecific ABPC that can bind HER2 and a second biological target when activated. Activatable bispecific anti-HER2 ABPCs may be monovalent or divalent, as described in more detail hereinbelow. The term “monovalent”, as used in connection with an activatable ABPC refers to an activatable ABPC that has a single binding domain that is specific for each particular biological target. The term “divalent” when used in connection with an activatable ABPC refers to an activatable ABPC that has two binding domains that are specific for each particular biological target.

In one aspect, the activatable ABPC is an activatable bispecific anti-HER2 that, in addition to having an MM1, a CM1, a LVD1 and HVD1 that together form the binding domain for HER2 (the first biological target (BD1)), also has a binding domain that specifically binds a second biological target (BD2), wherein the binding domain comprises a first heavy chain variable domain (HVD2) and a first light chain variable domain (LVD2). As used herein, the terms “HVD2” and “LVD2” refer herein to the variable domains that together form a binding domain that specifically binds the second biological target. In certain aspects, the activatable bispecific ABPC further comprises a masking moiety that attenuates binding of the second binding domain to the second biological target (MM2). In some embodiments, the activatable bispecific ABPC comprises an MM1, an MM2, an HVD1, an LVD1, an HVD2, an LVD2, a first CM, and a second CM that are disposed within one or two or more polypeptides. In certain embodiments, the activatable bispecific ABPC comprises a first MM1, a first HVD1, a first LVD1, a first HVD1, a first CM, a second MM1, a second HVD1, a second LVD1, a second CM, a first MM2, a first HVD2, a first LVD2, a third CM, a second MM2, a second HVD2, a second LVD2, and a fourth CM. In some embodiments, the first and second MM1s, HVD1s, and LVD1s are respectively, the same; and the first and second MM2s. HVD2s, and LVD2s are respectively the same. The activatable bispecific ABPCs of the present disclosure may further comprise one or more spacers, one or more Fe domains, one or more constant light chain domains (CL), one or more CH1 domains, one or more hinge domains, and one or more linkers. Spacers, Fc domains, CL domains, CH1 domains, hinge domains, and linkers that are suitable for use in the activatable bispecific ABPCs of the present disclosure are described in more detail hereinbelow.

In some embodiments, the BD2 is an immune cell surface antigen, such as, for example, cluster of differentiation 3 (e.g., CD3ε, and the like), such as, but not limited to B7H4, BTLA, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA. In a specific aspect, the activatable ABPC further comprises an anti-CD3 antibody heavy chain variable domain (HVD2), an anti-CD3 antibody light chain variable domain (LVD2), an anti-CD3 antibody masking moiety (MM2), and a second cleavable moiety (CM2). Any of a variety of known anti-CD3 heavy chain and light chain variable domains may be employed in the activatable anti-HER2, anti-CD3 bispecific ABPCs described herein. Exemplary anti-CD3 heavy chain variable and light chain variable domains include any that are known in the art, such as, for example, OKT3, SP34, and the like.

In a specific embodiment, the HVD2 (anti-CD3 HVD) comprises
(i) an HCDR1 comprising the amino acid sequence TYAMN (SEQ ID NO:581).
(ii) an HCDR2 comprising the amino acid sequence RIRSKYNNYATYYADSVKD (SEQ ID NO:582), and
(iii) an HCDR3 comprising the amino acid sequence HOGNFGNSYVSWFAY (SEQ ID NO:583), and
the LVD2 (anti-CD3 LVD) comprises
(i) an LCDR1 comprising the amino acid sequence RSSTGAVTTSNYAN (SEQ ID:584),
(ii) an LCDR2 comprising the amino acid sequence GTNKRAP (SEQ ID NO:585), and
(iii) an LCDR3 comprising the amino acid sequence ALWYSNLWV (SEQ ID NO:586); and the MM2 comprises an amino acid sequence that differs in from 0-2 amino acid residues from an amino acid sequence selected from the group consisting of MMYCGGNEVLCGPRV (SEQ ID NO. 598), GYLWGCEWNCGGITT (SEQ ID NO:599), and GYRWGCEWNCGGITT (SEQ ID NO:600). In some embodiments, the MM2 comprises an amino acid sequence selected from the group consisting of SEQ ID NOs:598, 599, and 600.

As used herein, the term “CD3 binding domain” and “CD3 antigen binding domain” are used interchangeably herein to refer to a binding domain that specifically binds a CD3. The term “anti-CD3 HVD” refers herein to the heavy chain variable domain of a binding domain that specifically binds a CD3. The term “anti-CD3 LVD” refers herein to the light chain variable domain of a binding domain that specifically binds a CD3.

The activatable bispecific anti-HER2, anti-CD3 ABPC may be in any of a variety of formats, and may, for example, be monovalent (i.e., comprising a single HER2 binding domain and a single CD3 binding domain) or divalent (i.e., comprising two HER2 binding domains and two CD3 binding domains) with respect to the HER2 binding domain and CD3 binding domain. The term “activatable bispecific anti-HER2, anti-CD3 ABPC” refers to an activatable ABPC that has a HER2 binding domain and a CD3 binding domain, together with at least a masking moiety that attenuates the binding of the HER2 binding domain.

In one aspect, the activatable bispecific anti-HER2, anti-CD3 ABPC comprises at least a first polypeptide and a second polypeptide, wherein:
(1) the first polypeptide comprises a (first) MM2, a (first) cleavable moiety (CM), a (first) HVD2, a (first) LVD2, and a (first) HVD1,
   wherein the (first) MM2 is an anti-CD3 masking moiety, the (first) HVD2 is an anti-CD3 HVD, the (first) LVD2 is an anti-CD3 LVD, and the (first) HVD1 is an anti-HER2 HVD; and
(2) the second polypeptide comprises a (first) MM1, a second CM, and a (first) LVD1;
   wherein:
      the MM1 is an anti-HER2 masking moiety,
      the LVD1 and HVD1 are an anti-HER2 LVD and anti-HER2 HVD, respectively, that together form a binding domain that specifically binds HER2,
      the HVD2 and LVD2 are an anti-CD3 LVD and an anti-CD3 HVD that together form a binding domain that specifically binds a CD3,
      the first cleavable moiety (CM) and the second CM are each independently a peptide that comprises a substrate for a protease, and
      the MM2 is an anti-CD3 masking moiety.

In some embodiments, the activatable bispecific anti-HER2, anti-CD3 ABPC is an activatable divalent, bispecific anti-HER2, anti-CD3 ABPC. In a specific embodiment, the present disclosure provides an activatable divalent, bispecific anti-HER2, anti-CD3 ABPC comprising a first polypeptide, a second polypeptide, a third polypeptide, and a fourth polypeptide, wherein
(1) the first polypeptide comprises a first MM2, a first CM, a first HVD2, a first LVD2, a first HVD1, and a first Fc domain (Fc1),
(2) the second polypeptide comprises a first MM1, a second CM, a first LVD1, and a first CL, (3) the third polypeptide comprises a second MM2, a third CM, a second HVD2, a second LVD2, a second HVD1, and a second Fc domain, and
(4) the fourth pols peptide comprises a second MM1, a fourth CM, a second LVD1, and a second CL, wherein:
   the first MM1 and the second MM1 are each independently an anti-HER masking moiety that comprises an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO: 14 (F3.19), SEQ ID NO:15 (F3.23), SEQ ID NO. 16 (F3.42), SEQ ID NO: 17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015):
the first MM2 and the second MM2 are each independently an anti-CD3 masking moiety,
each pair of the first HVD1 and the first LVD1, and the second HVD1 and the second LVD1 together forms a binding domain that specifically binds a HER2,
each pair of the first HVD2 and the first LVD2, and the second HVD2 and the second LVD2 together forms a binding domain that specifically binds a CD3,
the first, second, third, and fourth CM are each independently a cleavable moiety,
the first CL and the second CL are each independently a constant light chain domain,
wherein the Fc1 and the Fc2 bind each other.

As described above, the activatable ABPCs described herein may further have one or more other components, such as, for example, a spacer, a linker, a constant light chain domain (CL), a CH1 domain, and a hinge domain. In some of the above-described embodiments, the CH1 domain is a human IgG1 CH1 domain and the Fc domain is a human IgG1 Fc domain or variant thereof comprising from one to five mutations. In other embodiments, the CH1 domain is a human IgG4 CH1 domain and the Fc domain is a human IgG4 Fc domain or variant thereof comprising from one to five mutations. In certain embodiments. Fc1 and Fc2 comprise different amino acid sequences. In other embodiments. Fc1 and Fc2 comprise the same amino acid sequence

In some embodiments, the MM, CM, HVD, and LVD components are arranged, from N-terminal to C-terminal as follows:

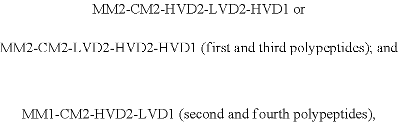

wherein each “-” is independently a direct or indirect (e.g., via a linker) linkage. In certain embodiments, the first and third polypeptides further comprise a CH1 domain. In a specific embodiment, the first, second, third, and fourth polypeptides having the following structure, from N- to C-terminus:

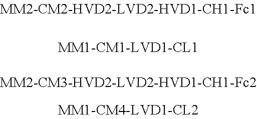

wherein each “-” is independently a direct or indirect (e.g., via a linker) linkage. In certain embodiments, the HVD2 and LVD2 components are disposed within an scFv moiety (i.e., having the structure HVD2-linker-LVD2 or LVD2-linker-HVD2), from N-terminus to C-terminus).

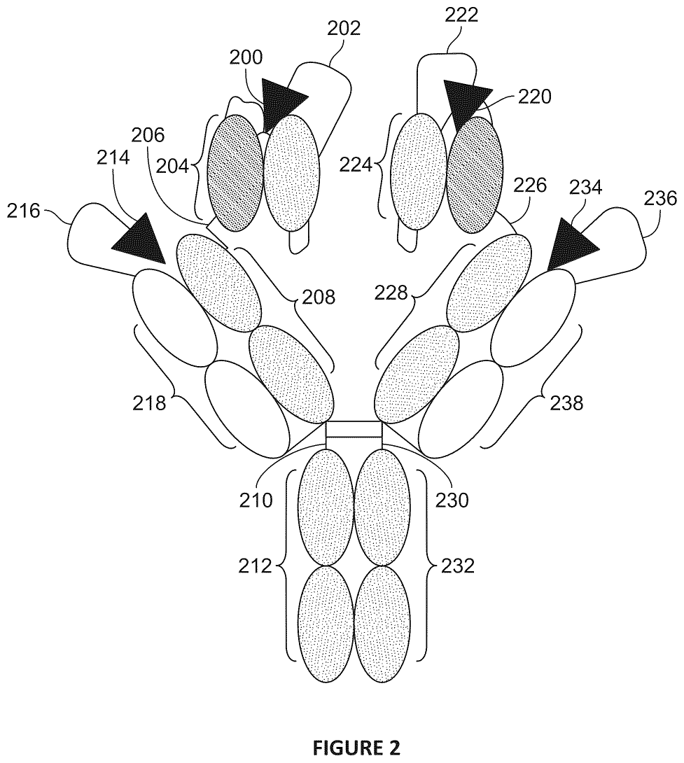

A schematic of an illustrative activatable ABPC having the above-described format is shown in FIG. 2, which depicts a first polypeptide comprising, from N-terminus to C-terminus, a first MM2 200, a first CM 202, a first BD2 scFv (i.e., comprising a first HVD2-first linker first LVD2 or first LVD2-first linker-first HVD2) 204, a second linker 206, a first HVD1-CH1 substituent 208, a first hinge domain 210, and a first Fc domain 212: a second polypeptide comprising, from N-terminus to C-terminus, a second polypeptide comprising, from N-terminus to C-terminus, a first MM1 214, a second CM 216, and a first LVD1-first CL substituent 218, a third polypeptide comprising a second MM2 220, a third CM 222, a second BD2 scFv (i.e., comprising a second HVD2 third linker-second LVD2 or second LVD2-third linker-second HVD2) 224, fourth linker 226, second HVD2-second CH1 substituent 228, a second hinge domain 230, and second Fc domain (Fc2) 232; and a fourth polypeptide comprising a second MM1 234, a fourth CM 236, and a second LVD1-second CL substituent 238.

In a specific embodiment:
(1) the first polypeptide has the following structure, from N-terminus to C-terminus.
S1-L1-first MM2-L2-first CM-L3-first HVD2-L4 first LVD2 L5 first HVD1-L6-first CH1-L7-first HR-L8-Fc1
(2) the second polypeptide has the structure, from N-terminus to C-terminus:
   S2-L9-first MM1L10-second CM-L11-first LVD1-L12-first CL
(3) the third polypeptide has the structure, from N-terminus to C-terminus:
   S3-L-first MM2-L14-third CM-115-HVD2-L16-LVD2-L17-HVD1-L18-second CH1-L19-second HR-120-Fc2.
(4) the fourth polypeptide has the structure, from N-terminus to C-terminus.
   S4-L21-second MM1-L22-fourth CM-L23-second LVD1-L24-second CL; wherein:
S1, S2, S3, and S4 are each independently an optional spacer that is either present or absent;
L1, L23, L4, L5, L6, L7, L8, L9, L10. L11. L12. L13, L14, L15, L16. L17, L18, L19, L120, L21, L22, L23, and L24 are each independently an optional linker that is either present or absent;
the first MM1 and the second MM1 are each independently an anti-HER2 masking moiety comprising an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO. 15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:4l (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015),
the first MM2 and the second MM2 are each independently an anti-CD3 masking moiety,
each pair of the first HVD1 and the first LVD1, and the second HVD1 and the second LVD1 together forms a binding domain that specifically binds a HER2,
each pair of the first HVD2 and the first LVD2, and the second HVD2 and the second LVD2 together forms a binding domain that specifically binds a CD3,
the first, second, third, and fourth CM are each independently a cleavable moiety, and
the first CL and the second CL are each independently a constant light chain domain, wherein the Fc1 and the Fc2 bind each other
the first HR and the second HR are each independently a hinge region.

In some of the described embodiments, L2, L3, L4 and L5 are each present in the first polypeptide; L10 and L11 are present in the second polypeptide; L14, L15, L16, and L17 are each present in the third polypeptide: L16 and L17 are each present in the fourth polypeptide. In certain of these embodiments, L1, L2, L3, L4, L5, and are each present in the first polypeptide; the L7, L8, and L9 are each present in the second polypeptide; the L10, L11, L12, L13, L14, and L15 are each present in the third polypeptide; and L22 and L23 is present in the fourth polypeptide. In some of the above-described embodiments, S1 and S3 are each present. In some of these embodiments, L1 and L13 are both present. In certain embodiments, S2 and S4 are each present. In certain of these embodiments, L9 and L21 are both present. In some embodiments, S1, S2, S3, S4, L1, L9, L13, and L21 are all present. In some of the above-described embodiments, L7 and L18 are both present Linker and spacer moieties that are suitable for use in these embodiments are described in more detail hereinbelow.

In certain embodiments of the above-described activatable divalent, bispecific ABPCs, the first MM1 and the second MM1 comprise the same amino acid sequence. In some embodiments, the first MM2 and the second MM2 comprise the same amino acid sequence. In certain of these embodiments, the first CM, the second CM, the third CM, and the fourth CM each comprise the same amino acid sequence. In certain of these embodiments, the first CM and the third CM comprise different amino acid sequences relative to the second CM, and the fourth CM.

In some embodiments, first MM1 and the second MM1 each independently comprises an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW00), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015);

In certain embodiments, the first MM1 and the second MM1 each independently comprises an amino acid sequence that differs in from 0-2 amino acid positions from an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

In some embodiments, the first MM1 and the second MM1 each independently comprises an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

In some of the above-described embodiments, the CH1 domain is a human IgG1 CH1 domain and the Fc domain is a human IgG1 Fc domain or variant thereof comprising from one to five mutations. In other embodiments, the CH1 domain is a human IgG4 CH1 domain and the Fc domain is a human IgG4 Fc domain or variant thereof comprising from one to five mutations. In certain embodiments, Fc1 and Fc2 comprise different amino acid sequences. In other embodiments, Fc1 and Fc2 comprise the same amino acid sequence. In one aspect, the CL is a human constant light chain domain.

In a specific embodiment, the first and third polypeptides comprise the same amino acid sequences, and the second and fourth polypeptides comprise the same amino acid sequences.

In some of these embodiments:
(A) the first HVD2 (anti-CD3 HVD) and the second HVD2 each comprise
   (i) an HCDR1 comprising the amino acid sequence TYAMN (SEQ ID NO:581).
   (ii) an HCDR2 comprising the amino acid sequence RIRSKYNNYATYYADSVKD (SEQ ID NO:582).
   (iii) an HCDR3 comprising the amino acid sequence HGNFGNSYVSWFAY (SEQ ID NO.583), and
(B) the first LVD2 (anti-CD3 LVD) and the second LVD2 each comprise
   (i) an LCDR1 comprising the amino acid sequence RSSTGAVTTSNYAN (SEQ ID NO:584),
   (ii) an LCDR2 comprising the amino acid sequence GTNKRAP (SEQ ID NO:585).
   (iii) an LCDR3 comprising the amino acid sequence ALWYSNLWV (SEQ ID NO:586).

In some embodiments, each pair of first HVD2 and first LVD2 and second HVD2 and second LV2, respectively, is independently selected from the group consisting of:
(1) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO. 587 and an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO: 588;
(2) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO: 587 and an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:589; and
(3) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:590 and an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO: 591.

In some instances, each of the first HVD2 and second HVD2 comprises the amino acid sequence of SEQ ID NO: 587 and each of the first LVD2 and the second LVD2 comprises the amino acid sequence of SEQ ID NO:588. In other embodiments, each of the first HVD2 and the second HVD2 comprises the amino acid sequence of SEQ ID NO:593 and each of the first LVD2 and the second LVD2 comprises the amino acid sequence of SEQ ID NO:589. In certain embodiments, each of the first HVD2 and the second HVD2 comprises the amino acid sequence of SEQ ID NO:590 and each of the first LVD2 and the second LVD2 comprises the amino acid sequence of SEQ ID NO:591.

In certain specific embodiments, the first HVD2 and the first LVD2 and/or the second HVD2 and the second LVD2 are disposed within an scFv having the structure, from N-terminus to C-terminus. HVD2-linker-LVD2 or LVD2-linker-HVD2. Any of a variety of known anti-CD3 scFvs may be employed in the activatable bispecific anti-HER2 ABPCs described herein, including, for example an scFv comprising an amino acid sequence selected from the group consisting of SEQ ID NO:592, SEQ ID NO:594, and SEQ ID NO:597.

In some of the above-described embodiments, each of the first HVD1 (anti-HER2 HVD) and the second HVD1 independently comprises:
(i) an HCDR1 comprising the amino acid sequence of SEQ ID NO:1,
(ii) an HCDR2 comprising the amino acid sequence of SEQ ID NO:2,
(iii) an HCDR3 comprising the amino acid sequence of SEQ ID NO:3, and
(iv) an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7, and each of the first LVD1 and second LVD1 (anti-HER2 LVD) comprises
(i) an LCDR1 comprising the amino acid sequence of SEQ ID NO:4,
(ii) an LCDR2 comprising the amino acid sequence of SEQ ID NO:5,
(iii) an LCDR3 comprising the amino acid sequence of SEQ ID NO:6, and
(iv) an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8.

In some of the above-described embodiments, each of the first and second MM2 independently comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600) In certain embodiments, each of the first and second MM2 independently comprises an amino acid sequence selected from the group consisting of SEQ ID NOs:598, 599, and 600.

In certain of the embodiments described herein, each of the first HVD1 (anti-HER2 HVD) and the second HVD1 comprises the amino acid sequence of SEQ ID NO:7 and each of the first LVD1 (anti-HER2 LVD) and the second LVD1 comprises the amino acid sequence of SEQ ID NO:8.

Illustrative activatable divalent, bispecific anti-HER2, ani-CD3 ABPCs are provided in Example 2 hereinbelow. In some embodiments, the activatable anti-HER2, anti-CD3 ABPC comprises a heavy chain polypeptide (1^{st} and 3^{rd} polypeptide) comprising the amino acid sequence of SEQ ID NO:638 and a light chain polypeptide (2^{nd} and 4^{th} polypeptide) comprising an amino acid sequence selected from the group consisting of SEQ ID NO:610, 620, 624, 626, and 627. In other embodiments, the activatable anti-HER2, anti-CD3 ABPC comprises a heavy chain polypeptide (1^{st} and 3^{rd} polypeptide) comprising the amino acid sequence of SEQ ID NO:646 and a light chain polypeptide (2^{nd} and 4^{th} polypeptide) comprising the amino acid sequence of SEQ ID NO:626. In certain embodiments, the activatable anti-HER2, anti-CD3 ABPC comprises a heavy chain polypeptide (1^{st} and 3^{rd} polypeptide) comprising the amino acid sequence of SEQ ID NO:670 and a light chain polypeptide (2^{nd} and 4^{th} polypeptide) comprising the amino acid sequence of SEQ ID NO:626. In further embodiments, the activatable anti-HER2, anti-CD3 ABPC comprises a heavy chain polypeptide (1^{st} and 3^{rd} polypeptide) comprising the amino acid sequence of SEQ ID NO:674 and a light chain polypeptide (2^{nd} and 4^{th} polypeptide) selected from the group consisting of SEQ ID NOs:676, 678, 680, 682, 684, 686, 688, 690, 692, 694, 696, 698, 700, 702, and 704.

In another aspect, the present disclosure provides an activatable monovalent, bispecific ABPC. In one embodiment, the activatable monovalent bispecific ABPC comprises a first polypeptide, a second polypeptide, and a third polypeptide wherein:
(1) the first polypeptide comprises a second masking moiety (MM2), a first cleavable moiety (CM1), a second heavy chain variable domain (HVD2), a second light chain variable domain (LVD2), and a first Fc domain (Fc1),
(2) the second polypeptide comprises a first masking moiety (MM1), a second cleavable moiety (CM2), a first light chain variable domain (LVD1) and a constant light chain domain (CL), and
(3) the third polypeptide comprises a first heavy chain variable domain (HVD1) and a second Fc domain (Fc2),
wherein:
the LVD1 and the HVD1 together form a first binding domain (BD1) that specifically binds a first biological target,
the LVD2 and the HVD2 together form a second binding domain (BD2) that specifically binds a second biological target,
MM1 comprises a peptide that attenuates binding of the BD1 to the first biological target,
MM2 comprises a peptide that attenuates binding of the BD2 to the second biological target,
the first CM and the second CM each independently comprises a substrate for a protease and
wherein the Fc1 and the Fc2 bind each other.

In some embodiments, the first biological target is an antigen associated with disease. For example, the biological target may be a cancer cell antigen, and the like. In these and other embodiments, the second biological target is an immune cell surface antigen, such as, for example, any of those described herein (e.g., HER2). In certain specific embodiments, the immune cell surface antigen is a CD3. In some embodiments, the first biological target is a cancer cell antigen (e.g., HER2), and the second biological target is a CD3.

In a specific aspect, the activatable monovalent, bispecific ABPC comprises a first polypeptide, a second polypeptide, and a third polypeptide wherein:
(1) the first polypeptide comprises an MM2, a first CM, an HVD2, an LVD2, and a first Fc domain (Fc1).
(2) the second polypeptide comprises an MM1, a second CM, an LVD1 and a constant light chain domain (CL), and
(3) the third polypeptide comprises an HVD1 and a second Fc domain (Fc2);
   wherein the HVD1 is an anti-HER2 HVD,
   wherein the LVD1 is an anti-HER2 LVD.
   wherein MM1 is an anti-HER2 masking moiety selected from the group consisting of comprise an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO: 14 (F3.19), SEQ ID NO:15 (F3.23), SEQ ID NO:16 (F3.42), SEQ ID NO:17 (F3.43), SEQ ID NO: 18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:3 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:4l (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015), and
   wherein the Fc1 and the Fc2 bind each other. In certain of the above-described embodiments, the anti-HER2 HVD comprises an HCDR1 comprising the amino acid sequence of SEQ ID NO:1, and HCDR2 comprising the amino acid sequence of SEQ ID NO:2, and an HCDR3 comprising an HVD CDR3 comprising the amino acid sequence of SEQ ID NO:3; and the anti-HER2 LVD comprises an LCDR1 comprising the amino acid sequence of SEQ ID NO:4, an LCDR2 comprising the amino acid sequence of SEQ ID NO:5, and an LCDR3 comprising the amino acid sequence of SEQ ID NO:6.

In some embodiments, the HVD2 and LVD2 together form a binding domain that is specific for a CD3 polypeptide In these embodiments, the MM2 may be any masking moiety that attenuates the binding of the CD3 binding domain to CD3. In some of the above-described embodiments, the MM2 comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600). In certain embodiments, the MM2 comprises an amino acid sequence selected from the group consisting of SEQ ID NOs:598, 599, and 600.

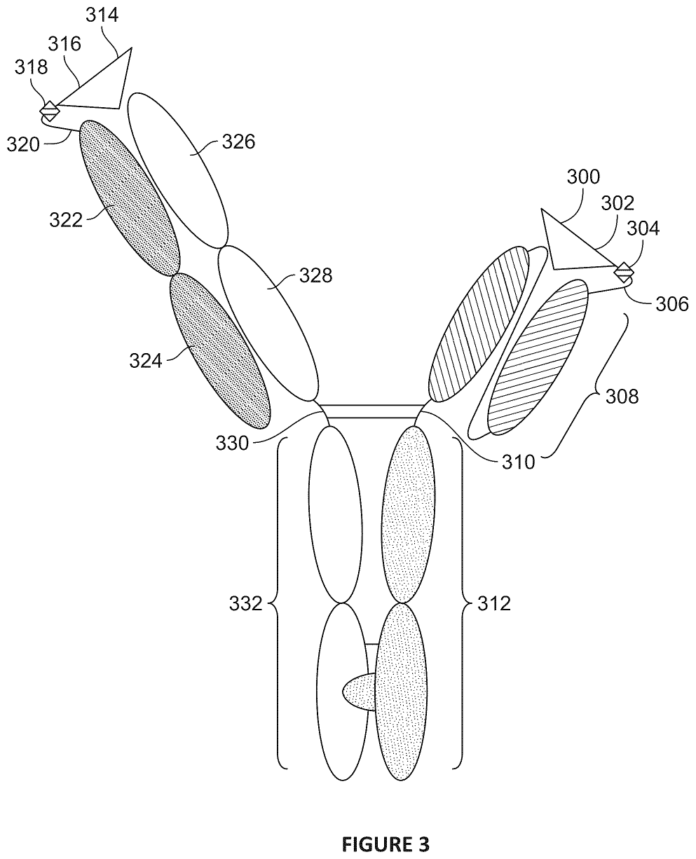

The activatable bispecific ABPCs of the present disclosure may further comprise one or more components, such as, for example, one or more of spacers, linkers, CH1 domains, hinge domains, and the like. A schematic of an illustrative activatable monovalent, bispecific ABPC having the above-described format is shown in FIG. 3, which depicts a first polypeptide comprising, from N-terminus to C-terminus, an MM2 300, a first linker 302, a first CM 304, a second linker 306, an scFv comprising an LVD2 and an HVD2 with a third linker disposed there between) 308, a first hinge domain 310, and a first Fc domain 312; a second polypeptide comprising, from N-terminus to C-terminus, an MM1 314, a fourth linker 316, a second CM 318, a fifth linker 320, an LVD1 322, and a CL 324; and a third polypeptide comprising an HVD1 326, a CH1 domain 328, a second hinge domain (hinge region) 330, and a second Fc domain 332.

In certain of these embodiments.
(1) the first polypeptide has the structure, from N-terminus to C-terminus
   S1-L1-MM2-L2-CM1-L3-HVD2-L4-LVD2-L5-first CH1-L6-first HR-L7-Fc1
(2) the second polypeptide has the structure, from N-terminus to C-terminus:
   S2-L8-MM1-L9-CM2-L10-LVD1-L11-CL
(3) the third polypeptide has the structure, from N-terminus to C-terminus:
   S3-L12-HVD1-L13-second CH1-L14-second HR-115-Fc2;
wherein:
   S1, S2, and S3 are each independently an optional spacer that is either present or absent.
   L1, L2, L3, L4, L5, L6, L7, L8, L9, L10, L11, L12, L13, L14, and L15 are each independently an optional linker that is either present or absent;
   the CM1 and the CM2 each independently comprise a substrate for a protease;
   the LVD1 and the HVD1 together form a first binding domain (BD1) that is specific for a first biological target;
   the LVD2 and the HVD2 together form a second binding domain (BD2) that is specific for a second biological target,
   the MM1 comprises a peptide that attenuates binding of the BD1 to the first biological target;
   the MM2 comprises a peptide that attenuates binding of the BD2 to the second biological target; and
   the Fc1 and Fc2 bind each other.

In some of these embodiments, L2, L3, L4 and L5 or L2, L3, and L4 are present in the first polypeptide, L9 is present in the second polypeptide: L9 and L10 are each present in the second polypeptide. In some of the above-described embodiments, S1, S2, and S3 are each present. In some of these embodiments, S1, S2, S3, L2, L3, L4, L9, and L10 are each present.

In certain embodiments, HVD1 and LVD1 together form a binding domain that is specific for HER2. In some embodiments. MM1 comprises an amino acid sequence selected from the group consisting of SEQ ID NO:11 (F3.11), SEQ ID NO:12 (F3.17), SEQ ID NO:13 (F3.18), SEQ ID NO: 14 (F3.19), SEQ ID NO: 15 (F3.23), SEQ ID NO:16 (F3.42), SEQ ID NO:17 (F3.43), SEQ ID NO:18 (F3.45), SEQ ID NO:19 (F4.2), SEQ ID NO. 20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015);

In certain embodiments, the MM1 comprises an amino acid sequence that differs in from 0-2 amino acid positions from an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

In some embodiments, the MM1 comprises an amino acid sequence selected from the group consisting of SEQ ID NO:20 (F4.03), SEQ ID NO:29 (F5.01), SEQ ID NO:30 (F5.11), SEQ ID NO:31 (F5.13), SEQ ID NO:32 (F5.15), SEQ ID NO:33 (F5.17), and SEQ ID NO:38 (F5.18). In certain embodiments, the MM1 comprises an amino acid sequence selected from the group consisting of SEQ ID NO:29 (F5.01), SEQ ID NO:30 (F5.11), SEQ ID NO:32 (F5.15), SEQ ID NO:33 (F5.17), and SEQ ID NO:38 (F5.18). In other embodiments, the MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

In some of these embodiments, the activatable bispecific anti-HER2 ABPC:
(A) the FHVD2 (anti-CD3 HVD) comprises
   (i) an HCDR1 comprising the amino acid sequence TYAMN (SEQ ID NO:581),
   (ii) an HCDR2 comprising the amino acid sequence RIRSKYNNYATYYADSVKD (SEQ ID NO:582),
   (iii) an HCDR3 comprising the amino acid sequence HGNFGNSYVSWFAY (SEQ ID NO:583), and
(B) the LVD2 (anti-CD3 LVD) comprise
   (i) an LCDR1 comprising the amino acid sequence RSSTGAVTTSNYAN (SEQ ID NO:584),
   (ii) an LCDR2 comprising the amino acid sequence GTNKRAP (SEQ ID NO:585),
   (iii) an LCDR3 comprising the amino acid sequence ALWYSNLWV (SEQ ID NO:586).

For example, in some embodiments, the activatable bispecific anti-HER2, anti-CD3 ABPC may comprise an HVD2 and an LVD2 selected from the group consisting of:
(1) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:587 and an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO: 588;
(2) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO: 587 and an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:589; and
(3) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:590 and an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO: 591.

In some instances, the activatable bispecific anti-HER2, anti-CD3 ABPC of the present disclosure has an HVD2 comprising the amino acid sequence of SEQ ID NO. 587 and a LVD2 comprising the amino acid sequence of SEQ ID NO:588. In other embodiments, the activatable bispecific anti-HER2, anti-CD3 ABPC has an HVD2 comprising the amino acid sequence of SEQ ID NO:593 and a LVD2 comprising the amino acid sequence of SEQ ID NO:589. In certain embodiments, the activatable bispecific anti-HER2, anti-CD3 ABPC has an HVD2 comprising the amino acid sequence of SEQ ID NO:590 and a LVD2 comprising the amino acid sequence of SEQ ID NO:591.

In certain specific embodiments, the HVD2 and the LVD2 are disposed within an scFv having the structure, from N-terminus to C-terminus, HVD2-linker-LVD2 or LVD2-linker-HVD2. Any of a variety of known anti-CD3 scFvs may be employed in the activatable bispecific anti-HER2 ABPCs described herein, including, for example an scFv comprising an amino acid sequence selected from the group consisting of SEQ ID NO:592. SEQ ID NO:594, and SEQ ID NO:597.

In some of the above-described embodiments, the MM2 comprises an amino acid sequence that differs in from 0-2 amino acid residues from an amino acid sequence selected from the group consisting of MMYCGGNEVLCGPRV (SEQ ID NO:598). GYLWGCEWNCGGITT (SEQ ID NO:599), and GYRWGCEWNCGGITT (SEQ ID NO:600). In certain embodiments, the MM2 comprises an amino acid sequence selected from the group consisting of SEQ ID NOs:598, 599, and 600.

In some of the above-described embodiments, the HVD1 comprises:
(i) an HCDR1 comprising the amino acid sequence of SEQ ID NO:1,
(ii) an HCDR2 comprising the amino acid sequence of SEQ ID NO:2,
(iii) an HCDR3 comprising the amino acid sequence of SEQ ID NO:3, and
(iv) an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7, and the LVD1 comprises:
(i) an LCDR1 comprising the amino acid sequence of SEQ ID NO:4,
(ii) an LCDR2 comprising the amino acid sequence of SEQ ID NO:5,
(iii) an LCDR3 comprising the amino acid sequence of SEQ ID NO:6, and
(iv) an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8. In certain of the embodiments described herein, the HVD1 comprises the amino acid sequence of SEQ ID NO:7 and the LVD1 comprises the amino acid sequence of SEQ ID NO:8.

In some of the above-described embodiments, the MM2 comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600). In certain embodiments, the MM2 comprises an amino acid sequence selected from the group consisting of SEQ ID NOs 598, 599, and 600.

Illustrative activatable monovalent, bispecific anti-HER2, anti-CD3 ABPCs are provided in Example 2 hereinbelow. In some embodiments, the activatable monovalent, bispecific anti-HER2, anti-CD3 ABPC comprises a first, a second, and a third polypeptide comprising the amino acid sequences of SEQ ID NOs:714, 626, and 706, respectively. In other embodiments, the bispecific anti-HER2, anti-CD3 ABPC comprises a first, a second, and a third polypeptide comprising the amino acid sequences of SEQ ID NOs:716, 626, and 706, respectively. In still further embodiments, the bispecific anti-HER2, anti-CD3 ABPC comprises a first, a second, and a third polypeptide comprising the amino acid sequences of SEQ ID NOs:718, 626, and 706, respectively.

### Cleavable Moieties (CMs)

Suitable CMs for use in the activatable ABPCs described herein include any of the protease substrates that are known the art. Exemplary substrates include those that are substrates for any one or more of the following proteases: a disintegrin and metalloprotease (ADAM), an ADAM-like, or a disintegrin and metalloproteinase with thrombospondin motifs (ADAMTS, such as, for example, ADAM8, ADAM9, ADAM10, ADAM12, ADAM15, ADAM17/TACE, ADAMDEC1, ADAMTS1, ADAMTS4, ADAMTS5); an aspartate protease (such as, for example, BACE, Renin, and the like); an aspartic cathepsin (such as, for example, Cathepsin D, Cathepsin E, and the like); a caspase (such as, for example. Caspase 1, Caspase 2, Caspase 3, Caspase 4, Caspase 5, Caspase 6, Caspase 7, Caspase 8, Caspase 9, Caspase 10, Caspase 14, and the like); a cysteine cathepsin (such as, for example, Cathepsin B, Cathepsin C, Cathepsin K, Cathepsin L, Cathepsin S, Cathepsin V/L2, Cathepsin X/Z/P): a cysteine proteinase (such as, for example, Cruzipain, Legumain, Otubain-2, and the like); a kallikrein-related peptidase (KLK) (such as, for example, KLK4, KLK5, KLK6, KLK7, KLK8, KLK10, KLK11, KLK13, KLK14, and the like); a metalloproteinase (such as, for example, Meprin, Neprilysin, prostate-specific membrane antigen (PSMA), bone morphogenetic protein 1 (BMP-1), and the like); a matrix metalloproteinase (MMP, such as, for example, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, and the like); a serine protease (such as, for example, activated protein C, Cathepsin A, Cathepsin G, Chymase, a coagulation factor protease (such as, for example, FVIIa, FIXa, FXa, FXIa, FXIIa, and the like); elastase, granzyme B, Guanidinobenzoatase, HtrA1, proteinase 3, Lactoferrin, Marapsin, NS3/4A, PACE4, Plasmin, prostate-specific antigen (PSA), tissue plasminogen activator (tPA), Thrombin, Tryptase, urokinase-type plasminogen activator (uPA), a Type II transmembrane Serine Protease (TSP) (such as, for example, DESC1, DPP-4, FAP, Hepsin, Matriptase-2, MT-SP1/Matriptase, TMPRSS2, TMPRSS3, TMPRSS4, TMPRSS5, TMPRSS6, TMPRSS7, TMPRSS8, TMPRSS9, TMPRSS10, TMPRSS11, and the like), and the like.

In some examples, the CM may comprise a substrate of a serine protease (e.g., u-type plasminogen activator (uPA, also referred to as urokinase), matriptase (also referred to herein as MT-SP1 or MTSP1). In some examples, the CM may comprise a substrate of a matrix metalloprotease (MMP). In some examples, the CM may comprise a substrate of cysteine protease (CP) (e.g., legumain). In certain embodiments, the CM comprises a substrate for at least one protease selected from the group consisting of a matrix metalloprotease (MMP), such as MMP2, thrombin, a cysteine protease, legumain, and a serine protease, such as matriptase (MT-SP1), and urokinase (uPA). In some embodiments, the CM comprises a substrate for at least one MMP. In certain of these embodiments, the MMP is selected from the group consisting of MMP1, MMP3, MMP9, MMP11, MMP13, MMP14, MMP17, and MMP19. In a specific embodiment, the CM comprises a substrate for MMP2. In certain specific embodiments, the CM comprises a substrate for MMP9.

Examples of CMs that are suitable for use in the above-described activatable ABPCs also include those described in WO 2010/081173, WO2021207669, WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, WO 2016/118629, WO 2015/116933, WO 2015/048329, and WO 2010/081173, each of which is incorporated herein by reference in its entirety for all purposes. Suitable CM substituents may be identified using any of a variety of known techniques including those described in U.S. Pat. No. 7,666,817, PCT Publication No. WO 2014/026136, and Boulware, et al., “Evolutionary optimization of peptide substrates for proteases that exhibit rapid hydrolysis kinetics,” Biotechnol. Bioeng. (2010) 106.3:339-46, each of which is incorporated by reference in their entireties.

Each CM (and substrate therein) employed in the design of the activatable ABPC may be selected based on a priori knowledge of specific proteases suspected of being active in the biological sample of interest. In some embodiments, the CM comprises a substrate for a protease that is active, e.g., upregulated or otherwise unregulated, in a disease condition or diseased tissue. Exemplary disease conditions include, for example, a cancer (e.g., where the diseased tissue is a tumor tissue) and an inflammatory or autoimmune condition (e.g., where the diseased tissue is inflamed tissue). In some embodiments, the CM comprises a substrate for an extracellular protease. In other embodiments, the CM comprises a substrate for an intracellular protease.

The CM of the activatable ABPC may be selected so that the CM comprises a substrate for one or more proteases, where the protease is co-localized with the target in a tissue (e.g., at a treatment site or diagnostic site in a subject). The protease may cleave the CM in the activatable ABPC (e.g., activatable anti-HER2 ABPC) when the activatable ABPC is exposed to the protease. In some embodiments, an activatable anti-HER2 ABPC may find particular use where, for example, one or more proteases capable of cleaving a site in the CM, is present at relatively higher levels in HER2 expressing tissue of a treatment site or diagnostic site than in tissue of non-treatment sites (for example in healthy tissue).

In some embodiments, the CMs described herein may comprise substrates for proteases that have been reported as being upregulated in a number of cancers. See, e.g., La Roca et al., British J. Cancer 90(7):1414-1421, 2004. Substrates suitable for use in the CM components employed herein include those which are more prevalently found in cancerous cells and tissue. Thus, in certain embodiments, the CM may comprise a substrate for a protease that is more prevalently found in diseased tissue associated with a cancer. Examples of such cancers include gastric cancer, breast cancer, osteosarcoma, esophageal cancer, and a HER2-positive cancer. In some embodiments, the disease is a HER2-associated disease (e.g., a HER2-positive cancer (e.g., breast cancer, ovarian cancer, bladder cancer, endometrial cancer, pancreatic cancer, non-small cell lung cancer, colorectal cancer, esophageal cancer, gallbladder cancer, glioma, head and neck carcinoma, uterine cancer, cervical cancer or testicular cancer). In some embodiments, the CM comprises a substrate for protease(s) that is/are more prevalent in tumor tissue. For example, the protease(s) may be produced by a tumor in a subject.

In some embodiments, the CM may comprise a total of from 3 amino acids to 25 amino acids. In some embodiments, the CM may comprise a total of from 3 to 25, 3 to 20, 3 to 15, 3 to 10, 3 to 5, 5 to 25, 5 to 20, 5 to 15, 5 to 10, 10 to 25, 10 to 20, 10 to 15, 15 to 25, 15 to 20, or 20 to 25 amino acids. In some embodiments, the CM may be specifically cleaved by at least a protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. The rate may be measured as substrate cleavage kinetics (kcat/Km) as disclosed in WO2016118629, which is incorporated herein by

In certain specific embodiments, each CM of the activatable ABPCs described herein may independently comprise an amino acid sequence selected from the group consisting of SEQ ID NOs: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163 164, 165, 166, 167, 168, 169, 170, 171, 172, 173, 174, 175, 176, 177, 178, 179, 180, 181, 183, 184, 185, 186, 187, 188,189, 190, 191, 192, 193, 194, 195, 196, 197, 198, 199, 200, 201, 202, 203, 204, 205, 206, 207, 208, 209, 210,211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, and 558. In some embodiments, the CM may be or comprise a sequence of LSGRSDDH (SEQ ID NO: 310) or ISSGLLSGRSDNH (SEQ ID NO: 246).

In some embodiments, the activatable ABPC (e.g., an activatable anti-HER ABPC) may comprise a CM between an AB and a MM. In some embodiments, the activatable ABPC (e.g, an activatable anti-HER2 ABPC) may comprise a first CM between the MM and the AB, and a second CM between a second MM and second AB. In an active state, both CMs may be cleaved so that the MM(s) are released from the AB(s). In some examples, the first and the second CMs may comprise the substrates of the same protease. In some examples, the first and the second CMs may comprise the substrates of different proteases. In some examples, the first and the second CMs may comprise or consist of the same sequence. In some examples, the first and the second CMs may comprise or consist of different sequences.

In some embodiments, the protease substrate in the CM may comprise a peptide sequence that is not substantially identical (e.g., no more than 90%, 80%, 70%, 60%, or 50% identical) to any polypeptide sequence that is naturally cleaved by the same protease or any CM described in the present disclosure. In some embodiments, the CM may be or comprise a combination, a C-terminal truncation variant, or an N-terminal truncation variant of the example sequences discussed above. Truncation variants of the aforementioned amino acid sequences that are suitable for use in a CM may be any that retain the recognition site for the corresponding protease. These include C-terminal and/or N-terminal truncation variants comprising at least 3 contiguous amino acids of the above-described amino acid sequences, or at least 4, 5, 6, 7, 8, 9, or 10 amino acids of the foregoing amino acid sequences that retain a recognition site for a protease. In certain embodiments, the truncation variant of the above-described amino acid sequences may be an amino acid sequence corresponding to any of the above, but that is C- and/or N-terminally truncated by from 1 to 10 amino acids, 1 to 9 amino acids, 1 to 8 amino acids, 1 to 7 amino acids, 1 to 6 amino acids, 1 to 5 amino acids, 1 to 4 amino acids, or from 1 to 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

### Optional Components of Activatable ABPCs

Activatable ABPCs of the present disclosure may include one or more linkers, spacers, CL domains, CH1 domains, hinge domains, and/or Fc domains. As described above, linkers may be deployed between otherwise adjacent components to impart, for example, flexibility, spatial separation between components, and the like, to the structure of the activatable ABPC. Linkers suitable for use in the activatable ABPCs described herein may be any of a variety of lengths. Suitable linkers include those having a length in the range of from about 1 to about 20 amino acids, or from about 1 to about 19 amino acids, or from about 1 to about 18 amino acids, or from about 1 to about 17 amino acids, or from about 1 to about 16 amino acids, or from about 1 to about 15 amino acids, or from about 2 to about 15 amino acids, or from about 3 to about 15 amino acids, or from about 3 to about 14 amino acids, or from about 3 to about 13 amino acids, or from about 3 to about 12 amino acids. In some embodiments, the ABPC comprises one or more linkers comprising 1, 2, 3, 4, 5, 6, 7, 8, 9, 10, 11, 12, 13, 14, 15, 16, 17, 18, 19, or 20 amino acids. Typically, the linker is a flexible linker. As used herein, the term “range” is intended to be inclusive of the endpoints which define the limits of the range.

Exemplary linkers that are suitable for use in the activatable ABPCs of the present disclosure include glycine homopolymers (G)ₙ, (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), glycine-serine polymers, including, for example, (GS)ₙ (wherein n is an integer that is at least 1), (GSGGS)ₙ (SEQ ID NO:559) (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), (GGGS)ₙ (SEQ ID NO:560) (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), GGSG (SEQ ID NO:561), GGSGG (SEQ ID NO:562), GSGSG (SEQ ID NO:563), GSGGG (SEQ ID NO:564). GGGSG (SEQ ID NO:565), GSSSG (SEQ ID NO:566), GSSGGSGGSGGSG (SEQ ID NO:567), GSSGGSGGSGG (SEQ ID NO:568), GSSGGSGGSGGS (SEQ ID NO:569), GSSGGSGGSGGSGGGS (SEQ ID NO:570), GSSGGSGGSG (SEQ ID NO:571). GSSGGSGGSGS (SEQ ID NO:572), GGGS (SEQ ID NO:575), GSSGT (SEQ ID NO:576), GSSG (SEQ ID NO:577), GGGSSGGSGGSGG (SEQ ID NO:578), GGGSSGGS (SEQ ID NO:573), GGS, and the like, and additionally, a glycine-alanine polymer, an alanine-serine polymer, and other flexible linkers known in the art.

Activatable ABPCs of the present disclosure may comprise a spacer located, for example, at the amino terminus of the prodomain (i.e., segment comprising an MM and CM). In some embodiments, the spacer is joined directly to the MM of the activatable binding polypeptide. In certain embodiments, the spacer is joined directly to the N-terminus of a polypeptide.in the activatable ABPC. In some embodiments, the spacer is linked directly to the N-terminus of an MM. Exemplary spacer moieties that may be employed in the activatable ABPCs of the present disclosure include, for example, spacer moieties having an amino acid sequence selected from the group consisting of SEQ ID NO:719, 720, 721, 722, 723, 724, 725, 726, 727, 728, 729, 743, SGQ, GQ, G, and Q. In some embodiments, the spacer includes at least the amino acid Q. In some embodiments, the activatable ABPC does not include a spacer sequence.

Light chain constant domains (CL) that are suitable for use in the activatable ABPCs of the present disclosure include any known CL, such as a human kappa or lambda chain (λ₁, λ₂, λ₃, or λ₇) or variant thereof. An illustrative CL domain suitable for use in the activatable ABPCs described herein is a human CL domain comprising the amino acid sequence of SEQ ID NO:732, and variants thereof that are at least about 90% identical, or at least about 91% identical, or at least about 92% identical, or at least about 93% identical, or at least about 94% identical, or at least about 95% identical, or at least about 96% identical, or at least about 97% identical, or at least about 98% identical, or at least about 99% identical to SEQ ID NO:732. In certain specific embodiments, each CL moiety in an activatable ABPC of the present disclosure comprises the amino acid sequence of SEQ ID NO:732.

CH1 domains that are suitable for use in the activatable ABPCs of the present disclosure include any human CH1 domain, such as, for example, the human IgG1 CH1 domain (SEQ ID NO:730), the human IgG4 CH1 domain (SEQ ID NO:731), and the like, as well as any variant thereof having from about 1 to about 5 mutations. In certain specific embodiments, the CH1 domain is a human IgG1 CH1 domain (SEQ ID NO:730). In other embodiments, the CH1 domain is the human IgG4 CH1 domain (SEQ ID NO:731). As described hereinabove, in certain embodiments, the activatable ABPC may comprise a CH1 domain linked directly or indirectly (e.g., via a linker) to an Fc domain. In some embodiments, the CH1 domain is linked directly to the Fc domain.

Any of a variety of Fc domains may be employed in the activatable ABPCs of the present disclosure. Suitable Fc domains include any of a variety of native (e.g., human) or engineered Fc domains that are known in the art. Exemplary Fc domains include, for example, human IgG1 Fc domain (SEQ ID NO:735) or isoform thereof (e.g., SEQ ID NO:736), human IgG4 Fc domain (SEQ ID NO:737), and the like, as well as variants thereof. When the Fc1 and the Fc2 comprise identical amino acid sequences, the activatable ABPC is referred to herein as an activatable “Fc-monomeric” ABPC. When the Fc1 and the Fc2 comprise different amino acid sequences, the activatable ABPC is referred to herein as an activatable “Fc-heterodimeric” ABPC. In some embodiments, the activatable ABPC of the present disclosure is an activatable Fc-monomeric ABPC. In other embodiments, the activatable ABPC is an activatable Fc-heterodimeric ABPC.

As illustrated in Example 7, the introduction of a particular set of mutations into the human IgG1 Fc and IgG4 Fc amino acid sequences resulted in a high yield of asymmetric activatable Fc-heterodimeric ABPCs with minimal aggregation. Accordingly, in a specific aspect, the present disclosure provides an activatable heterodimeric ABPC comprising at least one masking moiety (MM), at least one cleavable moiety (CM), at least one antigen-binding domain having specificity for a biological target, and a first Fc domain (Fc1) comprising [T366S+L368A+Y407V+Y349C] human IgG1 Fc and a second Fc binding domain (Fc2) comprising [T366W+S354C] human IgG1 Fc, wherein the MM comprises a peptide that attenuates binding of the antigen-binding domain to the biological target and the CM comprises a protease substrate. In a further specific aspect, the present disclosure provides an activatable ABPC comprising at least one masking moiety (MM), at least one cleavable moiety (CM), at least one antigen-binding domain, and an Fc1 comprises [S228P+T366S+L368A+Y407V+Y349C] huIgG4 Fc and Fc2 comprises [T366W+S354C] huIgG4 Fc, wherein the MM comprises a peptide that attenuates binding of the antigen-binding domain to the biological target and the CM comprises a protease substrate. These activatable multispecific ABPCs may further comprise one or more of a spacer, a linker, a CL domain, a CH1 domain, and a hinge domain.

The nomenclature used to describe the various Fc variants is described with reference to the parental Fc domain. For amino acid substitutions relative to an Fc domain, the following nomenclature is used: Original amino acid, position, substituted amino acid, wherein position numbering is determined in accordance with the Kabat numbering system, as described Abhinandan, K. R, and Martin, A. C. R. (2008) “Analysis and improvements to Kabat and structurally correct numbering of antibody variable domains”, Molecular Immunology, 45, 3832-3839, which is incorporated herein by reference. For example, “[T366S+L368A+Y407V+Y349C] huIgG1 Fc refers to a variant of the human IgG1 Fc domain that has the combination of substitutions indicated in accordance to EU numbering convention. The first amino acid residue of SEQ ID NOs:735 (human IgG1 Fc domain), 736 (human IgG1 Fc domain isoform), and 737 (human IgG4 Fc domain) corresponds to position number 237 in accordance with the Kabat numbering system.

In some embodiments, the activatable ABPC of the present disclosure (e.g., an activatable anti-HER2 ABPC) is an activatable Fc-heterodimeric ABPC comprising an Fc1 that comprises [T366S+L368A+Y407V+Y349C] huIgG1 Fc and an Fc2 that comprises [T366W+S354C] huIgG1 Fc. In other embodiments, the activatable ABPC comprises [S228P+T366S+L368A+Y407V+Y349C] huIgG4 Fc and an Fc2 that comprises [T366W+S354C] huIgG4 Fc. In certain of the above-described embodiments, the activatable Fc-heterodimeric ABPC is an activatable Fc-heterodimeric, anti-HER2 ABPC, such as, for example, an activatable Fc-heterodimeric, bispecific anti-HER2, anti-CD3 ABPC. Illustrative activatable bispecific anti-HER2 ABPCs are provided herein in Example 7.

### Nucleic Acids, Vectors, and Host Cells

The present disclosure also provides isolated or recombinant polynucleotides that encode the polypeptides of the activatable ABPCs described herein. Polynucleotides of the present disclosure include those which are codon optimized for optimal expression in a particular host organism by modifying the polynucleotides to conform with the optimum codon usage of the desired host organism.

The present disclosure also includes recombinant constructs comprising one or more of the polynucleotides that encode the polypeptide(s) of the activatable ABPCs described herein. The term “construct” or “nucleic acid construct” refers herein to a nucleic acid, either single- or double-stranded, which is isolated from a naturally occurring gene or which has been modified to contain segments of nucleic acids in a manner that would not otherwise exist in nature. The term “nucleic acid construct” is synonymous with the term “expression vector” when the nucleic acid construct contains the control sequences required for expression of a polynucleotide sequence. As used herein, the term “expression” includes any step involved in the production of the polypeptide including, but not limited to, transcription, post-transcriptional modification, translation, post-translational modification, and secretion. Exemplary control sequences include a leader sequence, a polyadenylation sequence, a promoter sequence, a signal peptide sequence, a transcription terminator sequence, and the like. In a specific aspect, the present disclosure also provides an expression vector comprising a polynucleotide of the present invention operably linked to a promoter sequence. The term “operably linked” refers herein to a configuration in which a control sequence is appropriately placed at a position relative to the coding sequence of the polynucleotide sequence such that the control sequence directs the expression of the corresponding polypeptide.

The present disclosure also provides recombinant host cells comprising a polynucleotide or nucleic acid construct (e.g., expression vector) or the present disclosure. The recombinant host cells are derived from host cells that have been transduced (transformed or transfected) with a vector or construct of the present disclosure using recombinant techniques. As used herein, the term “host cell” refers to any cell type which is susceptible to transformation with a nucleic acid construct of the present disclosure.

The (recombinant) host cell can be a eukaryotic cell, such as a mammalian cell (e.g., a Chinese Hamster Ovary (CHO) cell, and the like), a yeast cell, or a plant cell, or a prokaryotic cell, such as a bacterial cell (e.g., E, coli, Bacillus sp., Streptomyces, and the like). Introduction of the nucleic acid construct into the host cell can be effected by calcium phosphate transfection. DEAE-Dextran mediated transfection, electroporation, or any other well-known technique (see, e.g., Davis, L. et al. (1986) Basic Methods in Molecular Biology, which is incorporated herein by reference.

A host cell strain is optionally chosen for its ability to modulate the expression of the inserted sequences or to process the expressed polypeptide in the desired fashion. Such modifications of the polypeptide include, but are not limited to, acetylation, carboxylation, glycosylation, phosphorylation, lipidation, and/or acylation. Different host cells such as E, coli, Bacillus sp. Yeast or mammalian cells such as, for example, CHO, HeLa, BHK, MDCK, HEK 293, W138, and the like, have specific cellular machinery and characteristic mechanisms for such post-translational activities, and may be chosen to ensure the correct modification and processing of the introduced foreign polypeptide.

### Methods of Producing Activatable ABPCs

The present disclosure further provides methods for producing the activatable ABPCs described herein (e.g., activatable anti-HER2 ABPCs), wherein the methods comprise (a) culturing any of the recombinant host cells described herein in a culture medium under conditions sufficient to produce the activatable ABPC; and (2) recovering activatable ABPC from the recombinant host cell and/or the culture medium.

Methods of culturing cells are well known in the art. Cells can be maintained in vitro under conditions that favor cell proliferation, cell differentiation and cell growth. For example, cells can be cultured by contacting a cell with a cell culture medium that includes the necessary growth factors and supplements sufficient to support cell viability and growth. In some embodiments, the recovered activatable ABPC is a recovered activatable ABPC composition that is optionally purified. Any of a number of protein purification methods that are known in the art may be used, including, for example, ammonium sulfate or solvent precipitation, acid extraction, ion exchange chromatography, high performance liquid Chromatography (HPLC), phosphocellulose chromatography, hydrophobic interaction chromatography, affinity chromatography, hydroxylapatite chromatography, lectin chromatography, size-exclusion chromatography, and the like.

### Activatable ABPC Conjugates

The activatable ABPCs described herein (e.g., activatable anti-HER2 ABPCs) may further comprise additional moieties, (referred to herein as “conjugation moieties”) conjugated thereto that confer to the activatable ABPC, an additional property or function, such as, for example, extended half-life (by conjugation to a half-life extending moiety, such as, for example, polyethylene glycol (PEG) moiety, a human serum albumin (HSA) moiety, and the like), cytotoxicity (by conjugation to all or part of a toxin, such as, for example, a dolastin or derivative thereof (e.g., auristatin E, AFP, MMAF, MMAE, MMAD, DMAF, DMAE, and the like, and derivatives thereof); a maytansinoid or derivative thereof; DM1; DM4, a duocarmycin or derivative thereof; a calicheamicin or derivative thereof; a pyrrolobenzodiazepine or derivative or dimer thereof: a heavy metal (e.g., barium, gold, platinum, and the like), a pseudomonas toxin A variant (e.g., PE38, ZZ-PE38, and the like), ZJ-101,OSW-1, a 4-nitrobenzyloxycarbonyl derivative of 06-benzylguanine, a topoisomerase inhibitor, hemiasterlin, cephalotaxine, homoharringonine, a pyrrolobenzodiazepine dimer, a pyrrolobenzodiazepene, a functionalized pyrrolobenzodiazepene, a functionalized pyrrolobenzodiazepene dimer, a calicheamicin, a podophyllotoxin, a taxane, a vinca alkaloid, and the like)), as well as any of a variety of other known cytotoxic agents; anti-viral activity (e.g., by conjugation to all or a portion of Acyclovir, Vira A, Symetrel, Turbostatin, a Phenstatin, Hydroxyphenstatin, Spongistatin 5, Spongistatin 7, Halistatin 1, Halistatin 2, Halistatin 3, a modified bryostatin, a halocomstatin, pyrrolobenzimadazole, cibrostatin6, doxaliform, an anthracycline analogue, a cemadotin analogue (e.g., CemCH2-SH), and the like); antifungal activity (e.g., Nystatin, and the like); anti-neoplastic activity (e.g., by conjugation to Adriamycin, cerubidine, bleomycin, alkeran, velban, oncovin, fluorouracil, methotrexate, thiotepa, bisantrene, novantrone, thioguanine, procarbizine, cytarabine, and the like); anti-bacterial activity (e.g., by conjugation to an aminoglycoside, streptomycin, neomycin, kanamycin, amikacin, gentamicin, tobramycin, Streptomycin B, spectinomycin, ampicillin, sulfanilamide, polymyxin, chloramphenicol, and the like), anti-mycoplasmal activity (e.g., by conjugation to tyrosine, spectinomycin, and the like); detectability (e.g., by conjugation to, for example, a radioisotope (for example, ¹²³I, ⁸⁹Zr, ¹²³I, ¹³¹I, ⁹⁹mTc, ²⁰¹Tl, ⁶²Cu, ¹⁸F, ⁶⁸Ga, ¹³N, ¹⁵O, ³⁸K, ⁸²Rb, ⁹⁹mTc, and the like), a fluorescent agent, and the like); and other desirable other additional properties and functions. Moieties that impart such desired properties and functions can be readily conjugated to the activatable anti-HER2 ABPC using methods and linkers that are known in the art.

### Compositions

The present disclosure provides compositions comprising an activatable ABPC of the present invention (e.g., an activatable anti-HER2 ABPC) or conjugate thereof as described herein, and a pharmaceutically acceptable excipient. Suitable pharmaceutically acceptable excipients include diluents, carriers, processing agents, drug delivery modifiers and the like, such as, for example, calcium phosphate, magnesium stearate, talc, monosaccharides, disaccharides, starch, gelatin, cellulose (e.g., methyl cellulose, sodium carboxymethyl cellulose, and the like), dextrose, hydroxypropyl-β-cyclodextrin, polyvinylpyrrolidone, and the like. Other suitable pharmaceutically acceptable excipients are described in “Remington's Pharmaceutical Sciences”, 18^{th} edition, A. R. Gennaro, Ed. Mack Pub. Co. New Jersey (1991): “Pharmaceutical Excipients, 3^{rd} edition, A. Kibbe, Ed., Pharmaceutical Press (2000), each of which is incorporated herein by reference.

The composition (e.g., pharmaceutical composition) of the present disclosure may further comprise a pharmaceutically acceptable carrier. As used herein, the phrase “pharmaceutically acceptable carrier” refers to any and all solvents, dispersion media, coatings, antibacterial agents, antimicrobial agents, isotonic and absorption delaying agents, and the like, compatible with pharmaceutical administration. Suitable carriers include, but are not limited to: water, saline, ringer's solutions, dextrose solution, and about 5% human serum albumin.

In some embodiments, any of the compositions (e.g., pharmaceutical compositions) described herein can include one or more buffers (e.g., a neutral-buffered saline, a phosphate-buffered saline (PBS)), one or more proteins, polypeptides, or amino acids (e.g., glycine), one or more carbohydrates (e.g., glucose, mannose, sucrose, dextran, or mannitol), one or more antioxidants, one or more chelating agents (e.g., EDTA or glutathione), one or more preservatives, and/or a pharmaceutically acceptable carrier (e.g., bacteriostatic water, PBS, or saline)

Compositions (e.g., pharmaceutical compositions) containing an activatable ABPC (e.g., an activatable anti-HER2 ABPC), or conjugate thereof, may be in any form suitable for the intended method of administration, including, for example, a solution or a solid (e.g., a lyophilized composition for reconstitution prior to administration).

In some embodiments, the compositions (e.g, pharmaceutical compositions) that include any of the activatable ABPCs described herein can be formulated for different routes of administration (e.g., intravenous, subcutaneous, intramuscular, intradermal, oral (e.g., inhalation), transdermal (e.g., topical), transmucosal, or intratumoral).

In some embodiments, the pharmaceutical compositions described herein include any of the activatable antibodies or activatable ABPCs described herein can be prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collagen, polyorthoesters, and polylactic acid. Methods for preparation of such pharmaceutical compositions and formulations are apparent to those skilled in the art. The compositions (e.g, pharmaceutical compositions) that include any of the activatable antibodies or activatable ABPCs described herein can be disposed in a sterile vial or a pre-loaded syringe.

### Methods of Treatment

Also provided herein are methods of treating a disease or disorder in a subject (e.g., a cancer comprising administering to the subject, a therapeutically effective amount of an activatable ABPC of the present disclosure (e.g., an activatable anti-HER2 ABPC), or conjugate or composition thereof.

As used herein, the term “subject” refers to any mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human.

As used herein, the term “treat” includes reducing the severity, frequency or the number of one or more (e.g., 1, 2, 3, 4, or 5) symptoms or signs of a disease (e.g., a cancer) in the subject. In some embodiments where the disease is cancer, treating may result in reducing cancer growth, inhibiting cancer progression, inhibiting cancer metastasis, and/or reducing the risk of cancer recurrence in a subject having cancer.

In some embodiments, the subject has been previously identified or diagnosed as having the disease (e.g., cancer). In some embodiments of any of the methods described herein, the disease is a cancer. In some embodiments, the cancer is selected from the group consisting of: gastric cancer, breast cancer, osteosarcoma, a HER2-positive cancer and esophageal cancer. In some embodiments, the cancer is breast cancer.

In some embodiments, the disease is a HER2-associated disease (e.g., a HER2-positive cancer (e.g., breast cancer, ovarian cancer, bladder cancer, endometrial cancer, pancreatic cancer, non-small cell lung cancer, colorectal cancer, esophageal cancer, gallbladder cancer, glioma, head and neck carcinoma, uterine cancer, cervical cancer or testicular cancer)).

In some embodiments, a subject can be identified as having a mutation in a HER2 gene that increase the expression and/or activity of HER2 in a mammalian cell (e.g., any of the mammalian cells described herein). For example, a mutation in a HER2 gene that increases the expression and/or activity of HER2 in a mammalian cell can be a gene duplication, a mutation that results in the expression of a HER2 having one or more amino acid substitutions as compared to the wild type protein (e.g., one or more amino acid substitutions selected from the group consisting of: G309A, G309E, S310F, R678Q, L755S, L755W, 1767M, D769H, D769Y, V777L, Y835F, V8421, R896C, and G1201V). See, e.g., Weigelt and Reis-Filho, Cancer Discov. 2013, 3(2): 145-147.

Non-limiting examples of methods of detecting a HER2 associated disease in a subject include: immunohistochemistry, fluorescent in situ hybridization (FISH), chromogenic in situ hybridization (CISH). See, e.g., Yan et al., Cancer Metastasis Rev. 2015, 34: 157-164, which is incorporated herein by reference.

### Specific Embodiments of the Present Disclosure

Specific embodiments of the invention include the following:
1. An activatable antigen-binding protein construct (ABPC) comprising:
(A) an antigen binding polypeptide complex (ABPC) that comprises a first anti-HER2 heavy chain variable domain (HVD1) and a first anti-HER2 light chain variable domain (LVD1) that together form a HER-2 binding domain,
wherein the HVD1 comprises:
(i) an HVD1 CDR1 comprising the amino acid sequence DTYIH (SEQ ID NO:1).
(ii) an HVD2 CDR2 comprising the amino acid sequence RIYPTNGYTRYADSVKG (SEQ ID NO: 2), and
(iii) an HVD3 CDR3 comprising the amino acid sequence WGGDGFYAMDY (SEQ ID NO:3), and
wherein the LVD1 comprises
(i) an LVD1 CDR1 comprising the amino acid sequence RASQDVNTAVA (SEQ ID NO:4),
(ii) an LVD1 CDR2 comprising the amino acid sequence SASFLYS (SEQ ID NO:5), and
(iii) an LVD1 CDR3 comprising the amino acid sequence QQHYTTPPT (SEQ ID NO:6); and
(B) a first cleavable moiety (CM) that comprises a substrate for a protease, and
(C) a first anti-HER2 masking moiety (MM1) comprising an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:4l (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015),
wherein the MM1, first CM, first HVD1, and first LVD1 are disposed within one or more polypeptides.
2. The activatable anti-HER2 ABPC of embodiment 1, wherein the first MM1 is selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO: 17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
3. The activatable anti-HER2 ABPC of any of embodiments 1-2, wherein the first MM1 is selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
4. The activatable anti-HER2 ABPC of any of embodiment 1, wherein the first MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).
5. The activatable anti-HER2 ABPC of embodiment 4, wherein the first MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).
6. The activatable anti-HER2 ABPC of any of embodiments 1-5, wherein the HVD1 comprises an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7; and wherein the LVD1 comprises an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8.

The activatable anti-HER2 ABPC of embodiment 1, wherein the activatable anti-HER2 ABPC comprises:
(1) a first polypeptide comprising the first HVD1 and a first Fc domain (Fc1);
(2) a second polypeptide comprising the first MM1, the first CM, the first LVD1, and a first constant light domain (CL);
(3) a third polypeptide comprising a second HVD1 and a second Fc domain (Fc2),
wherein the second anti-HVD1 comprises (i) an HCDR1 having the amino acid sequence of SEQ ID NO:1, (ii) an HCDR2 having the amino acid sequence of SEQ ID NO:2, and (iii) an HCDR3 having the amino acid sequence of SEQ ID NO:3; and
(4) a fourth polypeptide comprising a second MM1, a second CM, a second LVD1 and a second constant light chain domain (CL),
wherein the second CM comprises a substrate for a second protease;
wherein the second LVD1 comprises (i) an LCDR1 comprising the amino acid sequence of SEQ ID NO:4, (ii) an LCDR2 comprising the amino acid sequence of SEQ ID NO:5, and (iii) an LCDR3 comprising the amino acid sequence of SEQ ID NO:6;
wherein the second MM1 comprises an amino acid sequence that differs in from 0-2 amino acid positions from an amino acid sequence selected from the group consisting of SEQ ID NO:11 (F3.11). SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01 b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW00), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015); and wherein the Fc1 and the Fc2 bind each other.
8. The activatable anti-HER2 ABPC of embodiment 7, wherein the first CM and the second CM each comprise a substrate for the same protease.
9. The activatable anti-HER2 ABPC of embodiment 7, wherein the first CM and the second CM comprise substrates for different proteases.
10. The activatable anti-HER2 ABPC of any of embodiments 7-9, wherein each of the first MM1 and the second MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:4l (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
11. The activatable anti-HER2 ABPC of embodiment 10, wherein each of the first MM1 and the second MM1 are each independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
12. The activatable anti-HER2 ABPC of any of embodiments 7-11, wherein each of the first MM1 and the second MM1 independently comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).
13. The activatable anti-HER2 ABPC of any of embodiments 7-12, wherein each of the first MM1 and the second MM1 independently comprises an amino acid sequence selected from the group consisting of SEQ ID NO:29 (F5.01), SEQ ID NO:30 (F5.11), SEQ ID NO:32 (F5.15), and SEQ ID NO:33 (F5.17).
14. The activatable anti-HER2 ABPC of any of embodiments 7-13, wherein the second polypeptide comprises a first CH1 domain, and the fourth polypeptide comprises a second CH1 domain.
15. The activatable anti-HER2 ABPC of any of embodiments 7-14, wherein the second polypeptide and the fourth polypeptide further comprise a first hinge domain and a second hinge domain, respectively.
16. The activatable anti-HER2 ABPC of any of embodiments 7-16, wherein the first MM1 and the second MM1 are the same.
17. The activatable anti-HER2 ABPC of any of embodiments 7-8 and 10-16, wherein the first and third polypeptides comprise the same amino acid sequence.
18. The activatable anti-HER2 ABPC of any of embodiments 7-8 and 10-17, wherein the second and fourth polypeptides comprise the same amino acid sequence.
19. The activatable anti-HER2 ABPC of embodiment 1, further comprising a second binding domain that specifically binds a second biological target (BD2), wherein the first BD2 comprises a first light chain variable domain (LVD2) and a first heavy chain variable domain (HVD2), and a first masking moiety that attenuates the binding of the BD2 to the second biological target (MM2), wherein the activatable anti-HER2 ABPC is an activatable bispecific ABPC.
20. The activatable bispecific ABPC of embodiment 19, further comprising a second anti-HER2 binding domain that comprises a second anti-HER2 heavy chain variable domain (HVD1) and a second anti-HER2 light chain variable domain (LVD1); and a second anti-BD2 comprising a second HVD2 and a second LVD2.
21. The activatable bispecific ABPC of embodiment 20, wherein the second biological target is an immune cell surface antigen.
22. The activatable bispecific ABPC of embodiment 21, wherein the immune cell surface antigen is a CD3.
23. The activatable bispecific ABPC of embodiment 22, wherein the activatable bispecific ABPC comprises a first polypeptide, a second polypeptide, a third polypeptide, and a fourth polypeptide, wherein:
(1) the first polypeptide comprises the first MM2, the first CM, the first HVD2, the first LVD2, the first HVD1, and a first Fc domain (Fc1);
(2) the second polypeptide comprises the first MM1, the second CM, the first LVD1, and a first constant light chain domain (CL);
(3) the third polypeptide comprises a second MM2, a third CM, a second HVD2, a second LVD2, a second HVD1, and a second Fc domain (Fc2); and
(4) the fourth polypeptide comprises a second MM1, a fourth CM, a second LVD1, and a second CL;
wherein the second LVD1 comprises (i) an LCDR1 comprising the amino acid sequence of SEQ ID NO:4, (ii) an LCDR2 comprising the amino acid sequence of SEQ ID NO:5, and (iii) an LCDR3 comprising the amino acid sequence of SEQ ID NO:6;
wherein the second HVD1 comprises (i) an HCDR1 having the amino acid sequence of SEQ ID NO:1, (ii) an HCDR2 having the amino acid sequence of SEQ ID NO:2, and (iii) an HCDR3 having the amino acid sequence of SEQ ID NO:3;
wherein the second MM1 comprises an amino acid sequence that differs in from 0-2 amino acid positions from an amino acid sequence selected from the group consisting of SEQ ID NO:11 (F3.11). SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.0a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW00), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015); and
wherein the Fc1 and the Fc2 bind each other.
24. The activatable bispecific ABPC of embodiment 23, wherein at least two of the first, second, third, and fourth CMs comprise substrates for the same protease.
25. The activatable bispecific ABPC of any of embodiments 23, wherein each of the first MM1 and the second MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:4l (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
26. The activatable anti-HER2 ABPC of any of embodiments 23-25, wherein each of the first MM1 and the second MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
27. The activatable bispecific ABPC of any of embodiments 23-26, wherein each of the first MM1 and the second MM1 independently comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).
28. The activatable bispecific ABPC of embodiment 27, wherein each of the first MM1 and the second MM1 independently comprises an amino acid sequence selected from the group consisting of SEQ ID NO:29 (F5.01), SEQ ID NO:30 (F5.11), SEQ ID NO:32 (F5.15), and SEQ ID NO:33 (F5.17).
29. The activatable bispecific ABPC of any of embodiments 23-28, wherein the first MM1 and the second MM1 are the same.
30. The activatable bispecific ABPC of any of embodiments 23-29, wherein the first HVD2 and the second HVD2 each comprise
(i) an HCDR1 comprising the amino acid sequence TYAMN (SEQ ID NO:581),
(ii) an HCDR2 comprising the amino acid sequence RIRSKYNNYATYYADSVKD (SEQ ID NO:582), and
(iii) an HCDR3 comprising the amino acid sequence HGNFGNSYVSWFAY (SEQ ID NO:583); and wherein the anti-CD3 LVD2 comprises
(i) an LCDR1 comprising the amino acid sequence RSSTGAVTTSNYAN (SEQ ID:584),
(ii) an LCDR2 comprising the amino acid sequence GTNKRAP (SEQ ID NO:585), and
(iii) an LCDR3 comprising the amino acid sequence ALWYSNLWV (SEQ ID NO:586); and
wherein the MM2 comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600).
31. The activatable bispecific ABPC of embodiment 23, wherein each of the pair of first HVD2 and first LVD2 and the second HVD2 the second LVD2 independently comprises an HVD2 and an LVD2 amino acid sequence, respectively, selected from the group consisting of
(A) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to the amino acid sequence of SEQ ID NO:587, SEQ ID NO: 190, and
   (ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:588;
(B) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:587,
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:589; and
(C) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:590, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:591.
32. The activatable bispecific ABPC of any of embodiments 23-31, wherein the first and third polypeptides comprise the same amino acid sequence.
33. The activatable bispecific ABPC of any of embodiments 23-32, wherein the second and fourth polypeptides comprise the same amino acid sequence.
34. The activatable bispecific ABPC of embodiment 19, wherein the activatable bispecific ABPC comprises a first polypeptide, a second polypeptide, and a third polypeptide wherein:
(1) the first polypeptide comprises the MM2, a first CM, the HVD2, the LVD2, and a first Fc domain (Fc1),
(2) the second polypeptide comprises an MM1, a second CM, the LVD1 and a constant light chain domain (CL), and
(3) the third polypeptide comprises the HVD1 and a second Fc domain (Fc2),
wherein MM1 comprises an amino acid sequence that differs in from 0 to 2 positions from an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015), and
wherein the Fc1 and the Fc2 bind each other.
35. The activatable bispecific ABPC of embodiment 34, wherein the first CM and the second CM comprise substrates for the same protease.
36. The activatable bispecific ABPC of any of embodiments 34-35, wherein the MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
37. The activatable anti-HER2 ABPC of any of embodiments 34-36, wherein the MM1 is selected from the group consisting of SEQ ID NO:11 (F3.11), SEQ ID NO:12 (F3.17), SEQ ID NO:13 (F3.18), SEQ ID NO: 14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).
38. The activatable bispecific ABPC of any of embodiments 34-37, wherein the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).
39. The activatable bispecific ABPC of embodiment 38, wherein the MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).
40. The activatable bispecific ABPC of any of embodiments 34-39, wherein the HVD2 comprises
(i) an HCDR1 comprising the amino acid sequence TYAMN (SEQ ID NO:581),
(ii) an HCDR2 comprising the amino acid sequence RIRSKYNNYATYYADSVKD (SEQ ID NO:582), and
(iii) an HCDR3 comprising the amino acid sequence HGNFGNSYVSWFAY (SEQ ID NO:583); and
wherein the LVD2 comprises
(i) an LCDR1 comprising the amino acid sequence RSSTGAVTTSNYAN (SEQ ID:584),
(ii) an LCDR2 comprising the amino acid sequence GTNKRAP (SEQ ID NO:585), and
(iii) an LCDR3 comprising the amino acid sequence ALWYSNLWV (SEQ ID NO:586); and
wherein the MM2 comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600).
41. The activatable bispecific ABPC of embodiment 40, wherein the HVD2 and the LVD2 are, selected from the group consisting of
(A) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to the amino acid sequence of SEQ ID NO:587, SEQ ID NO: 190, and
   (ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:588;
(B) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:587,
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:589; and
(C) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:590, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:591.
42. The activatable bispecific ABPC of any of embodiments 34-41, wherein the HVD1 comprises
an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7, and
the LVD1 comprises an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8.
43. The activatable bispecific ABPC of embodiment 42, wherein the HVD1 comprises the amino acid sequence of SEQ ID NO:7 and the LVD1 comprises the amino acid sequence of SEQ ID NO:8.
44. An activatable ABPC conjugate comprising the activatable ABPC of any of embodiments 1-43 and a conjugation moiety.
45. The activatable ABPC conjugate of embodiment 44, wherein the conjugation moiety is a half-life extending moiety.
46. The activatable ABPC conjugate of embodiment 44, wherein the conjugation moiety is a toxin.
47. The activatable ABPC conjugate of embodiment 44, wherein the conjugation moiety is a detectable moiety.
48. A composition comprising the activatable ABPC of any of embodiments 1-43 or activatable ABPC conjugate of any of embodiments 44-47 and a pharmaceutically acceptable excipient.
49. A method of treating a disease or disorder in a subject comprising administering to a subject in need thereof comprising administering to the subject a therapeutically effective amount of the activatable ABPC of any one of embodiments 1-43, or activatable ABPC conjugate of any one of embodiments 44-47, or composition of embodiment 48.
50. The method of embodiment 49, wherein the disease or disorder is a cancer.
51. A polynucleotide encoding at least one polypeptide of the activatable ABPC of any of embodiments 1-43.
52. A vector comprising the polynucleotide of embodiment 51.
53. The vector of embodiment 52, wherein the vector is an expression vector that further comprises a promoter operably linked to the polynucleotide.
54. A recombinant host cell comprising the polynucleotide of embodiment 51 or the vector of any of embodiments 52 or 53.
55. A method for producing an activatable ABPC of any of embodiments 1-43, wherein the method comprises:
(a) culturing the recombinant host cell of embodiment 54 in a culture medium under conditions sufficient to produce the activatable ABPC; and
(b) recovering the activatable ABPC from the recombinant host cell and/or the culture medium.

The following examples further illustrate the practice of the invention but should not be construed as limiting its scope in any way.

### EXAMPLES

The invention is further described in the following examples, which do not limit the scope of the invention described in the claims.

### Example 1: Activatable Anti-HER2 ABPCs

A set of peptides were identified as potential anti-HER2 masking moieties (MM1). The sequences of the candidate MM1s are provided in Table 1, below.

TABLE 1
Anti-HER2 Masking Moieties (MM1)

| | Mask SEQ ID | |
|---|---|---|
| Name | NO: | Sequence |
| F3.11 | 11 | MSELCLSEYRCWGLE |
| F3.17 | 12 | MTMCGEFMFLGCETR |
| F3.18 | 13 | GRMTCADHPKCNYIY |
| F3.19 | 14 | PICAFVYNDPECSWW |
| F3.23 | 15 | SWCMMHHEGNVCQTV |
| F3.42 | 16 | QFACFETRSQYCDWK |
| F3.43 | 17 | MLWCDTPDGSCWWRE |
| F3.45 | 18 | ICALTIFSTSVDCLY |
| F4.2 | 19 | DGYVCHWEWACRTAA |
| F4.3 | 20 | LDCVRTLYYSDCYHR |
| F4.10 | 21 | LCVMDHIYDTMNCNQ |
| F4.13 | 22 | PFKCDLTNPTCTILM |
| F4.22 | 23 | QFCRNYPLSMMCCKY |
| F4.35 | 24 | ICSYNWHFDTIDCNS |
| F5.01 | 25 | CCYVLLDGILMPCDC |
| F5.11 | 30 | SECMISQWGPDCTVA |
| F5.13 | 31 | YCLYNKLNDTINCSD |
| F5.15 | 32 | RCVMDPVTDTIDCNK |
| F5.17 | 33 | ALICCSDVSGLCRWC |
| F5.18 | 38 | SSCRTTGLMTTCDLD |
| mLW001 | 39 | LYCTEGRWYEGTCAG |
| mLW002 | 40 | LTKCATSTSTTCDLD |
| mLW003 | 41 | FGCGVVLDLDHCAPW |
| mLW004 | 42 | TPCWHMIMTNPECMR |
| mLW005 | 43 | YNACWFGPYDCWYRP |
| mLW006 | 44 | FLCEPPSWGPDCTMI |
| mLW007 | 45 | DLCSFGLWYRGTCEG |
| mLW008 | 46 | TCYHDMHKDTIHCNN |
| mLW009 | 47 | LYRDMCGGGCEDWHD |
| mLW010 | 48 | LGSLCDPFDCPGKNL |
| mLW011 | 49 | RGCSFGYWGNGTCLG |
| mLW012 | 50 | RACSFGVWWKGTCEG |
| mLW013 | 51 | ANVCWLGPYDCRWES |
| mLW014 | 52 | GMCTSGRWYGGDCTG |
| mLW015 | 53 | ACTHNTMTDKIYCNN |

### Example 2: Sequences, Vector Construction and Expression of Activatable Antibodies

The peptides from Example 1 were incorporated into activatable monospecific and bispecific anti-HER2 ABPCs as described below.

a. Activatable Divalent, Monospecific, Anti-HER2 ABPCs

Activatable divalent, monospecific, anti-HER2 ABPCs were prepared that incorporated the various peptides described in Example 1 as anti-HER2 masking moieties (MM1). Each of the activatable ABPCs had two identical light chains and two identical heavy chains within the structure depicted in FIG. 1. Each light chain had an anti-HER2 masking moiety (MM1) incorporated within it. The light chain had the following structure, from N-terminus to C-terminus:
S1 (SEQ ID NO:722)-MM1-L1 (SEQ ID NO:313)-CM1 (SEQ ID NO:246)-L2 (SEQ ID NO:575)-LVD1 (SEQ ID NO:8)-CL (SEQ ID NO:732)
where S1 is a spacer, MM1 is an anti-HER2 masking moiety, L1 and L2 are linkers, LVD1 is an anti-HER2 light chain variable domain, and CL is a constant light chain domain.

The heavy chain had the following structure:
HVD1 (SEQ ID NO:9)-CH1 (SEQ ID NO:730)-hinge (SEQ ID NO:733)-Fc domain (SEQ ID NO:735). The heavy chain amino acid sequence for each of the activatable ABPCs was the same.

DNA encoding the polypeptides of the activatable ABPCs were cloned separately into a mammalian expression vector using standard molecular biology techniques. Briefly, DNA fragments encoding the region of interest were commercially purchased (Integrated DNA Technologies, gBlocks). When necessary, DNA fragments encoding the region of interest were amplified with primers binding to the terminal ends and overlapping fragments were combined and amplified with flanking primers as needed to build the entire desired region. DNA fragments were subsequently cloned into the expression vector using a commercially available homologous recombination kit (MCLabs, South San Francisco, CA). The mammalian expression vector was a modified version of cDNA™ 3.1(+) from Invitrogen with a G418 or hygromycin selection marker.

Polypeptides encoding the activatable ABPCs were expressed in mammalian cells using a standard transfection kit (Life Technologies, Grand Island, NY). Briefly, HEK 293 cells were transfected with nucleic acids using a lipid-based system, following the manufacturer's recommended protocol. The activated ABPCs were purified from cell-free supernatant using Protein A beads (GE, Piscataway, NJ) and concentrated using standard buffer exchange columns (Millipore. Temecula, CA).

The sequences and MM1 component for each activatable ABPC are provided in Table 2.

TABLE 2
Activatable Divalent, Monospecific ABPCs that bind HER2

| | | | Heavy Chain Sequence, |
|---|---|---|---|
| | | Light Chain Sequence, | SEQ ID NO. |
| Molecule | | SEQ ID NO. | (Amino |
| Name | MM1 | (Amino Acid/Polynucleotide) | Acid/Polynucleotide) |
| CI360 | F3.23 | 603/604 | 601/602 |
| | (SEQ ID NO: 15) | | |
| CI361 | F4.3 | 605/607 | 601/602 |
| | (SEQ ID NO: 20) | | |
| CI362 | F4.35 | 608/609 | 601/602 |
| | (SEQ ID NO: 24) | | |
| CI363 | F5.01 | 610/611 | 601/602 |
| | (SEQ ID NO: 29) | | |
| CI364 | F5.01a | 612/614 | 601/602 |
| | (SEQ ID NO: 25) | | |
| CI365 | F5.01b | 614/615 | 601/602 |
| | (SEQ ID NO: 26) | | |
| CI366 | F5.01c | 616/617 | 601/602 |
| | (SEQ ID NO: 27) | | |
| CI367 | F5.01d | 618/619 | 601/602 |
| | (SEQ ID NO: 28) | | |
| CI368 | F5.11 | 620/621 | 601/602 |
| | (SEQ ID NO: 30) | | |
| CI369 | F5.13 | 622/623 | 601/602 |
| | (SEQ ID NO: 31) | | |
| CI370 | F5.15 | 624/625 | 601/602 |
| | (SEQ ID NO: 32) | | |
| CI371 | F5.17 | 626/627 | 601/602 |
| | (SEQ ID NO: 33) | | |
| CI372 | F5.17a | 628/629 | 601/602 |
| | (SEQ ID NO: 34) | | |
| CI373 | F5.17b | 630/631 | 601/602 |
| | (SEQ ID NO: 35) | | |
| CI374 | F5.17c | 632/633 | 601/602 |
| | (SEQ ID NO: 36) | | |
| CI375 | FS.17d | 634/635 | 601/602 |
| | (SEQ ID NO: 37) | | |
| CI376 | F5.18 | 638/637 | 601/602 |
| | (SEQ ID NO: 38) | | |

### B. Activatable Divalent, Bispecific Anti-HER2, Anti-CD3 ABPCs

Activatable divalent, bispecific anti-HER2, anti-CD3 ABPCs were prepared, each of which incorporated one of the various peptides described in Example 1 as an anti-HER2 masking moiety (MM1). A schematic of the structure of the activatable divalent, bispecific anti-HER2, anti-CD3 ABPCs is provided in FIG. 2. Each of the activatable divalent, bispecific anti-HER2, anti-CD3 ABPCs had four polypeptides: two identical heavy chain polypeptides each comprising a spacer (S1), an MM2 (anti-CD3 masking moiety), a CM2 (a cleavable moiety), an anti-CD3 scFv moiety (HVD2-linker-LVD2), an HVD1 (anti-HER2 heavy chain variable domain), CH1 domain, a hinge domain, and an Fc domain; and two identical light chain polypeptides each comprising a spacer, an MM1, a CM1, an LVD1 (anti-HER2 light chain variable domain), and a constant light chain domain (CL) polypeptide.

Each light chain had the structure, from N- to C-terminus:
S1-MM1-L1-CM1-HL2-LVD1 (SEQ ID NO:8)-CL where S1 is a spacer, MM1 is an anti-Her2 masking moiety L1 and L2 are linkers, CM1 is a cleavable moiety, LVD1 is an anti-HER2 light chain variable domain, and CL is a light chain constant domain.

Each heavy chain had the structure, from N- to C-terminus:
S2N-MM2-L3-CM2-L4-LVD2 (SEQ ID NO:591)-L5-HVD2 (SEQ ID NO:590)-L6
HVD1 (SEQ ID NO: 9)-CH1 (SEQ ID NO:730)-Hinge (SEQ ID NO:733)-Fc (SEQ ID NO:735), where S2 is a spacer, MM2 is an anti-CD3 masking moiety, CM2 is a second cleavable moiety. LVD2-L5-HVD2 is an anti-CD3 scFv moiety, HVD1 is an anti-HER2 heavy chain variable domain, CH1 is a hinge domain, and Fc is an Fc domain.

The molecules were prepared as described in part A, above. The activatable divalent, bispecific anti-HER2, anti-CD3 ABPCs are described below in Table 3

TABLE 3
Activatable Divalent, Bispecific, Anti-HER2, anti-CD3 ABPCs

| | Anti-HER2 | Anti-HER2 | Anti-CD3 | Anti-CD3 | SEQ ID chain | |
|---|---|---|---|---|---|---|
| Molecule | Mask | SEQ ID NO. | Mask | SEQ ID NO | AA/PN | |
| Name | (MM1) | VLD1/VHD1 | (MM2) | VLD2/VHD2 | LC^{Δ} | HC |
| CI377 | F5.01 | 8/7 | JF15865 | hSP34LvHv- | 610/611 | 638^{Δ}/639 |
| F5.01-2001- | (SEQ ID | | (SEQ ID | H-N | | |
| HER2-JF15865- | NO: 29) | | NO: 598) | | | |
| 2001- | | | | | | |
| hSP34LvHv-H-N | | | | | | |
| CI378 | F5.11 | 8/7 | JF15865 | hSP34LvHv- | 620/621 | 638^{Δ}/639 |
| F5.11-2001- | (SEQ ID | | (SEQ ID | H-N | | |
| HER2-JF15865- | NO: 30) | | NO: 598) | | | |
| 2001- | | | | | | |
| hSP34LvHv-H-N | | | | | | |
| CI379 | F5.15 | 8/7 | JF15865 | hSP34LvHv- | 624/625 | 638^{Δ}/639 |
| F5.15-2001- | (SEQ ID | | (SEQ ID | H-N | | |
| HER2-JF15865- | NO: 32) | | NO: 598) | | | |
| 2001- | | | | | | |
| hSP34LvHv-H-N | | | | | | |
| CI380 | F5.17 | 8/7 | JF15865 | hSP34LvHv- | 626/627 | 638^{Δ}/639 |
| F5.17-2001- | (SEQ ID | | (SEQ ID | H-N | | |
| HER2-JF15865- | NO: 33) | | NO: 598) | | | |
| 2001- | | | | | | |
| hSP34LvHv-H-N | | | | | | |
| CI101 | F5.17 | 8/7 | JF15865 | L15- | 627/628 | 638^{#}/639 |
| F5.17-HER2 | (SEQ ID | | (SEQ ID | hSP34Lv- | | |
| N297Q-JF15865- | NO: 33) | | NO: 598) | H-N | | |
| 2001-L15- | | | | | | |
| hSP34LvHv-H-N | | | | | | |
| CI124 | F5.17 | 8/7 | 20GG | L15-v12 sc | 626/627 | 646^{⋄}/647 |
| F5.17-HER2 | (SEQ ID | | (SEQ ID | | | |
| Fcmt4-20GG- | NO: 17) | | NO: 598) | | | |
| 2001- | | | | | | |
| L15-v12sc-H-N | | | | | | |
| CI156 | F5.17 | 8/7 | 20GG | L15-v12 sc | 626/627 | 670^{⋄}/671 |
| F5.17-0001-HER2 | (SEQ ID | | (SEQ ID | | | |
| Fcmt4-h20GG- | NO: 17) | | NO: 600) | | | |
| 0001- | | | | | | |
| L15-v12sc-H-N | | | | | | |

^{Δ}Spacer sequence SEQ ID NO: 722
^{⋄}Spacer sequence SEQ ID NO: 719
^{#}Spacer sequence SEQ ID NO: 726

Activated variants of CI101 and CI124, Act-101 and Act-124, respectively, were also prepared for further studies according to the methods described in part A. The sequences of the activated variants are provided in Table 4.

TABLE 4
Activated Variants

| | | | | Light Chain | Heavy Chain |
|---|---|---|---|---|---|
| Molecule | Molecule | Heavy Chain | Light Chain | SEQ ID NO. | SEQ ID NO. |
| Name | Component Parts | Vector | Vector | (AA/PN) | (AA/PN) |
| Act-101 | Activated F5.17- | Activated | Activated LC | 644/645 | 642/643 |
| | HER2 N297Q- | pLW255 | F5.17-2001- | | |
| | JF15865-2001-L15- | | HER2 | | |
| | hSP34LvHv-H-N | | | | |
| Act- | Activated F5.17- | Activated- | Activated- | 644/645 | 648/649 |
| CI124 | HER2 Fcmt4-20GG- | pLW321 | OPP038 | | |
| | 2001-L15-v12sc-H-N | | | | |

Additional activatable divalent, bispecific anti-HER2, anti-CD3 ABPCs were constructed. Each of the activatable ABPCs had the structure depicted in FIG. 2. Each molecule had two identical light chains and two identical heavy chains. Each of the activatable ABPCs had the same heavy chain sequences.

Each light chain had the structure, from N-terminus to C-terminus:

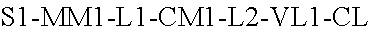

Each heavy chain has the structure, from N-terminus to C-terminus:

(SEQ ID NO: 719)
S2-MM2-L1-CM2-L2-[LVD2-L3-HVD2]-CH1-Hinge-Fc

The bracketed sequence elements correspond to an anti-CD3 scFv. A description of these activatable divalent, bispecific anti-HER2, anti-CD3 ABPCs is provided in Table 5.

TABLE 5
Activatable Divalent, Bispecific Anti-HER2, Anti-CD3ABPCs

| | | | Light Chain | Heavy Chain |
|---|---|---|---|---|
| | | Anti-HER2 | SEQ ID NO., | SEQ ID NO., |
| Molecule | Molecule Component | Mask | amino | amino |
| Name | Parts | (MM1) | acid/polynucleotide | acid/polynucleotide |
| CI185 | mLW001-2014- | mLW001 | 676/677 | 674/675 |
| | HER2Fcmt4-h20GG- | (SEQ ID | | |
| | 2014-L15-v12sc-H-N | NO: 39) | | |
| CI381 | mLW002-2014- | mLW002 | 678/679 | 674/675 |
| | HER2Fcmt4-h20GG- | (SEQ ID | | |
| | 2014-L15-v12sc-H-N | NO: 40) | | |
| CI382 | mLW003-2014- | mLW003 | 680/681 | 674/675 |
| | HER2Fcmt4-h20GG- | (SEQ ID | | |
| | 2014-L15-v12sc-H-N | NO: 41) | | |
| CI383 | mLW004-2014- | mLW004 | 682/683 | 674/675 |
| | HER2Fcmt4-h20GG- | (SEQ ID | | |
| | 2014-L15-v12sc-H-N | NO: 42) | | |
| CI384 | mLW005-2014- | mLW005 | 684/685 | 674/675 |
| | HER2Fcmt4-h20GG- | (SEQ ID | | |
| | 2014-L15-v12sc-H-N | NO: 43) | | |
| CI385 | mLW006-2014- | mLW006 | 686/687 | 674/675 |
| | HER2Fcmt4-h20GG- | (SEQ ID | | |
| | 2014-L15-v12sc-H-N | NO: 44) | | |
| CI386 | mLW007-2014- | mLW007 | 688/689 | 674/675 |
| | HER2Fcmt4-h20GG- | (SEQ ID | | |
| | 2014-L15-v12sc-H-N | NO: 45) | | |
| CI387 | mLW008-2014- | mLW008 | 690/691 | 674/675 |
| | HER2Fcmt4-h20GG- | (SEQ ID | | |
| | 2014-L15-v12sc-H-N | NO: 46) | | |
| CI388 | mLW009-2014- | mLW009 | 692/693 | 674/675 |
| | HER2Fcmt4-h20GG- | (SEQ ID | | |
| | 2014-L15-v12sc-H-N | NO: 47) | | |
| CI186 | mLW010-2014- | mLW010 | 694/695 | 674/675 |
| | HER2Fcmt4-h20GG- | (SEQ ID | | |
| | 2014-L15-v12sc-H-N | NO: 48) | | |
| CI187 | mLW011-2014- | mLW011 | 696/697 | 674/675 |
| | HER2Fcmt4-h20GG- | (SEQ ID | | |
| | 2014-L15-v12sc-H-N | NO: 49) | | |
| CI389 | mLW012-2014- | mLW012 | 698/699 | 674/675 |
| | HER2Fcmt4-h20GG- | (SEQ ID | | |
| | 2014-L15-v12sc-H-N | NO: 50) | | |
| CI390 | mLW013-2014- | mLW013 | 700/701 | 674/675 |
| | HER2Fcmt4-h20GG- | (SEQ ID | | |
| | 2014-L15-v12sc-H-N | NO: 51) | | |
| CI391 | mLW014-2014- | mLW014 | 702/703 | 674/675 |
| | HER2Fcmt4-h20GG- | (SEQ ID | | |
| | 2014-L15-v12sc-H-N | NO: 52) | | |
| CI392 | mLW015-2014- | mLW015 | 704/705 | 674/675 |
| | HER2Fcmt4-h20GG- | (SEQ ID | | |
| | 2014-L15-v12sc-H-N | NO: 53) | | |

### Example 3: Cell-Free Binding of Activatable Divalent, Monospecific Anti-HER2 ABPCs

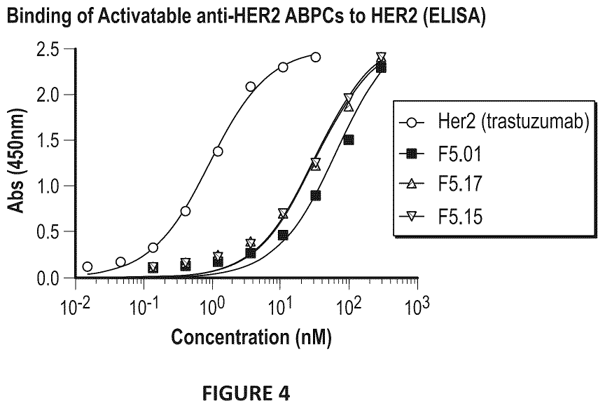

Masking efficiencies (ME) were evaluated by standard plate ELISA. Briefly, a 96-well ELISA plate was treated with human HER2 (hHER2) (R and D systems, Cat. No. 1129-ER). Anti-HER2 antibody (trastuzumab) and activatable ABPCs, each including one of the MM1s as described herein and having the structure of FIG. 1, were serially diluted and applied to the hHER2-coated plates. Bound anti-HER2 antibody (e.g., trastuzumab) and bound activatable anti-HER2 ABPC (trastuzumab) that is coupled with a MM as described herein and a CM) were detected using anti-human IgG-HRP conjugated, FAb-specific secondary antibody (Sigma) and visualized with the chromogenic substrate TMB (Thermo Scientific). Plots were generated in Prism (GraphPad). The data were fit to a model of single-site saturation binding (See FIG. 4 for representative graph for Activatable Divalent, Monospecific Anti-HER2 ABPC with F5.01, F5.17 and F5.15 masks), and the equilibrium dissociation constant (Kd) was determined. Masking efficiency (ME) was calculated by dividing the Kd of the activatable anti-HER2 ABPC (e.g., trastuzumab coupled with a MM and a CM) by the Kd of the parental antibody (e.g., trastuzumab) (Table 6).

TABLE 6
Masking Efficiency of Anti-HER2 Masks (MM1)

| | Anti-HER2 | | | |
|---|---|---|---|---|
| | Mask (MM1) | | | |
| Name | SEQ ID NO: | Sequence | Kd (nM) | ME |
| Plate 1 | N/A | Trastuzumab | 0.93 | 1.00 |
| Control | | (“Ab#1”) | | |
| F3.23 | 15 | SWCMMHHEGNVCQTV | 1.16 | 1.24 |
| F4.03 | 20 | LDCVRTLYYSDCYHR | 6.34 | 6.81 |
| F4.35 | 24 | ICSYNWHFDTIDCNS | 4.43 | 4.75 |
| F5.01 | 29 | CCYVLLDGILMPCDC | 31.28 | 33.57 |
| F5.01a | 25 | SCYVLLDGILMPSDC | 1.06 | 1.14 |
| F5.01b | 26 | SCYVLLDGILMPCDS | 1.40 | 1.51 |
| F5.01c | 27 | CSYVLLDGILMPSDC | 0.96 | 1.03 |
| F5.01d | 28 | CSYVLLDGILMPCDS | 1.08 | 1.16 |
| F5.11 | 30 | SECMISQWGPDCTVA | 17.90 | 19.21 |
| F5.13 | 31 | YCLYNKLNDTINCSD | 8.73 | 9.36 |
| F5.15 | 32 | RCVMDPVTDTIDCNK | 31.70 | 33.91 |
| F5.17 | 33 | ALICCSDVSGLCRWC | 23.5 ± 12.49^{⋄} | 25.2 ± 13.2^{⋄} |
| F5.17a | 34 | ALISCSDVSGLSRWC | 0.63 | 0.83 |
| F5.17b | 35 | ALISCSDVSGLCRWS | 2.06 | 2.69 |
| F5.17c | 36 | ALICSSDVSGLSRWC | 0.86 | 1.13 |
| F5.17d | 37 | ALICSSDVSGLCRWS | 1.28 | 1.67 |
| F5.18 | 38 | SSCRTTGLMTTCDLD | 12.26 | 16.01 |

^{⋄}multiple replicates

The results indicated a range of masking efficiencies, with several of the anti-HER2 masking moieties exhibiting substantially high ME.

### Example 4: Characterization of Activatable Divalent, Bispecific Anti-HER2 ABPCs

Four of the anti-HER2 masking moieties described in Example 1 were evaluated in the context of an activatable divalent, bispecific anti-HER2, anti-CD3 ABPC structure depicted in FIG. 2:
(1) CI377 with MM1, F5.01 (SEQ ID NO: 29);
(2) CI378 with MM1, F5.11 (SEQ ID NO: 30),
(3) CI379 with MM1, F5.15 (SEQ ID NO:32); and
(4) CI380 with MM1, F5.17 (SEQ ID NO:33).

The sequences and structure of these activatable ABPCs are described in Example 2.

### A. Binding to Her2+ Cells and CD3+ Jurkat Cells

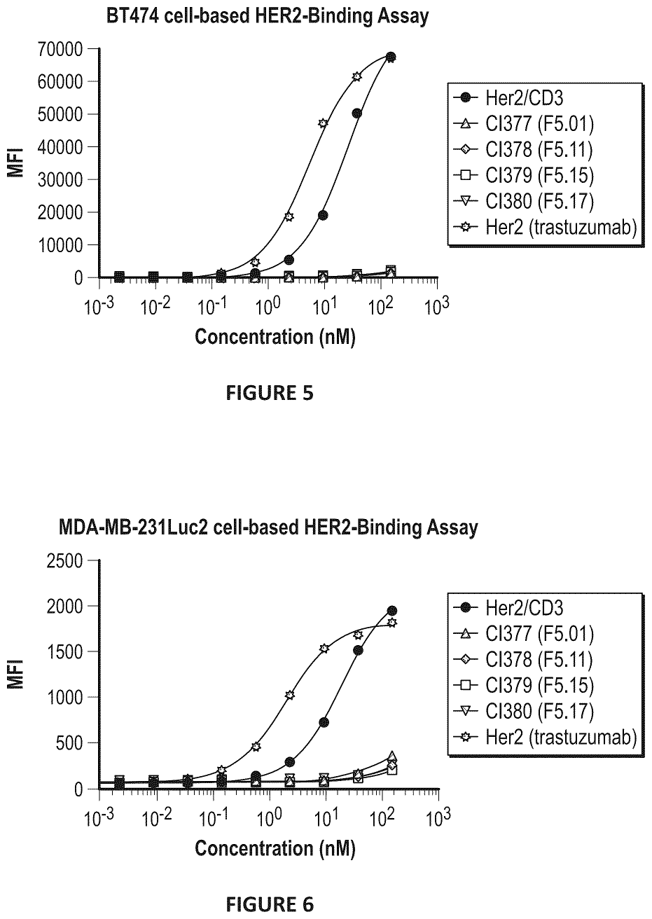

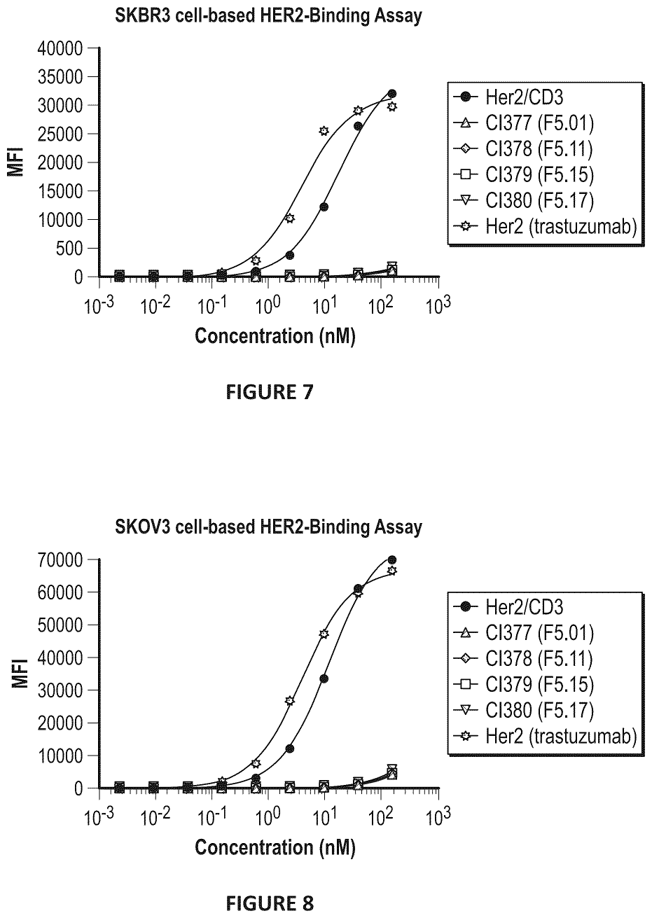

Binding inhibition of the activatable divalent, bispecific ABPCs was evaluated using a panel of HER2-expressing cell lines and CD3-expressing Jurkat cells. Briefly, HER2-expressing cells (e.g., BT474. MDA-MB-231-Luc2, SKBR3, and SKOV3 cell lines) and Jurkat cells (e.g., Clone E6-1, ATCC. TIB-152 cell lines) were cultured in RPMI-1640+glutamax (Life Technologies, Cat. No. 72400-047), 10% heat inactivated-fetal bovine serum (HI-FBS, Life Technologies, Cat. No. 10438-026), and 100 U/mL penicillin/100 μg/mL streptomycin (Life Technologies, Cat. No. 15140-122) according to manufacturer's guidelines. Adherent cell lines were detached using Versene®, washed, and incubated for one hour with the activatable ABPCs, as well as two controls, a corresponding unmasked form of the activatable ABPC, and trastuzumab. Cells were then washed and incubated with Alexa Fluor® 647-conjugated anti-human IgG Fc-gamma fragment specific antibody (Jackson ImmunoResearch Laboratories. Cat. No. 109-605-098) for 30 minutes. Cells were then washed and evaluated using flow cytometry. Plots were generated in Prism (GraphPad). The data were fit using a three parameters least squares fit of the log vs, response curve fit (FIGS. 5-8), and the effective concentration, EC50, was determined.

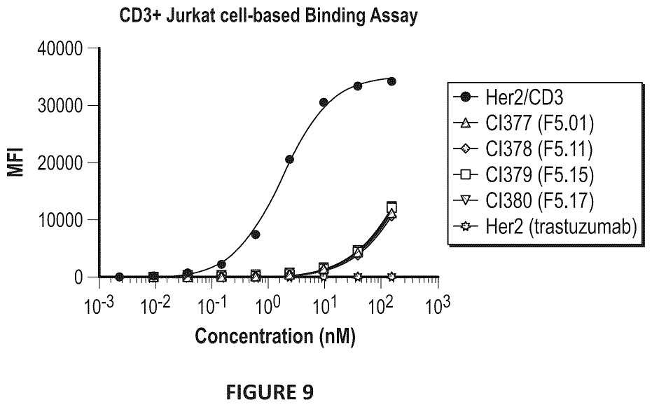

The HER2 binding curves (FIGS. 5-8) showed that the activatable ABPCs (i.e., CI377, CI378, CI379, CI380) exhibited significantly reduced binding to HER2-expressing cell lines relative to the controls. The CD3 binding curves (FIG. 9) further indicate that the masked anti-CD3 antigen-binding domain exhibits reduced binding to CD3 expressing cell lines relative to the controls.

### B. Aggregation and Yield of Activatable Divalent, Bispecific Anti-HER2, Anti-CD3

The monomer content of purified preparations of the above-described activatable ABPCs was analyzed using analytical SEC. Protein aliquots (25 μg) were injected onto Superdex 200 Increase 5/150 GL column (GE Healthcare Life Sciences, Catalog #28906561) that was run with 1×PBS pH7.2 at 0.45 ml/min. The percentage of monomer in each purified preparation is provided in Table 7.

TABLE 7
Percent Monomer in Purified Preparations

| | HER2 | | % | Protein A |
|---|---|---|---|---|
| Molecule | Mask | Protein | Monomer | Octet (mg/L) |
| CI377 | F5.01 | F5.01-2001-HER2-JF15865-2001- | 74.46 | 17.2 |
| | | hSP34LvHv-H-N | | |
| CI378 | F5.11 | F5.11-2001-HER2-JF15865-2001- | 67.05 | 67.6 |
| | | hSP34LvHv-H-N | | |
| CI379 | F5.15 | F5.15-2001-HER2-JF15865-2001- | 68.37 | 64.8 |
| | | hSP34LvHv-H-N | | |
| CI380 | F5.17 | F5.17-2001-HER2-JF15865-2001- | 68.8 | 64.6 |
| | | hSP34LvHv-H-N | | |

### C. Cytoxicity

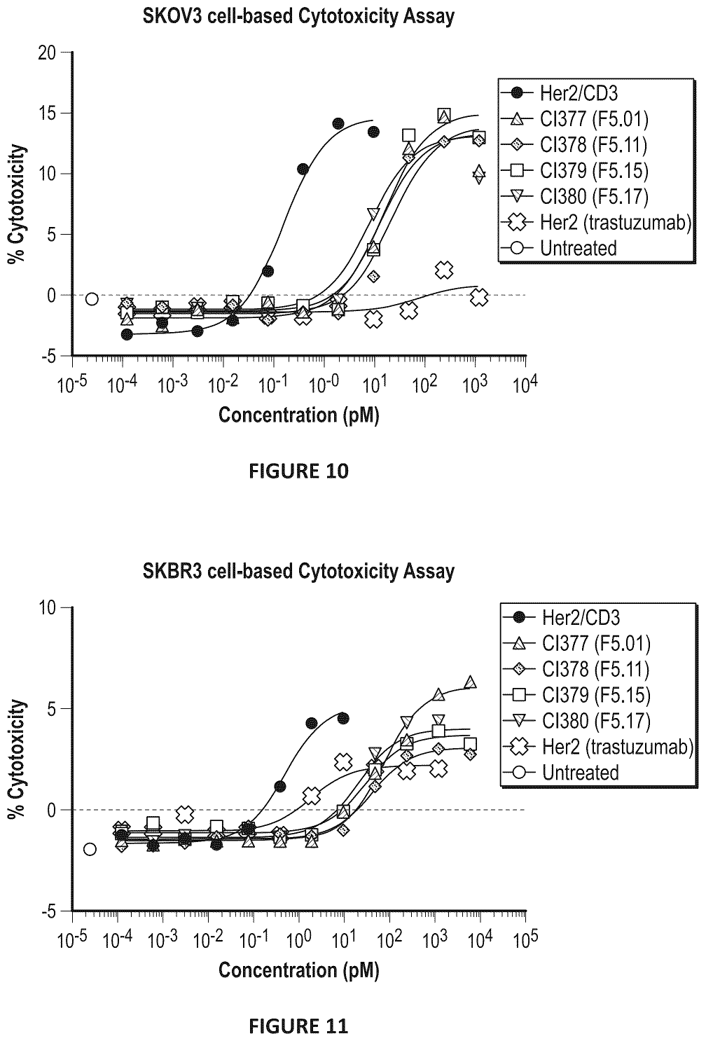

The cytotoxicity of the activatable divalent, bispecific anti-HER2, anti-CD3 ABPCs was determined using a cell-based cytotoxicity assay. In brief, human PBMCs were co-cultured with HER2-expressing cells (e.g., SKOV3 and SKBR3) at a ratio of 10:1 in RPMI-1640+glutamax supplemented with 5% heat inactivated human serum (Sigma, Cat. No. H3667). Titrations of the activatable ABPCs, the corresponding divalent, bispecific anti-HER2, anti-CD3 ABPC (without the anti-HER2 mask (MM1) and the anti-CD3 mask (MM2)) control, trastuzumab control, and untreated cells were tested. After 48 hours, cytotoxicity was evaluated using the CytoTox-Glo™ Cytotoxicity Assay (Promega, Cat. G9292). Luminescence was measured on the Infinite M200 Pro (Tecan). Percent cytotoxicity was calculated and plotted in Prism (Graph Pad) with curve fit analysis using three parameters least squares fit of the log vs. response curve fit, and the effective concentration. EC50, was determined. The cytotoxicity curves (FIGS. 10 and 11) showed that the activatable ABPCs exhibited have reduced cytotoxic activity compared the control. The magnitude in difference in binding between the Her2/CD3 bispecific control and the activatable ABPCs was quantified as a fold difference in binding computed as the EC50 of the activatable ABPC divided by the EC50 of the HER2/CD3 bispecific control. The results obtained for binding to HER2-expressing SKOV3 cells are provided in Table 8 and FIG. 10.

TABLE 8
Cytotoxicity (EC50) in an SKOV3 Cell-Based Assay

| | | | Fold Reduction |
|---|---|---|---|
| | | | in HER2 Binding |
| | | | Relative to Control |
| | HER2 | | [EC50(molecule)/ |
| Molecule | Mask | EC50, pM | EC50(control)] |
| Her2/CD3 control | N/A | 0.12 | 1.00 |
| CI377 | F5.01 | 12.13 | 104.66 |
| CI378 | F5.11 | 21.67 | 186.97 |
| CI379 | F5.15 | 15.62 | 134.77 |
| CI380 | F5.17 | 8.172 | 70.51 |

The results obtained for binding to HER2-expressing SKBR3 cells are provided in Table 9 and FIG. 11.

TABLE 9
Cytotoxicity (EC50) in an SKBR3 Cell-Based Assay

| | | | Fold Reduction in HER2 |
|---|---|---|---|
| | HER2 | | Binding Relative to Control |
| Molecule | Mask | EC50, pM | EC50(molecule)/EC50(control)] |
| Her2/CD3 control | N/A | 0.28 | 1 |
| CI377 | F5.01 | 76.6 | 278.55 |
| CI378 | F5.11 | 40.76 | 148.22 |
| CI379 | F5.15 | 29.99 | 109.05 |
| CI380 | F5.17 | 19.99 | 72.69 |
| Trastuzumab | N/A | 1.912 | 6.95 |

The results indicate that the anti-HER2 masking moieties (MM1s) were highly effective at interfering with the binding of the anti-HER2 binding domain of the activatable ABPC to the HER2 target.

### Example 5: Characterization of Activatable Divalent, Bispecific Anti-HER2 ABPCs

Seven additional anti-HER2 masking moieties described in Example 1 were evaluated in the context of an activatable divalent, bispecific anti-HER2, anti-CD3 ABPC having the structure depicted in FIG. 2:
(1) CI185 with MM1, mLW001 (SEQ ID NO:39);
(2) CI385 with MM1, mLW006 (SEQ IDs NO:44);
(3) CI388 with MM1, mLW009 (SEQ ID NO:47);
(4) CI186 with MM1, mLW010 (SEQ ID NO:48);
(5) CI187 with MM1, mLW011 (SEQ ID NO:49);
(6) CI389 with MM1, mLW012 (SEQ ID NO:50);
(7) CI391 with MM1, mLW014 (SEQ ID NO:52); and
(8) CI124 with MM1, F5.17 (SEQ ID NO:33, control).
   a. Binding to HER2⁺ cells and CD3⁺ Jurkat Cells

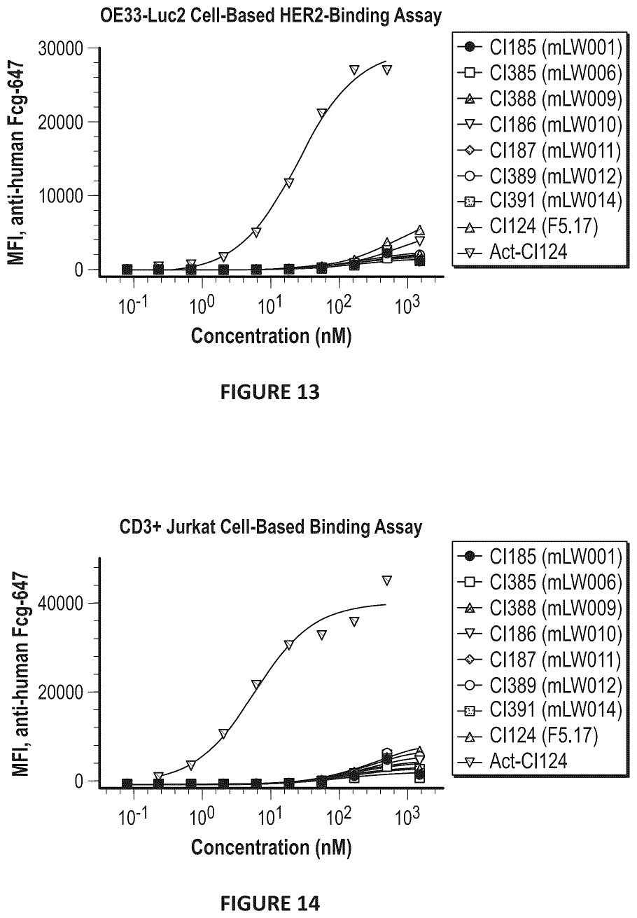

Binding inhibition of the activatable divalent, bispecific ABPCs was evaluated using a panel of HER2-expressing cells and CD3-expressing Jurkat cells. Briefly. HER2-expressing cells (OE33-Luc2) and CD3-expressing cells (Jurkat-NFAT cells (Signosis Inc., Cat. No. SL-0032)) were cultured in RPMI-1640+glutamax (Life Technologies. Cat. No. 72400-047), 10% Heat Inactivated-Fetal Bovine Serum (HI-FBS, Life Technologies, Cat. No. 10438-026) according to manufacturer guidelines. Adherent cell lines were detached using Accutase®, washed, and incubated for one hour with titrations of each activatable divalent, bispecific anti-HER2, anti-CD3 ABPC, as well as the activated CI124 control. Cells were then washed and incubated with Alexa Fluor®647-conjugated Anti-human IgG Fc-gamma fragment specific antibody (Jackson ImmunoResearch Laboratories, Cat #109-605-098) for 30 minutes. Cells were fixed in 1% PFA for 15 minutes and washed twice in PBS. Fixed cells were evaluated via flow cytometry. Plots were generated in Prism (GraphPad). The data were fit using three parameters least squares fit of the log vs, response curve fit (FIGS. 13 (binding of OE33-Luc2 cells) and 14 (binding of Jurkat-NFAT cells), and the effective concentration, EC₅₀, was determined. A comparison of the results for the activatable ABPC with the activated control (Act-CI124) indicated that the anti-HER2 masking moieties (MM1s) were effective at significantly reducing binding of the activatable ABPC to HER2 expressing cell lines.

### B. Aggregation and Yield of Activatable Divalent, Bispecific Anti-HER2, Anti-CD3 ABPCs

The monomer content of purified preparations of the above-described activatable ABPCs was analyzed using analytical SEC as described in Example 4B. The percentage in each purified preparation is provided in Table 10.

TABLE 10
Percent Monomer

| | HER2 | | Protein A | |
|---|---|---|---|---|
| Molecule | Mask | Protein | Octet (mg/L) | % Monomer |
| CI185 | mLW001 | mLW001-2014-HER2Fcmt4- | 39.4 | 90.7 |
| | | h20GG-2014-L15-v12sc-H-N | | |
| CI385 | mLW006 | mLW006-2014-HER2Fcmt4- | 27.3 | 93.4 |
| | | h20GG-2014-L15-v12sc-H-N | | |
| CI388 | mLW009 | mLW009-2014-HER2Fcmt4- | 35.6 | 92.0 |
| | | h20GG-2014-L15-v12sc-H-N | | |
| CI186 | mLW010 | mLW010-2014-HER2Fcmt4- | 52.1 | 91.5 |
| | | h20GG-2014-L15-v12sc-H-N | | |
| CI187 | mLW011 | mLW011-2014-HER2Fcmt4- | 45.7 | 92.7 |
| | | h20GG-2014-L15-v12sc-H-N | | |
| CI389 | mLW012 | mLW012-2014-HER2Fcmt4- | 56.8 | 88.6 |
| | | h20GG-2014-L15-v12sc-H-N | | |
| CI391 | mLW014 | mLW014-2014-HER2Fcmt4- | 70.1 | 89.1 |
| | | h20GG-2014-L15-v12sc-H-N | | |

The results indicate that the activatable divalent, bispecific anti-HER2, anti-CD3 ABPCs exhibits relatively low levels of aggregation.

### C. Cytotoxicity

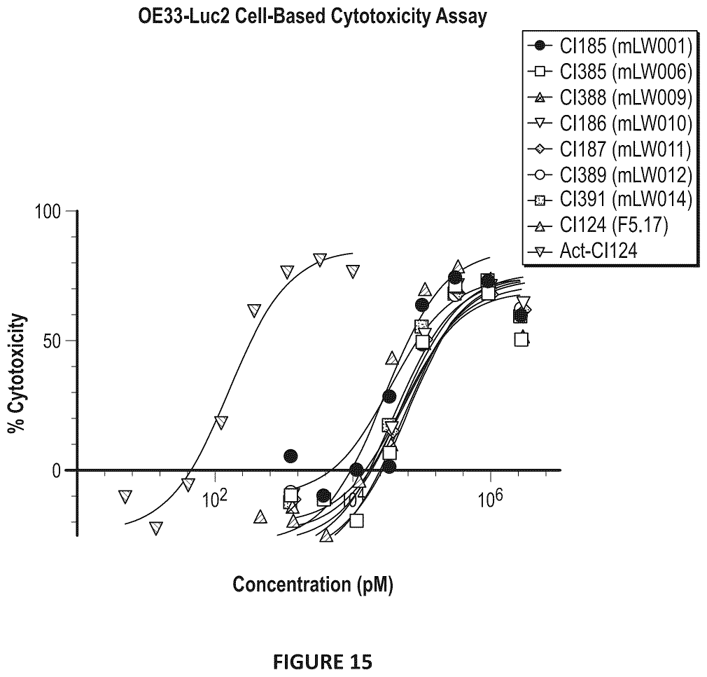

Human PBMCs were co-cultured with HER2-expressing cells (OE33-Luc2) at a ratio of 10:1 in RPMI-1640+glutamax supplemented with 5% heat-inactivated human serum (Sigma, Cat. No. H3667). Titrations of the activatable divalent, bispecific ABPCs and activated control were tested. After 48 hours, cytotoxicity was evaluated using the CytoTox-Glo™ Cytotoxicity Assay (Promega, Cat. No. G9292). Luminescence was measured on the Infinite M200 Pro (Tecan). Percent cytotoxicity was calculated and plotted in Prism (Graph Pad) with curve-fit analysis using three parameters least squares fit of the log vs. response curve fit, and the effective concentration. EC₅₀, was determined. The cytotoxicity curves (FIG. 15) show that the activatable divalent, bispecific ABPCs exhibited reduced cytotoxic activity compared to the activated control (Act-CI124). Quantification of the difference in cytotoxicity can be seen in the EC₅₀ fold-difference between multi-specific activatable antibodies and the Act-CI124 (Table 11).

TABLE 11
Cytotoxicity (EC50) in an OE33-Luc2 Cell-Based Assay

| | | | Fold Reduction in |
|---|---|---|---|
| | | | HER2 Binding |
| | | | Relative to Control |
| | | | [EC50(molecule)/ |
| Molecule | HER2 Mask | EC50 (pM) | EC50(control)] |
| Act-CI124 | N/A | 159.3 | 1.00 |
| control | | | |
| CI124 | F5.17 | 28664 | 179.94 |
| CI185 | mLW001 | 34955 | 219.43 |
| CI385 | mLW006 | 50657 | 318.00 |
| CI388 | mLW009 | 52393 | 328.90 |
| CI186 | mLW010 | 50180 | 315.00 |
| CI187 | mLW011 | 50688 | 318.19 |
| CI389 | mLW012 | 57953 | 363.80 |
| CI391 | mLW014 | 39045 | 245.10 |

### Example 6: In Vivo Tumor Regression

NSG mice with NCl-N87 tumors were used in this study. On day 0, each NSG mouse was inoculated subcutaneously with 1×10⁶ NCl-N87 cells in 100 μL RPMI+Glutamax, serum-free medium with Matrigel®. Previously frozen PBMCs from a single donor were administered (i.p.) on day 7 at a CD3⁺ T cell to tumor cell ratio of 1:1. When tumor volumes reached 150 mm³ (e.g., at approximately day 14), mice were randomized, assigned to treatment groups.

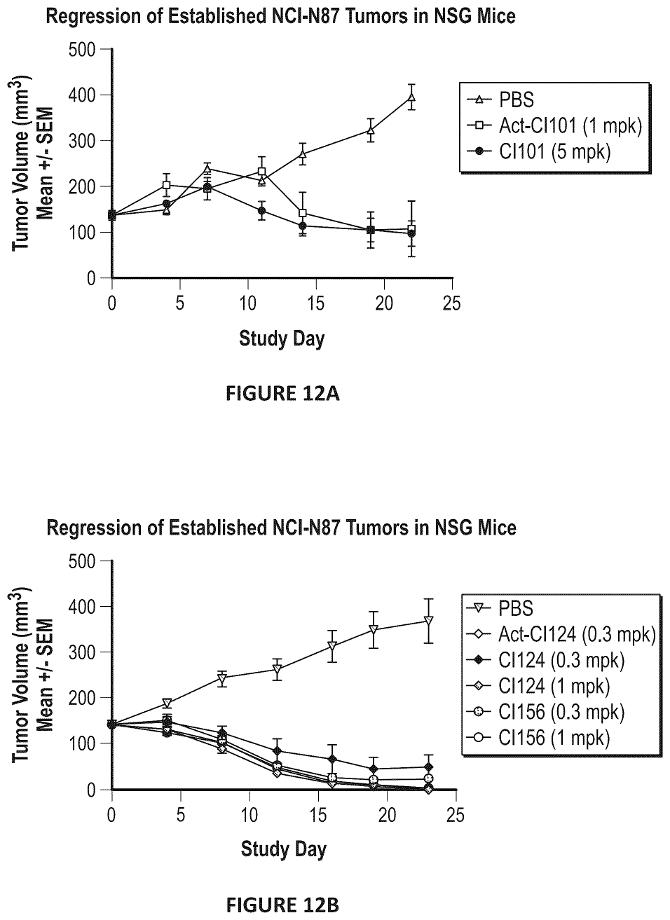

The NCG mice, with established NCl-N87 tumors and engrafted human PBMCs, were then treated intravenously with 1 mg/kg CI101 (an activatable divalent, bispecific, anti-HER2, anti-CD3 ABPC, 5 mg/kg activated CI101 (act-CI101), and a phosphate buffered saline (PBS) control are treated with multi-specific activatable antibody at Day 1, 8, and 15 for a total of three doses. Tumor volume and body weights were measured twice weekly. The results are depicted in FIG. 12A. The results showed that tumor volume for the CI1011 and Act-CI101-treated tumors decreased in volume, while the PBS-treated tumors increased in volume. The results indicate that, similar to the activated control, Act-CI01, the activatable ABPC effectively reduced tumor volume suggesting that unmasking of the anti-HER2 binding domain was successful, in vivo.

In another study, NSG mice with established NCl-N87 tumors were treated with the following:
(1) PBS, control
(2) Act-CI124, 0.3 mpk (mg/kg), control
(3) CI124 (0.8 mpk, 1 mpk)
(4) CI156 (0.3 mpk, 1 mpk)

The results are shown in FIG. 12B. The results indicate that, similar to the activated control, Act-CI124, the activatable ABPCs effectively reduced tumor volume suggesting that unmasking of the anti-HER2 binding domains in those molecules was successful, in vivo.

### Example 7: Expression, Purification, and Quality Assessment of Activatable Fc-Heterodimeric ABPCs

### A. Activatable Fc-Heterodimeric, Monovalent, Monospecific, Anti-HER2, ABPCs

Activatable Fc-heterodimeric, monovalent, monospecific, anti-HER2 ABPCs, each having Fe heterodimerization domains, were prepared. Each activatable ABPC had the structure depicted in FIG. 3. Mutations consisting of four mutations in the first Fc chain (Fc1 or HCA: T366S+L368A+Y407V+Y349C) and two complementary mutations in the second Fc chain (Fc2 or HCB: T366W+S354C) were introduced into either the human IgG1 Fc or the human IgG4 S228P Fc (human IgG4 Fc containing the S228P mutation). The numbering of the mutations is in accordance with the Kabat numbering system.

The proteins were expressed using transient transfection in Expi293® cells (Thermo Fisher Scientific, Waltham, MA, Catalog A14635). Synthetic DNA sequences encoding the proteins and signal peptides (Integrated DNA Technologies) were cloned into transient expression vectors containing the CMV promoter. Endotoxin-free plasmid DNA preparations were confirmed by DNA sequencing prior to use. Plasmid DNA (two heavy chains and one light chain in a 1:1:1 ratio) was transiently transfected into Expi293® cells using the manufacturer's recommended protocol. Two activatable Fc-heterodimeric monovalent, monospecific anti-HER2 ABPCs, “CI238” and “CI239”, were expressed using transient transfection in Expi293® cells (Thermo Fisher Scientific, Waltham, MA, Catalog A 14635). These heterodimeric monovalent, monospecific anti-HER2 ABPCs each had three polypeptides, as set forth below and in Table 12.

### First Polypeptide (“Heavy Chain A” or “HCA”):

HVD1 (SEQ ID NO:7)-CH1-Hinge (HR)-Fc1

### Second Polypeptide (“Light Chain”) (SEQ ID NO:626 (Amino Acid), SEQ ID NO:627 (Polynucleotide):

S1 (SEQ ID NO:722)-MM1 (F5.17, SEQ ID NO:33)-Linker 1-CM1 (SEQ ID NO:246)-LVD1-Linker 2-CL (SEQ ID NO:732),

### Third Polypeptide (“Heavy Chain B” or “HCB”):

Hinge-Fc2

Supernatants were harvested four days post-transfection and purified using Protein A chromatography. The purified protein samples were analyzed for monomer content and yield using analytical SEC. The monomeric population for expressed protein CI238 was purified using a second SEC step using a preparative-scale Superdex® 200 10/30 GL column (GE Healthcare Life Sciences, Catalog #17517501) in Ix PBS, pH 7.2 running buffer at 0.45 ml/min. A second purification step was used for 720 μg of CI238 which was purified by SEC resulting in 100% monomer.

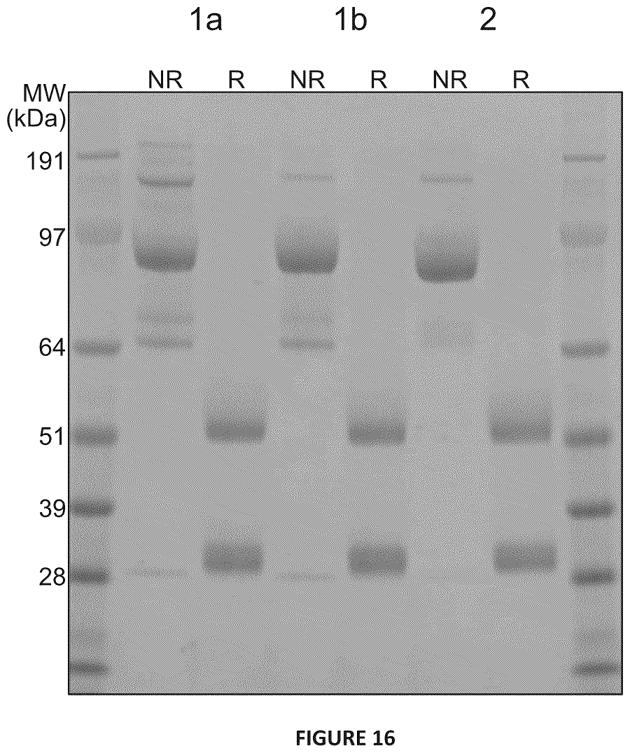

Purified proteins were analyzed by reducing and non-reducing SDS-PAGE. Protein aliquots (3-5 μg) were denatured for 10 min at 75° C., in sample buffer (with reducing agent added, as necessary) and separated on a 4-12% NuPAGE™ Bis-Tris gel (Thermo Fisher Scientific, Waltham, MA, Catalog #NP0321) in MOPS buffer for 1 hour at 15V and visualized after staining with InstantBlue™ for 1 hour followed by destaining in water for at least 4 hours. FIG. 16 depicts an image of the gel. The activatable heterodimeric, monovalent, monospecific ABPCs are listed below in Table 12 along with % monomer and yield. FIG. 16 SDS PAGE gel lanes 1a: CI238 heterodimeric-IgG1 monovalent HER2 after a single protein-A purification; 1b: sample 1a after size-exclusion chromatography (SEC) purification to enrich heterodimer; 2: CI239 heterodimeric-IgG4 monovalent HER2 after a single protein-A purification.

TABLE 12
Activatable Fc-heterodimeric, monovalent, monospecific
anti-HER2 ABPC, Monomer Content and Yield:

| | HCA SEQ | HCA | | HCB SEQ | | | | % |
|---|---|---|---|---|---|---|---|---|
| | ID NO: | CH1 - | HCA | ID NO: | HCB | HCB | | Monomer |
| Name | AA/PN | Hinge | Fc1 | AA/PN | Hinge | Fc2 | Purification | (Yield) |
| CI238 | 706/707 | huIgG1¹ | [T366S + | 708/709 | huIgG1¹ | [T366W + | Protein A | 86.4 |
| | | | L368A + | | | S354C] | | (24 μg/ml) |
| | | | Y407V + | | | IgG1 Fc | Protein A + | 100 |
| | | | Y349C] | | | | SEC SEC | (5 μg/ml) |
| | | | IgG1 Fc | | | | Input 720 | |
| | | | | | | | μg SEC | |
| | | | | | | | Output | |
| | | | | | | | 156 μg | |
| CI239 | 710/711 | huIgG4² | [S228P + | 712/713 | huIgG4² | [T366W + | Protein A | 96.5 |
| | | | T366S + | | | S354C] | | (15 μg/ml) |
| | | | L368A + | | | IgG4 Fc | | |
| | | | Y407V + | | | | | |
| | | | Y349C] | | | | | |
| | | | IgG4 | | | | | |

¹human IgG1 CH1 (SEQ ID NO: 730) and Hinge (SEQ ID NO: 733)
²human IgG4 CH1 (SEQ ID NO: 731) and huIgG4 Hinge (SEQ ID NO: 734)

All proteins were confirmed to contain three polypeptides of the expected, molecular weights on reducing gels (˜50 KDa, ˜25Ka and ˜25 KDa for the monovalent-antibody format, see FIG. 16). The purified preparations exhibited relatively high monomer content (i.e., relatively low levels of aggregation).

### B. Activatable Fc-Heterodimeric Monovalent, Bispecific, Anti-HER2, ABPCs

Activatable Fc-heterodimeric, monovalent, bispecific, anti-HER2, anti-CD3 ABPCs were prepared. Each activatable ABPC had the structure depicted in FIG. 3. Mutations consisting of four mutations in the first Fc chain (Fc1 or HCA: T366S+L368A+Y407V+Y349C) and two complementary mutations in the second Fc chain (Fc2 or HCB: T366W+S354C) were introduced into the human IgG4 S228P Fc (human IgG4 Fc containing the S228P mutation). The numbering of the mutations is in accordance with the Kabat numbering system.

First, second, and third polypeptides of activatable Fc-heterodimeric ABPCs were expressed using transient transfection in Expi293® cells (Thermo Fisher Scientific, Waltham. MA, Catalog A14635). Synthetic DNA sequences encoding the proteins and signal peptides (Integrated DNA Technologies) were cloned into transient expression vectors containing the CMV promoter. Endotoxin-free plasmid DNA preparations were confirmed by DNA sequencing prior to use. Plasmid DNA (two heavy chains and one light chain in a 1:1:1 ratio) was transiently transfected into Expi293® cells using the manufacturer's recommended protocol. Supernatants were harvested four days post-transfection and purified using Protein A chromatography. The monomeric population for each expressed protein was purified by SEC using a preparative-scale Superdex® 200 10/30 GL column (GE Healthcare Life Sciences, Catalog #17517501) in 1×PBS pH 7.2 running buffer.

Each of the activatable monovalent, bispecific ABPCs had the structure:

### 1st Polypeptide (Heavy Chain a (HCAD) (SEQ ID NO:706 (Amino Acid): SEQ ID NO:707 (Polynucleotide)):

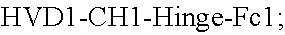

### 2^{nd} Polypeptide (Light Chain) (SEQ ID NO: 626 (Amino Acid): SEQ ID NO:627 (Polynucleotide)):

S1 (SEQ ID NO:722)-MM1 (F5.17, SEQ ID NO:33)-L1-CM-L2-LVD1 (SEQ ID NO:8)-CL (SEQ ID NO:732);

### 3^{rd} Polypeptide (Heavy Chain B (HCB))

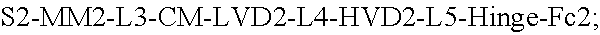

in which:
Fc1 is [S228P+T366S+L368A+Y407V+Y349C] huIgG4 Fc; and
Fc2 is [T366W+S354C] IgG4 Fc.

The HCB sequences for each of the activatable Fc-heterodimeric, monovalent, anti-HER2, anti-CD3 ABPCs were the same except for the linker between the HVD2 and the Hinge.

Monomer content of purified proteins was analyzed using analytical SEC. Protein aliquots (25 μg) were injected onto Superdex 200 Increase 5/150 GL column (GE Healthcare Life Sciences, Catalog #28906561) that was run with 1×PBS, pH 7.2 at 0.45 mL/min. The heterodimeric protein presence was confirmed by the presence of a predominant band at 100 KDa on non-reducing gels. A description of the molecules, their yield, and the percent monomer are provided in Table 13, below. The results indicate that this format provides good yield and low levels of aggregation.

TABLE 13
Activatable Fc-heterodimeric, monovalent,
dual-masked bispecific anti-HER2,
anti CD3 ABPC

| | HCB | | Yield | |
|---|---|---|---|---|
| | SEQ ID | | from | |
| Molecule | NO: | Linker, | Protein A | % |
| Name | AA/PN | L5 | (μg/ml) | Monomer |
| CI152 | 714/715 | GS | 12 | 94.4 |
| CI240 | 716/717 | GGGGSGS | 13 | 93.3 |
| | | (SEQ ID | | |
| | | NO: 738) | | |
| CI148 | 718/740 | GGGGSGGG | 11 | 94.3 |
| | | GSGGGGSG | | |
| | | S | | |
| | | (SEQ ID | | |
| | | NO: 739) | | |

### Example 8. HER2-Dependent Cytotoxicity of Exemplary Activatable Fc-Heterodimeric, Monovalent, Bispecific Anti-HER2, Anti-CD3 ABPCs

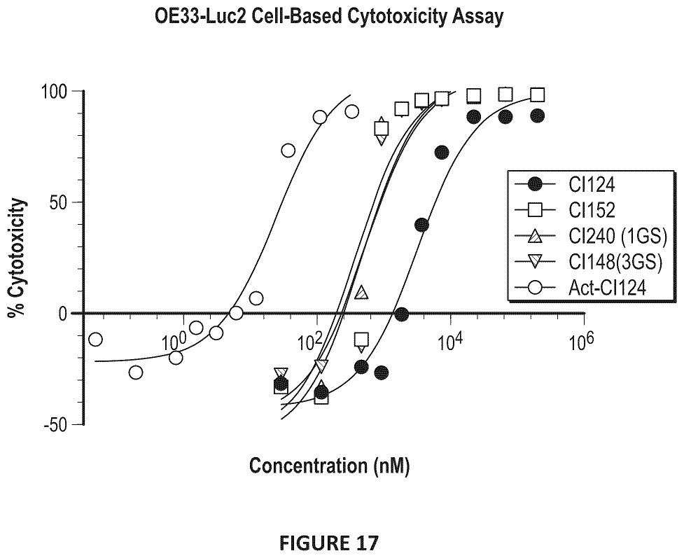

To determine if the activatable Fc-heterodimeric, monovalent, bispecific anti-HER2, anti-CD3 ABPCs (CI152, CI240, and CI148) could attenuate cell killing relative to the unmasked (protease-activated) forms (controls), a cytotoxicity assay was performed. Fresh human PBMCs were purchased (All Cells) and frozen in aliquots. Frozen PBMCs were thawed and co-cultured with HER2 expressing OE33-Luc2 cells at a ratio of 10:1 in RPMI-1640+glutamax supplemented with 5% heat inactivated human serum (Sigma, Catalog H3667). Titrations of the activatable ABPCs and controls were compared to activated and masked forms of CI124 an activatable Fc-homodimeric, divalent, bispecific anti-HER2, anti-CD3 ABPC. After 48 hours, cytotoxicity was evaluated using the ONE-Glo™ Luciferase Assay System (Promega, Catalog E6130). Luminescence was measured on the Infinite M200 Pro (Tecan). Percent cytotoxicity was calculated and plotted in GraphPad PRISM with curve fit analysis. The data in Table 14 and FIG. 17 shows that CI152, CI240, and CI148 ABPCs all induce cytotoxicity against the HER2 positive cells, and the cytotoxicity is attenuated compared to the activated Act-CI124 ABPC control.

TABLE 14
EC50

| pM | CI124 | CI152 | CI240 (1GS) | CI148 (3GS) | Act-CI124 |
|---|---|---|---|---|---|
| EC50 | 9527 | 1394 | 1145 | 1571 | 71.8 |
| Fold over | 132.69 | 19.42 | 15.95 | 21.88 | 1.00 |
| Act-CI124 | | | | | |

### Table of Exemplary Sequences

Provided below in Table 15 are exemplary sequences that can be used, in any combination, in any of the ABPCs described herein.

TABLE 15
Exemplary sequences

| | SEQ | |
|---|---|---|
| Name | ID NO: | SEQUENCE |
| Trastuzumab | 1 | DTYIH |
| HVD1 CDR1 | | |
| Trastuzumab | 2 | RIYPTNGYTRYADSVKG |
| HVD1 CDR2 | | |
| Trastuzumab | 3 | WGGDGFYAMDY |
| HVD1 CDR3 | | |
| Trastuzumab | 4 | RASQDVNTAVA |
| LVD1 CDR1 | | |
| Trastuzumab | 5 | SASFLYS |
| LVD1 CDR2 | | |
| Trastuzumab | 6 | QQHYTTPPT |
| LVD1 CDR3 | | |
| Trastuzumab | 7 | EVQLVESGGGLVQPGGSLRLSCAASGFNIKDTYIHWVRQAPGKGLEW |
| VH Domain | | VARIYPTNGYTRYADSVKGRFTISADTSKNTAYLQMNSLRAEDTAVYY |
| | | CSRWGGDGFYAMDYWGQGTLVTVSS |
| Trastuzumab | 8 | DIQMTQSPSSLSASVGDRVTITCRASQDVNTAVAWYQQKPGKAPKLLI |
| VL Domain | | YSASFLYSGVPSRFSGSRSGTDFTLTISSLQPEDFATYYCQQHYTTPPTF |
| | | GQGTKVEIK |
| Trastuzumab | 9 | EVQLVESGGGLVQPGGSLRLSCAASGFNIKDTYIHWVRQAPGKGLEW |
| HC | | VARIYPTNGYTRYADSVKGRFTISADTSKNTAYLQMNSLRAEDTAVYY |
| | | CSRWGGDGFYAMDYWGQGTLVTVSSASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPG |
| Trastuzumab | 10 | DIQMTQSPSSLSASVGDRVTITCRASQDVNTAVAWYQQKPGKAPKLLI |
| LC | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| MM1 | 11 | MSELCLSEYRCWGLE |
| MM1 | 12 | MTMCGEFMFLGCETR |
| MM1 | 13 | GRMTCADHPKCNYTY |
| MM1 | 14 | PICAFVYNDPECSWW |
| MM1 | 15 | SWCMMHHEGNVCQTV |
| MM1 | 16 | QFACFETRSQYCDWK |
| MM1 | 17 | MLWCDTPDGSCWWRE |
| MM1 | 18 | ICALTIFSTSVDCLY |
| MM1 | 19 | DGYVCHWEWACRTAA |
| MM1 | 20 | LDCVRTLYYSDCYHR |
| MM1 | 21 | LCVMDHIYDTMNCNQ |
| MM1 | 22 | PFKCDLTNPTCTILM |
| MM1 | 23 | QFCRNYPLSMMCCKY |
| MM1 | 24 | ICSYNWHFDTIDCNS |
| MM1 | 25 | SCYVLLDGILMPSDC |
| MM1 | 26 | SCYVLLDGILMPCDS |
| MM1 | 27 | CSYVLLDGILMPSDC |
| MM | 28 | CSYVLLDGILMPCDS |
| MM1 | 29 | CCYVLLDGILMPCDC |
| MM1 | 30 | SECMISQWGPDCTVA |
| MM1 | 31 | YCLYNKLNDTINCSD |
| MM1 | 32 | RCVMDPVTDTIDCNK |
| MM1 | 33 | ALICCSDVSGLCRWC |
| MM1 | 34 | ALISCSDVSGLSRWC |
| MM1 | 35 | ALISCSDVSGLCRWS |
| MM1 | 36 | ALICSSDVSGLSRWC |
| MM1 | 37 | ALICSSDVSGLCRWS |
| MM1 | 38 | SSCRTTGLMTTCDLD |
| MM1 | 39 | LYCTEGRWYEGTCA |
| MM1 | 40 | LTKCATSTSTTCDLD |
| MM1 | 41 | FGCGVVLDLDHCAPW |
| MM1 | 42 | TPCWHMIMTNPECMR |
| MM1 | 43 | YNACWFGPYDCWYRP |
| MM1 | 44 | FLCEPPSWGPDCTMI |
| MM1 | 45 | DLCSFGLWYRGTCEG |
| MM1 | 46 | TCYHDMHKDTIHCNN |
| MM1 | 47 | LYRDMCGGGCEDWHD |
| MM | 48 | LGSLCDPFDCPGKNL |
| MM1 | 49 | RGCSFGYWGNGTCLG |
| MM1 | 50 | RACSFGVWWKGTCEG |
| MM1 | 51 | ANVCWLGPYDCRWES |
| MM1 | 52 | GMCTSGRWYGGDCTG |
| MM1 | 53 | ACTHNTMTDKIYCNN |
| CM | 54 | AANALAHGLF |
| CM | 55 | AANL |
| CM | 56 | AANLGSGGSS |
| CM | 57 | AAPRS |
| CM | 58 | AAPRSF |
| CM | 59 | AARGPAIH |
| CM | 60 | AAYHLVSQ |
| CM | 61 | AFPDMRSVRS |
| CM | 62 | AFQALRM |
| CM | 63 | AFRHLR |
| CM | 64 | AGLGISST |
| CM | 65 | AGLGVVER |
| CM | 66 | AGPR |
| CM | 67 | AHGL |
| CM | 68 | AHGLF |
| CM | 69 | AHQALRM |
| CM | 70 | AIPRVRLFDV |
| CM | 71 | ALAHG |
| CM | 72 | ALAHGL |
| CM | 73 | ALAHGLF |
| CM | 74 | ALAHGLFAPRSF |
| CM | 75 | ALAHGLESGRSAN |
| CM | 76 | ALAHGLPTFVHL |
| CM | 77 | ALGLLRLP |
| CM | 78 | ALPSVKMVSE |
| CM | 79 | ALRAP |
| CM | 80 | ANQALRM |
| CM | 81 | ANQALRMA |
| CM | 82 | APPLVKSMVV |
| CM | 83 | APPSFKLVNA |
| CM | 84 | APRS |
| CM | 85 | APRSALAHGLF |
| CM | 86 | APRSF |
| CM | 87 | AQFVLTEG |
| CM | 88 | AQNLLGMV |
| CM | 89 | ARGP |
| CM | 90 | ARGPS |
| CM | 91 | ARGPSF |
| CM | 92 | ARGPSFK |
| CM | 93 | ASGLLRFP |
| CM | 94 | ASPTMKTVGL |
| CM | 95 | AVGLLAPP |
| CM | 96 | AVGLLAPPGGLSGRSANI |
| CM | 97 | AVGLLAPPGGLSGRSANP |
| CM | 98 | AVGLLAPPGGLSGRSDDH |
| CM | 99 | AVGLLAPPGGLSGRSDIH |
| CM | 100 | AVGLLAPPGGLSGRSDNH |
| CM | 101 | AVGLLAPPGGLSGRSDNI |
| CM | 102 | AVGLLAPPGGLSGRSDNP |
| CM | 103 | AVGLLAPPGGLSGRSDQH |
| CM | 104 | AVGLLAPPGGLSGRSDTH |
| CM | 105 | AVGLLAPPGGLSGRSDYH |
| CM | 106 | AVGLLAPPGGLSGRSNI |
| CM | 107 | AVGLLAPPGGLSGRSNIG |
| CM | 108 | AVGLLAPPGGLSGRSNIGS |
| CM | 109 | AVGLLAPPGGTSTSGRSANPRG |
| CM | 110 | AVGLLAPPSGRSANPRG |
| CM | 111 | AVGLLAPPTSGRSANPRG |
| CM | 112 | AVPKVRVVPE |
| CM | 113 | CGPPLGR |
| CM | 114 | CSPPLGR |
| CM | 115 | DEVDGSGGSS |
| CM | 116 | DISHWRRS |
| CM | 117 | DLAHPLL |
| CM | 118 | DLPLVKSLPS |
| CM | 119 | DRLSGRSANHKK |
| CM | 120 | DRLSGRSDNHKK |
| CM | 121 | DRPEMKSLSG |
| CM | 122 | DRPKVKTMDF |
| CM | 123 | DVAQFVLT |
| CM | 124 | DVPPMKTLRP |
| CM | 125 | DWLYWMGI |
| CM | 126 | DWLYWMSI |
| CM | 127 | DWLYWPGI |
| CM | 128 | DWLYWPSI |
| CM | 129 | EAPKVKALPK |
| CM | 130 | EHPRVKVVSE |
| CM | 131 | EKPRMKLFQG |
| CM | 132 | EPQALAMS |
| CM | 133 | EQPEVKMVKG |
| CM | 134 | ERPGVKSLVL |
| CM | 135 | ESLPVVAV |
| CM | 136 | ESPVMKSMAL |
| CM | 137 | ESRRW |
| CM | 138 | ESRRWM |
| CM | 139 | ESRRWMP |
| CM | 140 | ETPSVKTMGR |
| CM | 141 | ETPSVKTMGRSS |
| CM | 142 | FPRPLGITGL |
| CM | 143 | FRLLDWQW |
| CM | 144 | GCGPPLGR |
| CM | 145 | GCSPPLGR |
| CM | 146 | GFPHMKTFQH |
| CM | 147 | GFPHMKTFQHSS |
| CM | 148 | GGGPPLGR |
| CM | 149 | GGPPLGR |
| CM | 150 | GGQPSGMWGW |
| CM | 151 | GGSIDGR |
| CM | 152 | GGSPPLGR |
| CM | 153 | GGWHTGRN |
| CM | 154 | GIAGQ |
| CM | 155 | GLGTPRGLFA |
| CM | 156 | GLPTFV |
| CM | 157 | GLPTFVH |
| CM | 158 | GLPTFVHL |
| CM | 159 | GLPTFVHLPRQV |
| CM | 160 | GLSGRSDNHGGAVGLLAPP |
| CM | 161 | GLSGRSDNHGGVHMPLGFLGP |
| CM | 162 | GLSGRSDNHGSS |
| CM | 163 | GPEGLRVG |
| CM | 164 | GPLGIAGI |
| CM | 165 | GPLNGRSDNHKA |
| CM | 166 | GPLNGRSDNHKK |
| CM | 167 | GPLNGRSDNHKR |
| CM | 168 | GPLNGRSDNHQA |
| CM | 169 | GPLNGRSDNHQK |
| CM | 170 | GPLNGRSDNHQR |
| CM | 171 | GPLNGRSDNHRA |
| CM | 172 | GPLNGRSDNHRK |
| CM | 173 | GPLNGRSDNHRR |
| CM | 174 | GPLSGRSDNHKA |
| CM | 175 | GPLSGRSDNHKK |
| CM | 176 | GPLSGRSDNHKR |
| CM | 177 | GPLSGRSDNHQA |
| CM | 178 | GPLSGRSDNHQK |
| CM | 179 | GPLSGRSDNHQR |
| CM | 180 | GPLSGRSDNHRA |
| CM | 181 | GPLSGRSDNHRK |
| CM | 183 | GPLSGRSDNHRR |
| CM | 184 | GPPLGR |
| CM | 185 | GPQGIAGQ |
| CM | 186 | GPQGLLGA |
| CM | 187 | GPRSFG |
| CM | 188 | GPRSFGL |
| CM | 189 | GPSHLVLT |
| CM | 190 | GPTN |
| CM | 191 | GPTNALAHGLF |
| CM | 192 | GRSML |
| CM | 193 | GRSMLL |
| CM | 194 | GRSMLLG |
| CM | 195 | GRSMLLGG |
| CM | 196 | GRSMLLGP |
| CM | 197 | GRSMLLGS |
| CM | 198 | GRSMLLP |
| CM | 199 | GRSMLLPG |
| CM | 200 | GRSMLLPP |
| CM | 201 | GRSMLLPS |
| CM | 202 | GRSMLLS |
| CM | 203 | GRSMLLSG |
| CM | 204 | GRSMLLSP |
| CM | 205 | GRSMLLSS |
| CM | 206 | GRSMLM |
| CM | 207 | GRSMLMG |
| CM | 208 | GRSMLMGG |
| CM | 209 | GRSMLMGP |
| CM | 210 | GRSMLMGS |
| CM | 211 | GRSMLMP |
| CM | 212 | GRSMLMPG |
| CM | 213 | GRSMLMPP |
| CM | 214 | GRSMLMPS |
| CM | 215 | GRSMLMS |
| CM | 216 | GRSMLMSG |
| CM | 217 | GRSMLMSP |
| CM | 218 | GRSMLMSS |
| CM | 219 | GSGPPLGR |
| CM | 220 | GSPPLGR |
| CM | 221 | GSSPPLGR |
| CM | 222 | GTGRGPSWVGSS |
| CM | 223 | HMMQYARH |
| CM | 224 | HTGRSGAL |
| CM | 225 | HVPRQ |
| CM | 226 | HVPRQV |
| CM | 227 | HVPRQVAPRSF |
| CM | 228 | HVPRQVLSGRS |
| CM | 229 | HVPRQVLSGRSAN |
| CM | 230 | HWHLGPPT |
| CM | 231 | IANLLSMV |
| CM | 232 | IDGR |
| CM | 233 | IEGR |
| CM | 234 | ILNLLSMV |
| CM | 235 | ILPRSPAF |
| CM | 236 | IPFSWSRF |
| CM | 237 | IQNLLSMV |
| CM | 238 | ISSGL |
| CM | 239 | ISSGLL |
| CM | 240 | ISSGLLS |
| CM | 241 | ISSGLLSGRSANI |
| CM | 242 | ISSGLLSGRSANP |
| CM | 243 | ISSGLLSGRSANPRG |
| CM | 244 | ISSGLLSGRSDDH |
| CM | 245 | ISSGLLSGRSDIH |
| CM | 246 | ISSGLLSGRSDNH |
| CM | 247 | ISSGLLSGRSDNI |
| CM | 248 | ISSGLLSGRSDNP |
| CM | 249 | ISSGLLSGRSDQH |
| CM | 250 | ISSGLLSGRSDTH |
| CM | 251 | ISSGLLSGRSDYH |
| CM | 252 | ISSGLLSGRSGNH |
| CM | 253 | ISSGLLSGRSNI |
| CM | 254 | ISSGLLSGRSNIG |
| CM | 255 | ISSGLLSGRSNIGS |
| CM | 256 | ISSGLLSS |
| CM | 257 | ISSGLLSSGGSGGSLSGRSDNH |
| CM | 258 | ISSGLLSSGGSGGSLSGRSGNH |
| CM | 259 | ISSGLSS |
| CM | 260 | IVSRSA |
| CM | 261 | KGLTGRSDRHQA |
| CM | 262 | KGPKVKVVTL |
| CM | 263 | KNLYGRSENNGN |
| CM | 264 | KRMPVQFL |
| CM | 265 | LAAPLGLL |
| CM | 266 | LAHG |
| CM | 267 | LAHGL |
| CM | 268 | LAHGLF |
| CM | 269 | LAPLGLQRR |
| CM | 270 | LARAG |
| CM | 271 | LARAGI |
| CM | 272 | LARAGL |
| CM | 273 | LKAAPRWA |
| CM | 274 | LKAAPRWF |
| CM | 275 | LKAAPVWA |
| CM | 276 | LKAAPVWF |
| CM | 277 | LKGRSYYY |
| CM | 278 | LLAPSHRA |
| CM | 279 | LLEALRAL |
| CM | 280 | LLESLRAL |
| CM | 281 | LLLPAHGG |
| CM | 282 | LLLPLLGS |
| CM | 283 | LLNALRAL |
| CM | 284 | LLNSLRAL |
| CM | 285 | LLQALRAL |
| CM | 286 | LLQSLRAL |
| CM | 287 | LLSALRAL |
| CM | 288 | LLSSLRAL |
| CM | 289 | LNGRSDNH |
| CM | 290 | LPAGLLL |
| CM | 291 | LPAGLLLR |
| CM | 292 | LPAHLVLL |
| CM | 293 | LPAHLVLV |
| CM | 294 | LPGGLSPW |
| CM | 295 | LPSHLVLL |
| CM | 296 | LPSHLVLV |
| CM | 297 | LPTFV |
| CM | 298 | LPTFVH |
| CM | 299 | LPTFVHL |
| CM | 300 | LRSGW |
| CM | 301 | LSGR |
| CM | 302 | LSGRS |
| CM | 303 | LSGRSA |
| CM | 304 | LSGRSALAHGLF |
| CM | 305 | LSGRSAN |
| CM | 306 | LSGRSANI |
| CM | 307 | LSGRSANP |
| CM | 308 | LSGRSD |
| CM | 309 | LSGRSDD |
| CM | 310 | LSGRSDDH |
| CM | 311 | LSGRSDI |
| CM | 312 | LSGRSDIH |
| CM | 313 | LSGRSDN |
| CM | 314 | LSGRSDNH |
| CM | 315 | LSGRSDNHGGAVGLLAPP |
| CM | 316 | LSGRSDNHGGSGGSISSGLLSS |
| CM | 317 | LSGRSDNHGGSGGSQNQALRMA |
| CM | 318 | LSGRSDNHGGVHMPLGFLGP |
| CM | 319 | LSGRSDNI |
| CM | 320 | LSGRSDNP |
| CM | 321 | LSGRSDQ |
| CM | 322 | LSGRSDQH |
| CM | 323 | LSGRSDT |
| CM | 324 | LSGRSDTH |
| CM | 325 | LSGRSDY |
| CM | 326 | LSGRSDYH |
| CM | 327 | LSGRSENH |
| CM | 328 | LSGRSG |
| CM | 329 | LSGRSGN |
| CM | 330 | LSGRSGNH |
| CM | 331 | LSGRSGNHGGSGGSISSGLLSS |
| CM | 332 | LSGRSGNHGGSGGSQNQALRMA |
| CM | 333 | LSGRSGNP |
| CM | 334 | LSGRSVTQ |
| CM | 335 | LSQARWRK |
| CM | 336 | LTFPTYIF |
| CM | 337 | LTFPTYWF |
| CM | 338 | LTGRSDRH |
| CM | 339 | LTGRSGA |
| CM | 340 | LYAAPRWA |
| CM | 341 | LYAAPRWF |
| CM | 342 | LYAAPVWA |
| CM | 343 | LYAAPVWF |
| CM | 344 | LYGRSENN |
| CM | 345 | MDAFLESS |
| CM | 346 | MGLFSEAG |
| CM | 347 | MGPWFM |
| CM | 348 | MIAPVAYR |
| CM | 349 | MLRSGW |
| CM | 350 | MLRSGWR |
| CM | 351 | MLRSGWRG |
| CM | 352 | MLRSGWRL |
| CM | 353 | MLRSGWRS |
| CM | 354 | MTFPTYIF |
| CM | 355 | MTFPTYWF |
| CM | 356 | MVLGRSLL |
| CM | 357 | NHRIGRSDNHRR |
| CM | 358 | NMPSFKLVTG |
| CM | 359 | NTLSGRSENHSG |
| CM | 360 | NTLSGRSGNHGS |
| CM | 361 | NZPRVRLVLP |
| CM | 362 | PAGLWLDP |
| CM | 363 | PAGRR |
| CM | 364 | PAGRRS |
| CM | 365 | PAGRRSL |
| CM | 366 | PASLWYTQ |
| CM | 367 | PESRRWMP |
| CM | 368 | PFHLSR |
| CM | 369 | PHGFFQ |
| CM | 370 | PLARAGI |
| CM | 371 | PLARAGL |
| CM | 372 | PLGL |
| CM | 373 | PLGLAG |
| CM | 374 | PLGLWA |
| CM | 375 | PLGVRGK |
| CM | 376 | PLTGRSGG |
| CM | 377 | PLTGRSGGGGSS |
| CM | 378 | PPLGR |
| CM | 379 | PPPDMKLFPG |
| CM | 380 | PPPEVRSFSV |
| CM | 381 | PPPVLKLLEW |
| CM | 382 | PPSIARSDNLAN |
| CM | 383 | PQHRIVSF |
| CM | 384 | PRFKIIGG |
| CM | 385 | PRFRIIGG |
| CM | 386 | PRPFVKSVDQ |
| CM | 387 | PRQV |
| CM | 388 | PRSF |
| CM | 389 | PSPPVKMMPE |
| CM | 390 | PTNGGSGGSS |
| CM | 391 | PTNL |
| CM | 392 | PTNLGSGGSS |
| CM | 393 | PVGYTSSL |
| CM | 394 | PVPRLKLIKD |
| CM | 395 | PVQPIGPQ |
| CM | 396 | QALAMSAI |
| CM | 397 | QFQALRM |
| CM | 398 | QGPMFKSLWD |
| CM | 399 | QGRAITFI |
| CM | 400 | QHQALRM |
| CM | 401 | QNQALRIA |
| CM | 402 | QNQALRM |
| CM | 403 | QNQALRMA |
| CM | 404 | QNQALRMAGGSGGSLSGRSDNH |
| CM | 405 | QNQALRMAGGSGGSLSGRSGNH |
| CM | 406 | QSRRVP |
| CM | 407 | QSRRVPL |
| CM | 408 | QSRRVPV |
| CM | 409 | QTRRVP |
| CM | 410 | QTRRVPL |
| CM | 411 | QTRRVPV |
| CM | 412 | QYIVSRSA |
| CM | 413 | RALRAP |
| CM | 414 | REPFMKSLPW |
| CM | 415 | RFPLKV |
| CM | 416 | RFPSLKSFPL |
| CM | 417 | RFPYGVW |
| CM | 418 | RFYRNQFF |
| CM | 419 | RGPA |
| CM | 420 | RGPAFNPM |
| CM | 421 | RGPATPIM |
| CM | 422 | RGPKLYW |
| CM | 423 | RHLAKL |
| CM | 424 | RIGRSDNH |
| CM | 425 | RKMPNITV |
| CM | 426 | RKSSIIIRMRDVVL |
| CM | 427 | RKTVQHWW |
| CM | 428 | RLGRSDNN |
| CM | 429 | RMHLRSLG |
| CM | 430 | RPLARAGI |
| CM | 431 | RPLARAGL |
| CM | 432 | RPLNGRSDNHKA |
| CM | 433 | RPLNGRSDNHKK |
| CM | 435 | RPLNGRSDNHKR |
| CM | 436 | RPLNGRSDNHQA |
| CM | 437 | RPLNGRSDNHQK |
| CM | 438 | RPLNGRSDNHQR |
| CM | 439 | RPLNGRSDNHRA |
| CM | 440 | RPLNGRSDNHRK |
| CM | 441 | RPLNGRSDNHRR |
| CM | 442 | RPLSGRSDNHKA |
| CM | 443 | RPLSGRSDNHKK |
| CM | 444 | RPLSGRSDNHKR |
| CM | 445 | RPLSGRSDNHQA |
| CM | 446 | RPLSGRSDNHQK |
| CM | 447 | RPLSGRSDNHQR |
| CM | 448 | RPLSGRSDNHRA |
| CM | 449 | RPLSGRSDNHRK |
| CM | 450 | RPLSGRSDNHRR |
| CM | 451 | RPSPMWAY |
| CM | 452 | RRHDGLRA |
| CM | 453 | RRHDGLRS |
| CM | 454 | RSLVFAPI |
| CM | 455 | RSPSRLKC |
| CM | 456 | RVPKVKVMLD |
| CM | 457 | SAGFSLPA |
| CM | 458 | SAPAVESE |
| CM | 459 | SAPYFRMMDM |
| CM | 460 | SARGPSRW |
| CM | 461 | SCGPPLGR |
| CM | 462 | SCSPPLGR |
| CM | 463 | SGGPLGVR |
| CM | 464 | SGGPPLGR |
| CM | 465 | SGPPLGR |
| CM | 466 | SGRS |
| CM | 467 | SGRSA |
| CM | 468 | SGRSAN |
| CM | 469 | SGRSANI |
| CM | 470 | SGRSANP |
| CM | 471 | SGRSANPRG |
| CM | 472 | SGRSD |
| CM | 473 | SGRSDD |
| CM | 474 | SGRSDDH |
| CM | 475 | SGRSDI |
| CM | 476 | SGRSDIH |
| CM | 477 | SGRSDN |
| CM | 478 | SGRSDNI |
| CM | 479 | SGRSDNP |
| CM | 480 | SGRSDQ |
| CM | 481 | SGRSDQH |
| CM | 482 | SGRSDT |
| CM | 483 | SGRSDTH |
| CM | 484 | SGRSDY |
| CM | 485 | SGRSDYH |
| CM | 486 | SGRSG |
| CM | 487 | SGRSGN |
| CM | 488 | SGRSGNH |
| CM | 489 | SGSPPLGR |
| CM | 490 | SIARSDNL |
| CM | 491 | SISSGLLSGRSDNI |
| CM | 492 | SMLRSMPL |
| CM | 493 | SNPFKY |
| CM | 494 | SPLPLRVP |
| CM | 495 | SPLTGRSG |
| CM | 496 | SPPLGR |
| CM | 497 | SPRSIMLA |
| CM | 498 | SRRVP |
| CM | 499 | SRRVPL |
| CM | 500 | SRRVPV |
| CM | 501 | SSGPPLGR |
| CM | 502 | SSPPLGR |
| CM | 503 | SSRGPAYL |
| CM | 504 | SSRHRRALD |
| CM | 505 | SSSFDKGKYKKGDDA |
| CM | 506 | SSSFDKGKYKRGDDA |
| CM | 507 | SSSPPLGR |
| CM | 508 | STFPFGMF |
| CM | 509 | STVFHM |
| CM | 510 | SVHHLI |
| CM | 511 | SVSGLLSH |
| CM | 512 | SVSGLLSS |
| CM | 513 | SVSGLRSH |
| CM | 514 | SVSGLRSS |
| CM | 515 | TARG |
| CM | 516 | TARGP |
| CM | 517 | TARGPALAHGLF |
| CM | 518 | TARGPS |
| CM | 519 | TARGPSF |
| CM | 520 | TARGPSFK |
| CM | 521 | TARGPSW |
| CM | 522 | TARGPVPRQV |
| CM | 523 | TFVH |
| CM | 524 | TGLSGRSVTQTS |
| CM | 525 | TGRGPSWV |
| CM | 526 | TLRLGRSDNNKN |
| CM | 527 | TLSGLRSP |
| CM | 528 | TSGRSANP |
| CM | 529 | TSGRSGNP |
| CM | 530 | TSLSGRSANPRG |
| CM | 531 | TSLSGRSGNPRG |
| CM | 532 | TSSGLRSP |
| CM | 533 | TSTSGRSANPRG |
| CM | 534 | TSTSGRSANPRGGGAVGLLAPP |
| CM | 535 | TSTSGRSANPRGGGVHMPLGFLGP |
| CM | 536 | TSTSGRSGNPRG |
| CM | 537 | TVSGLRSP |
| CM | 538 | VAGRSMRP |
| CM | 539 | VAPQLKSLVP |
| CM | 540 | VAQFVLTE |
| CM | 541 | VHMPLGFLGP |
| CM | 542 | VHMPLGFLGPGGLSGRSDNH |
| CM | 543 | VHMPLGFLGPGGTSTSGRSANPRG |
| CM | 544 | VLPELRSVFS |
| CM | 545 | VLSKQMSF |
| CM | 546 | VPAGRRS |
| CM | 547 | VPAGRRSL |
| CM | 548 | VPRQ |
| CM | 549 | VPRQV |
| CM | 550 | VSRSA |
| CM | 551 | VVPEGRRS |
| CM | 552 | WATPRPMR |
| CM | 553 | WDHPISLL |
| CM | 554 | YDPZVKVVLA |
| CM | 555 | YGAGLGVV |
| CM | 556 | YIVSRSA |
| CM | 557 | YKKFVGSL |
| CM | 558 | YVPRVKALEM |
| Linker | 559 | GSGGS |
| Linker | 560 | GGGS |
| Linker | 561 | GGSG |
| Linker | 562 | GGSGG |
| Linker | 563 | GSGSG |
| Linker | 564 | GSGGG |
| Linker | 565 | GGGSG |
| Linker | 566 | GSSSG |
| Linker | 567 | GSSGGSGGSGGSG |
| Linker | 568 | GSSGGSGGSGG |
| Linker | 569 | GSSGGSGGSGGS |
| Linker | 570 | GSSGGSGGSGGSGGGS |
| Linker | 571 | GSSGGSGGSG |
| Linker | 572 | GSSGGSGGSGS |
| Linker | 573 | GGGSSGGS |
| Linker | 574 | GGGGSGGGGSGGGGS |
| Linker | 575 | GGGS |
| Linker | 576 | GSSGT |
| Linker | 577 | GSSG |
| Linker | 578 | GGGSSGGSGGSGG |
| V12/SP34LvHv | 581 | TYAMN |
| HCDR1 | | |
| V12/SP34LvHv | 582 | RIRSKYNNYATYYADSVKD |
| HCDR2 | | |
| V12/SP34LvHv | 583 | HGNFGNSYVSWFAY |
| HCDR3 | | |
| V12/SP34LvHv | 584 | RSSTGAVTTSNYAN |
| LCDR1 | | |
| V12/SP34LvHv | 585 | GTNKRAP |
| LCDR2 | | |
| V12/SP34LvHv | 586 | ALWYSNLWV |
| LCDR3 | | |
| V12/V16 | 587 | EVQLVESGGGLVQPGGSLRLSCAASGFTFSTYAMNWVRQAPGKGLEW |
| HVD2 | | VARIRSKYNNYATYYADSVKDRFTISRDDSKNSLYLQMNSLKTEDTAV |
| | | YYCVRHGNFGNSYVSWFAYWGQGTLVTVSS |
| V12 | 588 | QTVVTQEPSFSVSPGGTVTLTCRSSTGAVTTSNYANWVQQTPGQAPRG |
| LVD2 | | LIGGTNKRAPGVPDRFSGSILGNKAALTITGAQADDESDYYCALWYSN |
| | | LWVFGGGTKLTVL |
| V16 | 589 | EVQLVESGGGLVQPGGSLKLSCAASGFTFSTYAMNWVRQASGKGLEW |
| LVD2 | | VGRIRSKYNNYATYYADSVKDRFTISRDDSKNTAYLQMNSLKTEDTAV |
| | | YYCTRHGNFGNSYVSWFAYWGQGTLVTVSS |
| SP34 | 590 | EVQLVESGGGLVQPGGSLKLSCAASGFTFNTYAMNWVRQAPGKGLE |
| HVD2 | | WVARIRSKYNNYATYYADSVKDRFTISRDDSKNTAYLQMNNLKTEDT |
| | | AVYYCVRHGNFGNSYVSWFAYWGQGTLVTVS |
| SP34 | 591 | QTVVTQEPSLTVSPGGTVTLTCRSSTGAVTTSNYANWVQQKPGQAPRG |
| LVD2 | | LIGGTNKRAPGTPARFSGSLLGGKAALTLSGVQPEDEAEYYCALWYSN |
| | | LWVFGGGTKLTVL |
| scFv LV12-L3- | 592 | QTVVTQEPSFSVSPGGTVTLTCRSSTGAVTTSNYANWVQQTPGQAPRG |
| HV12 | | LIGGTNKRAPGVPDRFSGSILGNKAALTITGAQADDESDYYCALWYSN |
| | | LWVFGGGTKLTVLGGGGSGGGGSGGGGSEVQLVESGGGLVQPGGSLR |
| | | LSCAASGFTFSTYAMNWVRQAPGKGLEWVARIRSKYNNYATYYADSV |
| | | KDRFTISRDDSKNSLYLQMNSLKTEDTAVYYCVRHGNFGNSYVSWFA |
| | | YWGQGTLVTVSS |
| V16/V19/V20 | 593 | EVQLVESGGGLVQPGGSLKLSCAASGFTFSTYAMNWVRQASGKGLEW |
| HVD2 | | VGRIRSKYNNYATYYADSVKDRFTISRDDSKNTAYLQMNSLKTEDTAV |
| | | YYCTRHGNFGNSYVSWFAYWGQGTLVTVSS |
| V16 scFv | 594 | QTVVTQEPSFSVSPGGTVTLTCRSSTGAVTTSNYANWVQQTPGQAPRG |
| | | LIGGTNKRAPGVPDRFSGSILGNKAALTITGAQADDESDYYCALWYSN |
| | | LWVFGGGTKLTVLGGGGSGGGGSGGGGSEVQLVESGGGLVQPGGSLK |
| | | LSCAASGFTFSTYAMNWVRQASGKGLEWVGRIRSKYNNYATYYADSV |
| | | KDRFTISRDDSKNTAYLQMNSLKTEDTAVYYCTRHGNFGNSYVSWFA |
| | | YWGQGTLVTVSS |
| V19 | 595 | QAVVTQEPSLTVSPGGTVTLTCRSSTGAVTTSNYANWVQQKPGQAPR |
| LVD | | GLIGGTNKRAPGTPARFSGSLIGGKAALTLSGAQPEDEAEYYCALWYS |
| | | NLWVFGGGTKLTVL |
| V19 scFv | 596 | QAVVTQEPSLTVSPGGTVTLTCRSSTGAVTTSNYANWVQQKPGQAPR |
| | | GLIGGTNKRAPGTPARFSGSLIGGKAALTLSGAQPEDEAEYYCALWYS |
| | | NLWVFGGGTKLTVLGGGGSGGGGSGGGGSEVQLVESGGGLVQPGGSL |
| | | KLSCAASGFTFSTYAMNWVRQASGKGLEWVGRIRSKYNNYATYYADS |
| | | VKDRFTISRDDSKNTAYLQMNSLKTEDTAVYYCTRHGNFGNSYVSWF |
| | | AYWGQGTLVTVSS |
| V26 scFv | 597 | QAVVTQEPSLTVSPGGTVTLTCRSSTGAVTTSNYANWVQQKPGQAPR |
| | | GLIGGTNKRAPGTPARFSGSLIGGKAALTLSGAQPEDEAEYYCALWYS |
| | | NLWVFGGGTKLTVLGGGGSGGGGSGGGGSEVQLVESGGGLVQPGGSL |
| | | RLSCAASGFTFSTYAMNWVRQAPGKGLEWVARIRSKYNNYATYYADS |
| | | VKDRFTISRDDSKNSLYLQMNSLKTEDTAVYYCVRHGNFGNSYVSWF |
| | | AYWGQGTLVTVSS |
| MM2 | 598 | MMYCGGNEVLCGPRV |
| MM2 | 599 | GYLWGCEWNCGGITT |
| MM2 | 600 | GYRWGCEWNCGGITT |
| HC HER2 | 601 | EVQLVESGGGLVQPGGSLRLSCAASGFNIKDTYIHWVRQAPGKGLEW |
| amino acid | | VARIYPTNGYTRYADSVKGRFTISADTSKNTAYLQMNSLRAEDTAVYY |
| sequence | | CSRWGGDGFYAMDYWGQGTLVTVSSASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPGK |
| HC HER2 | 602 | GAAGTGCAGCTGGTGGAGTCCGGAGGCGGTCTGGTACAACCCGGCG |
| nucleic acid | | GCTCCCTGCGCCTGAGTTGCGCCGCTAGTGGATTCAATATAAAGGA |
| sequence | | CACCTACATCCATTGGGTAAGGCAAGCACCTGGCAAGGGTCTGGAG |
| | | TGGGTGGCTCGCATCTACCCAACCAACGGCTATACCAGGTACGQGG |
| | | ATTCAGTTAAGGGGCGGTTCACTATCAGCGCCGACACCAGCAAGAA |
| | | TACAGCTTACCTGCAGATGAATAGCCTGCGCGCAGAGGATACTGCG |
| | | GTGTATTACTGCTCTAGATGGGGGGGCGATGGATTTTACGCAATGG |
| | | ACTACTGGGGGCAGGGAACCTTGGTGACCGTGTCCAGCGCTAGCAC |
| | | CAAGGGCCCATCGGTCTTCCCCCTGGCACCCTCCTCCAAGAGCACCT |
| | | CTGGGGGCACAGCGGCCCTGGGCTGCCTGGTCAAGGACTACTTCCC |
| | | CGAACCGGTGACGGTGTCGTGGAACTCAGGCGCCCTGACCAGCGGC |
| | | GTGCACACCTTCCCGGCTGTCCTACAGTCCTCAGGACTCTACTCCCT |
| | | CAGCAGCGTGGTGACCGTGCCCTCCAGCAGCTTGGGCACCCAGACC |
| | | TACATCTGCAACGTGAATCACAAGCCCAGCAACACCAAGGTGGACA |
| | | AGAAAGTTGAGCCCAAATCTTGTGACAAAACTCACACATGCCCACC |
| | | GTGCCCAGCACCTGAACTCCTGGGGGGACCGTCAGTCTTCCTCTTCC |
| | | CCCCAAAACCCAAGGACACCCTCATGATCTCCCGGACCCCTGAGGT |
| | | CACATGCGTGGTGGTGGACGTGAGCCACGAAGACCCTGAGGTCAAG |
| | | TTCAACTGGTACGTGGACGGCGTGGAGGTGCATAATGCCAAGACAA |
| | | AGCCGCGGGAGGAGCAGTACAACAGCACGTACCGTGTGGTCAGCGT |
| | | CCTCACCGTCCTGCACCAGGACTGGCTGAATGGCAAGGAGTACAAG |
| | | TGCAAGGTCTCCAACAAAGCCCTCCCAGCCCCCATCGAGAAAACCA |
| | | TCTCCAAAGCCAAAGGGCAGCCCCGAGAACCACAGGTGTACACCCT |
| | | GCCCCCATCCCGGGAGGAGATGACCAAGAACCAGGTCAGCCTGACC |
| | | TGCCTGGTCAAAGGCTTCTATCCCAGCGACATCGCCGTGGAGTGGG |
| | | AGAGCAATGGGCAGCCGGAGAACAACTACAAGACCACGCCTCCCGT |
| | | GCTGGACTCCGACGGCTCCTTCTTCCTCTACAGCAAGCTCACCGTGG |
| | | ACAAGAGCAGGTGGCAGCAGGGGAACGTCTTCTCATGCTCCGTGAT |
| | | GCATGAGGCTCTGCACAACCACTACACGCAGAAGAGCCTCTCCCTG |
| | | TCTCCGGGTAAA |
| CI360 LC | 603 | QGQSGQGISWCMMHHEGNVCQTVGGGSSGGSISSGLLSGRSDNHGGG |
| amino acid | | SDIQMTQSPSSLSASVGDRVTITCRASQDVNTAVAWYQQKPGKAPKLL |
| sequence | | IYSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI360 LC | 604 | CAGGGACAGTCCGGCCAGGGCTCATGGTGCATGATGCACCACGAGG |
| nucleic acid | | GGAATGTGTGCCAGACCGTGGGCGGQGGATCCTCTGGCGGTTCAAT |
| sequence | | CTCATCAGGACTGCTGTCTGGGCGGAGCGATAATCATGGCGGAGGC |
| | | AGTGATATACAGATGACACAGAGCCCAAGTAGTCTTAGCGCCAGCG |
| | | TGGGCGATCGGGTGACTATCACTTGCCGGGCTTCTCAGGATGTCAAT |
| | | ACCGCAGTTGCTTGGTATCAGCAGAAGCCTGGGAAAGCTCCGAAGC |
| | | TGCTTATTTATTCTGCAAGTTTCCTGTACTCCGGGGTACCCTCAAGA |
| | | TTCAGCGGGTCTAGAAGTGGGACCGACTTCACCCTCACAATCAGCA |
| | | GCCTGCAGCCCGAAGATTTCGCCACCTACTATTGTCAGCAGCATTAC |
| | | ACGACCCCACCTACTTTCGGGCAGGGAACCAAGGTGGAGATAAAGC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI361 LC | 605 | QGQSGQGLDCVRTLYYSDCYHRGGGSSGGSISSGLLSGRSDNHGGGSD |
| amino acid | | IQMTQSPSSLSASVGDRVTITCRASQDVNTAVAWYQQKPGKAPKLLIY |
| sequence | | SASFLYSGVPSRFSGSRSGTDFTLTISSLQPEDFATYYCQQHYTTPPTFG |
| | | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSENRGEC |
| CI361 LC | 607 | CAGGGACAGAGCGGCCAAGGGTTGGACTGCGTGCGGACTCTCTACT |
| nucleic acid | | ACTCCGATTGTTACCACAGGGGCGGAGGTTCTTCCGGCGGGTCCATT |
| sequence | | TCCTCAGGTCTCCTCAGTGGCCGGTCCGATAACCACGGGGGGGGCA |
| | | GCGATATCCAGATGACTCAGAGCCCCTCATCCCTCTCCGCTTCCGTT |
| | | GGCGACCGGGTGACTATCACTTGCAGGGCTTCACAGGACGTTAACA |
| | | CAGCAGTGGCGTGGTATCAGCAGAAACCTGGCAAGGCCCCAAAACT |
| | | GCTGATCTATAGCGCGAGCTTCCTCTACTCCGGGGTACCCAGCAGAT |
| | | TTTCAGGCAGCCGGAGCGGCACCGACTTTACATTGACTATCAGCAG |
| | | TTTGCAACCCGAGGATTTCGCAACCTACTACTGTCAGCAGCATTACA |
| | | CAACCCCCCCCACGTTTGGACAGGGTACTAAGGTGGAGATCAAGCG |
| | | TACGGTGGCTGCACCATCTGTCTTCATCTTCCCGCCATCTGATGAGC |
| | | AGTTGAAATCTGGAACTGCCTCTGTTGTGTGCCTGCTGAATAACTTC |
| | | TATCCCAGAGAGGCCAAAGTACAGTGGAAGGTGGATAACGCCCTCC |
| | | AATCGGGTAACTCCCAGGAGAGTGTCACAGAGCAGGACAGCAAGG |
| | | ACAGCACCTACAGCCTCAGCAGCACCCTGACGCTGAGCAAAGCAGA |
| | | CTACGAGAAACACAAAGTCTACGCCTGCGAAGTCACCCATCAGGGC |
| | | CTGAGCTCGCCCGTCACAAAGAGCTTCAACAGGGGAGAGTGT |
| CI362 LC | 608 | QGQSGQGICSYNWHFDTIDCNSGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLILSKADYEKHKVYACEVT |
| | | HQGLSSPVTKSFNRGEC |
| CI362 LC | 609 | CAAGGACAGAGCGGCCAGGGCATCTGCTCATACAATTGGCATTTCG |
| nucleic acid | | ACACCATTGATTGTAATAGTGGGGGTGGAAGTTCAGGGGGCTCTAT |
| sequence | | CTCCAGCGGTCTCCTCTCAGGGAGAAGCGACAACCATGGCGGGGG |
| | | AGCGACATCCAGATGACCCAGAGCCCTAGCTCTTTGAGCGCCAGTG |
| | | TAGGAGATAGGGTCACTATCACCTGCAGGGCGTCACAAGATGTAAA |
| | | CACGGCTGTTGCTTGGTATCAGCAGAAGCCTGGCAAGGCTCCCAAA |
| | | CTGCTGATTTACTCCGCAAGTTTTCTCTATTCCGGGGTGCCCAGCAG |
| | | GTTTTCCGGATCTCGGTCTGGGACCGATTTCACTCTCACAATCTCCA |
| | | GTCTTCAGCCTGAGGATTTTGCCACATACTACTGCCAGCAACATTAC |
| | | ACCACCCCACCCACGTTCGGCCAGGGCACAAAAGTTGAGATAAAGC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI363 LC | 610 | QGQSGQGCCYVLLDGILMPCDCGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI363 LC | 611 | CAGGGCCAAAGTGGCCAGGGATGCTGTTACGTGCTCCTGGATGGCA |
| nucleic acid | | TCTTGATGCCATGCGATTGTGGCGGGGGCTCTTCAGGTGGCAGCATT |
| sequence | | TCTAGCGGCCTGCTCTCTGGGAGGTCCGACAATCATGGGGGGGGT |
| | | CAGACATTCAGATGACACAGTCCCCGAGCAGTCTCTCAGCCTCAGT |
| | | GGGCGACAGGGTGACAATAACTTGTAGGGCATCTCAGGATGTTAAC |
| | | ACTGCCGTAGCATGGTACCAGCAGAAGCCGGGGAAAGCTCCAAAGC |
| | | TGCTTATCTATAGTGCCAGTTTTCTGTATAGCGGGGTGCCTAGCCGA |
| | | TTCAGCGGCAGCCGGAGCGGCACTGATTTCACCCTCACCATTTCAAG |
| | | TTTGCAGCCAGAGGATTTCGCCACGTACTATTGTCAGCAACACTACA |
| | | CAACACCACCGACCTTCGGACAGGGAACGAAGGTGGAAATCAAGC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI364 LC | 612 | QGQSGQGSCYVLLDGILMPSDCGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI364 LC | 613 | CAGGGCCAGTCTGGCCAGGGCTCTTGTTACGTCTTGTTGGACGGGAT |
| nucleic acid | | CCTCATGCCTTCCGATTGCGGAGGTGGTAGCAGTGGTGGCTCCATTT |
| sequence | | CATCCGGCCTGCTGTCTGGCAGGTCAGATAACCACGGCGGGGGCTC |
| | | TGATATCCAGATGACCCAGTCCCCAAGTTCCCTGTCCGCCAGCGTAG |
| | | GCGATAGAGTGACCATCACGTGCCGGGCCAGTCAGGACGTGAACAC |
| | | GGCTGTTGCATGGTATCAACAAAAACCAGGAAAAGCCCCCAAATTG |
| | | CTGATCTATTCCGCGAGTTTCCTTTACAGTGGAGTTCCCTCCCGATTT |
| | | TCCGGAAGCCGGTCCGGAACTGATTTTACTCTTACTATCTCCTCACT |
| | | GCAACCGGAGGATTTCGCAACGTATTATTGCCAACAGCATTATACC |
| | | ACCCCACCTACATTCGGCCAGGGAACAAAGGTGGAGATCAAACGTA |
| | | CGGTGGCTGCACCATCTGTCTTCATCTTCCCGCCATCTGATGAGCAG |
| | | TTGAAATCTGGAACTGCCTCTGTTGTGTGCCTGCTGAATAACTTCTA |
| | | TCCCAGAGAGGCCAAAGTACAGTGGAAGGTGGATAACGCCCTCCAA |
| | | TCGGGTAACTCCCAGGAGAGTGTCACAGAGCAGGACAGCAAGGAC |
| | | AGCACCTACAGCCTCAGCAGCACCCTGACGCTGAGCAAAGCAGACT |
| | | ACGAGAAACACAAAGTCTACGCCTGCGAAGTCACCCATCAGGGCCT |
| | | GAGCTCGCCCGTCACAAAGAGCTTCAACAGGGGAGAGTGT |
| CI365 LC | 614 | QGQSGQGSCYVLLDGILMPCDSGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI365 LC | 615 | CAGGGCCAGTCAGGGCAGGGAAGTTGCTATGTTCTGCTCGACGGGA |
| nucleic acid | | TCCTGATGCCTTGTGATAGCGGCGGAGGTAGCTCCGGCGGGTCCAT |
| sequence | | CAGTTCAGGGCTGCTGTCAGGGCGATCCGACAACCATGGCGGAGGC |
| | | TCTGATATCCAAATGACCCAGAGTCCGTCAAGCCTGAGCGCTAGCG |
| | | TGGGTGATCGAGTTACCATTACATGCAGGGCTAGCCAGGACGTCAA |
| | | TACCGCGGTAGCCTGGTACCAACAAAAGCCTGGAAAAGCCCCTAAA |
| | | CTCCTCATCTATTCTGCGAGTTTCCTGTACAGTGGCGTGCCAAGTAG |
| | | ATTCAGCGGATCTCGCTCAGGCACTGACTTTACTCTCACGATATCTA |
| | | GTTTGCAGCCAGAGGACTTTGCAACCTATTACTGCCAGCAGCATTAT |
| | | ACAACCCCCCCTACCTTCGGGCAAGGAACTAAGGTGGAAATCAAAC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI366 LC | 616 | QGQSGQGCSYVLLDGILMPSDCGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI366 LC | 617 | CAGGGCCAGTCTGGACAGGGTTGTTCTTATGTTCTGCTCGACGGCAT |
| nucleic acid | | CCTGATGCCAAGTGATTGCGGAGGAGGGAGCTCAGGTGGATCCATC |
| sequence | | TCCTCTGGGCTCCTTTCAGGTAGGTCAGACAACCACGGCGGAGGCT |
| | | CTGACATTCAGATGACTCAATCCCCCAGCTCCCTGTCCGCCAGTGTT |
| | | GGGGACCGCGTAACCATCACCTGTAGGGCCTCTCAGGACGTCAACA |
| | | CCGCTGTGGCTTGGTATCAACAGAAGCCAGGCAAAGCTCCCAAGCT |
| | | CTTGATCTACTCAGCTAGCTTCCTTTATTCCGGCGTCCCCAGCCGCTT |
| | | TAGCGGCAGCCGCAGTGGTACAGATTTTACATTGACAATCAGCTCC |
| | | CTTCAGCCTGAGGATTTCGCAACCTATTATTGCCAACAGCATTACAC |
| | | CACACCGCCCACTTTTGGCCAGGGGACTAAAGTCGAGATTAAACGT |
| | | ACGGTGGCTGCACCATCTGTCTTCATCTTCCCGCCATCTGATGAGCA |
| | | GTTGAAATCTGGAACTGCCTCTGTTGTGTGCCTGCTGAATAACTTCT |
| | | ATCCCAGAGAGGCCAAAGTACAGTGGAAGGTGGATAACGCCCTCCA |
| | | ATCGGGTAACTCCCAGGAGAGTGTCACAGAGCAGGACAGCAAGGA |
| | | CAGCACCTACAGCCTCAGCAGCACCCTGACGCTGAGCAAAGCAGAC |
| | | TACGAGAAACACAAAGTCTACGCCTGCGAAGTCACCCATCAGGGCC |
| | | TGAGCTCGCCCGTCACAAAGAGCTTCAACAGGGGAGAGTGT |
| CI367 LC | 618 | QGQSGQGCSYVLLDGILMPCDSGGGSSGGSISSGLLSGRSDNHGGGSDI |
| LC amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI367 LC | 619 | CAGGGGCAGTCAGGCCAGGGGTGCAGCTACGTGCTGCTGGATGGAA |
| nucleic acid | | TCCTTATGCCTTGCGACAGTGGTGGCGGCAGCAGCGGGGGCTCCAT |
| sequence | | CAGTAGTGGGCTGCTGTCCGGAAGGTCAGATAATCATGGTGGGGGA |
| | | AGCGATATACAGATGACACAGAGCCCCTCTAGCCTGTCCGCGAGCG |
| | | TCGGGGACCGAGTTACGATCACCTGTAGGGCCAGTCAGGACGTCAA |
| | | CACAGCGGTGGCATGGTACCAGCAGAAGCCCGGCAAGGCCCCCAA |
| | | ACTCCTGATTTATAGTGCTAGCTTCCTGTATTCCGGGGTTCCAAGTC |
| | | GGTTCTCCGGGTCTAGAAGCGGCACCGACTTCACGCTGACAATATC |
| | | ATCACTGCAGCCTGAAGACTTCGCCACATACTACTGTCAGCAGCACT |
| | | ACACCACTCCTCCAACCTTCGGGCAGGGGACCAAGGTCGAGATTAA |
| | | GCGTACGGTGGCTGCACCATCTGTCTTCATCTTCCCGCCATCTGATG |
| | | AGCAGTTGAAATCTGGAACTGCCTCTGTTGTGTGCCTGCTGAATAAC |
| | | TTCTATCCCAGAGAGGCCAAAGTACAGTGGAAGGTGGATAACGCCC |
| | | TCCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI368 LC | 620 | QGQSGQGSECMISQWGPDCTVAGGGSSGGSISSGLLSGRSDNHGGGSD |
| amino acid | | IQMTQSPSSLSASVGDRVTITCRASQDVNTAVAWYQQKPGKAPKLLIY |
| sequence | | SASFLYSGVPSRFSGSRSGTDFTLTISSLQPEDFATYYCQQHYTTPPTFG |
| | | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| CI368 LC | 621 | CAGGGCCAGTCTGGGCAGGGGTCCGAGTGTATGATCTCCCAGTGGG |
| nucleic acid | | GTCCCGACTGTACCGTTGCGGGAGGGGGCAGTTCCGGCGGCAGTAT |
| sequence | | TTCCAGTGGATTGCTGTCTGGCAGATCTGATAACCATGGTGGTGGTA |
| | | GCGACATCCAGATGACCCAAAGCCCCTCAAGCCTGTCTGCATCCGT |
| | | CGGGGATCGGGTGACTATCACGTGTAGAGCCTCCCAGGATGTGAAT |
| | | ACAGCCGTCGCCTGGTATCAGCAGAAGCCCGGGAAGGCTCCCAAGT |
| | | TGTTGATCTATTCCGCCTCTTTTTTGTATTCCGGCGTGCCTTCAAGAT |
| | | TCAGCGGTTCCAGATCAGGTACGGACTTTACCCTCACAATCAGCTCT |
| | | CTGCAACCAGAAGACTTCGCGACCTATTATTGTCAGCAACATTACAC |
| | | CACACCGCCAACCTTTGGGCAGGGCACAAAGGTGGAAATCAAGCGT |
| | | ACGGTGGCTGCACCATCTGTCTTCATCTTCCCGCCATCTGATGAGCA |
| | | GTTGAAATCTGGAACTGCCTCTGTTGTGTGCCTGCTGAATAACTTCT |
| | | ATCCCAGAGAGGCCAAAGTACAGTGGAAGGTGGATAACGCCCTCCA |
| | | ATCGGGTAACTCCCAGGAGAGTGTCACAGAGCAGGACAGCAAGGA |
| | | CAGCACCTACAGCCTCAGCAGCACCCTGACGCTGAGCAAAGCAGAC |
| | | TACGAGAAACACAAAGTCTACGCCTGCGAAGTCACCCATCAGGGCC |
| | | TGAGCTCGCCCGTCACAAAGAGCTTCAACAGGGGAGAGTGT |
| CI369 LC | 622 | QGQSGQGYCLYNKLNDTINCSDGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI369 LC | 623 | CAGGGCCAGAGCGGCCAAGGATACTGCCTCTACAACAAACTGAATG |
| nucleic acid | | ACACTATCAACTGTTCCGACGGAGGTGGATCCAGCGGGGGATCAAT |
| sequence | | CAGCAGCGGGCTTCTCAGTGGGCGGAGCGACAATCACGGCGGGGG |
| | | ATCCGACATTCAGATGACACAGTCTCCATCTAGCCTTTCTGCCAGTG |
| | | TGGGTGACCGGGTCACGATCACATGCAGAGCCAGCCAGGACGTGAA |
| | | TACCGCAGTCGCCTGGTACCAGCAGAAGCCCGGGAAAGCGCCGAAG |
| | | CTTCTGATCTATTCTGCTAGTTTTCTCTATTCAGGAGTCCCAAGCCGA |
| | | TTCTCTGGATCTAGATCTGGTACGGACTTTACACTCACGATTAGCAG |
| | | TCTCCAACCCGAAGACTTCGCTACCTATTATTGCCAGCAACATTATA |
| | | CTACCCCTCCAACCTTCGGACAGGGAACCAAGGTGGAAATCAAGCG |
| | | TACGGTGGCTGCACCATCTGTCTTCATCTTCCCGCCATCTGATGAGC |
| | | AGTTGAAATCTGGAACTGCCTCTGTTGTGTGCCTGCTGAATAACTTC |
| | | TATCCCAGAGAGGCCAAAGTACAGTGGAAGGTGGATAACGCCCTCC |
| | | AATCGGGTAACTCCCAGGAGAGTGTCACAGAGCAGGACAGCAAGG |
| | | ACAGCACCTACAGCCTCAGCAGCACCCTGACGCTGAGCAAAGCAGA |
| | | CTACGAGAAACACAAAGTCTACGCCTGCGAAGTCACCCATCAGGGC |
| | | CTGAGCTCGCCCGTCACAAAGAGCTTCAACAGGGGAGAGTGT |
| CI370 LC | 624 | QGQSGQGRCVMDPVTDTIDCNKGGGSSGGSISSGLLSGRSDNHGGGSD |
| amino acid | | IQMTQSPSSLSASVGDRVTITCRASQDVNTAVAWYQQKPGKAPKLLIY |
| sequence | | SASFLYSGVPSRFSGSRSGTDFTLTISSLQPEDFATYYCQQHYTTPPTFG |
| | | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| CI370 LC | 625 | CAGGGCCAATCCGGTCAGGGACGGTGTGTTATGGATCCCGTCACCG |
| nucleic acid | | ATACAATCGACTGCAATAAAGGAGGAGGCTCTTCTGGGGGAAGCAT |
| sequence | | TTCTAGTGGGCTTCTGTCCGGACGGTCCGACAACCATGGAGGCGGA |
| | | TCCGACATTCAGATGACTCAATCTCCCTCATCTCTGAGTGCTAGCGT |
| | | TGGCGATCGCGTCACGATCACTTGCCGCGCTTCACAGGACGTGAAT |
| | | ACCGCCGTGGCGTGGTACCAGCAGAAGCCTGGAAAGGCCCCCAAGT |
| | | TGCTTATATATAGCGCCTCCTTTCTGTATTCTGGTGTCCCTTCCCGCT |
| | | TCTCTGGTTCCCGGTCCGGCACCGACTTCACACTTACAATCTCCTCC |
| | | CTGCAGCCAGAAGATTTCGCTACTTATTATTGTCAGCAACACTATAC |
| | | AACACCTCCTACTTTTGGGCAAGGAACTAAAGTCGAAATCAAGCGT |
| | | ACGGTGGCTGCACCATCTGTCTTCATCTTCCCGCCATCTGATGAGCA |
| | | GTTGAAATCTGGAACTGCCTCTGTTGTGTGCCTGCTGAATAACTTCT |
| | | ATCCCAGAGAGGCCAAAGTACAGTGGAAGGTGGATAACGCCCTCCA |
| | | ATCGGGTAACTCCCAGGAGAGTGTCACAGAGCAGGACAGCAAGGA |
| | | CAGCACCTACAGCCTCAGCAGCACCCTGACGCTGAGCAAAGCAGAC |
| | | TACGAGAAACACAAAGTCTACGCCTGCGAAGTCACCCATCAGGGCC |
| | | TGAGCTCGCCCGTCACAAAGAGCTTCAACAGGGGAGAGTGT |
| LC | 626 | QGQSGQGALICCSDVSGLCRWCGGGSSGGSISSGLLSGRSDNHGGGSDI |
| CI371 | | QMTQSPSSLSASVGDRVTITCRASQDVNTAVAWYQQKPGKAPKLLIYS |
| CI101 | | ASFLYSGVPSRFSGSRSGTDFTLTISSLQPEDFATYYCQQHYTTPPTFGQ |
| CI152 CI240 CI148 | | GTKVEIKRTVAAPSVFIFPPSDEQLKSGTASVVCLLNNFYPREAKVQWK |
| CI153 | | VDNALQSGNSQESVTEQDSKDSTYSLSSTLTLSKADYEKHKVYACEVT |
| CI241 | | HQGLSSPVTKSFNRGEC |
| CI242 | | |
| CI243 | | |
| CI124 | | |
| amino acid | | |
| sequence | | |
| CI371 | 627 | CAGGGCCAGTCAGGGCAGGGAGCCCTGATCTGCTGTAGCGACGTGT |
| CI101 | | CAGGGCTTTGTAGATGGTGCGGGGGAGGATCCTCTGGTGGATCTAT |
| CI152 | | CTCTAGCGGACTGCTGAGCGGGCGCAGCGACAACCACGGCGGCGGA |
| CI240 | | AGTGACATACAGATGACCCAGAGTCCCAGCTCTCTTTCTGCTAGTGT |
| CI148 | | CGGCGACAGGGTGACAATCACGTGCCGCGCATCCCAAGACGTCAAC |
| CI153 | | ACCGCCGTGGCATGGTACCAGCAAAAACCAGGCAAAGCCCCTAAAC |
| CI241 | | TGTTGATTTATTCCGCTTCATTCCTGTATAGTGGGGTACCCTCCCGAT |
| CI242 | | TTAGCGGCTCCCGGTCCGGAACAGATTTCACTCTCACTATAAGCTCA |
| CI243 | | CTTCAGCCAGAAGATTTCGCAACATACTATTGTCAACAGCATTACAC |
| nucleic acid | | AACCCCGCCAACCTTCGGCCAGGGGACCAAAGTCGAGATCAAACGT |
| sequence | | ACGGTGGCTGCACCATCTGTCTTCATCTTCCCGCCATCTGATGAGCA |
| | | GTTGAAATCTGGAACTGCCTCTGTTGTGTGCCTGCTGAATAACTTCT |
| | | ATCCCAGAGAGGCCAAAGTACAGTGGAAGGTGGATAACGCCCTCCA |
| | | ATCGGGTAACTCCCAGGAGAGTGTCACAGAGCAGGACAGCAAGGA |
| | | CAGCACCTACAGCCTCAGCAGCACCCTGACGCTGAGCAAAGCAGAC |
| | | TACGAGAAACACAAAGTCTACGCCTGCGAAGTCACCCATCAGGGCC |
| | | TGAGCTCGCCCGTCACAAAGAGCTTCAACAGGGGAGAGTGT |
| CI372 LC | 628 | QGQSGQGALISCSDVSGLSRWCGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI372 LC | 629 | CAGGGGCAATCCGGTCAAGGGGCACTGATCAGTTGCAGCGATGTCA |
| nucleic acid | | GTGGCCTCAGTAGATGGTGCGGTGGCGGCTCATCTGGCGGCAGCAT |
| sequence | | CTCTAGTGGGCTGCTCTCTGGTAGAAGCGATAACCACGGCGGAGGC |
| | | AGTGATATCCAGATGACTCAGTCTCCCTCCAGTCTCTCTGCTTCTGT |
| | | GGGAGACAGAGTTACCATAACTTGTCGAGCTTCCCAGGATGTTAAC |
| | | ACAGCAGTCGCATGGTATCAGCAGAAGCCCGGCAAAGCTCCCAAGC |
| | | TGCTCATATACAGCGCCTCCTTTCTGTACTCCGGGGTCCCTAGCAGA |
| | | TTTAGCGGGAGTAGGTCCGGCACCGATTTCACACTCACAATCTCCAG |
| | | CCTGCAGCCCGAGGACTTCGCAACATATTATTGTCAGCAGCACTAC |
| | | ACTACACCGCCCACTTTCGGCCAAGGCACGAAAGTTGAAATCAAAC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI373 LC | 630 | QGQSGQGALISCSDVSGLCRWSGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI373 LC | 631 | CAGGGACAGTCCGGGCAAGGTGCTCTGATCTCCTGCTCAGATGTTA |
| nucleic acid | | GTGGTCTCTGTAGATGGAGCGGGGGGGGAGTTCCGGAGGCAGCAT |
| sequence | | TTCTTCCGGCCTGCTCTCTGGGAGATCAGACAACCATGGCGGCGGTT |
| | | CTGATATACAGATGACCCAGTCTCCATCCTCCCTCAGCGCATCCGTG |
| | | GGTGATCGGGTGACTATCACTTGTCGCGCCTCTCAGGACGTGAACA |
| | | CCGCTGTGGCCTGGTACCAGCAGAAACCCGGCAAAGCTCCAAAACT |
| | | CCTTATTTACAGCGCCAGCTTCCTCTATTCTGGGGTCCCTTCACGGTT |
| | | TTCTGGGAGCCGGTCCGGGACCGACTTTACCCTCACAATCAGTTCCC |
| | | TCCAGCCTGAGGACTTCGCTACTTATTATTGTCAACAACACTATACC |
| | | ACACCCCCTACTTTTGGCCAGGGGACAAAGGTTGAGATTAAGCGTA |
| | | CGGTGGCTGCACCATCTGTCTTCATCTTCCCGCCATCTGATGAGCAG |
| | | TTGAAATCTGGAACTGCCTCTGTTGTGTGCCTGCTGAATAACTTCTA |
| | | TCCCAGAGAGGCCAAAGTACAGTGGAAGGTGGATAACGCCCTCCAA |
| | | TCGGGTAACTCCCAGGAGAGTGTCACAGAGCAGGACAGCAAGGAC |
| | | AGCACCTACAGCCTCAGCAGCACCCTGACGCTGAGCAAAGCAGACT |
| | | ACGAGAAACACAAAGTCTACGCCTGCGAAGTCACCCATCAGGGCCT |
| | | GAGCTCGCCCGTCACAAAGAGCTTCAACAGGGGAGAGTGT |
| CI374 LC | 632 | QGQSGQGALICSSDVSGLSRWCGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI374 LC | 633 | CAGGGACAGTCAGGGCAGGGAGCACTGATATGCAGCAGCGACGTCT |
| nucleic acid | | CCGGGCTCTCCCGGTGGTGCGGGGGTGGATCCAGTGGAGGGAGTAT |
| sequence | | CTCAAGTGGACTCCTGAGCGGCAGATCTGACAACCACGGCGGGGGC |
| | | TCAGATATTCAAATGACCCAGTCACCCAGTAGTCTGTCTGCCAGTGT |
| | | GGGAGACCGGGTGACTATCACATGTCGGGCCAGTCAGGATGTGAAT |
| | | ACGGCGGTGGCATGGTATCAGCAGAAGCCAGGCAAAGCCCCGAAA |
| | | CTGCTTATCTATTCCGCGTCTTTCCTGTACTCTGGCGTGCCCAGTCGG |
| | | TTTAGCGGCTCCCGGTCAGGAACAGACTTTACACTGACCATCAGTTC |
| | | ACTGCAGCCAGAAGACTTCGCCACCTACTACTGCCAGCAGCATTAT |
| | | ACAACACCCCCAACGTTTGGCCAGGGAACCAAGGTTGAGATCAAGC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI375 LC | 634 | QGQSGQGALICSSDVSGLCRWSGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI375 LC | 635 | CAAGGGCAGTCTGGGCAGGGTGCGTTGATATGTTCTTCTGACGTATC |
| nucleic acid | | AGGTCTGTGTCGGTGGTCCGGCGGGGGCAGCTCAGGTGGCTCCATT |
| sequence | | AGCAGTGGGCTGCTCAGCGGGCGCTCCGATAATCACGGGGGGGGCT |
| | | CTGACATTCAGATGACCCAATCTCCCTCCTCCCTGTCTGCGTCTGTC |
| | | GGCGATAGAGTCACCATCACCTGCAGGGCCAGCCAAGACGTAAACA |
| | | CAGCTGTGGCATGGTATCAGCAAAAACCCGGGAAGGCACCAAAACT |
| | | CCTCATCTATTCCGCTTCATTTTTGTATAGCGGGGTGCCATCTCGGTT |
| | | TAGTGGGTCCCGGTCCGGCACTGACTTCACTCTGACAATCTCAAGTC |
| | | TCCAACCCGAAGACTTCGCAACCTATTATTGTCAGCAGCATTATACC |
| | | ACACCTCCAACATTTGGCCAGGGAACTAAGGTGGAAATCAAACGTA |
| | | CGGTGGCTGCACCATCTGTCTTCATCTTCCCGCCATCTGATGAGCAG |
| | | TTGAAATCTGGAACTGCCTCTGTTGTGTGCCTGCTGAATAACTTCTA |
| | | TCCCAGAGAGGCCAAAGTACAGTGGAAGGTGGATAACGCCCTCCAA |
| | | TCGGGTAACTCCCAGGAGAGTGTCACAGAGCAGGACAGCAAGGAC |
| | | AGCACCTACAGCCTCAGCAGCACCCTGACGCTGAGCAAAGCAGACT |
| | | ACGAGAAACACAAAGTCTACGCCTGCGAAGTCACCCATCAGGGCCT |
| | | GAGCTCGCCCGTCACAAAGAGCTTCAACAGGGGAGAGTGT |
| CI376 LC | 636 | QGQSGQGSSCRTTGLMTTCDLDGGGSSGGSISSGLLSGRSDNHGGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI376 LC | 637 | CAGGGCCAGTCCGGCCAGGGTAGTAGCTGTAGGACCACAGGCCTTA |
| nucleic acid | | TGACCACGTGTGACTTGGATGGCGGCGGGTCCTCTGGGGGTTCCAT |
| sequence | | ATCTAGTGGCCTGTTGTCTGGGCGAAGCGATAATCATGGCGGAGGT |
| | | AGCGATATCCAAATGACACAGTCACCTAGTTCACTGTCTGCATCCGT |
| | | GGGCGACAGGGTCACCATCACTTGTCGCGCTTCCCAGGATGTCAAT |
| | | ACCGCAGTTGCCTGGTATCAACAGAAACCCGGCAAGGCTCCAAAGC |
| | | TCCTGATCTACAGCGCAAGTTTCCTTTATAGTGGTGTACCCAGCCGC |
| | | TTTTCTGGCAGCAGGAGTGGAACTGATTTCACCCTGACTATCTCCAG |
| | | CCTGCAGCCTGAAGATTTTGCCACTTATTACTGCCAACAGCACTATA |
| | | CAACTCCACCTACATTTGGTCAGGGCACGAAGGTGGAAATCAAACG |
| | | TACGGTGGCTGCACCATCTGTCTTCATCTTCCCGCCATCTGATGAGC |
| | | AGTTGAAATCTGGAACTGCCTCTGTTGTGTGCCTGCTGAATAACTTC |
| | | TATCCCAGAGAGGCCAAAGTACAGTGGAAGGTGGATAACGCCCTCC |
| | | AATCGGGTAACTCCCAGGAGAGTGTCACAGAGCAGGACAGCAAGG |
| | | ACAGCACCTACAGCCTCAGCAGCACCCTGACGCTGAGCAAAGCAGA |
| | | CTACGAGAAACACAAAGTCTACGCCTGCGAAGTCACCCATCAGGGC |
| | | CTGAGCTCGCCCGTCACAAAGAGCTTCAACAGGGGAGAGTGT |
| CI377-CI380 | 638 | QGQSGQMMYCGGNEVLCGPRVGSSGGSGGSGGISSGLLSGRSDNHGG |
| HC | | GSQTVVTQEPSLTVSPGGTVTLTCRSSTGAVTTSNYANWVQQKPGQAP |
| amino acid | | RGLIGGTNKRAPGTPARFSGSLLGGKAALTLSGVQPEDEAEYYCALWY |
| sequence | | SNLWVFGGGTKLTVLGGGGSGGGGSGGGGSEVQLVESGGGLVQPGGS |
| | | LKLSCAASGFTFNTYAMNWVRQAPGKGLEWVARIRSKYNNYATYYA |
| | | DSVKDRFTISRDDSKNTAYLQMNNLKTEDTAVYYCVRHGNFGNSYVS |
| | | WFAYWGQGTLVTVSSGGGGSEVQLVESGGGLVQPGGSLRLSCAASGF |
| | | NIKDTYIHWVRQAPGKGLEWVARIYPTNGYTRYADSVKGRFTISADTS |
| | | KNTAYLQMNSLRAEDTAVYYCSRWGGDGFYAMDYWGQGTLVTVSS |
| | | ASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVD |
| | | VSHEDPEVKFNWYVDGVEVHNAKTKPREEQYQSTYRVVSVLTVLHQD |
| | | WLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| CI377-CI380 | 639 | CAAGGCCAGTCTGGCCAAATGATGTATTGCGGTGGGAATGAGGTGT |
| HC | | TGTGCGGGCCGCGGGTTGGCTCGAGCGGTGGCAGCGGTGGCTCTGG |
| nucleic acid | | TGGTATCTCTTCCGGACTGCTGTCCGGCAGATCCGACAATCACGGCG |
| sequence | | GCGGTTCTCAGACCGTGGTCACACAGGAGCCCTCACTGACAGTGAG |
| | | CCCTGGCGGGACCGTCACACTGACTTGTCGCAGTTCAACTGGCGCC |
| | | GTGACTACCAGCAATTACGCTAACTGGGTCCAGCAGAAACCAGGAC |
| | | AGGCACCACGAGGACTGATCGGAGGAACTAATAAGAGAGCACCAG |
| | | GAACCCCTGCAAGGTTCTCCGGATCTCTGCTGGGGGGAAAAGCCGC |
| | | TCTGACACTGAGCGGCGTGCAGCCTGAGGACGAAGCTGAGTACTAT |
| | | TGCGCACTGTGGTACTCCAACCTGTGGGTGTTTGGGGGGGGAACTA |
| | | AGCTGACCGTCCTGGGAGGAGGAGGAAGCGGAGGAGGAGGGAGCG |
| | | GAGGAGGAGGATCCGAAGTGCAGCTGGTCGAGAGCGGAGGAGGAC |
| | | TGGTGCAGCCAGGAGGATCCCTGAAGCTGTCTTGTGCAGCCAGTGG |
| | | CTTCACCTTCAACACTTACGCAATGAACTGGGTGCGGCAGGCACCT |
| | | GGGAAGGGACTGGAATGGGTCGCCCGGATCAGATCTAAATACAATA |
| | | ACTATGCCACCTACTATGCTGACAGTGTGAAGGATAGGTTCACCATT |
| | | TCACGCGACGATAGCAAAAACACAGCTTATCTGCAGATGAATAACC |
| | | TGAAGACCGAGGATACAGCAGTGTACTATTGCGTCAGACACGGCAA |
| | | TTTCGGGAACTCTTACGTGAGTTGGTTTGCCTATTGGGGACAGGGGA |
| | | CACTGGTCACCGTCTCCTCAGGAGGTGGTGGATCCGAAGTGCAGCT |
| | | GGTGGAGTCCGGAGGCGGTCTGGTACAACCCGGCGGCTCCCTGCGC |
| | | CTGAGTTGCGCCGCTAGTGGATTCAATATAAAGGACACCTACATCC |
| | | ATTGGGTAAGGCAAGCACCTGGCAAGGGTCTGGAGTGGGTGGCTCG |
| | | CATCTACCCAACCAACGGCTATACCAGGTACGCGGATTCAGTTAAG |
| | | GGGCGGTTCACTATCAGCGCCGACACCAGCAAGAATACAGCTTACC |
| | | TGCAGATGAATAGCCTGCGCGCAGAGGATACTGCGGTGTATTACTG |
| | | CTCTAGATGGGGGGGCGATGGATTTTACGCAATGGACTACTGGGGG |
| | | CAGGGAACCTTGGTGACCGTGTCCAGCGCTAGCACCAAGGGCCCAT |
| | | CGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGA |
| | | CGGTGTCGTGGAACTCAGGCGCCCTGACCAGCGGCGTGCACACCTT |
| | | CCCGGCTGTCCTACAGTCCTCAGGACTCTACTCCCTCAGCAGCGTGG |
| | | TGACCGTGCCCTCCAGCAGCTTGGGCACCCAGACCTACATCTGCAA |
| | | CGTGAATCACAAGCCCAGCAACACCAAGGTGGACAAGAAAGTTGA |
| | | GCCCAAATCTTGTGACAAAACTCACACATGCCCACCGTGCCCAGCA |
| | | CCTGAACTCCTGGGGGGACCGTCAGTCTTCCTCTTCCCCCCAAAACC |
| | | CAAGGACACCCTCATGATCTCCCGGACCCCTGAGGTCACATGCGTG |
| | | GTGGTGGACGTGAGCCACGAAGACCCTGAGGTCAAGTTCAACTGGT |
| | | ACGTGGACGGCGTGGAGGTGCATAATGCCAAGACAAAGCCGCGGG |
| | | AGGAGCAGTACCAGAGCACGTACCGTGTGGTCAGCGTCCTCACCGT |
| | | CCTGCACCAGGACTGGCTGAATGGCAAGGAGTACAAGTGCAAGGTC |
| | | TCCAACAAAGCCCTCCCAGCCCCCATCGAGAAAACCATCTCCAAAG |
| | | CCAAAGGGCAGCCCCGAGAACCACAGGTGTACACCCTGCCCCCATC |
| | | CCGGGAGGAGATGACCAAGAACCAGGTCAGCCTGACCTGCCTGGTC |
| | | AAAGGCTTCTATCCCAGCGACATCGCCGTGGAGTGGGAGAGCAATG |
| | | GGCAGCCGGAGAACAACTACAAGACCACGCCTCCCGTGCTGGACTC |
| | | CGACGGCTCCTTCTTCCTCTACAGCAAGCTCACCGTGGACAAGAGC |
| | | AGGTGGCAGCAGGGGAACGTCTTCTCATGCTCCGTGATGCATGAGG |
| | | CTCTGCACAACCACTACACGCAGAAGAGCCTCTCCCTGTCTCCGGGT |
| | | AAA |
| pLW255: HC | 640 | QGQSGQMMYCGGNEVLCGPRVGSSGGSGGSGGISSGLLSGRSDNHGG |
| amino acid | | GSQTVVTQEPSLTVSPGGTVTLTCRSSTGAVTTSNYANWVQQKPGQAP |
| sequence | | RGLIGGTNKRAPGTPARFSGSLLGGKAALTLSGVQPEDEAEYYCALWY |
| | | SNLWVFGGGTKLTVLGGGGSGGGGSGGGGSEVQLVESGGGLVQPGGS |
| | | LKLSCAASGFTENTYAMNWVRQAPGKGLEWVARIRSKYNNYATYYA |
| | | DSVKDRFTISRDDSKNTAYLQMNNLKTEDTAVYYCVRHGNFGNSYVS |
| | | WFAYWGQGTLVTVSSGGGGSGGGGSGGGGSEVQLVESGGGLVQPGG |
| | | SLRLSCAASGFNIKDTYIHWVRQAPGKGLEWVARIYPTNGYTRYADSV |
| | | KGRFTISADTSKNTAYLQMNSLRAEDTAVYYCSRWGGDGFYAMDYW |
| | | GQGTLVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISR |
| | | TPEVTCVVVDVSHEDPEVKFNWYVDGVEVHNAKTKPREEQYQSTYRV |
| | | VSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | K |
| pLW255: HC | 641 | CAAGGCCAGTCTGGCCAAATGATGTATTGCGGTGGGAATGAGGTGT |
| nucleic acid | | TGTGCGGGCCGCGGGTTGGCTCGAGCGGTGGCAGCGGTGGCTCTGG |
| sequence | | TGGTATCTCTTCCGGACTGCTGTCCGGCAGATCCGACAATCACGGCG |
| | | GCGGTTCTCAGACCGTGGTCACACAGGAGCCCTCACTGACAGTGAG |
| | | CCCTGGCGGGACCGTCACACTGACTTGTCGCAGTTCAACTGGCGCC |
| | | GTGACTACCAGCAATTACGCTAACTGGGTCCAGCAGAAACCAGGAC |
| | | AGGCACCACGAGGACTGATCGGAGGAACTAATAAGAGAGCACCAG |
| | | GAACCCCTGCAAGGTTCTCCGGATCTCTGCTGGGGGGAAAAGCCGC |
| | | TCTGACACTGAGCGGCGTGCAGCCTGAGGACGAAGCTGAGTACTAT |
| | | TGCGCACTGTGGTACTCCAACCTGTGGGTGTTTGGCGGGGGAACTA |
| | | AGCTGACCGTCCTGGGAGGAGGAGGAAGCGGAGGAGGAGGGAGCG |
| | | GAGGAGGAGGATCCGAAGTACAGTTGGTTGAGTCAGGAGGGGGTCT |
| | | CGTGCAACCGGGAGGCAGCTTGAAATTGTCTTGCGCAGCCTCCGGT |
| | | TTCACCTTCAATACGTATGCTATGAACTGGGTGCGCCAAGCGCCTGG |
| | | TAAAGGGCTCGAATGGGTTGCACGGATAAGAAGCAAATATAACAAC |
| | | TACGCAACTTATTACGCTGATAGCGTAAAAGATAGGTTCACCATTTC |
| | | ACGAGACGATTCCAAAAATACAGCGTATTTGCAAATGAATAATCTC |
| | | AAAACCGAAGATACCGCTGTATATTATTGTGTTCGGCACGGAAACT |
| | | TCGGAAACTCTTACGTTAGCTGGTTCGCATACTGGGGCCAAGGGAC |
| | | GCTCGTGACAGTATCCTCAGGAGGTGGTGGATCTGGAGGTGGTGGA |
| | | TCTGGAGGTGGTGGATCCGAAGTGCAGCTGGTGGAGTCCGGAGGCG |
| | | GTCTGGTACAACCCGGCGGCTCCCTGCGCCTGAGTTGCGCCGCTAGT |
| | | GGATTCAATATAAAGGACACCTACATCCATTGGGTAAGGCAAGCAC |
| | | CTGGCAAGGGTCTGGAGTGGGTGGCTCGCATCTACCCAACCAACGG |
| | | CTATACCAGGTACGCGGATTCAGTTAAGGGGCGGTTCACTATCAGC |
| | | GCCGACACCAGCAAGAATACAGCTTACCTGCAGATGAATAGCCTGC |
| | | GCGCAGAGGATACTGCGGTGTATTACTGCTCTAGATGGGGGGGCGA |
| | | TGGATTTTACGCAATGGACTACTGGGGGCAGGGAACCTTGGTGACC |
| | | GTGTCCAGCGCTAGCACCAAGGGCCCATCGGTCTTCCCCCTGGCACC |
| | | CTCCTCCAAGAGCACCTCTGGGGGCACAGCGGCCCTGGGCTGQCTG |
| | | GTCAAGGACTACTTCCCCGAACCGGTGACGGTGTCGTGGAACTCAG |
| | | GCGCCCTGACCAGCGGCGTGCACACCTTCCCGGCTGTCCTACAGTCC |
| | | TCAGGACTCTACTCCCTCAGCAGCGTGGTGACCGTGCCCTCCAGCAG |
| | | CTTGGGCACCCAGACCTACATCTGCAACGTGAATCACAAGCCCAGC |
| | | AACACCAAGGTGGACAAGAAAGTTGAGCCCAAATCTTGTGACAAAA |
| | | CTCACACATGCCCACCGTGCCCAGCACCTGAACTCCTGGGGGGACC |
| | | GTCAGTCTTCCTCTTCCCCCCAAAACCCAAGGACACCCTCATGATCT |
| | | CCCGGACCCCTGAGGTCACATGCGTGGTGGTGGACGTGAGCCACGA |
| | | AGACCCTGAGGTCAAGTTCAACTGGTACGTGGACGGCGTGGAGGTG |
| | | CATAATGCCAAGACAAAGCCGCGGGAGGAGCAGTACCAGAGCACG |
| | | TACCGTGTGGTCAGCGTCCTCACCGTCCTGCACCAGGACTGGCTGAA |
| | | TGGCAAGGAGTACAAGTGCAAGGTCTCCAACAAAGCCCTCCCAGCC |
| | | CCCATCGAGAAAACCATCTCCAAAGCCAAAGGGCAGCCCCGAGAAC |
| | | CACAGGTGTACACCCTGCCCCCATCCCGGGAGGAGATGACCAAGAA |
| | | CCAGGTCAGCCTGACCTGCCTGGTCAAAGGCTTCTATCCCAGCGAC |
| | | ATCGCCGTGGAGTGGGAGAGCAATGGGCAGCCGGAGAACAACTAC |
| | | AAGACCACGCCTCCCGTGCTGGACTCCGACGGCTCCTTCTTCCTCTA |
| | | CAGCAAGCTCACCGTGGACAAGAGCAGGTGGCAGCAGGGGAACGT |
| | | CTTCTCATGCTCCGTGATGCATGAGGCTCTGCACAACCACTACACGC |
| | | AGAAGAGCCTCTCCCTGTCTCCGGGTAAA |
| Activated | 642 | SDNHGGGSQTVVTQEPSLTVSPGGTVTLTCRSSTGAVTTSNYANWVQQ |
| pLW255: HC | | KPGQAPRGLIGGTNKRAPGTPARFSGSLLGGKAALTLSGVQPEDEAEY |
| amino acid | | YCALWYSNLWVFGGGTKLTVLGGGGSGGGGSGGGGSEVQLVESGGG |
| sequence | | LVQPGGSLKLSCAASGFTFNTYAMNWVRQAPGKGLEWVARIRSKYNN |
| | | YATYYADSVKDRFTISRDDSKNTAYLQMNNLKTEDTAVYYCVRHGNF |
| | | GNSYVSWFAYWGQGTLVTVSSGGGGSGGGGSGGGGSEVQLVESGGG |
| | | LVQPGGSLRLSCAASGFNIKDTYIHWVRQAPGKGLEWVARIYPTNGYT |
| | | RYADSVKGRFTISADTSKNTAYLQMNSLRAEDTAVYYCSRWGGDGFY |
| | | AMDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKKVEPKSCDKTHTCPPCPAPELLGGPSVFLFPPKPKD |
| | | TLMISRTPEVTCVVVDVSHEDPEVKFNWYVDGVEVHNAKTKPREEQY |
| | | QSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKS |
| | | LSLSPGK |
| Activated | 643 | TCCGACAATCACGGCGGCGGTTCTCAGACCGTGGTCACACAGGAGC |
| pLW255: HC | | CCTCACTGACAGTGAGCCCTGGCGGGACCGTCACACTGACTTGTCG |
| nucleic acid | | CAGTTCAACTGGCGCCGTGACTACCAGCAATTACGCTAACTGGGTC |
| sequence | | CAGCAGAAACCAGGACAGGCACCACGAGGACTGATCGGAGGAACT |
| | | AATAAGAGAGCACCAGGAACCCCTGCAAGGTTCTCCGGATCTCTGC |
| | | TGGGGGGAAAAGCCGCTCTGACACTGAGCGGCGTGCAGCCTGAGGA |
| | | CGAAGCTGAGTACTATTGCGCACTGTGGTACTCCAACCTGTGGGTGT |
| | | TTGGCGGGGGAACTAAGCTGACCGTCCTGGGAGGAGGAGGAAGCG |
| | | GAGGAGGAGGGAGCGGAGGAGGAGGATCCGAAGTACAGTTGGTTG |
| | | AGTCAGGAGGGGGTCTCGTGCAACCGGGAGGCAGCTTGAAATTGTC |
| | | TTGCGCAGCCTCCGGTTTCACCTTCAATACGTATGCTATGAACTGGG |
| | | TGCGCCAAGCGCCTGGTAAAGGGCTCGAATGGGTTGCACGGATAAG |
| | | AAGCAAATATAACAACTACGCAACTTATTACGCTGATAGCGTAAAA |
| | | GATAGGTTCACCATTICACGAGACGATTCCAAAAATACAGCGTATTT |
| | | GCAAATGAATAATCTCAAAACCGAAGATACCGCTGTATATTATTGT |
| | | GTTCGGCACGGAAACTTCGGAAACTCTTACGTTAGCTGGTTCGCATA |
| | | CTGGGGCCAAGGGACGCTCGTGACAGTATCCTCAGGAGGTGGTGGA |
| | | TCTGGAGGTGGTGGATCTGGAGGTGGTGGATCCGAAGTGCAGCTGG |
| | | TGGAGTCCGGAGGCGGTCTGGTACAACCCGGCGGCTCCCTGCGCCT |
| | | GAGTTGCGCCGCTAGTGGATTCAATATAAAGGACACCTACATCCAT |
| | | TGGGTAAGGCAAGCACCTGGCAAGGGTCTGGAGTGGGTGGCTCGCA |
| | | TCTACCCAACCAACGGCTATACCAGGTACGCGGATTCAGTTAAGGG |
| | | GCGGTTCACTATCAGCGCCGACACCAGCAAGAATACAGCTTACCTG |
| | | CAGATGAATAGCCTGCGCGCAGAGGATACTGCGGTGTATTACTGCT |
| | | CTAGATGGGGGGGCGATGGATTTTACGCAATGGACTACTGGGGGCA |
| | | GGGAACCTTGGTGACCGTGTCCAGCGCTAGCACCAAGGGCCCATCG |
| | | GTCTTCCCCCTGGCACCCTCCTCCAAGAGCACCTCTGGGGGCACAGC |
| | | GGCCCTGGGCTGCCTGGTCAAGGACTACTTCCCCGAACCGGTGACG |
| | | GTGTCGTGGAACTCAGGCGCCCTGACCAGCGGCGTGCACACCTTCC |
| | | CGGCTGTCCTACAGTCCTCAGGACTCTACTCCCTCAGCAGCGTGGTG |
| | | ACCGTGCCCTCCAGCAGCTTGGGCACCCAGACCTACATCTGCAACG |
| | | TGAATCACAAGCCCAGCAACACCAAGGTGGACAAGAAAGTTGAGC |
| | | CCAAATCTTGTGACAAAACTCACACATGCCCACCGTGCCCAGCACC |
| | | TGAACTCCTGGGGGGACCGTCAGTCTTCCTCTTCCCCCCAAAACCCA |
| | | AGGACACCCTCATGATCTCCCGGACCCCTGAGGTCACATGCGTGGT |
| | | GGTGGACGTGAGCCACGAAGACCCTGAGGTCAAGTTCAACTGGTAC |
| | | GTGGACGGCGTGGAGGTGCATAATGCCAAGACAAAGCCGCGGGAG |
| | | GAGCAGTACCAGAGCACGTACCGTGTGGTCAGCGTCCTCACCGTCC |
| | | TGCACCAGGACTGGCTGAATGGCAAGGAGTACAAGTGCAAGGTCTC |
| | | CAACAAAGCCCTCCCAGCCCCCATCGAGAAAACCATCTCCAAAGCC |
| | | AAAGGGCAGCCCCGAGAACCACAGGTGTACACCCTGCCCCCATCCC |
| | | GGGAGGAGATGACCAAGAACCAGGTCAGCCTGACCTGCCTGGTCAA |
| | | AGGCTTCTATCCCAGCGACATCGCCGTGGAGTGGGAGAGCAATGGG |
| | | CAGCCGGAGAACAACTACAAGACCACGCCTCCCGTGCTGGACTCCG |
| | | ACGGCTCCTTCTTCCTCTACAGCAAGCTCACCGTGGACAAGAGCAG |
| | | GTGGCAGCAGGGGAACGTCTTCTCATGCTCCGTGATGCATGAGGCT |
| | | CTGCACAACCACTACACGCAGAAGAGCCTCTCCCTGTCTCCGGGTA |
| | | AA |
| Act101 LC | 644 | SDNHGGGSDIQMTQSPSSLSASVGDRVTITCRASQDVNTAVAWYQQKP |
| Act124 LC | | GKAPKLLIYSASFLYSGVPSRFSGSRSGTDFTLTISSLQPEDFATYYCQQ |
| amino acid | | HYTTPPTFGQGTKVEIKRTVAAPSVFIFPPSDEQLKSGTASVVCLLNNFY |
| sequence | | PREAKVQWKVDNALQSGNSQESVTEQDSKDSTYSLSSTLTLSKADYEK |
| | | HKVYACEVTHQGLSSPVTKSFNRGEC |
| Act101 LC | 645 | AGCGACAACCACGGCGGCGGAAGTGACATACAGATGACCCAGAGT |
| Act124 LC | | CCCAGCTCTCTTTCTGCTAGTGTCGGCGACAGGGTGACAATCACGTG |
| nucleic acid | | CCGCGCATCCCAAGACGTCAACACCGCCGTGGCATGGTACCAGCAA |
| sequence | | AAACCAGGCAAAGCCCCTAAACTGTTGATTTATTCCGCTTCATTCCT |
| | | GTATAGTGGGGTACCCTCCCGATTTAGCGGCTCCCGGTCCGGAACA |
| | | GATTTCACTCTCACTATAAGCTCACTTCAGCCAGAAGATTTCGCAAC |
| | | ATACTATTGTCAACAGCATTACACAACCCCGCCAACCTTCGGCCAG |
| | | GGGACCAAAGTCGAGATCAAACGTACGGTGGCTGCACCATCTGTCT |
| | | TCATCTTCCCGCCATCTGATGAGCAGTTGAAATCTGGAACTGCCTCT |
| | | GTTGTGTGCCTGCTGAATAACTTCTATCCCAGAGAGGCCAAAGTAC |
| | | AGTGGAAGGTGGATAACGCCCTCCAATCGGGTAACTCCCAGGAGAG |
| | | TGTCACAGAGCAGGACAGCAAGGACAGCACCTACAGCCTCAGCAGC |
| | | ACCCTGACGCTGAGCAAAGCAGACTACGAGAAACACAAAGTCTACG |
| | | CCTGCGAAGTCACCCATCAGGGCCTGAGCTCGCCCGTCACAAAGAG |
| | | CTTCAACAGGGGAGAGTGT |
| pLW321: HC | 646 | QGQSGSGYLWGCEWNCGGITTGSSGGSGGSGGISSGLLSGRSDNHGGG |
| amino acid | | SQTVVTQEPSFSVSPGGTVTLTCRSSTGAVTTSNYANWVQQTPGQAPR |
| sequence | | GLIGGTNKRAPGVPDRFSGSILGNKAALTITGAQADDESDYYCALWYS |
| | | NLWVFGGGTKLTVLGGGGSGGGGSGGGGSEVQLVESGGGLVQPGGSL |
| | | RLSCAASGFTFSTYAMNWVRQAPGKGLEWVARIRSKYNNYATYYADS |
| | | VKDRFTISRDDSKNSLYLQMNSLKTEDTAVYYCVRHGNFGNSYVSWF |
| | | AYWGQGTLVTVSSGGGGSGGGGSGGGGSEVQLVESGGGLVQPGGSLR |
| | | LSCAASGFNIKDTYIHWVRQAPGKGLEWVARIYPTNGYTRYADSVKG |
| | | RFTISADTSKNTAYLQMNSLRAEDTAVYYCSRWGGDGFYAMDYWGQ |
| | | GTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPS |
| | | NTKVDKKVEPKSCDKTHTCPPCPAPEFEGGPSVFLFPPKPKDTLMISRTP |
| | | EVTCVVVDVSHEDPEVKFNWYVDGVEVHNAKTKPREEQYQSTYRVV |
| | | SVLTVLHQDWLNGKEYKCKVSNKALPASIEKTISKAKGQPREPQVYTL |
| | | PPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| pLW321: HC | 647 | CAAGGCCAGTCTGGTTCAGGTTATCTGTGGGGTTGCGAGTGGAATT |
| nucleic acid | | GCGGAGGGATCACTACAGGATCTAGTGGTGGCAGCGGTGGCTCTGG |
| sequence | | TGGTATATCGAGTGGATTGCTGTCTGGCAGATCTGACAATCACGGC |
| | | GGCGGATCCCAGACGGTAGTGACTCAGGAGCCATCATTTTCTGTCTC |
| | | TCCTGGAGGTACTGTGACACTCACATGTAGAAGCTCAACTGGTGCA |
| | | GTCACCACTTCAAATTACGCGAATTGGGTCCAGCAGACCCCTGGGC |
| | | AGGCTCCGAGAGGGTTGATTGGAGGTACTAACAAACGGGCACCGGG |
| | | AGTGCCTGATAGGTTTTCCGGTTCTATTCTCGGAAACAAGGCGGCTC |
| | | TCACGATCACGGGTGCGCAGGCCGACGATGAATCAGACTATTACTG |
| | | CGCTTTGTGGTACTCAAACCTGTGGGTATTCGGAGGGGGCACCAAG |
| | | CTGACGGTGTTGGGTGGGGGGGGCTCTGGGGGAGGGGGAAGCGGA |
| | | GGTGGGGGCAGCGAGGTTCAGCTTGTTGAAAGTGGTGGCGGACTCG |
| | | TACAACCGGGTGGAAGTCTTAGACTCTCATGTGCAGCATCTGGATTT |
| | | ACTTTTTCTACTTATGCTATGAACTGGGTAAGACAGGCACCGGGGA |
| | | AAGGGCTGGAATGGGTTGCACGCATTCGATCTAAATACAATAACTA |
| | | TGCTACATACTACGCCGATAGTGTTAAGGATCGATTCACTATATCTC |
| | | GGGACGACAGTAAGAACTCACTTTACCTGCAGATGAATTCCTTGAA |
| | | AACTGAGGACACGGCCGTTTATTATTGTGTACGGCACGGGAATTTC |
| | | GGCAATTCTTACGTTTCCTGGTTCGCCTATTGGGGGCAAGGTACCCT |
| | | CGTGACAGTATCCTCAGGAGGTGGTGGATCAGGAGGTGGTGGTTCT |
| | | GGAGGTGGTGGAAGTGAAGTGCAGCTGGTGGAGTCCGGAGGCGGT |
| | | CTGGTACAACCCGGCGGCTCCCTGCGCCTGAGTTGCGCCGCTAGTG |
| | | GATTCAATATAAAGGACACCTACATCCATTGGGTAAGGCAAGCACC |
| | | TGGCAAGGGTCTGGAGTGGGTGGCTCGCATCTACCCAACCAACGGC |
| | | TATACCAGGTACGCGGATTCAGTTAAGGGGCGGTTCACTATCAGCG |
| | | CCGACACCAGCAAGAATACAGCTTACCTGCAGATGAATAGCCTGCG |
| | | CGCAGAGGATACTGCGGTGTATTACTGCAGTAGATGGGGGGGCGAT |
| | | GGATTTTACGCAATGGACTACTGGGGGCAGGGAACCTTGGTGACCG |
| | | TGTCCAGCGCTAGCACCAAGGGCCCATCGGTCTTCCCCCTGGCACCC |
| | | TCCTCCAAGAGCACCTCTGGGGGCACAGCGGCCCTGGGCTGCCTGG |
| | | TCAAGGACTACTTCCCCGAACCGGTGACGGTGTCGTGGAACTCAGG |
| | | CGCCCTGACCAGCGGCGTGCACACCTTCCCGGCTGTCCTACAGTCCT |
| | | CAGGACTCTACTCCCTCAGCAGCGTGGTGACCGTGCCCTCCAGCAG |
| | | CTTGGGCACCCAGACCTACATCTGCAACGTGAATCACAAGCCCAGC |
| | | AACACCAAGGTGGACAAGAAAGTTGAGCCCAAATCTTGTGACAAAA |
| | | CTCACACATGCCCACCGTGCCCAGCACCTGAATTTGAAGGGGGACC |
| | | GTCAGTCTTCCTCTTCCCCCCAAAACCCAAGGACACCCTCATGATCT |
| | | CCCGGACCCCTGAGGTCACATGCGTGGTGGTGGACGTGAGCCACGA |
| | | AGACCCTGAGGTCAAGTTCAACTGGTACGTGGACGGCGTGGAGGTG |
| | | CATAATGCCAAGACAAAGCCGCGGGAGGAGCAGTACCAGAGCACG |
| | | TACCGTGTGGTCAGCGTCCTCACCGTCCTGCACCAGGACTGGCTGAA |
| | | TGGCAAGGAGTACAAGTGCAAGGTCTCCAACAAAGCCCTQCCAGCC |
| | | TCAATCGAGAAAACCATCTCCAAAGCCAAAGGGCAGCCCCGAGAAC |
| | | CACAGGTGTACACCCTGCCCCCATCCCGGGAGGAGATGACCAAGAA |
| | | CCAGGTCAGCCTGACCTGCCTGGTCAAAGGCTTCTATCCCAGCGAC |
| | | ATCGCCGTGGAGTGGGAGAGCAATGGGCAGCCGGAGAACAACTAC |
| | | AAGACCACGCCTCCCGTGCTGGACTCCGACGGCTCCTTCTTCCTCTA |
| | | CAGCAAGCTCACCGTGGACAAGAGCAGGTGGCAGCAGGGGAACGT |
| | | CTTCTCATGCTCCGTGATGCATGAGGCTCTGCACAACCACTACACGC |
| | | AGAAGAGCCTCTCCCTGTCTCCGGGTAAA |
| Activated- | 648 | SDNHGGGSQTVVTQEPSFSVSPGGTVTLTCRSSTGAVTTSNYANWVQQ |
| pLW321 HC | | TPGQAPRGLIGGTNKRAPGVPDRFSGSILGNKAALTITGAQADDESDYY |
| amino acid | | CALWYSNLWVFGGGTKLTVLGGGGSGGGGSGGGGSEVQLVESGGGL |
| sequence | | VQPGGSLRLSCAASGFTFSTYAMNWVRQAPGKGLEWVARIRSKYNNY |
| | | ATYYADSVKDRFTISRDDSKNSLYLQMNSLKTEDTAVYYCVRHGNFG |
| | | NSYVSWFAYWGQGTLVTVSSGGGGSGGGGSGGGGSEVQLVESGGGL |
| | | VQPGGSLRLSCAASGFNIKDTYIHWVRQAPGKGLEWVARIYPTNGYTR |
| | | YADSVKGRFTISADTSKNTAYLQMNSLRAEDTAVYYCSRWGGDGFYA |
| | | MDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYIC |
| | | NVNHKPSNTKVDKKVEPKSCDKTHTCPPCPAPEFEGGPSVFLFPPKPKD |
| | | TLMISRTPEVTCVVVDVSHEDPEVKFNWYVDGVEVHNAKTKPREEQY |
| | | QSTYRVVSVLTVLHQDWLNGKEYKCKVSNKALPASIEKTISKAKGQPR |
| | | EPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKS |
| | | LSLSPGK |
| Activated- | 649 | TCTGACAATCACGGCGGCGGATCCCAGACGGTAGTGACTCAGGAGC |
| pLW321 HC | | CATCATTTTCTGTCTCTCCTGGAGGTACTGTGACACTCACATGTAGA |
| nucleic acid | | AGCTCAACTGGTGCAGTCACCACTTCAAATTACGCGAATTGGGTCC |
| sequence | | AGCAGACCCCTGGGCAGGCTCCGAGAGGGTTGATTGGAGGTACTAA |
| | | CAAACGGGCACCGGGAGTGCCTGATAGGTTTTCCGGTTCTATTCTCG |
| | | GAAACAAGGCGGCTCTCACGATCACGGGTGCGCAGGCCGACGATGA |
| | | ATCAGACTATTACTGCGCTTTGTGGTACTCAAACCTGTGGGTATTCG |
| | | GAGGGGGCACCAAGCTGACGGTGTTGGGTGGGGGGGGCTCTGGGG |
| | | GAGGGGGAAGCGGAGGTGGGGGCAGCGAGGTTCAGCTTGTTGAAA |
| | | GTGGTGGCGGACTCGTACAACCGGGTGGAAGTCTTAGACTCTCATG |
| | | TGCAGCATCTGGATTTACTTTTTCTACTTATGCTATGAACTGGGTAA |
| | | GACAGGCACCGGGGAAAGGGCTGGAATGGGTTGCACGCATTCGATC |
| | | TAAATACAATAACTATGCTACATACTACGCCGATAGTGTTAAGGAT |
| | | CGATTCACTATATCTCGGGACGACAGTAAGAACTCACTTTACCTGCA |
| | | GATGAATTCCTTGAAAACTGAGGACACGGCCGTTTATTATTGTGTAC |
| | | GGCACGGGAATTTCGGCAATTCTTACGTTTCCTGGTTCGCCTATTGG |
| | | GGGCAAGGTACCCTCGTGACAGTATCCTCAGGAGGTGGTGGATCAG |
| | | GAGGTGGTGGTTCTGGAGGTGGTGGAAGTGAAGTGCAGCTGGTGGA |
| | | GTCCGGAGGCGGTCTGGTACAACCCGGCGGCTCCCTGCGCCTGAGT |
| | | TGCGCCGCTAGTGGATTCAATATAAAGGACACCTACATCCATTGGG |
| | | TAAGGCAAGCACCTGGCAAGGGTCTGGAGTGGGTGGCTCGCATCTA |
| | | CCCAACCAACGGCTATACCAGGTACGCGGATTCAGTTAAGGGGCGG |
| | | TTCACTATCAGCGCCGACACCAGCAAGAATACAGCTTACCTGCAGA |
| | | TGAATAGCCTGCGCGCAGAGGATACTGCGGTGTATTACTGCAGTAG |
| | | ATGGGGGGGGATGGATTTTACGCAATGGACTACTGGGGGCAGGGA |
| | | ACCTTGGTGACCGTGTCCAGCGCTAGCACCAAGGGCCCATCGGTCTT |
| | | CCCCCTGGCACCCTCCTCCAAGAGCACCTCTGGGGGCACAGCGGCC |
| | | CTGGGCTGCCTGGTCAAGGACTACTTCCCCGAACCGGTGACGGTGT |
| | | CGTGGAACTCAGGCGCCCTGACCAGCGGCGTGCACACCTTCCCGGC |
| | | TGTCCTACAGTCCTCAGGACTCTACTCCCTCAGCAGCGTGGTGACCG |
| | | TGCCCTCCAGCAGCTTGGGCACCCAGACCTACATCTGCAACGTGAA |
| | | TCACAAGCCCAGCAACACCAAGGTGGACAAGAAAGTTGAGCCCAA |
| | | ATCTTGTGACAAAACTCACACATGCCCACCGTGCCCAGCACCTGAA |
| | | TTTGAAGGGGGACCGTCAGTCTTCCTCTTCCCCCCAAAACCCAAGGA |
| | | CACCCTCATGATCTCCCGGACCCCTGAGGTCACATGCGTGGTGGTGG |
| | | ACGTGAGCCACGAAGACCCTGAGGTCAAGTTCAACTGGTACGTGGA |
| | | CGGCGTGGAGGTGCATAATGCCAAGACAAAGCCGCGGGAGGAGCA |
| | | GTACCAGAGCACGTACCGTGTGGTCAGCGTCCTCACCGTCCTGCACC |
| | | AGGACTGGCTGAATGGCAAGGAGTACAAGTGCAAGGTCTCCAACAA |
| | | AGCCCTQCCAGCCTCAATCGAGAAAACCATCTCCAAAGCCAAAGGG |
| | | CAGCCCCGAGAACCACAGGTGTACACCCTGCCCCCATCCCGGGAGG |
| | | AGATGACCAAGAACCAGGTCAGCCTGACCTGCCTGGTCAAAGGCTT |
| | | CTATCCCAGCGACATCGCCGTGGAGTGGGAGAGCAATGGGCAGCCG |
| | | GAGAACAACTACAAGACCACGCCTCCCGTGCTGGACTCCGACGGCT |
| | | CCTTCTTCCTCTACAGCAAGCTCACCGTGGACAAGAGCAGGTGGCA |
| | | GCAGGGGAACGTCTTCTCATGCTCCGTGATGCATGAGGCTCTGCAC |
| | | AACCACTACACGCAGAAGAGCCTCTCCCTGTCTCCGGGTAAA |
| CI156 HC | 670 | QGQSGSGYLWGCEWNCGGITTGSSGGSGGSGGLSGRSDNHGGGSQTV |
| amino acid | | VTQEPSFSVSPGGTVTLTCRSSTGAVTTSNYANWVQQTPGQAPRGLIG |
| sequence | | GTNKRAPGVPDRFSGSILGNKAALTITGAQADDESDYYCALWYSNLW |
| | | VFGGGTKLTVLGGGGSGGGGSGGGGSEVQLVESGGGLVQPGGSLRLS |
| | | CAASGFTFSTYAMNWVRQAPGKGLEWVARIRSKYNNYATYYADSVK |
| | | DRFTISRDDSKNSLYLQMNSLKTEDTAVYYCVRHGNFGNSYVSWFAY |
| | | WGQGTLVTVSSGGGGSGGGGSGGGGSEVQLVESGGGLVQPGGSLRLS |
| | | CAASGFNIKDTYIHWVRQAPGKGLEWVARIYPINGYTRYADSVKGRF |
| | | TISADTSKNTAYLQMNSLRAEDTAVYYCSRWGGDGFYAMDYWGQGT |
| | | LVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNT |
| | | KVDKKVEPKSCDKTHTCPPCPAPEFEGGPSVFLFPPKPKDTLMISRTPEV |
| | | TCVVVDVSHEDPEVKFNWYVDGVEVHNAKTKPREEQYQSTYRVVSV |
| | | LTVLHQDWLNGKEYKCKVSNKALPASIEKTISKAKGQPREPQVYTLPP |
| | | SREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| CI156 HC | 671 | CAAGGCCAGTCTGGTTCAGGTTATCTGTGGGGTTGCGAGTGGAATT |
| nucleic acid | | GCGGAGGGATCACTACAGGATCTAGTGGTGGCAGCGGTGGCTCTGG |
| sequence | | TGGTCTGAGCGGCCGTTCCGATAATCATGGCGGCGGATCCCAGACG |
| | | GTAGTGACTCAGGAGCCATCATTTTCTGTCTCTCCTGGAGGTACTGT |
| | | GACACTCACATGTAGAAGCTCAACTGGTGCAGTCACCACTTCAAAT |
| | | TACGCGAATTGGGTCCAGCAGACCCCTGGGCAGGCTCCGAGAGGGT |
| | | TGATTGGAGGTACTAACAAACGGGCACCGGGAGTGCCTGATAGGTT |
| | | TTCCGGTTCTATTCTCGGAAACAAGGCGGCTCTCACGATCACGGGTG |
| | | CGCAGGCCGACGATGAATCAGACTATTACTGCGCTTTGTGGTACTCA |
| | | AACCTGTGGGTATTCGGAGGGGGCACCAAGCTGACGGTGTTGGGTG |
| | | GGGGGGGCTCTGGGGGAGGGGGAAGCGGAGGTGGGGGCAGCGAGG |
| | | TTCAGCTTGTTGAAAGTGGTGGCGGACTCGTACAACCGGGTGGAAG |
| | | TCTTAGACTCTCATGTGCAGCATCTGGATTTACTTTTTCTACTTATGC |
| | | TATGAACTGGGTAAGACAGGCACCGGGGAAAGGGCTGGAATGGGT |
| | | TGCACGCATTCGATCTAAATACAATAACTATGCTACATACTACGCCG |
| | | ATAGTGTTAAGGATCGATTCACTATATCTCGGGACGACAGTAAGAA |
| | | CTCACTTTACCTGCAGATGAATTCCTTGAAAACTGAGGACACGGCC |
| | | GTTTATTATTGTGTACGGCACGGGAATTTCGGCAATTCTTACGTTTC |
| | | CTGGTTCGCCTATTGGGGGCAAGGTACCCTCGTGACAGTATCCTCAG |
| | | GAGGTGGTGGATCAGGAGGTGGTGGTTCTGGAGGTGGTGGAAGTGA |
| | | AGTGCAGCTGGTGGAGTCCGGAGGCGGTCTGGTACAACCCGGCGGC |
| | | TCCCTGCGCCTGAGTTGCGCCGCTAGTGGATTCAATATAAAGGACA |
| | | CCTACATCCATTGGGTAAGGCAAGCACCTGGCAAGGGTCTGGAGTG |
| | | GGTGGCTCGCATCTACCCAACCAACGGCTATACCAGGTACGCGGAT |
| | | TCAGTTAAGGGGCGGTTCACTATCAGCGCCGACACCAGCAAGAATA |
| | | CAGCTTACCTGCAGATGAATAGCCTGCGCGCAGAGGATACTGCGGT |
| | | GTATTACTGCAGTAGATGGGGGGGCGATGGATTTTACGCAATGGAC |
| | | TACTGGGGGCAGGGAACCTTGGTGACCGTGTCCAGCGCTAGCACCA |
| | | AGGGCCCATCGGTCTTCCCCCTGGCACCCTCCTCCAAGAGCACCTCT |
| | | GGGGGCACAGCGGCCCTGGGCTGCCTGGTCAAGGACTACTTCCCCG |
| | | AACCGGTGACGGTGTCGTGGAACTCAGGCGCCCTGACCAGCGGCGT |
| | | GCACACCTTCCCGGCTGTCCTACAGTCCTCAGGACTCTACTCCCTCA |
| | | GCAGCGTGGTGACCGTGCCCTCCAGCAGCTTGGGCACCCAGACCTA |
| | | CATCTGCAACGTGAATCACAAGCCCAGCAACACCAAGGTGGACAAG |
| | | AAAGTTGAGCCCAAATCTTGTGACAAAACTCACACATGCCCACCGT |
| | | GCCCAGCACCTGAATTTGAAGGGGGACCGTCAGTCTTCCTCTTCCCC |
| | | CCAAAACCCAAGGACACCCTCATGATCTCCCGGACCCCTGAGGTCA |
| | | CATGCGTGGTGGTGGACGTGAGCCACGAAGACCCTGAGGTCAAGTT |
| | | CAACTGGTACGTGGACGGCGTGGAGGTGCATAATGCCAAGACAAAG |
| | | CCGCGGGAGGAGCAGTACCAGAGCACGTACCGTGTGGTCAGCGTCC |
| | | TCACCGTCCTGCACCAGGACTGGCTGAATGGCAAGGAGTACAAGTG |
| | | CAAGGTCTCCAACAAAGCCCTCCCAGCCTCAATCGAGAAAACCATC |
| | | TCCAAAGCCAAAGGGCAGCCCCGAGAACCACAGGTGTACACCCTGC |
| | | CCCCATCCCGGGAGGAGATGACCAAGAACCAGGTCAGCCTGACCTG |
| | | CCTGGTCAAAGGCTTCTATCCCAGCGACATCGCCGTGGAGTGGGAG |
| | | AGCAATGGGCAGCCGGAGAACAACTACAAGACCACGCCTCCCGTGC |
| | | TGGACTCCGACGGCTCCTTCTTCCTCTACAGCAAGCTCACCGTGGAC |
| | | AAGAGCAGGTGGCAGCAGGGGAACGTCTTCTCATGCTCCGTGATGC |
| | | ATGAGGCTCTGCACAACCACTACACGCAGAAGAGCCTCTCCCTGTC |
| | | TCCGGGTAAA |
| pLW348: LC | 672 | QGQSGQGALICCSDVSGLCRWCGGGSSGGSLSGRSDNHGGGSDIQMT |
| amino acid | | QSPSSLSASVGDRVTITCRASQDVNTAVAWYQQKPGKAPKLLIYSASFL |
| sequence | | YSGVPSRFSGSRSGTDFTLTISSLQPEDFATYYCQQHYTTPPTFGQGTKV |
| | | EIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGL |
| | | SSPVTKSFNRGEC |
| pLW348: LC | 673 | CAGGGCCAGTCAGGGCAGGGAGCCCTGATCTGCTGTAGCGACGTGT |
| nucleic acid | | CAGGGCTTTGTAGATGGTGCGGGGGAGGATCCTCTGGTGGATCTCT |
| sequence | | GAGCGGCCGTTCCGATAATCATGGCGGCGGAAGTGACATACAGATG |
| | | ACCCAGAGTCCCAGCTCTCTTTCTGCTAGTGTCGGCGACAGGGTGAC |
| | | AATCACGTGCCGCGCATCCCAAGACGTCAACACCGCCGTGGCATGG |
| | | TACCAGCAAAAACCAGGCAAAGCCCCTAAACTGTTGATTTATTCCG |
| | | CTTCATTCCTGTATAGTGGGGTACCCTCCCGATTTAGCGGCTCCCGG |
| | | TCCGGAACAGATTTCACTCTCACTATAAGCTCACTTCAGCCAGAAGA |
| | | TTTCGCAACATACTATTGTCAACAGCATTACACAACCCCGCCAACCT |
| | | TCGGCCAGGGGACCAAAGTCGAGATCAAACGTACGGTGGCTGCACC |
| | | ATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCC |
| | | AAAGTACAGTGGAAGGTGGATAACGCCCTCCAATCGGGTAACTCCC |
| | | AGGAGAGTGTCACAGAGCAGGACAGCAAGGACAGCACCTACAGCC |
| | | TCAGCAGCACCCTGACGCTGAGCAAAGCAGACTACGAGAAACACA |
| | | AAGTCTACGCCTGCGAAGTCACCCATCAGGGCCTGAGCTCGCCCGT |
| | | CACAAAGAGCTTCAACAGGGGAGAGTGT |
| CI185-CI392 | 674 | QGQSGSGYLWGCEWNCGGITTGSSGGSGGSGGISSGLLSGRSDNIGGG |
| HC | | SQTVVTQEPSFSVSPGGTVTLTCRSSTGAVTTSNYANWVQQTPGQAPR |
| amino acid | | GLIGGTNKRAPGVPDRFSGSILGNKAALTITGAQADDESDYYCALWYS |
| sequence | | NLWVFGGGTKLTVLGGGGSGGGGSGGGGSEVQLVESGGGLVQPGGSL |
| | | RLSCAASGFTFSTYAMNWVRQAPGKGLEWVARIRSKYNNYATYYADS |
| | | VKDRFTISRDDSKNSLYLQMNSLKTEDTAVYYCVRHGNFGNSYVSWF |
| | | AYWGQGTLVTVSSGGGGSGGGGSGGGGSEVQLVESGGGLVQPGGSLR |
| | | LSCAASGFNIKDTYIHWVRQAPGKGLEWVARIYPTNGYTRYADSVKG |
| | | RFTISADTSKNTAYLQMNSLRAEDTAVYYCSRWGGDGFYAMDYWGQ |
| | | GTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPS |
| | | NTKVDKKVEPKSCDKTHTCPPCPAPEFEGGPSVFLFPPKPKDTLMISRTP |
| | | EVTCVVVDVSHEDPEVKFNWYVDGVEVHNAKTKPREEQYQSTYRVV |
| | | SVLTVLHQDWLNGKEYKCKVSNKALPASIEKTISKAKGQPREPQVYTL |
| | | PPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| CI185-CI392 | 675 | CAAGGCCAGTCTGGTTCAGGTTATCTGTGGGGTTGCGAGTGGAATT |
| HC | | GCGGAGGGATCACTACAGGATCTAGTGGTGGCAGCGGTGGCTCTGG |
| nucleic acid | | TGGTATATCGAGTGGATTGCTGTCTGGCAGATCTGACAATATTGGCG |
| sequence | | GCGGATCCCAGACGGTAGTGACTCAGGAGCCATCATTTTCTGTCTCT |
| | | CCTGGAGGTACTGTGACACTCACATGTAGAAGCTCAACTGGTGCAG |
| | | TCACCACTTCAAATTACGCGAATTGGGTCCAGCAGACCCCTGGGCA |
| | | GGCTCCGAGAGGGTTGATTGGAGGTACTAACAAACGGGCACCGGGA |
| | | GTGCCTGATAGGTTTTCCGGTTCTATTCTCGGAAACAAGGCGGCTCT |
| | | CACGATCACGGGTGCGCAGGCCGACGATGAATCAGACTATTACTGC |
| | | GCTTTGTGGTACTCAAACCTGTGGGTATTCGGAGGGGGCACCAAGC |
| | | TGACGGTGTTGGGTGGGGGGGGCTCTGGGGGAGGGGGAAGCGGAG |
| | | GTGGGGGCAGCGAGGTTCAGCTTGTTGAAAGTGGTGGCGGACTCGT |
| | | ACAACCGGGTGGAAGTCTTAGACTCTCATGTGCAGCATCTGGATTTA |
| | | CTTTTTCTACTTATGCTATGAACTGGGTAAGACAGGCACCGGGGAA |
| | | AGGGCTGGAATGGGTTGCACGCATTCGATCTAAATACAATAACTAT |
| | | GCTACATACTACGCCGATAGTGTTAAGGATCGATTCACTATATCTCG |
| | | GGACGACAGTAAGAACTCACTTTACCTGCAGATGAATTCCTTGAAA |
| | | ACTGAGGACACGGCCGTTTATTATTGTGTACGGCACGGGAATTTCG |
| | | GCAATTCTTACGTTTCCTGGTTCGCCTATTGGGGGCAAGGTACCCTC |
| | | GTGACAGTATCCTCAGGAGGTGGTGGATCAGGAGGTGGTGGTTCTG |
| | | GAGGTGGTGGAAGTGAAGTGCAGCTGGTGGAGTCCGGAGGCGGTCT |
| | | GGTACAACCCGGCGGCTCCCTGCGCCTGAGTTGCGCCGCTAGTGGA |
| | | TTCAATATAAAGGACACCTACATCCATTGGGTAAGGCAAGCACCTG |
| | | GCAAGGGTCTGGAGTGGGTGGCTCGCATCTACCCAACCAACGGCTA |
| | | TACCAGGTACGCGGATTCAGTTAAGGGGCGGTTCACTATCAGCGCC |
| | | GACACCAGCAAGAATACAGCTTACCTGCAGATGAATAGCCTGCGCG |
| | | CAGAGGATACTGCGGTGTATTACTGCAGTAGATGGGGGGGCGATGG |
| | | ATTTTACGCAATGGACTACTGGGGGCAGGGAACCTTGGTGACCGTG |
| | | TCCAGCGCTAGCACCAAGGGCCCATCGGTCTTCCCCCTGGCACCCTC |
| | | CTCCAAGAGCACCTCTGGGGGCACAGCGGCCCTGGGCTGCCTGGTC |
| | | AAGGACTACTTCCCCGAACCGGTGACGGTGTCGTGGAACTCAGGCG |
| | | CCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTT |
| | | GGGCACCCAGACCTACATCTGCAACGTGAATCACAAGCCCAGCAAC |
| | | ACCAAGGTGGACAAGAAAGTTGAGCCCAAATCTTGTGACAAAACTC |
| | | ACACATGCCCACCGTGCCCAGCACCTGAATTTGAAGGGGGACCGTC |
| | | AGTCTTCCTCTTCCCCCCAAAACCCAAGGACACCCTCATGATCTCCC |
| | | GGACCCCTGAGGTCACATGCGTGGTGGTGGACGTGAGCCACGAAGA |
| | | CCCTGAGGTCAAGTTCAACTGGTACGTGGACGGCGTGGAGGTGCAT |
| | | AATGCCAAGACAAAGCCGCGGGAGGAGCAGTACCAGAGCACGTAC |
| | | CGTGTGGTCAGCGTCCTCACCGTCCTGCACCAGGACTGGCTGAATG |
| | | GCAAGGAGTACAAGTGCAAGGTCTCCAACAAAGCCCTCCCAGCCTC |
| | | AATCGAGAAAACCATCTCCAAAGCCAAAGGGCAGCCCCGAGAACC |
| | | ACAGGTGTACACCCTGCCCCCATCCCGGGAGGAGATGACCAAGAAC |
| | | CAGGTCAGCCTGACCTGCCTGGTCAAAGGCTTCTATCCCAGCGACAT |
| | | CGCCGTGGAGTGGGAGAGCAATGGGCAGCCGGAGAACAACTACAA |
| | | GACCACGCCTCCCGTGCTGGACTCCGACGGCTCCTTCTTCCTCTACA |
| | | GCAAGCTCACCGTGGACAAGAGCAGGTGGCAGCAGGGGAACGTCTT |
| | | CTCATGCTCCGTGATGCATGAGGCTCTGCACAACCACTACACGCAG |
| | | AAGAGCCTCTCCCTGTCTCCGGGTAAA |
| CI185 LC | 676 | QSGQLYCTEGRWYEGTCAGGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI185 LC | 677 | CAGTCAGGGCAGCTTTACTGCACGGAGGGTCGTTGGTATGAGGGAA |
| nucleic acid | | CTTGTGCTGGAGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI381 LC | 678 | QSGQLTKCATSTSTTCDLDGSSGGSGGSGGSGISSGLLSGRSDNIGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI381 LC | 679 | CAGTCAGGGCAGTTGACGAAATGCGCTACCAGCACATCCACTACTT |
| nucleic acid | | GTGATCTTGATGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI382 LC | 680 | QSGQ[FGCGVVLDLDHCAPWGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI382 LC | 681 | CAGTCAGGGCAGTTCGGTTGTGGTGTTGTGTTGGACCTTGACCATTG |
| nucleic acid | | TGCACCATGGGGTTCCAGTGGTGGTTCTGGAGGATCAGGAGGAAGC |
| sequence | | GGTATCTCTAGCGGACTGCTGAGCGGGCGCAGCGACAACATTGGCG |
| | | GATCCGACATACAGATGACCCAGAGTCCCAGCTCTCTTTCTGCTAGT |
| | | GTCGGCGACAGGGTGACAATCACGTGCCGCGCATCCCAAGACGTCA |
| | | ACACCGCCGTGGCATGGTACCAGCAAAAACCAGGCAAAGCCCCTAA |
| | | ACTGTTGATTTATTCCGCTTCATTCCTGTATAGTGGGGTACCCTCCCG |
| | | ATTTAGCGGCTCCCGGTCCGGAACAGATTTCACTCTCACTATAAGCT |
| | | CACTTCAGCCAGAAGATTTCGCAACATACTATTGTCAACAGCATTAC |
| | | ACAACCCCGCCAACCTTCGGCCAGGGGACCAAAGTCGAGATCAAAC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI383 LC | 682 | QSGQTPCWHMIMTNPECMRGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI383 LC | 683 | CAGTCAGGGCAGACTCCTTGTTGGCATATGATAATGACTAACCCTG |
| nucleic acid | | AGTGCATGAGGGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI384 LC | 684 | QSGQYNACWFGPYDCWYRPGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI384 LC | 685 | CAGTCAGGGCAGTACAACGCTTGCTGGTTTGGTCCTTATGACTGCTG |
| pLW363: LC | | GTATCGACCTGGTTCCAGTGGTGGTTCTGGAGGATCAGGAGGAAGC |
| nucleic acid | | GGTATCTCTAGCGGACTGCTGAGCGGGCGCAGCGACAACATTGGCG |
| sequence | | GATCCGACATACAGATGACCCAGAGTCCCAGCTCTCTTTCTGCTAGT |
| | | GTCGGCGACAGGGTGACAATCACGTGCCGCGCATCCCAAGACGTCA |
| | | ACACCGCCGTGGCATGGTACCAGCAAAAACCAGGCAAAGCCCCTAA |
| | | ACTGTTGATTTATTCCGCTTCATTCCTGTATAGTGGGGTACCCTCCCG |
| | | ATTTAGCGGCTCCCGGTCCGGAACAGATTTCACTCTCACTATAAGCT |
| | | CACTTCAGCCAGAAGATTTCGCAACATACTATTGTCAACAGCATTAC |
| | | ACAACCCCGCCAACCTTCGGCCAGGGGACCAAAGTCGAGATCAAAC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI385 LC | 686 | QSGQFLCEPPSWGPDCTMIGSSGGSGGSGGSGISSGLLSGRSDNIGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI385 LC | 687 | CAGTCAGGGCAGTTCCTGTGCGAGCCTCCATCTTGGGGACCAGACT |
| nucleic acid | | GTACCATGATAGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI386 LC | 688 | QSGQDLCSFGLWYRGTCEGGSSGGSGGSGGSGISSGLLSGRSDNIGGSD |
| amino acid | | IQMTQSPSSLSASVGDRVTITCRASQDVNTAVAWYQQKPGKAPKLLIY |
| sequence | | SASFLYSGVPSRFSGSRSGTDFTLTISSLQPEDFATYYCQQHYTTPPTFG |
| | | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| CI386 LC | 689 | CAGTCAGGGCAGGATTIGTGCTCTTTCGGACTCTGGTACCGAGGTAC |
| nucleic acid | | TTGCGAAGGTGGTTCCAGTGGTGGTTCTGGAGGATCAGGAGGAAGC |
| sequence | | GGTATCTCTAGCGGACTGCTGAGCGGGCGCAGCGACAACATTGGCG |
| | | GATCCGACATACAGATGACCCAGAGTCCCAGCTCTCTTTCTGCTAGT |
| | | GTCGGCGACAGGGTGACAATCACGTGCCGCGCATCCCAAGACGTCA |
| | | ACACCGCCGTGGCATGGTACCAGCAAAAACCAGGCAAAGCCCCTAA |
| | | ACTGTTGATTTATTCCGCTTCATTCCTGTATAGTGGGGTACCCTCCCG |
| | | ATTTAGCGGCTCCCGGTCCGGAACAGATTTCACTCTCACTATAAGCT |
| | | CACTTCAGCCAGAAGATTTCGCAACATACTATTGTCAACAGCATTAC |
| | | ACAACCCCGCCAACCTTCGGCCAGGGGACCAAAGTCGAGATCAAAC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI387 LC | 690 | QSGQTCYHDMHKDTIHCNNGSSGGSGGSGGSGISSGLLSGRSDNIGGSD |
| amino acid | | IQMTQSPSSLSASVGDRVTITCRASQDVNTAVAWYQQKPGKAPKLLIY |
| sequence | | SASFLYSGVPSRFSGSRSGTDFTLTISSLQPEDFATYYCQQHYTTPPTFG |
| | | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| CI387 LC | 691 | CAGTCAGGGCAGACTTGTTATCACGATATGCACAAAGACACGATAC |
| nucleic acid | | ACTGCAATAACGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI388 LC | 692 | QSGQLYRDMCGGGCEDWHDGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI388 LC | 693 | CAGTCAGGGCAGCTTTATCGAGACATGTGCGGTGGAGGTTGCGAAG |
| nucleic acid | | ATTGGCATGACGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI186 LC | 694 | QSGQLGSLCDPFDCPGKNLGSSGGSGGSGGSGISSGLLSGRSDNIGGSDI |
| amino acid | | QMTQSPSSLSASVGDRVTITCRASQDVNTAVAWYQQKPGKAPKLLIYS |
| sequence | | ASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| CI186 LC | 695 | CAGTCAGGGCAGCTTGGAAGCCTTTGTGATCCTTTCGATTGCCCTGG |
| nucleic acid | | TAAGAATCTCGGTTCCAGTGGTGGTTCTGGAGGATCAGGAGGAAGC |
| sequence | | GGTATCTCTAGCGGACTGCTGAGCGGGCGCAGCGACAACATTGGCG |
| | | GATCCGACATACAGATGACCCAGAGTCCCAGCTCTCTTTCTGCTAGT |
| | | GTCGGCGACAGGGTGACAATCACGTGCCGCGCATCCCAAGACGTCA |
| | | ACACCGCCGTGGCATGGTACCAGCAAAAACCAGGCAAAGCCCCTAA |
| | | ACTGTTGATTTATTCCGCTTCATTCCTGTATAGTGGGGTACCCTQCCG |
| | | ATTTAGCGGCTCCCGGTCCGGAACAGATTTCACTCTCACTATAAGCT |
| | | CACTTCAGCCAGAAGATTTCGCAACATACTATTGTCAACAGCATTAC |
| | | ACAACCCCGCCAACCTTCGGCCAGGGGACCAAAGTCGAGATCAAAC |
| | | GTACGGTGGCTGCACCATCTGTCTTCATCTTCCCGCCATCTGATGAG |
| | | CAGTTGAAATCTGGAACTGCCTCTGTTGTGTGCCTGCTGAATAACTT |
| | | CTATCCCAGAGAGGCCAAAGTACAGTGGAAGGTGGATAACGCCCTC |
| | | CAATCGGGTAACTCCCAGGAGAGTGTCACAGAGCAGGACAGCAAG |
| | | GACAGCACCTACAGCCTCAGCAGCACCCTGACGCTGAGCAAAGCAG |
| | | ACTACGAGAAACACAAAGTCTACGCCTGCGAAGTCACCCATCAGGG |
| | | CCTGAGCTCGCCCGTCACAAAGAGCTTCAACAGGGGAGAGTGT |
| CI187 LC | 696 | QSGQRGCSFGYWGNGTCLGGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI187 LC | 69 | CAGTCAGGGCAGAGGGGATGCAGCTTTGGTTATTGGGGTAACGGTA |
| nucleic acid | | CATGCTTGGGTGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI389 LC | 698 | QSGQRACSFGVWWKGTCEGGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI389 LC | 699 | CAGTCAGGGCAGCGTGCATGTAGCTTCGGAGTTTGGTGGAAGGGAA |
| nucleic acid | | CTTGCGAGGGAGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI390 LC | 700 | QSGQANVCWLGPYDCRWESGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI390 LC | 701 | CAGTCAGGGCAGGCTAATGTCTGCTGGCTTGGACCATATGACTGCC |
| nucleic acid | | GATGGGAGTCAGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI391 LC | 702 | QSGQGMCTSGRWYGGDCTGGSSGGSGGSGGSGISSGLLSGRSDNIGGS |
| amino acid | | DIQMTQSPSSLSASVGDRVTITCRASQDVNTAVAWYQQKPGKAPKLLI |
| sequence | | YSASFLYSGVPSRFSGSRSGTDFTLTISSLQPEDFATYYCQQHYTTPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| CI391 LC | 703 | CAGTCAGGGCAGGGTATGTGTACCTCTGGTCGATGGTATGGTGGTG |
| nucleic acid | | ACTGCACCGGTGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI392 LC | 704 | QSGQACTHNTMTDKIYCNNGSSGGSGGSGGSGISSGLLSGRSDNIGGSD |
| amino acid | | IQMTQSPSSLSASVGDRVTITCRASQDVNTAVAWYQQKPGKAPKLLIY |
| sequence | | SASFLYSGVPSRFSGSRSGTDFTLTISSLQPEDFATYYCQQHYTTPPTFG |
| | | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| CI392 LC | 705 | CAGTCAGGGCAGGCTTGCACGCACAACACGATGACCGACAAAATAT |
| nucleic acid | | ATTGTAACAACGGTTCCAGTGGTGGTTCTGGAGGATCAGGAGGAAG |
| sequence | | CGGTATCTCTAGCGGACTGCTGAGCGGGCGCAGCGACAACATTGGC |
| | | GGATCCGACATACAGATGACCCAGAGTCCCAGCTCTCTTTCTGCTAG |
| | | TGTCGGCGACAGGGTGACAATCACGTGCCGCGCATCCCAAGACGTC |
| | | AACACCGCCGTGGCATGGTACCAGCAAAAACCAGGCAAAGCCCCTA |
| | | AACTGTTGATTTATTCCGCTTCATTCCTGTATAGTGGGGTACCCTCCC |
| | | GATTTAGCGGCTCCCGGTCCGGAACAGATTTCACTCTCACTATAAGC |
| | | TCACTTCAGCCAGAAGATTTCGCAACATACTATTGTCAACAGCATTA |
| | | CACAACCCCGCCAACCTTCGGCCAGGGGACCAAAGTCGAGATCAAA |
| | | CGTACGGTGGCTGCACCATCTGTCTTCATCTTCCCGCCATCTGATGA |
| | | GCAGTTGAAATCTGGAACTGCCTCTGTTGTGTGCCTGCTGAATAACT |
| | | TCTATCCCAGAGAGGCCAAAGTACAGTGGAAGGTGGATAACGCCCT |
| | | CCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |
| CI238 HCA | 706 | EVQLVESGGGLVQPGGSLRLSCAASGFNIKDTYIHWVRQAPGKGLEW |
| amino acid | | VARTYPINGYTRYADSVKGRFTISADTSKNTAYLQMNSLRAEDTAVYY |
| sequence | | CSRWGGDGFYAMDYWGQGTLVTVSSASTKGPSVFPLAPSSKSTSGGTAA |
| | | LGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAK |
| | | GQPREPQVCTLPPSRDELTKNQVSLSCAVKGFYPSDIAVEWESNGQPEN |
| | | NYKTTPPVLDSDGSFFLVSKLTVDKSRWQQGNVFSCSVMHEALHNHY |
| | | TQKSLSLSPGK |
| CI238 HCA | 707 | GAAGTGCAGCTGGTGGAGTCCGGAGGCGGTCTGGTACAACCCGGCG |
| nucleic acid | | GCTCCCTGCGCCTGAGTTGCGCCGCTAGTGGATTCAATATAAAGGA |
| sequence | | CACCTACATCCATTGGGTAAGGCAAGCACCTGGCAAGGGTCTGGAG |
| | | TGGGTGGCTCGCATCTACCCAACCAACGGCTATACCAGGTACGCGG |
| | | ATTCAGTTAAGGGGCGGTTCACTATCAGCGCCGACACCAGCAAGAA |
| | | TACAGCTTACCTGCAGATGAATAGCCTGCGCGCAGAGGATACTGCG |
| | | GTGTATTACTGCAGTAGATGGGGGGGCGATGGATTTTACGCAATGG |
| | | ACTACTGGGGGCAGGGAACCTTGGTGACCGTGTCCAGCGCTAGCAC |
| | | CAAGGGCCCATCGGTCTTCCCCCTGGCACCCTCCTCCAAGAGCACCT |
| | | CTGGGGGCACAGCGGCCCTGGGCTGCCTGGTCAAGGACTACTTCCC |
| | | CGAACCGGTGACGGTGTCGTGGAACTCAGGCGCCCTGACCAGCGGC |
| | | GTGCACACCTTCCCGGCTGTCCTACAGTCCTCAGGACTCTACTCCCT |
| | | CAGCAGCGTGGTGACCGTGCCCTCCAGCAGCTTGGGCACCCAGACC |
| | | TACATCTGCAACGTGAATCACAAGCCCAGCAACACCAAGGTGGACA |
| | | AGAAAGTTGAGCCCAAATCTTGTGACAAAACTCACACATGCCCACC |
| | | GTGCCCAGCACCTGAACTCCTGGGGGGACCGTCAGTCTTCCTCTTCC |
| | | CCCCAAAACCCAAGGACACCCTCATGATCTCCCGGACCCCTGAGGT |
| | | CACATGCGTGGTGGTGGACGTGAGCCACGAAGACCCTGAGGTCAAG |
| | | TTCAACTGGTACGTGGACGGCGTGGAGGTGCATAATGCCAAGACAA |
| | | AGCCGCGGGAGGAGCAGTACAACAGCACGTACCGTGTGGTCAGCGT |
| | | CCTCACCGTCCTGCACCAGGACTGGCTGAATGGCAAGGAGTACAAG |
| | | TGCAAGGTCTCCAACAAAGCCCTCCCAGCCCCCATCGAGAAAACCA |
| | | TCTCCAAAGCCAAAGGGCAGCCCCGAGAACCACAGGTGTGCACCCT |
| | | GCCCCCATCCCGGGACGAGCTGACCAAGAACCAGGTCAGCCTGAGC |
| | | TGCGCCGTCAAAGGCTTCTATCCCAGCGACATCGCCGTGGAGTGGG |
| | | AGAGCAATGGGCAGCCGGAGAACAACTACAAGACCACGCCTCCCGT |
| | | GCTGGACTCCGACGGCTCCTTCTTCCTCGTCAGCAAGCTCACCGTGG |
| | | ACAAGAGCAGGTGGCAGCAGGGGAACGTCTTCTCATGCTCCGTGAT |
| | | GCATGAGGCTCTGCACAACCACTACACGCAGAAGAGCCTCTCCCTG |
| | | TCTCCGGGTAAA |
| CI238 HCB | 708 | GSSPKSCDKTHTCPPCPAPELLGGPSVFLFPPKPKDTLMISRTPEVTCVV |
| amino acid | | VDVSHEDPEVKFNWYVDGVEVHNAKTKPREEQYNSTYRVVSVLTVLH |
| sequence | | QDWLNGKEYKCKVSNKALPAPIEKTISKAKGQPREPQVYTLPPCRDEL |
| | | TKNQVSLWCLVKGFYPSDIAVEWESNGQPENNYKTTPPVLDSDGSFFL |
| | | YSKLTVDKSRWQQGNVFSCSVMHEALHNHYTQKSLSLSPGK |
| CI238 HCB | 709 | GGCTCGAGCCCCAAATCTTGTGACAAAACTCACACATGCCCACCGT |
| nucleic acid | | GCCCAGCACCTGAACTCCTGGGGGGACCGTCAGTCTTCCTCTTCCCC |
| sequence | | CCAAAACCCAAGGACACCCTCATGATCTCCCGGACCCCTGAGGTCA |
| | | CATGCGTGGTGGTGGACGTGAGCCACGAAGACCCTGAGGTCAAGTT |
| | | CAACTGGTACGTGGACGGCGTGGAGGTGCATAATGCCAAGACAAAG |
| | | CCGCGGGAGGAGCAGTACAACAGCACGTACCGTGTGGTCAGCGTCC |
| | | TCACCGTCCTGCACCAGGACTGGCTGAATGGCAAGGAGTACAAGTG |
| | | CAAGGTCTCCAACAAAGCCCTCCCAGCCCCCATCGAGAAAACCATC |
| | | TCCAAAGCCAAAGGGCAGCCCCGAGAACCACAGGTGTACACCCTGC |
| | | CCCCATGCCGGGACGAGCTGACCAAGAACCAGGTCAGCCTGTGGTG |
| | | CCTGGTCAAAGGCTTCTATCCCAGCGACATCGCCGTGGAGTGGGAG |
| | | AGCAATGGGCAGCCGGAGAACAACTACAAGACCACGCCTCCCGTGC |
| | | TGGACTCCGACGGCTCCTTCTTCCTCTACAGCAAGCTCACCGTGGAC |
| | | AAGAGCAGGTGGCAGCAGGGGAACGTCTTCTCATGCTCCGTGATGC |
| | | ATGAGGCTCTGCACAACCACTACACGCAGAAGAGCCTCTCCCTGTC |
| | | TCCGGGTAAA |
| pEF1071:HER2- | 710 | EVQLVESGGGLVQPGGSLRLSCAASGFNIKDTYIHWVRQAPGKGLEW |
| VH-HCA- | | VARIYPINGYTRYADSVKGRFTISADTSKNTAYLQMNSLRAEDTAVYY |
| hlgG4 | | CSRWGGDGFYAMDYWGQGTLVTVSSASTKGPSVEPLAPCSRSTSESTAA |
| amino acid | | LGCLVKDYFPEPVTVSWNSGALTSGVHTFPAVLQSSGLYSLSSVVTVPSSSLG |
| sequence | | TKTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQ |
| | | FNSTYRVVSVLTVLHQDWLNGKEYKCKVSNKGLPSSIEKTISKAKGQP |
| | | REPQVCTLPPSQEEMTKNQVSLSCAVKGFYPSDIAVEWESNGQPENNY |
| | | KTTPPVLDSDGSFFLVSRLTVDKSRWQEGNVFSCSVMHEALHNHYTQK |
| | | SLSLSLGK |
| pEF1071:HER2- | 711 | GAAGTGCAGCTGGTGGAGTCCGGAGGCGGTCTGGTACAACCCGGCG |
| VH-HCA- | | GCTCCCTGCGCCTGAGTTGCGCCGCTAGTGGATTCAATATAAAGGA |
| blgG4 | | CACCTACATCCATTGGGTAAGGCAAGCACCTGGCAAGGGTCTGGAG |
| nucleic acid | | TGGGTGGCTCGCATCTACCCAACCAACGGCTATACCAGGTACGCGG |
| sequence | | ATTCAGTTAAGGGGCGGTTCACTATCAGCGCCGACACCAGCAAGAA |
| | | TACAGCTTACCTGCAGATGAATAGCCTGCGCGCAGAGGATACTGCG |
| | | GTGTATTACTGCAGTAGATGGGGGGGCGATGGATTTTACGCAATGG |
| | | ACTACTGGGGGCAGGGAACCTTGGTGACCGTGTCCAGCGCTAGCAC |
| | | CAAGGGCCCATCGGTCTTCCCCCTGGCACCCTGTAGCAGAAGCACC |
| | | AGCGAGTCTACAGCGGCCCTGGGCTGCCTGGTCAAGGACTACTTCC |
| | | CCGAACCGGTGACGGTGTCGTGGAACTCAGGCGCCCTGACCAGCGG |
| | | CGTGCACACCTTCCCGGCTGTCCTACAGTCCTCAGGACTCTACTCCC |
| | | TCAGCAGCGTGGTGACCGTGCCCTCCAGCAGCTTGGGCACCAAGAC |
| | | CTACACCTGTAACGTGGACCACAAGCCCAGCAACACCAAGGTGGAC |
| | | AAGCGGGTGGAATCTAAGTACGGCCCTCCCTGCCCTCCTTGCCCAGC |
| | | CCCTGAATTTCTGGGGGGACCGTCAGTCTTCCTCTTCCCCCCAAAAC |
| | | CCAAGGACACCCTCATGATCTCCCGGACCCCTGAGGTCACATGCGT |
| | | GGTGGTGGACGTGAGCCAGGAAGACCCTGAGGTCCAGTTCAACTGG |
| | | TACGTGGACGGCGTGGAGGTGCATAATGCCAAGACAAAGCCGCGG |
| | | GAGGAGCAGTTCAACAGCACGTACCGTGTGGTCAGCGTCCTCACCG |
| | | TCCTGCACCAGGACTGGCTGAATGGCAAGGAGTACAAGTGCAAGGT |
| | | CTCCAACAAAGGCCTGCCCAGCTCCATCGAGAAAACCATCTCCAAA |
| | | GCCAAAGGGCAGCCCCGAGAACCACAGGTGTGCACCCTGCCCCCAT |
| | | CCCAGGAGGAGATGACCAAGAACCAGGTCAGCCTGAGCTGCGCAGT |
| | | CAAAGGCTTCTATCCCAGCGACATCGCCGTGGAGTGGGAGAGCAAT |
| | | GGGCAGCCGGAGAACAACTACAAGACCACGCCTCCCGTGCTGGACT |
| | | CCGACGGCTCCTTCTTCCTCGTCAGCAGACTCACCGTGGACAAGAGC |
| | | AGGTGGCAGGAAGGGAACGTCTTCTCATGCTCCGTGATGCATGAGG |
| | | CTCTGCACAACCACTACACGCAGAAGAGCCTCTCCCTGTCTCTGGGT |
| | | AAA |
| CI239 HCB | 712 | GSSKYGPPCPPCPAPEFLGPEFLGGPSVFLFPPKPKDTLMISRTPEVTCVV |
| amino acid | | VDVSQEDPEVQFNWYVDGVEVHNAKTKPREEQFNSTYRVVSVLTVLH |
| sequence | | QDWLNGKEYKCKVSNKGLPSSIEKTISKAKGQPREPQVYTLPPCQEEM |
| | | TKNQVSLWCLVKGFYPSDIAVEWESNGQPENNYKTTPPVLDSDGSFFL |
| | | YSRLTVDKSRWQEGNVFSCSVMHEALHNHYTQKSLSLSLGK |
| CI239 HCB | 713 | GGCTCGAGCAAGTACGGCCCTCCCTGCCCTCCTTGCCCAGCCCCTGA |
| nucleic acid | | ATTTCTGGGGGGACCGTCAGTCTTCCTCTTCCCCCCAAAACCCAAGG |
| sequence | | ACACCCTCATGATCTCCCGGACCCCTGAGGTCACATGCGTGGTGGTG |
| | | GACGTGAGCCAGGAAGACCCTGAGGTCCAGTTCAACTGGTACGTGG |
| | | ACGGCGTGGAGGTGCATAATGCCAAGACAAAGCCGCGGGAGGAGC |
| | | AGTTCAACAGCACGTACCGTGTGGTCAGCGTCCTCACCGTCCTGCAC |
| | | CAGGACTGGCTGAATGGCAAGGAGTACAAGTGCAAGGTCTCCAACA |
| | | AAGGCCTGCCCAGCTCCATCGAGAAAACCATCTCCAAAGCCAAAGG |
| | | GCAGCCCCGAGAACCACAGGTGTACACCCTGCCCCCATGCCAGGAG |
| | | GAGATGACCAAGAACCAGGTCAGCCTGTGGTGCCTGGTCAAAGGCT |
| | | TCTATCCCAGCGACATCGCCGTGGAGTGGGAGAGCAATGGGCAGCC |
| | | GGAGAACAACTACAAGACCACGCCTCCCGTGCTGGACTCCGACGGC |
| | | TCCTTCTTCCTCTACAGCAGACTCACCGTGGACAAGAGCAGGTGGC |
| | | AGGAAGGGAACGTCTTCTCATGCTCCGTGATGCATGAGGCTCTGCA |
| | | CAACCACTACACGCAGAAGAGCCTCTCCCTGTCTCTGGGTAAA |
| CI152 HCB | 714 | QGQSGSGYLWGCEWNCGGITTGSSGGSGGSGGISSGLLSGRSDNHGGG |
| amino acid | | SQTVVTQEPSFSVSPGGTVTLTCRSSTGAVTTSNYANWVQQTPGQAPR |
| sequence | | GLIGGTNKRAPGVPDRFSGSILGNKAALTITGAQADDESDYYCALWYS |
| | | NLWVFGGGTKLTVLGGGGSGGGGSGGGGSEVQLVESGGGLVQPGGSL |
| | | RLSCAASGFTFSTYAMNWVRQAPGKGLEWVARIRSKYNNYATYYADS |
| | | VKDRFTISRDDSKNSLYLQMNSLKTEDTAVYYCVRHGNFGNSYVSWF |
| | | AYWGQGTLVTVSSGSSKYGPPCPPCPAPEFLGGPSVFLFPPKPKDTLMIS |
| | | RTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVY |
| | | TLPPCQEEMTKNQVSLWCLVKGFYPSDIAVEWESNGQPENNYKTTPPV |
| | | LDSDGSFFLYSRLTVDKSRWQEGNVFSCSVMHEALHNHYTQKSLSLSL |
| | | GK |
| CI152 HCB | 715 | CAAGGCCAGTCTGGTTCAGGTTATCTGTGGGGTTGCGAGTGGAATT |
| nucleic acid | | GCGGAGGGATCACTACAGGATCTAGTGGTGGCAGCGGTGGCTCTGG |
| sequence | | TGGTATATCGAGTGGATTGCTGTCTGGCAGATCTGACAATCACGGC |
| | | GGCGGATCCCAGACGGTAGTGACTCAGGAGCCATCATTTTCTGTCTC |
| | | TCCTGGAGGTACTGTGACACTCACATGTAGAAGCTCAACTGGTGCA |
| | | GTCACCACTTCAAATTACGCGAATTGGGTCCAGCAGACCCCTGGGC |
| | | AGGCTCCGAGAGGGTTGATTGGAGGTACTAACAAACGGGCACCGGG |
| | | AGTGCCTGATAGGTTTTCCGGTTCTATTCTCGGAAACAAGGCGGCTC |
| | | TCACGATCACGGGTGCGCAGGCCGACGATGAATCAGACTATTACTG |
| | | CGCTTTGTGGTACTCAAACCTGTGGGTATTCGGAGGGGGCACCAAG |
| | | CTGACGGTGTTGGGTGGGGGGGGCTCTGGGGGAGGGGGAAGCGGA |
| | | GGTGGGGGCAGCGAGGTTCAGCTTGTTGAAAGTGGTGGCGGACTCG |
| | | TACAACCGGGTGGAAGTCTTAGACTCTCATGTGCAGCATCTGGATTT |
| | | ACTTTTTCTACTTATGCTATGAACTGGGTAAGACAGGCACCGGGGA |
| | | AAGGGCTGGAATGGGTTGCACGCATTCGATCTAAATACAATAACTA |
| | | TGCTACATACTACGCCGATAGTGTTAAGGATCGATTCACTATATCTC |
| | | GGGACGACAGTAAGAACTCACTTTACCTGCAGATGAATTCCTTGAA |
| | | AACTGAGGACACGGCCGTTTATTATTGTGTACGGCACGGGAATTTC |
| | | GGCAATTCTTACGTTTCCTGGTTCGCCTATTGGGGGCAAGGTACCCT |
| | | CGTGACAGTATCCTCAGGCTCGAGCAAGTACGGCCCTCCCTGCCCTC |
| | | CTTGCCCAGCCCCTGAATTTCTGGGGGGACCGTCAGTCTTCCTCTTC |
| | | CCCCCAAAACCCAAGGACACCCTCATGATCTCCCGGACCCCTGAGG |
| | | TCACATGCGTGGTGGTGGACGTGAGCCAGGAAGACCCTGAGGTCCA |
| | | GTTCAACTGGTACGTGGACGGCGTGGAGGTGCATAATGCCAAGACA |
| | | AAGCCGCGGGAGGAGCAGTTCAACAGCACGTACCGTGTGGTCAGCG |
| | | TCCTCACCGTCCTGCACCAGGACTGGCTGAATGGCAAGGAGTACAA |
| | | GTGCAAGGTCTCCAACAAAGGCCTGCCCAGCTCCATCGAGAAAACC |
| | | ATCTCCAAAGCCAAAGGGCAGCCCCGAGAACCACAGGTGTACACCC |
| | | TGCCCCCATGCCAGGAGGAGATGACCAAGAACCAGGTCAGCCTGTG |
| | | GTGCCTGGTCAAAGGCTTCTATCCCAGCGACATCGCCGTGGAGTGG |
| | | GAGAGCAATGGGCAGCCGGAGAACAACTACAAGACCACGCCTCCC |
| | | GTGCTGGACTCCGACGGCTCCTTCTTCCTCTACAGCAGACTCACCGT |
| | | GGACAAGAGCAGGTGGCAGGAAGGGAACGTCTTCTCATGCTCCGTG |
| | | ATGCATGAGGCTCTGCACAACCACTACACGCAGAAGAGCCTCTCCC |
| | | TGTCTCTGGGTAAA |
| CI240 HCB | 716 | QGQSGSGYLWGCEWNCGGITTGSSGGSGGSGGISSGLLSGRSDNHGGG |
| amino acid | | SQTVVTQEPSFSVSPGGTVTLTCRSSTGAVTTSNYANWVQQTPGQAPR |
| sequence | | GLIGGTNKRAPGVPDRFSGSILGNKAALTITGAQADDESDYYCALWYS |
| | | NLWVFGGGTKLTVLGGGGSGGGGSGGGGSEVQLVESGGGLVQPGGSL |
| | | RLSCAASGFTFSTYAMNWVRQAPGKGLEWVARIRSKYNNYATYYADS |
| | | VKDRFTISRDDSKNSLYLQMNSLKTEDTAVYYCVRHGNFGNSYVSWF |
| | | AYWGQGTLVTVSSGGGGSGSSKYGPPCPPCPAPEFLGGPSVFLFPPKPK |
| | | DTLMISRTPEVTCVVVDVSQEDPEVQFNWYVDGVEVHNAKTKPREEQ |
| | | FNSTYRVVSVLTVLHQDWLNGKEYKCKVSNKGLPSSIEKTISKAKGQP |
| | | REPQVYTLPPCQEEMTKNQVSLWCLVKGFYPSDIAVEWESNGQPENN |
| | | YKTTPPVLDSDGSFFLYSRLTVDKSRWQEGNVFSCSVMHEALHNHYTQ |
| | | KSLSLSLGK |
| CI240 HCB | 717 | CAAGGCCAGTCTGGTTCAGGTTATCTGTGGGGTTGCGAGTGGAATT |
| nucleic acid | | GCGGAGGGATCACTACAGGATCTAGTGGTGGCAGCGGTGGCTCTGG |
| sequence | | TGGTATATCGAGTGGATTGCTGTCTGGCAGATCTGACAATCACGGC |
| | | GGCGGATCCCAGACGGTAGTGACTCAGGAGCCATCATTTTCTGTCTC |
| | | TCCTGGAGGTACTGTGACACTCACATGTAGAAGCTCAACTGGTGCA |
| | | GTCACCACTTCAAATTACGCGAATTGGGTCCAGCAGACCCCTGGGC |
| | | AGGCTCCGAGAGGGTTGATTGGAGGTACTAACAAACGGGCACCGGG |
| | | AGTGCCTGATAGGTTTTCCGGTTCTATTCTCGGAAACAAGGCGGCTC |
| | | TCACGATCACGGGTGCGCAGGCCGACGATGAATCAGACTATTACTG |
| | | CGCTTTGTGGTACTCAAACCTGTGGGTATTCGGAGGGGGCACCAAG |
| | | CTGACGGTGTTGGGTGGGGGGGGCTCTGGGGGAGGGGGAAGCGGA |
| | | GGTGGGGGCAGCGAGGTTCAGCTTGTTGAAAGTGGTGGCGGACTCG |
| | | TACAACCGGGTGGAAGTCTTAGACTCTCATGTGCAGCATCTGGATTT |
| | | ACTTTTTCTACTTATGCTATGAACTGGGTAAGACAGGCACCGGGGA |
| | | AAGGGCTGGAATGGGTTGCACGCATTCGATCTAAATACAATAACTA |
| | | TGCTACATACTACGCCGATAGTGTTAAGGATCGATTCACTATATCTC |
| | | GGGACGACAGTAAGAACTCACTTTACCTGCAGATGAATTCCTTGAA |
| | | AACTGAGGACACGGCCGTTTATTATTGTGTACGGCACGGGAATTTC |
| | | GGCAATTCTTACGTTTCCTGGTTCGCCTATTGGGGGCAAGGTACCCT |
| | | CGTGACAGTATCCTCAGGAGGTGGTGGATCAGGCTCGAGCAAGTAC |
| | | GGCCCTCCCTGCCCTCCTTGCCCAGCCCCTGAATTTCTGGGGGGACC |
| | | GTCAGTCTTCCTCTTCCCCCCAAAACCCAAGGACACCCTCATGATCT |
| | | CCCGGACCCCTGAGGTCACATGCGTGGTGGTGGACGTGAGCCAGGA |
| | | AGACCCTGAGGTCCAGTTCAACTGGTACGTGGACGGCGTGGAGGTG |
| | | CATAATGCCAAGACAAAGCCGCGGGAGGAGCAGTTCAACAGCACG |
| | | TACCGTGTGGTCAGCGTCCTCACCGTCCTGCACCAGGACTGGCTGAA |
| | | TGGCAAGGAGTACAAGTGCAAGGTCTCCAACAAAGGCCTGCCCAGC |
| | | TCCATCGAGAAAACCATCTCCAAAGCCAAAGGGCAGCCCCGAGAAC |
| | | CACAGGTGTACACCCTGCCCCCATGCCAGGAGGAGATGACCAAGAA |
| | | CCAGGTCAGCCTGTGGTGCCTGGTCAAAGGCTTCTATCCCAGCGAC |
| | | ATCGCCGTGGAGTGGGAGAGCAATGGGCAGCCGGAGAACAACTAC |
| | | AAGACCACGCCTCCCGTGCTGGACTCCGACGGCTCCTTCTTCCTCTA |
| | | CAGCAGACTCACCGTGGACAAGAGCAGGTGGCAGGAAGGGAACGT |
| | | CTTCTCATGCTCCGTGATGCATGAGGCTCTGCACAACCACTACACGC |
| | | AGAAGAGCCTCTCCCTGTCTCTGGGTAAA |
| CI148 HCB | 718 | QGQSGSGYLWGCEWNCGGITTGSSGGSGGSGGISSGLLSGRSDNHGGG |
| amino acid | | SQTVVTQEPSFSVSPGGTVTLTCRSSTGAVTTSNYANWVQQTPGQAPR |
| sequence | | GLIGGTNKRAPGVPDRFSGSILGNKAALTITGAQADDESDYYCALWYS |
| | | NLWVFGGGTKLTVLGGGGSGGGGSGGGGSEVQLVESGGGLVQPGGSL |
| | | RLSCAASGFTFSTYAMNWVRQAPGKGLEWVARIRSKYNNYATYYADS |
| | | VKDRFTISRDDSKNSLYLQMNSLKTEDTAVYYCVRHGNFGNSYVSWF |
| | | AYWGQGTLVTVSSGGGGSGGGGSGGGGSGSSKYGPPCPPCPAPEFLGG |
| | | PSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIE |
| | | KTISKAKGQPREPQVYTLPPCQEEMTKNQVSLWCLVKGFYPSDIAVEW |
| | | ESNGQPENNYKTTPPVLDSDGSFFLYSRLTVDKSRWQEGNVFSCSVMH |
| | | EALHNHYTQKSLSLSLGK |
| Spacer | 719 | QGQSGS |
| Spacer | 720 | GQSGS |
| Spacer | 721 | QSGS |
| Spacer | 722 | QGQSGQG |
| Spacer | 723 | GQSGQG |
| Spacer | 724 | QSGQG |
| Spacer | 725 | SGQG |
| Spacer | 726 | QGQSGQ |
| Spacer | 727 | GQSGQ |
| Spacer | 728 | QSGQ |
| Spacer | 729 | QGQSG |
| hu IgGI CH1 | 730 | ASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKV |
| | | E |
| hu IgG4 CH1 | 731 | ASTKGPSVFPLAPCSRSTSESTAALGCLVKDYFPEPVTVSWNSGALTSG |
| | | VHTFPAVLQSSGLYSLSSVVTVPSSSLGTKTYTCNVDHKPSNTKVDKR |
| | | VE |
| hu constant light | 732 | RTVAAPSVFIFPPSDEQLKSGTASVVCLLNNFYPREAKVQWKVDNALQ |
| domain | | SGNSQESVTEQDSKDSTYSLSSTLTLSKADYEKHKVYACEVTHQGLSSP |
| | | VTKSFNRGEC |
| IgG1 hinge | 733 | PKSCDKTHTCPPCPAPELLG |
| IgG4 hinge | 734 | SKYGPPCPPCPAPEFLG |
| IgG1 Fc | 735 | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPG |
| IgG1 isoform | 736 | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPG |
| IgG4Fc | 737 | GPSVFLFPPKPKDTLMISRTPEVTCVVVDVSQEDPEVQFNWYVDGVEV |
| | | HNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMH |
| | | EALHNHYTQKSLSLSLG |
| Linker | 738 | GGGGSGS |
| Linker | 739 | GGGGSGGGGSGGGGSGS |
| CI148 HCB | 740 | CAAGGCCAGTCTGGTTCAGGTTATCTGTGGGGTTGCGAGTGGAATT |
| nucleic acid | | GCGGAGGGATCACTACAGGATCTAGTGGTGGCAGCGGTGGCTCTGG |
| sequence | | TGGTATATCGAGTGGATTGCTGTCTGGCAGATCTGACAATCACGGC |
| | | GGCGGATCCCAGACGGTAGTGACTCAGGAGCCATCATTTTCTGTCTC |
| | | TCCTGGAGGTACTGTGACACTCACATGTAGAAGCTCAACTGGTGCA |
| | | GTCACCACTTCAAATTACGCGAATTGGGTCCAGCAGACCCCTGGGC |
| | | AGGCTCCGAGAGGGTTGATTGGAGGTACTAACAAACGGGCACCGGG |
| | | AGTGCCTGATAGGTTTTCCGGTTCTATTCTCGGAAACAAGGCGGCTC |
| | | TCACGATCACGGGTGCGCAGGCCGACGATGAATCAGACTATTACTG |
| | | CGCTTTGTGGTACTCAAACCTGTGGGTATTCGGAGGGGGCACCAAG |
| | | CTGACGGTGTTGGGTGGGGGGGGCTCTGGGGGAGGGGGAAGCGGA |
| | | GGTGGGGGCAGCGAGGTTCAGCTTGTTGAAAGTGGTGGCGGACTCG |
| | | TACAACCGGGTGGAAGTCTTAGACTCTCATGTGCAGCATCTGGATTT |
| | | ACTTTTTCTACTTATGCTATGAACTGGGTAAGACAGGCACCGGGGA |
| | | AAGGGCTGGAATGGGTTGCACGCATTCGATCTAAATACAATAACTA |
| | | TGCTACATACTACGCCGATAGTGTTAAGGATCGATTCACTATATCTC |
| | | GGGACGACAGTAAGAACTCACTTTACCTGCAGATGAATTCCTTGAA |
| | | AACTGAGGACACGGCCGTTTATTATTGTGTACGGCACGGGAATTTC |
| | | GGCAATTCTTACGTTTCCTGGTTCGCCTATTGGGGGCAAGGTACCCT |
| | | CGTGACAGTATCCTCAGGAGGTGGTGGATCAGGAGGTGGTGGTTCT |
| | | GGAGGTGGTGGAAGTGGCTCGAGCAAGTACGGCCCTCCCTGCCCTC |
| | | CTTGCCCAGCCCCTGAATTTCTGGGGGGACCGTCAGTCTTCCTCTTC |
| | | CCCCCAAAACCCAAGGACACCCTCATGATCTCCCGGACCCCTGAGG |
| | | TCACATGCGTGGTGGTGGACGTGAGCCAGGAAGACCCTGAGGTQCA |
| | | GTTCAACTGGTACGTGGACGGCGTGGAGGTGCATAATGCCAAGACA |
| | | AAGCCGCGGGAGGAGCAGTTCAACAGCACGTACCGTGTGGTCAGCG |
| | | TCCTCACCGTCCTGCACCAGGACTGGCTGAATGGCAAGGAGTACAA |
| | | GTGCAAGGTCTCCAACAAAGGCCTGCCCAGCTCCATCGAGAAAACC |
| | | ATCTCCAAAGCCAAAGGGCAGCCCCGAGAACCACAGGTGTACACCC |
| | | TGCCCCCATGCCAGGAGGAGATGACCAAGAACCAGGTCAGCCTGTG |
| | | GTGCCTGGTCAAAGGCTTCTATCCCAGCGACATCGCCGTGGAGTGG |
| | | GAGAGCAATGGGCAGCCGGAGAACAACTACAAGACCACGCCTCCC |
| | | GTGCTGGACTCCGACGGCTCCTTCTTCCTCTACAGCAGACTCACCGT |
| | | GGACAAGAGCAGGTGGCAGGAAGGGAACGTCTTCTCATGCTCCGTG |
| | | ATGCATGAGGCTCTGCACAACCACTACACGCAGAAGAGCCTCTCCC |
| | | TGTCTCTGGGTAAA |
| CI153 HCB | 741 | QTVVTQEPSFSVSPGGTVTLTCRSSTGAVTTSNYANWVQQTPGQAPRG |
| amino acid | | LIGGTNKRAPGVPDRFSGSILGNKAALTITGAQADDESDYYCALWYSN |
| sequence | | LWVFGGGTKLTVLGGGGSGGGGSGGGGSEVQLVESGGGLVQPGGSLR |
| | | LSCAASGFTFSTYAMNWVRQAPGKGLEWVARIRSKYNNYATYYADSV |
| | | KDRFTISRDDSKNSLYLQMNSLKTEDTAVYYCVRHGNFGNSYVSWFA |
| | | YWGQGTLVTVSSGSSKYGPPCPPCPAPEFLGGPSVFLFPPKPKDTLMISR |
| | | TPEVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQVYT |
| | | LPPCQEEMTKNQVSLWCLVKGFYPSDIAVEWESNGQPENNYKTTPPVL |
| | | DSDGSFFLYSRLTVDKSRWQEGNVFSCSVMHEALHNHYTQKSLSLSLG |
| | | K |
| CI153 HCB | 742 | CAGACGGTAGTGACTCAGGAGCCATCATTTTCTGTCTCTCCTGGAGG |
| nucleic acid | | TACTGTGACACTCACATGTAGAAGCTCAACTGGTGCAGTCACCACTT |
| sequence | | CAAATTACGCGAATTGGGTCCAGCAGACCCCTGGGCAGGCTCCGAG |
| | | AGGGTTGATTGGAGGTACTAACAAACGGGCACCGGGAGTGCCTGAT |
| | | AGGTTTTCCGGTTCTATTCTCGGAAACAAGGCGGCTCTCACGATCAC |
| | | GGGTGCGCAGGCCGACGATGAATCAGACTATTACTGCGCTTTGTGG |
| | | TACTCAAACCTGTGGGTATTCGGAGGGGGCACCAAGCTGACGGTGT |
| | | TGGGTGGGGGGGGCTCTGGGGGAGGGGGAAGCGGAGGTGGGGGCA |
| | | GCGAGGTTCAGCTTGTTGAAAGTGGTGGCGGACTCGTACAACCGGG |
| | | TGGAAGTCTTAGACTCTCATGTGCAGCATCTGGATTTACTTTTTCTA |
| | | CTTATGCTATGAACTGGGTAAGACAGGCACCGGGGAAAGGGCTGGA |
| | | ATGGGTTGCACGCATTCGATCTAAATACAATAACTATGCTACATACT |
| | | ACGCCGATAGTGTTAAGGATCGATTCACTATATCTCGGGACGACAG |
| | | TAAGAACTCACTTTACCTGCAGATGAATTCCTTGAAAACTGAGGAC |
| | | ACGGCCGTTTATTATTGTGTACGGCACGGGAATTTCGGCAATTCTTA |
| | | CGTTTCCTGGTTCGCCTATTGGGGGCAAGGTACCCTCGTGACAGTAT |
| | | CCTCAGGCTCGAGCAAGTACGGCCCTCCCTGCCCTCCTTGCCCAGCC |
| | | CCTGAATTTCTGGGGGGACCGTCAGTCTTCCTCTTCCCCCCAAAACC |
| | | CAAGGACACCCTCATGATCTCCCGGACCCCTGAGGTCACATGCGTG |
| | | GTGGTGGACGTGAGCCAGGAAGACCCTGAGGTCCAGTTCAACTGGT |
| | | ACGTGGACGGCGTGGAGGTGCATAATGCCAAGACAAAGCCGCGGG |
| | | AGGAGCAGTTCAACAGCACGTACCGTGTGGTCAGCGTCCTCACCGT |
| | | CCTGCACCAGGACTGGCTGAATGGCAAGGAGTACAAGTGCAAGGTC |
| | | TCCAACAAAGGCCTGCCCAGCTCCATCGAGAAAACCATCTCCAAAG |
| | | CCAAAGGGCAGCCCCGAGAACCACAGGTGTACACCCTGCCCCCATG |
| | | CCAGGAGGAGATGACCAAGAACCAGGTCAGCCTGTGGTGCCTGGTC |
| | | AAAGGCTTCTATCCCAGCGACATCGCCGTGGAGTGGGAGAGCAATG |
| | | GGCAGCCGGAGAACAACTACAAGACCACGCCTCCCGTGCTGGACTC |
| | | CGACGGCTCCTTCTTCCTCTACAGCAGACTCACCGTGGACAAGAGC |
| | | AGGTGGCAGGAAGGGAACGTCTTCTCATGCTCCGTGATGCATGAGG |
| | | CTCTGCACAACCACTACACGCAGAAGAGCCTCTCCCTGTCTCTGGGT |
| | | AAA |
| Spacer | 743 | QGQS |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. An activatable antigen-binding protein construct (ABPC) comprising:
(A) an antigen binding polypeptide complex (ABPC) that comprises a first anti-HER2 heavy chain variable domain (HVD1) and a first anti-HER2 light chain variable domain (LVD1) that together form a HER-2 binding domain,
wherein the HVD1 comprises:
(i) an HVD1 CDR1 comprising the amino acid sequence DTYIH (SEQ ID NO:1),
(ii) an HVD2 CDR2 comprising the amino acid sequence RIYPTNGYTRYADSVKG (SEQ ID NO:2), and
(iii) an HVD3 CDR3 comprising the amino acid sequence WGGDGFYAMDY (SEQ ID NO:3), and
wherein the LVD1 comprises
(i) an LVD1 CDR1 comprising the amino acid sequence RASQDVNTAVA (SEQ ID NO:4),
(ii) an LVD1 CDR2 comprising the amino acid sequence SASFLYS (SEQ ID NO:5), and
(iii) an LVD1 CDR3 comprising the amino acid sequence QQHYTTPPT (SEQ ID NO:6); and
(B) a first cleavable moiety (CM) that comprises a substrate for a protease, and
(C) a first anti-HER2 masking moiety (MM1) comprising an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW00 l), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015),
wherein the MM1, first CM, first HVD1, and first LVD1 are disposed within one or more polypeptides.

2. The activatable ABPC of claim 1, wherein the first MM1 is selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

3. The activatable ABPC of any of claims 1-2, wherein the first MM1 is selected from the group consisting of SEQ ID NO:11 (F3.11), SEQ ID NO: 12 (F3.17), SEQ ID NO: 13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

4. The activatable ABPC of any of claim 1, wherein the first MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

5. The activatable ABPC of claim 4, wherein the first MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

6. The activatable ABPC of any of claims 1-5, wherein the HVD1 comprises
an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7; and wherein the LVD1 comprises an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8.

7. The activatable ABPC of claim 1, wherein the activatable ABPC comprises:
(1) a first polypeptide comprising the first HVD1 and a first Fc domain (Fc1);
(2) a second polypeptide comprising the first MM1, the first CM, the first LVD1, and a first constant light domain (CL);
(3) a third polypeptide comprising a second HVD1 and a second Fc domain (Fc2),
wherein the second anti-HVD1 comprises (i) an HCDR1 having the amino acid sequence of SEQ ID NO:1, (ii) an HCDR2 having the amino acid sequence of SEQ ID NO:2, and (iii) an HCDR3 having the amino acid sequence of SEQ ID NO:3; and
(4) a fourth polypeptide comprising a second MM1, a second CM, a second LVD1 and a second constant light chain domain (CL),
wherein the second CM comprises a substrate for a second protease;
wherein the second LVD1 comprises (i) an LCDR1 comprising the amino acid sequence of SEQ ID NO:4, (ii) an LCDR2 comprising the amino acid sequence of SEQ ID NO:5, and (iii) an LCDR3 comprising the amino acid sequence of SEQ ID NO:6;
wherein the second MM1 comprises an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:4l (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015); and
wherein the Fc1 and the Fc2 bind each other.

8. The activatable ABPC of claim 7, wherein the first CM and the second CM each comprise a substrate for the same protease.

9. The activatable ABPC of claim 7, wherein the first CM and the second CM comprise substrates for different proteases.

10. The activatable ABPC of any of claims 7-9, wherein each of the first MM1 and the second MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

11. The activatable ABPC of claim 10, wherein each of the first MM1 and the second MM1 are each independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO: 17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

12. The activatable ABPC of any of claims 7-11, wherein each of the first MM1 and the second MM1 independently comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

13. The activatable ABPC of any of claims 7-12, wherein each of the first MM1 and the second MM1 independently comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

14. The activatable ABPC of any of claims 7-13, wherein the first polypeptide comprises a first CH1 domain, and the third polypeptide comprises a second CH1 domain.

15. The activatable ABPC of any of claims 7-14, wherein the second polypeptide and the third polypeptide further comprise a first hinge domain and a second hinge domain, respectively.

16. The activatable ABPC of any of claims 7-15 wherein the first MM1 and the second MM1 are the same.

17. The activatable ABPC of any of claims 7-8 and 10-16, wherein the first and third polypeptides comprise the same amino acid sequence.

18. The activatable ABPC of any of claims 7-8 and 10-17, wherein the second and fourth polypeptides comprise the same amino acid sequence.

19. The activatable ABPC of claim 1, further comprising a second binding domain that specifically binds a second biological target (BD2), wherein the first BD2 comprises a first light chain variable domain (LVD2) and a first heavy chain variable domain (HVD2), a second CM (CM2) and a first masking moiety that attenuates the binding of the BD2 to the second biological target (MM2), wherein the activatable ABPC is an activatable bispecific ABPC.

20. The activatable bispecific ABPC of claim 19, further comprising a second anti-HER2 binding domain that comprises a second anti-HER2 heavy chain variable domain (HVD1) and a second anti-HER2 light chain variable domain (LVD1) and a second anti-BD2 comprising a second HVD2 and a second LVD2.

21. The activatable bispecific ABPC of any of claims 19-20, wherein the second biological target is an immune cell surface antigen.

22. The activatable bispecific ABPC of claim 21, wherein the immune cell surface antigen is a CD3.

23. The activatable bispecific ABPC of claim 22, wherein the activatable bispecific ABPC comprises a first polypeptide, a second polypeptide, a third polypeptide, and a fourth polypeptide, wherein:
(1) the first polypeptide comprises the first MM2, a first CM, the first HVD2, the first LVD2, the first HVD1, and a first Fc domain (Fc1);
(2) the second polypeptide comprises the first MM1, a second CM, the first LVD1, and a first constant light chain domain (CL);
(3) the third polypeptide comprises a second MM2, a third CM, a second HVD2, a second LVD2, a second HVD1, and a second Fc domain (Fc2); and
(4) the fourth polypeptide comprises a second MM1, a fourth CM, a second LVD1, and a second CL;
wherein the second HVD1 comprises (i) an HCDR1 having the amino acid sequence of SEQ ID NO:1, (ii) an HCDR2 having the amino acid sequence of SEQ ID NO:2, and (iii) an HCDR3 having the amino acid sequence of SEQ ID NO:3
wherein the second LVD1 comprises (i) an LCDR1 comprising the amino acid sequence of SEQ ID NO:4, (ii) an LCDR2 comprising the amino acid sequence of SEQ ID NO:5, and (iii) an LCDR3 comprising the amino acid sequence of SEQ ID NO:6, and
wherein the second HVD1 comprises (i) an HVD1 CDR1 comprising the amino acid sequence DTYIH (SEQ ID NO:1),
(ii) an HVD2 CDR2 comprising the amino acid sequence RIYPTNGYTRYADSVKG (SEQ ID NO:2), and
(iii) an HVD3 CDR3 comprising the amino acid sequence WGGDGFYAMDY (SEQ ID NO:3),
wherein the second MM1 comprises an amino acid sequence that differs in from 0-2 amino acid positions from an amino acid sequence selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.0a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015); and
wherein the Fc1 and the Fc2 bind each other.

24. The activatable bispecific ABPC of claim 23, wherein at least two of the first, second, third, and fourth CMs comprise substrates for the same protease.

25. The activatable bispecific ABPC of any of claims 23-24, wherein each of the first MM1 and the second MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F501d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

26. The activatable anti-HER2 ABPC of any of claims 23-25, wherein each of the first MM1 and the second MM1 is independently selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW00), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

27. The activatable bispecific ABPC of any of claims 23-24, wherein each of the first MM1 and the second MM1 independently comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

28. The activatable bispecific ABPC of claim 27, wherein each of the first MM1 and the second MM1 independently comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

29. The activatable bispecific ABPC of any of claims 23-28, wherein the first MM1 and the second MM1 are the same.

30. The activatable bispecific ABPC of any of claims 23-29, wherein the first HVD2 and the second HVD2 each comprise
(i) an HCDR1 comprising the amino acid sequence TYAMN (SEQ ID NO:581),
(ii) an HCDR2 comprising the amino acid sequence RIRSKYNNYATYYADSVKD (SEQ ID NO:582), and
(iii) an HCDR3 comprising the amino acid sequence HGNFGNSYVSWFAY (SEQ ID NO:583);
and wherein the first LVD2 and the second LVD2 each comprise
(i) an LCDR1 comprising the amino acid sequence RSSTGAVTTSNYAN (SEQ ID:584),
(ii) an LCDR2 comprising the amino acid sequence GTNKRAP (SEQ ID NO:585), and
(iii) an LCDR3 comprising the amino acid sequence ALWYSNLWV (SEQ ID NO:586); and
Wherein at least one of the first MM2 and the second MM2 comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600).

31. The activatable bispecific ABPC of claim 23, wherein each of the pair of first HVD2 and first LVD2 and the second HVD2 the second LVD2 independently comprises an HVD2 and an LVD2 amino acid sequence, respectively, selected from the group consisting of:
(A) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to the amino acid sequence of SEQ ID NO:587, SEQ ID NO: 190, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:588;
(B) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:587,
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:589; and
(C) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:590, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:591.

32. The activatable bispecific ABPC of any of claims 23-31, wherein the first and third polypeptides comprise the same amino acid sequence.

33. The activatable bispecific ABPC of any of claims 23-32, wherein the second and fourth polypeptides comprise the same amino acid sequence.

34. An activatable bispecific ABPC comprising a first polypeptide, a second polypeptide, and a third polypeptide wherein:
(1) the first polypeptide comprises a second masking moiety (MM2), a first cleavable moiety (CM1), a second heavy chain variable domain (HVD2), a second light chain variable domain (LVD2), and a first Fc domain (Fc1),
(2) the second polypeptide comprises a first masking moiety (MM1), a second cleavable moiety (CM2), a first light chain variable domain (LVD1) and a constant light chain domain (CL), and
(3) the third polypeptide comprises a first heavy chain variable domain (HVD1) and a second Fc domain (Fc2),
wherein:
the LVD1 and the HVD1 together form a first binding domain (BD1) that specifically binds a first biological target,
the LVD2 and the HVD2 together form a second binding domain (BD2) that specifically binds a second biological target,
MM1 comprises a peptide that attenuates binding of the BD1 to the first biological target,
MM2 comprises a peptide that attenuates binding of the BD2 to the second biological target,
the first CM and the second CM each independently comprises a substrate for a protease and
wherein the Fc1 and the Fc2 bind each other.

35. The activatable bispecific ABPC of claim 34, wherein at least one of the first biological target and the second biological target is an immune effector cell target.

36. The activatable bispecific ABPC of any of claims 34-35, wherein at least one of the first biological target and the second biological target is a cancer cell antigen.

37. The activatable bispecific ABPC of any one of claims 34-36, wherein the first polypeptide comprises a linker between the HVD2 and the LVD2.

38. The activatable bispecific ABPC of any one of claims 34-37, wherein the first biological target is a cancer cell antigen.

39. The activatable bispecific ABPC of any one of claims 34-38, wherein the second target is an immune effector cell target.

40. The activatable bispecific ABPC of any one of claims 34-39, wherein the first target is HER2 and wherein the HVD1 comprises:
(i) an HCDR1 comprising the amino acid sequence DTYIH (SEQ ID NO:1),
(ii) an HCDR2 comprising the amino acid sequence RIYPTNGYTRYADSVKG (SEQ ID NO:2), and
(iii) an HCDR3 comprising the amino acid sequence WGGDGFYAMDY (SEQ ID NO:3), and
wherein the LVD1 comprises
(i) an LCDR1 comprising the amino acid sequence RASQDVNTAVA (SEQ ID NO:4),
(ii) an LCDR2 comprising the amino acid sequence SASFLYS (SEQ ID NO:5), and
(iii) an LCDR3 comprising the amino acid sequence QQHYTTPPT (SEQ ID NO:6).

41. The activatable HBPC of any of claims 34-40, wherein the MM1 comprises an amino acid sequence that differs in from 0 to 2 positions from an amino acid sequence selected from the group consisting of SEQ ID NO: 11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

42. The activatable bispecific ABPC of any of claims 34-41, wherein the first CM and the second CM comprise substrates for the same protease.

43. The activatable bispecific ABPC of any of claims 34-42 wherein the MM1 is independently selected from the group consisting of SEQ ID NO:11 (F3.11), SEQ ID NO:12 (F3.17), SEQ ID NO: 13 (F3.18), SEQ ID NO:14 (F3.19), SEQ ID NO:15 (F3.23), SEQ ID NO: 16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:25 (F5.01a), SEQ ID NO:26 (F5.01b), SEQ ID NO:27 (F5.01c), SEQ ID NO:28 (F5.01d), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:34 (F5.17a), SEQ ID NO:35 (F5.17b), SEQ ID NO:36 (F5.17c), SEQ ID NO:37 (F5.17d), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW01), SEQ ID NO:50 (mLW012), SEQ ID NO:51 (mLW013), SEQ ID NO:52 (mLW014), and SEQ ID NO:53 (mLW015).

44. The activatable bispecific ABPC of any of claims 34-43, wherein the MM1 is selected from the group consisting of SEQ ID NO:11 (F3.11), SEQ ID NO:12 (F3.17), SEQ ID NO:13 (F3.18), SEQ ID NO:14 (F3.19), SEQ ID NO:15 (F3.23), SEQ ID NO:16 (F3.42), SEQ ID NO:17 (F3.43), SEQ ID NO:18 (F3.45), SEQ ID NO:19 (F4.2), SEQ ID NO:20 (F4.3), SEQ ID NO:21 (F4.10), SEQ ID NO:22 (F4.13), SEQ ID NO:23 (F4.22), SEQ ID NO:24 (F4.35), SEQ ID NO:29 (F5.01), SEQ ID NO:30 (F5.11), SEQ ID NO:31 (F5.13), SEQ ID NO:32 (F5.15), SEQ ID NO:33 (F5.17), SEQ ID NO:38 (F5.18), SEQ ID NO:39 (mLW001), SEQ ID NO:40 (mLW002), SEQ ID NO:41 (mLW003), SEQ ID NO:42 (mLW004), SEQ ID NO:43 (mLW005), SEQ ID NO:44 (mLW006), SEQ ID NO:45 (mLW007), SEQ ID NO:46 (mLW008), SEQ ID NO:47 (mLW009), SEQ ID NO:48 (mLW010), SEQ ID NO:49 (mLW011), SEQ ID NO:50 (mLW012), SEQ ID NO:51 (mLW013), SEQ ID NO:52 (mLW014), and SEQ ID NO:53 (mLW015).

45. The activatable bispecific ABPC of claim 34, wherein the MM1 comprises an amino acid sequence that differs in from 0-2 amino acid positions from an amino acid sequence selected from the group consisting of SEQ ID NO:29 (F5.01), SEQ ID NO:30 (F5.11), SEQ ID NO:32 (F5.15), and SEQ ID NO:33 (F5.17).

46. The activatable bispecific ABPC of any of claims 43-45, wherein the MM1 comprises an amino acid sequence selected from the group consisting of SEQ ID NO:29 (F5.01), SEQ ID NO:30 (F5.11), SEQ ID NO:32 (F5.15), and SEQ ID NO:33 (F5.17).

47. The activatable bispecific ABPC of any of claims 34-46, wherein the HVD2 comprises
(i) an HCDR1 comprising the amino acid sequence TYAMN (SEQ ID NO:581),
(ii) an HCDR2 comprising the amino acid sequence RIRSKYNNYATYYADSVKD (SEQ ID NO:582), and
(iii) an HCDR3 comprising the amino acid sequence HGNFGNSYVSWFAY (SEQ ID NO:583); and
wherein the LVD2 comprises
(i) an LCDR1 comprising the amino acid sequence RSSTGAVTTSNYAN (SEQ ID:584),
(ii) an LCDR2 comprising the amino acid sequence GTNKRAP (SEQ ID NO:585), and
(iii) an LCDR3 comprising the amino acid sequence ALWYSNLWV (SEQ ID NO:586); and
wherein the MM2 comprises an amino acid sequence that differs in from 0-2 amino acid residues from an amino acid sequence selected from the group consisting of MMYCGGNEVLCGPRV (SEQ ID NO:598), GYLWGCEWNCGGITT (SEQ ID NO:599), and GYRWGCEWNCGGITT (SEQ ID NO:600).

48. The activatable bispecific ABPC of any of claims 34-47, wherein the HVD2 and the LVD2 are selected from the group consisting of:
(A) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to the amino acid sequence of SEQ ID NO:587, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:588;
(B) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:587, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:589; and
(C) (i) an HVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:590, and
(ii) an LVD2 comprising an amino acid sequence that is at least about 90%, or at least 91%, or at least 92%, or at least 93%, or at least 94%, or at least 95%, or at least 96%, or at least 97%, or at least 98%, or at least 99% identical to SEQ ID NO:591.

49. The activatable bispecific ABPC of any of claims 34-48, wherein the HVD1 comprises
an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:7, and
the LVD1 comprises an amino acid sequence that is at least about 90%, or at least 91%, or at least 92%, or at least 93%, or at least 94%, or at least 95%, or at least 96%, or at least 97%, or at least 98%, or at least 99% identical to an amino acid sequence of SEQ ID NO:8.

50. The activatable bispecific ABPC of claim 49, wherein the HVD1 comprises the amino acid sequence of SEQ ID NO:7 and the LVD1 comprises the amino acid sequence of SEQ ID NO:8.

51. An activatable ABPC conjugate comprising the activatable ABPC of any of claims 1-50 and a conjugation moiety.

52. The activatable ABPC conjugate of claim 51, wherein the conjugation moiety is a half-life extending moiety.

53. The activatable ABPC conjugate of claim 51, wherein the conjugation moiety is a toxin.

54. The activatable ABPC conjugate of claim 51, wherein the conjugation moiety is a detectable moiety.

55. A composition comprising the activatable ABPC of any of claims 1-50 or activatable ABPC conjugate of any of claims 51-54 and a pharmaceutically acceptable excipient.

56. A method of treating a disease or disorder in a subject comprising administering to a subject in need thereof a therapeutically effective amount of the activatable ABPC of any one of claims 1-50, or activatable ABPC conjugate of any one of claims 51-54, or composition of claim 55.

57. The method of claim 56, wherein the disease or disorder is a cancer.

58. A polynucleotide encoding at least one polypeptide of the activatable ABPC of any of claims 1-50.

59. A vector comprising the polynucleotide of claim 58.

60. The vector of claim 59, wherein the vector is an expression vector that further comprises a promoter operably linked to the polynucleotide.

61. A recombinant host cell comprising the polynucleotide of claim 58 or the vector of any of claims 59-60.

62. A method for producing an activatable ABPC of any of claims 1-50, wherein the method comprises:
(a) culturing the recombinant host cell of claim 61 in a culture medium under conditions sufficient to produce the activatable ABPC; and
(b) recovering the activatable ABPC from the recombinant host cell and/or the culture medium.


SEQUENCE LISTING (743 sequences)

SEQ ID NO: 1 - protein, 5 aa, synthetic construct
DTYIH

SEQ ID NO: 2 - protein, 17 aa, synthetic construct
RIYPTNGYTRYADSVKG

SEQ ID NO: 3 - protein, 11 aa, synthetic construct
WGGDGFYAMDY

SEQ ID NO: 4 - protein, 11 aa, synthetic construct
RASQDVNTAVA

SEQ ID NO: 5 - protein, 7 aa, synthetic construct
SASFLYS

SEQ ID NO: 6 - protein, 9 aa, synthetic construct
QQHYTTPPT

SEQ ID NO: 7 - protein, 120 aa, synthetic construct [sequence omitted]

SEQ ID NO: 8 - protein, 107 aa, synthetic construct [sequence omitted]

SEQ ID NO: 9 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 15 aa, synthetic construct
MSELCLSEYRCWGLE

SEQ ID NO: 12 - protein, 15 aa, synthetic construct
MTMCGEFMFLGCETR

SEQ ID NO: 13 - protein, 15 aa, synthetic construct
GRMTCADHPKCNYIY

SEQ ID NO: 14 - protein, 15 aa, synthetic construct
PICAFVYNDPECSWW

SEQ ID NO: 15 - protein, 15 aa, synthetic construct
SWCMMHHEGNVCQTV

SEQ ID NO: 16 - protein, 15 aa, synthetic construct
QFACFETRSQYCDWK

SEQ ID NO: 17 - protein, 15 aa, synthetic construct
MLWCDTPDGSCWWRE

SEQ ID NO: 18 - protein, 15 aa, synthetic construct
ICALTIFSTSVDCLY

SEQ ID NO: 19 - protein, 15 aa, synthetic construct
DGYVCHWEWACRTAA

SEQ ID NO: 20 - protein, 15 aa, synthetic construct
LDCVRTLYYSDCYHR

SEQ ID NO: 21 - protein, 15 aa, synthetic construct
LCVMDHIYDTMNCNQ

SEQ ID NO: 22 - protein, 15 aa, synthetic construct
PFKCDLTNPTCTILM

SEQ ID NO: 23 - protein, 15 aa, synthetic construct
QFCRNYPLSMMCCKY

SEQ ID NO: 24 - protein, 15 aa, synthetic construct
ICSYNWHFDTIDCNS

SEQ ID NO: 25 - protein, 15 aa, synthetic construct
SCYVLLDGILMPSDC

SEQ ID NO: 26 - protein, 15 aa, synthetic construct
SCYVLLDGILMPCDS

SEQ ID NO: 27 - protein, 15 aa, synthetic construct
CSYVLLDGILMPSDC

SEQ ID NO: 28 - protein, 15 aa, synthetic construct
CSYVLLDGILMPCDS

SEQ ID NO: 29 - protein, 15 aa, synthetic construct
CCYVLLDGILMPCDC

SEQ ID NO: 30 - protein, 15 aa, synthetic construct
SECMISQWGPDCTVA

SEQ ID NO: 31 - protein, 15 aa, synthetic construct
YCLYNKLNDTINCSD

SEQ ID NO: 32 - protein, 15 aa, synthetic construct
RCVMDPVTDTIDCNK

SEQ ID NO: 33 - protein, 15 aa, synthetic construct
ALICCSDVSGLCRWC

SEQ ID NO: 34 - protein, 15 aa, synthetic construct
ALISCSDVSGLSRWC

SEQ ID NO: 35 - protein, 15 aa, synthetic construct
ALISCSDVSGLCRWS

SEQ ID NO: 36 - protein, 15 aa, synthetic construct
ALICSSDVSGLSRWC

SEQ ID NO: 37 - protein, 15 aa, synthetic construct
ALICSSDVSGLCRWS

SEQ ID NO: 38 - protein, 15 aa, synthetic construct
SSCRTTGLMTTCDLD

SEQ ID NO: 39 - protein, 14 aa, synthetic construct
LYCTEGRWYEGTCA

SEQ ID NO: 40 - protein, 15 aa, synthetic construct
LTKCATSTSTTCDLD

SEQ ID NO: 41 - protein, 15 aa, synthetic construct
FGCGVVLDLDHCAPW

SEQ ID NO: 42 - protein, 15 aa, synthetic construct
TPCWHMIMTNPECMR

SEQ ID NO: 43 - protein, 15 aa, synthetic construct
YNACWFGPYDCWYRP

SEQ ID NO: 44 - protein, 15 aa, synthetic construct
FLCEPPSWGPDCTMI

SEQ ID NO: 45 - protein, 15 aa, synthetic construct
DLCSFGLWYRGTCEG

SEQ ID NO: 46 - protein, 15 aa, synthetic construct
TCYHDMHKDTIHCNN

SEQ ID NO: 47 - protein, 15 aa, synthetic construct
LYRDMCGGGCEDWHD

SEQ ID NO: 48 - protein, 15 aa, synthetic construct
LGSLCDPFDCPGKNL

SEQ ID NO: 49 - protein, 15 aa, synthetic construct
RGCSFGYWGNGTCLG

SEQ ID NO: 50 - protein, 15 aa, synthetic construct
RACSFGVWWKGTCEG

SEQ ID NO: 51 - protein, 15 aa, synthetic construct
ANVCWLGPYDCRWES

SEQ ID NO: 52 - protein, 15 aa, synthetic construct
GMCTSGRWYGGDCTG

SEQ ID NO: 53 - protein, 15 aa, synthetic construct
ACTHNTMTDKIYCNN

SEQ ID NO: 54 - protein, 10 aa, synthetic construct
AANALAHGLF

SEQ ID NO: 55 - protein, 4 aa, synthetic construct
AANL

SEQ ID NO: 56 - protein, 10 aa, synthetic construct
AANLGSGGSS

SEQ ID NO: 57 - protein, 5 aa, synthetic construct
AAPRS

SEQ ID NO: 58 - protein, 6 aa, synthetic construct
AAPRSF

SEQ ID NO: 59 - protein, 8 aa, synthetic construct
AARGPAIH

SEQ ID NO: 60 - protein, 8 aa, synthetic construct
AAYHLVSQ

SEQ ID NO: 61 - protein, 10 aa, synthetic construct
AFPDMRSVRS

SEQ ID NO: 62 - protein, 7 aa, synthetic construct
AFQALRM

SEQ ID NO: 63 - protein, 6 aa, synthetic construct
AFRHLR

SEQ ID NO: 64 - protein, 8 aa, synthetic construct
AGLGISST

SEQ ID NO: 65 - protein, 8 aa, synthetic construct
AGLGVVER

SEQ ID NO: 66 - protein, 4 aa, synthetic construct
AGPR

SEQ ID NO: 67 - protein, 4 aa, synthetic construct
AHGL

SEQ ID NO: 68 - protein, 5 aa, synthetic construct
AHGLF

SEQ ID NO: 69 - protein, 7 aa, synthetic construct
AHQALRM

SEQ ID NO: 70 - protein, 10 aa, synthetic construct
AIPRVRLFDV

SEQ ID NO: 71 - protein, 5 aa, synthetic construct
ALAHG

SEQ ID NO: 72 - protein, 6 aa, synthetic construct
ALAHGL

SEQ ID NO: 73 - protein, 7 aa, synthetic construct
ALAHGLF

SEQ ID NO: 74 - protein, 12 aa, synthetic construct
ALAHGLFAPRSF

SEQ ID NO: 75 - protein, 13 aa, synthetic construct
ALAHGLFSGRSAN

SEQ ID NO: 76 - protein, 12 aa, synthetic construct
ALAHGLPTFVHL

SEQ ID NO: 77 - protein, 8 aa, synthetic construct
ALGLLRLP

SEQ ID NO: 78 - protein, 10 aa, synthetic construct
ALPSVKMVSE

SEQ ID NO: 79 - protein, 5 aa, synthetic construct
ALRAP

SEQ ID NO: 80 - protein, 7 aa, synthetic construct
ANQALRM

SEQ ID NO: 81 - protein, 8 aa, synthetic construct
ANQALRMA

SEQ ID NO: 82 - protein, 10 aa, synthetic construct
APPLVKSMVV

SEQ ID NO: 83 - protein, 10 aa, synthetic construct
APPSFKLVNA

SEQ ID NO: 84 - protein, 4 aa, synthetic construct
APRS

SEQ ID NO: 85 - protein, 11 aa, synthetic construct
APRSALAHGLF

SEQ ID NO: 86 - protein, 5 aa, synthetic construct
APRSF

SEQ ID NO: 87 - protein, 8 aa, synthetic construct
AQFVLTEG

SEQ ID NO: 88 - protein, 8 aa, synthetic construct
AQNLLGMV

SEQ ID NO: 89 - protein, 4 aa, synthetic construct
ARGP

SEQ ID NO: 90 - protein, 5 aa, synthetic construct
ARGPS

SEQ ID NO: 91 - protein, 6 aa, synthetic construct
ARGPSF

SEQ ID NO: 92 - protein, 7 aa, synthetic construct
ARGPSFK

SEQ ID NO: 93 - protein, 8 aa, synthetic construct
ASGLLRFP

SEQ ID NO: 94 - protein, 10 aa, synthetic construct
ASPTMKTVGL

SEQ ID NO: 95 - protein, 8 aa, synthetic construct
AVGLLAPP

SEQ ID NO: 96 - protein, 18 aa, synthetic construct
AVGLLAPPGGLSGRSANI

SEQ ID NO: 97 - protein, 18 aa, synthetic construct
AVGLLAPPGGLSGRSANP

SEQ ID NO: 98 - protein, 18 aa, synthetic construct
AVGLLAPPGGLSGRSDDH

SEQ ID NO: 99 - protein, 18 aa, synthetic construct
AVGLLAPPGGLSGRSDIH

SEQ ID NO: 100 - protein, 18 aa, synthetic construct
AVGLLAPPGGLSGRSDNH

SEQ ID NO: 101 - protein, 18 aa, synthetic construct
AVGLLAPPGGLSGRSDNI

SEQ ID NO: 102 - protein, 18 aa, synthetic construct
AVGLLAPPGGLSGRSDNP

SEQ ID NO: 103 - protein, 18 aa, synthetic construct
AVGLLAPPGGLSGRSDQH

SEQ ID NO: 104 - protein, 18 aa, synthetic construct
AVGLLAPPGGLSGRSDTH

SEQ ID NO: 105 - protein, 18 aa, synthetic construct
AVGLLAPPGGLSGRSDYH

SEQ ID NO: 106 - protein, 17 aa, synthetic construct
AVGLLAPPGGLSGRSNI

SEQ ID NO: 107 - protein, 18 aa, synthetic construct
AVGLLAPPGGLSGRSNIG

SEQ ID NO: 108 - protein, 19 aa, synthetic construct
AVGLLAPPGGLSGRSNIGS

SEQ ID NO: 109 - protein, 22 aa, synthetic construct
AVGLLAPPGGTSTSGRSANPRG

SEQ ID NO: 110 - protein, 17 aa, synthetic construct
AVGLLAPPSGRSANPRG

SEQ ID NO: 111 - protein, 18 aa, synthetic construct
AVGLLAPPTSGRSANPRG

SEQ ID NO: 112 - protein, 10 aa, synthetic construct
AVPKVRVVPE

SEQ ID NO: 113 - protein, 7 aa, synthetic construct
CGPPLGR

SEQ ID NO: 114 - protein, 7 aa, synthetic construct
CSPPLGR

SEQ ID NO: 115 - protein, 10 aa, synthetic construct
DEVDGSGGSS

SEQ ID NO: 116 - protein, 8 aa, synthetic construct
DISHWRRS

SEQ ID NO: 117 - protein, 7 aa, synthetic construct
DLAHPLL

SEQ ID NO: 118 - protein, 10 aa, synthetic construct
DLPLVKSLPS

SEQ ID NO: 119 - protein, 12 aa, synthetic construct
DRLSGRSANHKK

SEQ ID NO: 120 - protein, 12 aa, synthetic construct
DRLSGRSDNHKK

SEQ ID NO: 121 - protein, 10 aa, synthetic construct
DRPEMKSLSG

SEQ ID NO: 122 - protein, 10 aa, synthetic construct
DRPKVKTMDF

SEQ ID NO: 123 - protein, 8 aa, synthetic construct
DVAQFVLT

SEQ ID NO: 124 - protein, 10 aa, synthetic construct
DVPPMKTLRP

SEQ ID NO: 125 - protein, 8 aa, synthetic construct
DWLYWMGI

SEQ ID NO: 126 - protein, 8 aa, synthetic construct
DWLYWMSI

SEQ ID NO: 127 - protein, 8 aa, synthetic construct
DWLYWPGI

SEQ ID NO: 128 - protein, 8 aa, synthetic construct
DWLYWPSI

SEQ ID NO: 129 - protein, 10 aa, synthetic construct
EAPKVKALPK

SEQ ID NO: 130 - protein, 10 aa, synthetic construct
EHPRVKVVSE

SEQ ID NO: 131 - protein, 10 aa, synthetic construct
EKPRMKLFQG

SEQ ID NO: 132 - protein, 8 aa, synthetic construct
EPQALAMS

SEQ ID NO: 133 - protein, 10 aa, synthetic construct
EQPEVKMVKG

SEQ ID NO: 134 - protein, 10 aa, synthetic construct
ERPGVKSLVL

SEQ ID NO: 135 - protein, 8 aa, synthetic construct
ESLPVVAV

SEQ ID NO: 136 - protein, 10 aa, synthetic construct
ESPVMKSMAL

SEQ ID NO: 137 - protein, 5 aa, synthetic construct
ESRRW

SEQ ID NO: 138 - protein, 6 aa, synthetic construct
ESRRWM

SEQ ID NO: 139 - protein, 7 aa, synthetic construct
ESRRWMP

SEQ ID NO: 140 - protein, 10 aa, synthetic construct
ETPSVKTMGR

SEQ ID NO: 141 - protein, 12 aa, synthetic construct
ETPSVKTMGRSS

SEQ ID NO: 142 - protein, 10 aa, synthetic construct
FPRPLGITGL

SEQ ID NO: 143 - protein, 8 aa, synthetic construct
FRLLDWQW

SEQ ID NO: 144 - protein, 8 aa, synthetic construct
GCGPPLGR

SEQ ID NO: 145 - protein, 8 aa, synthetic construct
GCSPPLGR

SEQ ID NO: 146 - protein, 10 aa, synthetic construct
GFPHMKTFQH

SEQ ID NO: 147 - protein, 12 aa, synthetic construct
GFPHMKTFQHSS

SEQ ID NO: 148 - protein, 8 aa, synthetic construct
GGGPPLGR

SEQ ID NO: 149 - protein, 7 aa, synthetic construct
GGPPLGR

SEQ ID NO: 150 - protein, 10 aa, synthetic construct
GGQPSGMWGW

SEQ ID NO: 151 - protein, 7 aa, synthetic construct
GGSIDGR

SEQ ID NO: 152 - protein, 8 aa, synthetic construct
GGSPPLGR

SEQ ID NO: 153 - protein, 8 aa, synthetic construct
GGWHTGRN

SEQ ID NO: 154 - protein, 5 aa, synthetic construct
GIAGQ

SEQ ID NO: 155 - protein, 10 aa, synthetic construct
GLGTPRGLFA

SEQ ID NO: 156 - protein, 6 aa, synthetic construct
GLPTFV

SEQ ID NO: 157 - protein, 7 aa, synthetic construct
GLPTFVH

SEQ ID NO: 158 - protein, 8 aa, synthetic construct
GLPTFVHL

SEQ ID NO: 159 - protein, 12 aa, synthetic construct
GLPTFVHLPRQV

SEQ ID NO: 160 - protein, 19 aa, synthetic construct
GLSGRSDNHGGAVGLLAPP

SEQ ID NO: 161 - protein, 21 aa, synthetic construct
GLSGRSDNHGGVHMPLGFLGP

SEQ ID NO: 162 - protein, 12 aa, synthetic construct
GLSGRSDNHGSS

SEQ ID NO: 163 - protein, 8 aa, synthetic construct
GPEGLRVG

SEQ ID NO: 164 - protein, 8 aa, synthetic construct
GPLGIAGI

SEQ ID NO: 165 - protein, 12 aa, synthetic construct
GPLNGRSDNHKA

SEQ ID NO: 166 - protein, 12 aa, synthetic construct
GPLNGRSDNHKK

SEQ ID NO: 167 - protein, 12 aa, synthetic construct
GPLNGRSDNHKR

SEQ ID NO: 168 - protein, 12 aa, synthetic construct
GPLNGRSDNHQA

SEQ ID NO: 169 - protein, 12 aa, synthetic construct
GPLNGRSDNHQK

SEQ ID NO: 170 - protein, 12 aa, synthetic construct
GPLNGRSDNHQR

SEQ ID NO: 171 - protein, 12 aa, synthetic construct
GPLNGRSDNHRA

SEQ ID NO: 172 - protein, 12 aa, synthetic construct
GPLNGRSDNHRK

SEQ ID NO: 173 - protein, 12 aa, synthetic construct
GPLNGRSDNHRR

SEQ ID NO: 174 - protein, 12 aa, synthetic construct
GPLSGRSDNHKA

SEQ ID NO: 175 - protein, 12 aa, synthetic construct
GPLSGRSDNHKK

SEQ ID NO: 176 - protein, 12 aa, synthetic construct
GPLSGRSDNHKR

SEQ ID NO: 177 - protein, 12 aa, synthetic construct
GPLSGRSDNHQA

SEQ ID NO: 178 - protein, 12 aa, synthetic construct
GPLSGRSDNHQK

SEQ ID NO: 179 - protein, 12 aa, synthetic construct
GPLSGRSDNHQR

SEQ ID NO: 180 - protein, 12 aa, synthetic construct
GPLSGRSDNHRA

SEQ ID NO: 181 - protein, 12 aa, synthetic construct
GPLSGRSDNHRK

SEQ ID NO: 182 - skipped

SEQ ID NO: 183 - protein, 12 aa, synthetic construct
GPLSGRSDNHRR

SEQ ID NO: 184 - protein, 6 aa, synthetic construct
GPPLGR

SEQ ID NO: 185 - protein, 8 aa, synthetic construct
GPQGIAGQ

SEQ ID NO: 186 - protein, 8 aa, synthetic construct
GPQGLLGA

SEQ ID NO: 187 - protein, 6 aa, synthetic construct
GPRSFG

SEQ ID NO: 188 - protein, 7 aa, synthetic construct
GPRSFGL

SEQ ID NO: 189 - protein, 8 aa, synthetic construct
GPSHLVLT

SEQ ID NO: 190 - protein, 4 aa, synthetic construct
GPTN

SEQ ID NO: 191 - protein, 11 aa, synthetic construct
GPTNALAHGLF

SEQ ID NO: 192 - protein, 5 aa, synthetic construct
GRSML

SEQ ID NO: 193 - protein, 6 aa, synthetic construct
GRSMLL

SEQ ID NO: 194 - protein, 7 aa, synthetic construct
GRSMLLG

SEQ ID NO: 195 - protein, 8 aa, synthetic construct
GRSMLLGG

SEQ ID NO: 196 - protein, 8 aa, synthetic construct
GRSMLLGP

SEQ ID NO: 197 - protein, 8 aa, synthetic construct
GRSMLLGS

SEQ ID NO: 198 - protein, 7 aa, synthetic construct
GRSMLLP

SEQ ID NO: 199 - protein, 8 aa, synthetic construct
GRSMLLPG

SEQ ID NO: 200 - protein, 8 aa, synthetic construct
GRSMLLPP

SEQ ID NO: 201 - protein, 8 aa, synthetic construct
GRSMLLPS

SEQ ID NO: 202 - protein, 7 aa, synthetic construct
GRSMLLS

SEQ ID NO: 203 - protein, 8 aa, synthetic construct
GRSMLLSG

SEQ ID NO: 204 - protein, 8 aa, synthetic construct
GRSMLLSP

SEQ ID NO: 205 - protein, 8 aa, synthetic construct
GRSMLLSS

SEQ ID NO: 206 - protein, 6 aa, synthetic construct
GRSMLM

SEQ ID NO: 207 - protein, 7 aa, synthetic construct
GRSMLMG

SEQ ID NO: 208 - protein, 8 aa, synthetic construct
GRSMLMGG

SEQ ID NO: 209 - protein, 8 aa, synthetic construct
GRSMLMGP

SEQ ID NO: 210 - protein, 8 aa, synthetic construct
GRSMLMGS

SEQ ID NO: 211 - protein, 7 aa, synthetic construct
GRSMLMP

SEQ ID NO: 212 - protein, 8 aa, synthetic construct
GRSMLMPG

SEQ ID NO: 213 - protein, 8 aa, synthetic construct
GRSMLMPP

SEQ ID NO: 214 - protein, 8 aa, synthetic construct
GRSMLMPS

SEQ ID NO: 215 - protein, 7 aa, synthetic construct
GRSMLMS

SEQ ID NO: 216 - protein, 8 aa, synthetic construct
GRSMLMSG

SEQ ID NO: 217 - protein, 8 aa, synthetic construct
GRSMLMSP

SEQ ID NO: 218 - protein, 8 aa, synthetic construct
GRSMLMSS

SEQ ID NO: 219 - protein, 8 aa, synthetic construct
GSGPPLGR

SEQ ID NO: 220 - protein, 7 aa, synthetic construct
GSPPLGR

SEQ ID NO: 221 - protein, 8 aa, synthetic construct
GSSPPLGR

SEQ ID NO: 222 - protein, 12 aa, synthetic construct
GTGRGPSWVGSS

SEQ ID NO: 223 - protein, 8 aa, synthetic construct
HMMQYARH

SEQ ID NO: 224 - protein, 8 aa, synthetic construct
HTGRSGAL

SEQ ID NO: 225 - protein, 5 aa, synthetic construct
HVPRQ

SEQ ID NO: 226 - protein, 6 aa, synthetic construct
HVPRQV

SEQ ID NO: 227 - protein, 11 aa, synthetic construct
HVPRQVAPRSF

SEQ ID NO: 228 - protein, 11 aa, synthetic construct
HVPRQVLSGRS

SEQ ID NO: 229 - protein, 13 aa, synthetic construct
HVPRQVLSGRSAN

SEQ ID NO: 230 - protein, 8 aa, synthetic construct
HWHLGPPT

SEQ ID NO: 231 - protein, 8 aa, synthetic construct
IANLLSMV

SEQ ID NO: 232 - protein, 4 aa, synthetic construct
IDGR

SEQ ID NO: 233 - protein, 4 aa, synthetic construct
IEGR

SEQ ID NO: 234 - protein, 8 aa, synthetic construct
ILNLLSMV

SEQ ID NO: 235 - protein, 8 aa, synthetic construct
ILPRSPAF

SEQ ID NO: 236 - protein, 8 aa, synthetic construct
IPFSWSRF

SEQ ID NO: 237 - protein, 8 aa, synthetic construct
IQNLLSMV

SEQ ID NO: 238 - protein, 5 aa, synthetic construct
ISSGL

SEQ ID NO: 239 - protein, 6 aa, synthetic construct
ISSGLL

SEQ ID NO: 240 - protein, 7 aa, synthetic construct
ISSGLLS

SEQ ID NO: 241 - protein, 13 aa, synthetic construct
ISSGLLSGRSANI

SEQ ID NO: 242 - protein, 13 aa, synthetic construct
ISSGLLSGRSANP

SEQ ID NO: 243 - protein, 15 aa, synthetic construct
ISSGLLSGRSANPRG

SEQ ID NO: 244 - protein, 13 aa, synthetic construct
ISSGLLSGRSDDH

SEQ ID NO: 245 - protein, 13 aa, synthetic construct
ISSGLLSGRSDIH

SEQ ID NO: 246 - protein, 13 aa, synthetic construct
ISSGLLSGRSDNH

SEQ ID NO: 247 - protein, 13 aa, synthetic construct
ISSGLLSGRSDNI

SEQ ID NO: 248 - protein, 13 aa, synthetic construct
ISSGLLSGRSDNP

SEQ ID NO: 249 - protein, 13 aa, synthetic construct
ISSGLLSGRSDQH

SEQ ID NO: 250 - protein, 13 aa, synthetic construct
ISSGLLSGRSDTH

SEQ ID NO: 251 - protein, 13 aa, synthetic construct
ISSGLLSGRSDYH

SEQ ID NO: 252 - protein, 13 aa, synthetic construct
ISSGLLSGRSGNH

SEQ ID NO: 253 - protein, 12 aa, synthetic construct
ISSGLLSGRSNI

SEQ ID NO: 254 - protein, 13 aa, synthetic construct
ISSGLLSGRSNIG

SEQ ID NO: 255 - protein, 14 aa, synthetic construct
ISSGLLSGRSNIGS

SEQ ID NO: 256 - protein, 8 aa, synthetic construct
ISSGLLSS

SEQ ID NO: 257 - protein, 22 aa, synthetic construct
ISSGLLSSGGSGGSLSGRSDNH

SEQ ID NO: 258 - protein, 22 aa, synthetic construct
ISSGLLSSGGSGGSLSGRSGNH

SEQ ID NO: 259 - protein, 7 aa, synthetic construct
ISSGLSS

SEQ ID NO: 260 - protein, 6 aa, synthetic construct
IVSRSA

SEQ ID NO: 261 - protein, 12 aa, synthetic construct
KGLTGRSDRHQA

SEQ ID NO: 262 - protein, 10 aa, synthetic construct
KGPKVKVVTL

SEQ ID NO: 263 - protein, 12 aa, synthetic construct
KNLYGRSENNGN

SEQ ID NO: 264 - protein, 8 aa, synthetic construct
KRMPVQFL

SEQ ID NO: 265 - protein, 8 aa, synthetic construct
LAAPLGLL

SEQ ID NO: 266 - protein, 4 aa, synthetic construct
LAHG

SEQ ID NO: 267 - protein, 5 aa, synthetic construct
LAHGL

SEQ ID NO: 268 - protein, 6 aa, synthetic construct
LAHGLF

SEQ ID NO: 269 - protein, 9 aa, synthetic construct
LAPLGLQRR

SEQ ID NO: 270 - protein, 5 aa, synthetic construct
LARAG

SEQ ID NO: 271 - protein, 6 aa, synthetic construct
LARAGI

SEQ ID NO: 272 - protein, 6 aa, synthetic construct
LARAGL

SEQ ID NO: 273 - protein, 8 aa, synthetic construct
LKAAPRWA

SEQ ID NO: 274 - protein, 8 aa, synthetic construct
LKAAPRWF

SEQ ID NO: 275 - protein, 8 aa, synthetic construct
LKAAPVWA

SEQ ID NO: 276 - protein, 8 aa, synthetic construct
LKAAPVWF

SEQ ID NO: 277 - protein, 8 aa, synthetic construct
LKGRSYYY

SEQ ID NO: 278 - protein, 8 aa, synthetic construct
LLAPSHRA

SEQ ID NO: 279 - protein, 8 aa, synthetic construct
LLEALRAL

SEQ ID NO: 280 - protein, 8 aa, synthetic construct
LLESLRAL

SEQ ID NO: 281 - protein, 8 aa, synthetic construct
LLLPAHGG

SEQ ID NO: 282 - protein, 8 aa, synthetic construct
LLLPLLGS

SEQ ID NO: 283 - protein, 8 aa, synthetic construct
LLNALRAL

SEQ ID NO: 284 - protein, 8 aa, synthetic construct
LLNSLRAL

SEQ ID NO: 285 - protein, 8 aa, synthetic construct
LLQALRAL

SEQ ID NO: 286 - protein, 8 aa, synthetic construct
LLQSLRAL

SEQ ID NO: 287 - protein, 8 aa, synthetic construct
LLSALRAL

SEQ ID NO: 288 - protein, 8 aa, synthetic construct
LLSSLRAL

SEQ ID NO: 289 - protein, 8 aa, synthetic construct
LNGRSDNH

SEQ ID NO: 290 - protein, 7 aa, synthetic construct
LPAGLLL

SEQ ID NO: 291 - protein, 8 aa, synthetic construct
LPAGLLLR

SEQ ID NO: 292 - protein, 8 aa, synthetic construct
LPAHLVLL

SEQ ID NO: 293 - protein, 8 aa, synthetic construct
LPAHLVLV

SEQ ID NO: 294 - protein, 8 aa, synthetic construct
LPGGLSPW

SEQ ID NO: 295 - protein, 8 aa, synthetic construct
LPSHLVLL

SEQ ID NO: 296 - protein, 8 aa, synthetic construct
LPSHLVLV

SEQ ID NO: 297 - protein, 5 aa, synthetic construct
LPTFV

SEQ ID NO: 298 - protein, 6 aa, synthetic construct
LPTFVH

SEQ ID NO: 299 - protein, 7 aa, synthetic construct
LPTFVHL

SEQ ID NO: 300 - protein, 5 aa, synthetic construct
LRSGW

SEQ ID NO: 301 - protein, 4 aa, synthetic construct
LSGR

SEQ ID NO: 302 - protein, 5 aa, synthetic construct
LSGRS

SEQ ID NO: 303 - protein, 6 aa, synthetic construct
LSGRSA

SEQ ID NO: 304 - protein, 12 aa, synthetic construct
LSGRSALAHGLF

SEQ ID NO: 305 - protein, 7 aa, synthetic construct
LSGRSAN

SEQ ID NO: 306 - protein, 8 aa, synthetic construct
LSGRSANI

SEQ ID NO: 307 - protein, 8 aa, synthetic construct
LSGRSANP

SEQ ID NO: 308 - protein, 6 aa, synthetic construct
LSGRSD

SEQ ID NO: 309 - protein, 7 aa, synthetic construct
LSGRSDD

SEQ ID NO: 310 - protein, 8 aa, synthetic construct
LSGRSDDH

SEQ ID NO: 311 - protein, 7 aa, synthetic construct
LSGRSDI

SEQ ID NO: 312 - protein, 8 aa, synthetic construct
LSGRSDIH

SEQ ID NO: 313 - protein, 7 aa, synthetic construct
LSGRSDN

SEQ ID NO: 314 - protein, 8 aa, synthetic construct
LSGRSDNH

SEQ ID NO: 315 - protein, 18 aa, synthetic construct
LSGRSDNHGGAVGLLAPP

SEQ ID NO: 316 - protein, 22 aa, synthetic construct
LSGRSDNHGGSGGSISSGLLSS

SEQ ID NO: 317 - protein, 22 aa, synthetic construct
LSGRSDNHGGSGGSQNQALRMA

SEQ ID NO: 318 - protein, 20 aa, synthetic construct
LSGRSDNHGGVHMPLGFLGP

SEQ ID NO: 319 - protein, 8 aa, synthetic construct
LSGRSDNI

SEQ ID NO: 320 - protein, 8 aa, synthetic construct
LSGRSDNP

SEQ ID NO: 321 - protein, 7 aa, synthetic construct
LSGRSDQ

SEQ ID NO: 322 - protein, 8 aa, synthetic construct
LSGRSDQH

SEQ ID NO: 323 - protein, 7 aa, synthetic construct
LSGRSDT

SEQ ID NO: 324 - protein, 8 aa, synthetic construct
LSGRSDTH

SEQ ID NO: 325 - protein, 7 aa, synthetic construct
LSGRSDY

SEQ ID NO: 326 - protein, 8 aa, synthetic construct
LSGRSDYH

SEQ ID NO: 327 - protein, 8 aa, synthetic construct
LSGRSENH

SEQ ID NO: 328 - protein, 6 aa, synthetic construct
LSGRSG

SEQ ID NO: 329 - protein, 7 aa, synthetic construct
LSGRSGN

SEQ ID NO: 330 - protein, 8 aa, synthetic construct
LSGRSGNH

SEQ ID NO: 331 - protein, 22 aa, synthetic construct
LSGRSGNHGGSGGSISSGLLSS

SEQ ID NO: 332 - protein, 22 aa, synthetic construct
LSGRSGNHGGSGGSQNQALRMA

SEQ ID NO: 333 - protein, 8 aa, synthetic construct
LSGRSGNP

SEQ ID NO: 334 - protein, 8 aa, synthetic construct
LSGRSVTQ

SEQ ID NO: 335 - protein, 8 aa, synthetic construct
LSQARWRK

SEQ ID NO: 336 - protein, 8 aa, synthetic construct
LTFPTYIF

SEQ ID NO: 337 - protein, 8 aa, synthetic construct
LTFPTYWF

SEQ ID NO: 338 - protein, 8 aa, synthetic construct
LTGRSDRH

SEQ ID NO: 339 - protein, 7 aa, synthetic construct
LTGRSGA

SEQ ID NO: 340 - protein, 8 aa, synthetic construct
LYAAPRWA

SEQ ID NO: 341 - protein, 8 aa, synthetic construct
LYAAPRWF

SEQ ID NO: 342 - protein, 8 aa, synthetic construct
LYAAPVWA

SEQ ID NO: 343 - protein, 8 aa, synthetic construct
LYAAPVWF

SEQ ID NO: 344 - protein, 8 aa, synthetic construct
LYGRSENN

SEQ ID NO: 345 - protein, 8 aa, synthetic construct
MDAFLESS

SEQ ID NO: 346 - protein, 8 aa, synthetic construct
MGLFSEAG

SEQ ID NO: 347 - protein, 6 aa, synthetic construct
MGPWFM

SEQ ID NO: 348 - protein, 8 aa, synthetic construct
MIAPVAYR

SEQ ID NO: 349 - protein, 6 aa, synthetic construct
MLRSGW

SEQ ID NO: 350 - protein, 7 aa, synthetic construct
MLRSGWR

SEQ ID NO: 351 - protein, 8 aa, synthetic construct
MLRSGWRG

SEQ ID NO: 352 - protein, 8 aa, synthetic construct
MLRSGWRL

SEQ ID NO: 353 - protein, 8 aa, synthetic construct
MLRSGWRS

SEQ ID NO: 354 - protein, 8 aa, synthetic construct
MTFPTYIF

SEQ ID NO: 355 - protein, 8 aa, synthetic construct
MTFPTYWF

SEQ ID NO: 356 - protein, 8 aa, synthetic construct
MVLGRSLL

SEQ ID NO: 357 - protein, 12 aa, synthetic construct
NHRIGRSDNHRR

SEQ ID NO: 358 - protein, 10 aa, synthetic construct
NMPSFKLVTG

SEQ ID NO: 359 - protein, 12 aa, synthetic construct
NTLSGRSENHSG

SEQ ID NO: 360 - protein, 12 aa, synthetic construct
NTLSGRSGNHGS

SEQ ID NO: 361 - protein, 10 aa, synthetic construct
NZPRVRLVLP

SEQ ID NO: 362 - protein, 8 aa, synthetic construct
PAGLWLDP

SEQ ID NO: 363 - protein, 5 aa, synthetic construct
PAGRR

SEQ ID NO: 364 - protein, 6 aa, synthetic construct
PAGRRS

SEQ ID NO: 365 - protein, 7 aa, synthetic construct
PAGRRSL

SEQ ID NO: 366 - protein, 8 aa, synthetic construct
PASLWYTQ

SEQ ID NO: 367 - protein, 8 aa, synthetic construct
PESRRWMP

SEQ ID NO: 368 - protein, 6 aa, synthetic construct
PFHLSR

SEQ ID NO: 369 - protein, 6 aa, synthetic construct
PHGFFQ

SEQ ID NO: 370 - protein, 7 aa, synthetic construct
PLARAGI

SEQ ID NO: 371 - protein, 7 aa, synthetic construct
PLARAGL

SEQ ID NO: 372 - protein, 4 aa, synthetic construct
PLGL

SEQ ID NO: 373 - protein, 6 aa, synthetic construct
PLGLAG

SEQ ID NO: 374 - protein, 6 aa, synthetic construct
PLGLWA

SEQ ID NO: 375 - protein, 7 aa, synthetic construct
PLGVRGK

SEQ ID NO: 376 - protein, 8 aa, synthetic construct
PLTGRSGG

SEQ ID NO: 377 - protein, 12 aa, synthetic construct
PLTGRSGGGGSS

SEQ ID NO: 378 - protein, 5 aa, synthetic construct
PPLGR

SEQ ID NO: 379 - protein, 10 aa, synthetic construct
PPPDMKLFPG

SEQ ID NO: 380 - protein, 10 aa, synthetic construct
PPPEVRSFSV

SEQ ID NO: 381 - protein, 10 aa, synthetic construct
PPPVLKLLEW

SEQ ID NO: 382 - protein, 12 aa, synthetic construct
PPSIARSDNLAN

SEQ ID NO: 383 - protein, 8 aa, synthetic construct
PQHRIVSF

SEQ ID NO: 384 - protein, 8 aa, synthetic construct
PRFKIIGG

SEQ ID NO: 385 - protein, 8 aa, synthetic construct
PRFRIIGG

SEQ ID NO: 386 - protein, 10 aa, synthetic construct
PRPFVKSVDQ

SEQ ID NO: 387 - protein, 4 aa, synthetic construct
PRQV

SEQ ID NO: 388 - protein, 4 aa, synthetic construct
PRSF

SEQ ID NO: 389 - protein, 10 aa, synthetic construct
PSPPVKMMPE

SEQ ID NO: 390 - protein, 10 aa, synthetic construct
PTNGGSGGSS

SEQ ID NO: 391 - protein, 4 aa, synthetic construct
PTNL

SEQ ID NO: 392 - protein, 10 aa, synthetic construct
PTNLGSGGSS

SEQ ID NO: 393 - protein, 8 aa, synthetic construct
PVGYTSSL

SEQ ID NO: 394 - protein, 10 aa, synthetic construct
PVPRLKLIKD

SEQ ID NO: 395 - protein, 8 aa, synthetic construct
PVQPIGPQ

SEQ ID NO: 396 - protein, 8 aa, synthetic construct
QALAMSAI

SEQ ID NO: 397 - protein, 7 aa, synthetic construct
QFQALRM

SEQ ID NO: 398 - protein, 10 aa, synthetic construct
QGPMFKSLWD

SEQ ID NO: 399 - protein, 8 aa, synthetic construct
QGRAITFI

SEQ ID NO: 400 - protein, 7 aa, synthetic construct
QHQALRM

SEQ ID NO: 401 - protein, 8 aa, synthetic construct
QNQALRIA

SEQ ID NO: 402 - protein, 7 aa, synthetic construct
QNQALRM

SEQ ID NO: 403 - protein, 8 aa, synthetic construct
QNQALRMA

SEQ ID NO: 404 - protein, 22 aa, synthetic construct
QNQALRMAGGSGGSLSGRSDNH

SEQ ID NO: 405 - protein, 22 aa, synthetic construct
QNQALRMAGGSGGSLSGRSGNH

SEQ ID NO: 406 - protein, 6 aa, synthetic construct
QSRRVP

SEQ ID NO: 407 - protein, 7 aa, synthetic construct
QSRRVPL

SEQ ID NO: 408 - protein, 7 aa, synthetic construct
QSRRVPV

SEQ ID NO: 409 - protein, 6 aa, synthetic construct
QTRRVP

SEQ ID NO: 410 - protein, 7 aa, synthetic construct
QTRRVPL

SEQ ID NO: 411 - protein, 7 aa, synthetic construct
QTRRVPV

SEQ ID NO: 412 - protein, 8 aa, synthetic construct
QYIVSRSA

SEQ ID NO: 413 - protein, 6 aa, synthetic construct
RALRAP

SEQ ID NO: 414 - protein, 10 aa, synthetic construct
REPFMKSLPW

SEQ ID NO: 415 - protein, 6 aa, synthetic construct
RFPLKV

SEQ ID NO: 416 - protein, 10 aa, synthetic construct
RFPSLKSFPL

SEQ ID NO: 417 - protein, 7 aa, synthetic construct
RFPYGVW

SEQ ID NO: 418 - protein, 8 aa, synthetic construct
RFYRNQFF

SEQ ID NO: 419 - protein, 4 aa, synthetic construct
RGPA

SEQ ID NO: 420 - protein, 8 aa, synthetic construct
RGPAFNPM

SEQ ID NO: 421 - protein, 8 aa, synthetic construct
RGPATPIM

SEQ ID NO: 422 - protein, 7 aa, synthetic construct
RGPKLYW

SEQ ID NO: 423 - protein, 6 aa, synthetic construct
RHLAKL

SEQ ID NO: 424 - protein, 8 aa, synthetic construct
RIGRSDNH

SEQ ID NO: 425 - protein, 8 aa, synthetic construct
RKMPNITV

SEQ ID NO: 426 - protein, 14 aa, synthetic construct
RKSSIIIRMRDVVL

SEQ ID NO: 427 - protein, 8 aa, synthetic construct
RKTVQHWW

SEQ ID NO: 428 - protein, 8 aa, synthetic construct
RLGRSDNN

SEQ ID NO: 429 - protein, 8 aa, synthetic construct
RMHLRSLG

SEQ ID NO: 430 - protein, 8 aa, synthetic construct
RPLARAGI

SEQ ID NO: 431 - protein, 8 aa, synthetic construct
RPLARAGL

SEQ ID NO: 432 - protein, 12 aa, synthetic construct
RPLNGRSDNHKA

SEQ ID NO: 433 - protein, 12 aa, synthetic construct
RPLNGRSDNHKK

SEQ ID NO: 434 - skipped

SEQ ID NO: 435 - protein, 12 aa, synthetic construct
RPLNGRSDNHKR

SEQ ID NO: 436 - protein, 12 aa, synthetic construct
RPLNGRSDNHQA

SEQ ID NO: 437 - protein, 12 aa, synthetic construct
RPLNGRSDNHQK

SEQ ID NO: 438 - protein, 12 aa, synthetic construct
RPLNGRSDNHQR

SEQ ID NO: 439 - protein, 12 aa, synthetic construct
RPLNGRSDNHRA

SEQ ID NO: 440 - protein, 12 aa, synthetic construct
RPLNGRSDNHRK

SEQ ID NO: 441 - protein, 12 aa, synthetic construct
RPLNGRSDNHRR

SEQ ID NO: 442 - protein, 12 aa, synthetic construct
RPLSGRSDNHKA

SEQ ID NO: 443 - protein, 12 aa, synthetic construct
RPLSGRSDNHKK

SEQ ID NO: 444 - protein, 12 aa, synthetic construct
RPLSGRSDNHKR

SEQ ID NO: 445 - protein, 12 aa, synthetic construct
RPLSGRSDNHQA

SEQ ID NO: 446 - protein, 12 aa, synthetic construct
RPLSGRSDNHQK

SEQ ID NO: 447 - protein, 12 aa, synthetic construct
RPLSGRSDNHQR

SEQ ID NO: 448 - protein, 12 aa, synthetic construct
RPLSGRSDNHRA

SEQ ID NO: 449 - protein, 12 aa, synthetic construct
RPLSGRSDNHRK

SEQ ID NO: 450 - protein, 12 aa, synthetic construct
RPLSGRSDNHRR

SEQ ID NO: 451 - protein, 8 aa, synthetic construct
RPSPMWAY

SEQ ID NO: 452 - protein, 8 aa, synthetic construct
RRHDGLRA

SEQ ID NO: 453 - protein, 8 aa, synthetic construct
RRHDGLRS

SEQ ID NO: 454 - protein, 8 aa, synthetic construct
RSLVFAPI

SEQ ID NO: 455 - protein, 8 aa, synthetic construct
RSPSRLKC

SEQ ID NO: 456 - protein, 10 aa, synthetic construct
RVPKVKVMLD

SEQ ID NO: 457 - protein, 8 aa, synthetic construct
SAGFSLPA

SEQ ID NO: 458 - protein, 8 aa, synthetic construct
SAPAVESE

SEQ ID NO: 459 - protein, 10 aa, synthetic construct
SAPYFRMMDM

SEQ ID NO: 460 - protein, 8 aa, synthetic construct
SARGPSRW

SEQ ID NO: 461 - protein, 8 aa, synthetic construct
SCGPPLGR

SEQ ID NO: 462 - protein, 8 aa, synthetic construct
SCSPPLGR

SEQ ID NO: 463 - protein, 8 aa, synthetic construct
SGGPLGVR

SEQ ID NO: 464 - protein, 8 aa, synthetic construct
SGGPPLGR

SEQ ID NO: 465 - protein, 7 aa, synthetic construct
SGPPLGR

SEQ ID NO: 466 - protein, 4 aa, synthetic construct
SGRS

SEQ ID NO: 467 - protein, 5 aa, synthetic construct
SGRSA

SEQ ID NO: 468 - protein, 6 aa, synthetic construct
SGRSAN

SEQ ID NO: 469 - protein, 7 aa, synthetic construct
SGRSANI

SEQ ID NO: 470 - protein, 7 aa, synthetic construct
SGRSANP

SEQ ID NO: 471 - protein, 9 aa, synthetic construct
SGRSANPRG

SEQ ID NO: 472 - protein, 5 aa, synthetic construct
SGRSD

SEQ ID NO: 473 - protein, 6 aa, synthetic construct
SGRSDD

SEQ ID NO: 474 - protein, 7 aa, synthetic construct
SGRSDDH

SEQ ID NO: 475 - protein, 6 aa, synthetic construct
SGRSDI

SEQ ID NO: 476 - protein, 7 aa, synthetic construct
SGRSDIH

SEQ ID NO: 477 - protein, 6 aa, synthetic construct
SGRSDN

SEQ ID NO: 478 - protein, 7 aa, synthetic construct
SGRSDNI

SEQ ID NO: 479 - protein, 7 aa, synthetic construct
SGRSDNP

SEQ ID NO: 480 - protein, 6 aa, synthetic construct
SGRSDQ

SEQ ID NO: 481 - protein, 7 aa, synthetic construct
SGRSDQH

SEQ ID NO: 482 - protein, 6 aa, synthetic construct
SGRSDT

SEQ ID NO: 483 - protein, 7 aa, synthetic construct
SGRSDTH

SEQ ID NO: 484 - protein, 6 aa, synthetic construct
SGRSDY

SEQ ID NO: 485 - protein, 7 aa, synthetic construct
SGRSDYH

SEQ ID NO: 486 - protein, 5 aa, synthetic construct
SGRSG

SEQ ID NO: 487 - protein, 6 aa, synthetic construct
SGRSGN

SEQ ID NO: 488 - protein, 7 aa, synthetic construct
SGRSGNH

SEQ ID NO: 489 - protein, 8 aa, synthetic construct
SGSPPLGR

SEQ ID NO: 490 - protein, 8 aa, synthetic construct
SIARSDNL

SEQ ID NO: 491 - protein, 14 aa, synthetic construct
SISSGLLSGRSDNI

SEQ ID NO: 492 - protein, 8 aa, synthetic construct
SMLRSMPL

SEQ ID NO: 493 - protein, 6 aa, synthetic construct
SNPFKY

SEQ ID NO: 494 - protein, 8 aa, synthetic construct
SPLPLRVP

SEQ ID NO: 495 - protein, 8 aa, synthetic construct
SPLTGRSG

SEQ ID NO: 496 - protein, 6 aa, synthetic construct
SPPLGR

SEQ ID NO: 497 - protein, 8 aa, synthetic construct
SPRSIMLA

SEQ ID NO: 498 - protein, 5 aa, synthetic construct
SRRVP

SEQ ID NO: 499 - protein, 6 aa, synthetic construct
SRRVPL

SEQ ID NO: 500 - protein, 6 aa, synthetic construct
SRRVPV

SEQ ID NO: 501 - protein, 8 aa, synthetic construct
SSGPPLGR

SEQ ID NO: 502 - protein, 7 aa, synthetic construct
SSPPLGR

SEQ ID NO: 503 - protein, 8 aa, synthetic construct
SSRGPAYL

SEQ ID NO: 504 - protein, 9 aa, synthetic construct
SSRHRRALD

SEQ ID NO: 505 - protein, 15 aa, synthetic construct
SSSFDKGKYKKGDDA

SEQ ID NO: 506 - protein, 15 aa, synthetic construct
SSSFDKGKYKRGDDA

SEQ ID NO: 507 - protein, 8 aa, synthetic construct
SSSPPLGR

SEQ ID NO: 508 - protein, 8 aa, synthetic construct
STFPFGMF

SEQ ID NO: 509 - protein, 6 aa, synthetic construct
STVFHM

SEQ ID NO: 510 - protein, 6 aa, synthetic construct
SVHHLI

SEQ ID NO: 511 - protein, 8 aa, synthetic construct
SVSGLLSH

SEQ ID NO: 512 - protein, 8 aa, synthetic construct
SVSGLLSS

SEQ ID NO: 513 - protein, 8 aa, synthetic construct
SVSGLRSH

SEQ ID NO: 514 - protein, 8 aa, synthetic construct
SVSGLRSS

SEQ ID NO: 515 - protein, 4 aa, synthetic construct
TARG

SEQ ID NO: 516 - protein, 5 aa, synthetic construct
TARGP

SEQ ID NO: 517 - protein, 12 aa, synthetic construct
TARGPALAHGLF

SEQ ID NO: 518 - protein, 6 aa, synthetic construct
TARGPS

SEQ ID NO: 519 - protein, 7 aa, synthetic construct
TARGPSF

SEQ ID NO: 520 - protein, 8 aa, synthetic construct
TARGPSFK

SEQ ID NO: 521 - protein, 7 aa, synthetic construct
TARGPSW

SEQ ID NO: 522 - protein, 10 aa, synthetic construct
TARGPVPRQV

SEQ ID NO: 523 - protein, 4 aa, synthetic construct
TFVH

SEQ ID NO: 524 - protein, 12 aa, synthetic construct
TGLSGRSVTQTS

SEQ ID NO: 525 - protein, 8 aa, synthetic construct
TGRGPSWV

SEQ ID NO: 526 - protein, 12 aa, synthetic construct
TLRLGRSDNNKN

SEQ ID NO: 527 - protein, 8 aa, synthetic construct
TLSGLRSP

SEQ ID NO: 528 - protein, 8 aa, synthetic construct
TSGRSANP

SEQ ID NO: 529 - protein, 8 aa, synthetic construct
TSGRSGNP

SEQ ID NO: 530 - protein, 12 aa, synthetic construct
TSLSGRSANPRG

SEQ ID NO: 531 - protein, 12 aa, synthetic construct
TSLSGRSGNPRG

SEQ ID NO: 532 - protein, 8 aa, synthetic construct
TSSGLRSP

SEQ ID NO: 533 - protein, 12 aa, synthetic construct
TSTSGRSANPRG

SEQ ID NO: 534 - protein, 22 aa, synthetic construct
TSTSGRSANPRGGGAVGLLAPP

SEQ ID NO: 535 - protein, 24 aa, synthetic construct
TSTSGRSANPRGGGVHMPLGFLGP

SEQ ID NO: 536 - protein, 12 aa, synthetic construct
TSTSGRSGNPRG

SEQ ID NO: 537 - protein, 8 aa, synthetic construct
TVSGLRSP

SEQ ID NO: 538 - protein, 8 aa, synthetic construct
VAGRSMRP

SEQ ID NO: 539 - protein, 10 aa, synthetic construct
VAPQLKSLVP

SEQ ID NO: 540 - protein, 8 aa, synthetic construct
VAQFVLTE

SEQ ID NO: 541 - protein, 10 aa, synthetic construct
VHMPLGFLGP

SEQ ID NO: 542 - protein, 20 aa, synthetic construct
VHMPLGFLGPGGLSGRSDNH

SEQ ID NO: 543 - protein, 24 aa, synthetic construct
VHMPLGFLGPGGTSTSGRSANPRG

SEQ ID NO: 544 - protein, 10 aa, synthetic construct
VLPELRSVFS

SEQ ID NO: 545 - protein, 8 aa, synthetic construct
VLSKQMSF

SEQ ID NO: 546 - protein, 7 aa, synthetic construct
VPAGRRS

SEQ ID NO: 547 - protein, 8 aa, synthetic construct
VPAGRRSL

SEQ ID NO: 548 - protein, 4 aa, synthetic construct
VPRQ

SEQ ID NO: 549 - protein, 5 aa, synthetic construct
VPRQV

SEQ ID NO: 550 - protein, 5 aa, synthetic construct
VSRSA

SEQ ID NO: 551 - protein, 8 aa, synthetic construct
VVPEGRRS

SEQ ID NO: 552 - protein, 8 aa, synthetic construct
WATPRPMR

SEQ ID NO: 553 - protein, 8 aa, synthetic construct
WDHPISLL

SEQ ID NO: 554 - protein, 10 aa, synthetic construct
YDPZVKVVLA

SEQ ID NO: 555 - protein, 8 aa, synthetic construct
YGAGLGVV

SEQ ID NO: 556 - protein, 7 aa, synthetic construct
YIVSRSA

SEQ ID NO: 557 - protein, 8 aa, synthetic construct
YKKFVGSL

SEQ ID NO: 558 - protein, 10 aa, synthetic construct
YVPRVKALEM

SEQ ID NO: 559 - protein, 5 aa, synthetic construct
GSGGS

SEQ ID NO: 560 - protein, 4 aa, synthetic construct
GGGS

SEQ ID NO: 561 - protein, 4 aa, synthetic construct
GGSG

SEQ ID NO: 562 - protein, 5 aa, synthetic construct
GGSGG

SEQ ID NO: 563 - protein, 5 aa, synthetic construct
GSGSG

SEQ ID NO: 564 - protein, 5 aa, synthetic construct
GSGGG

SEQ ID NO: 565 - protein, 5 aa, synthetic construct
GGGSG

SEQ ID NO: 566 - protein, 5 aa, synthetic construct
GSSSG

SEQ ID NO: 567 - protein, 13 aa, synthetic construct
GSSGGSGGSGGSG

SEQ ID NO: 568 - protein, 11 aa, synthetic construct
GSSGGSGGSGG

SEQ ID NO: 569 - protein, 12 aa, synthetic construct
GSSGGSGGSGGS

SEQ ID NO: 570 - protein, 16 aa, synthetic construct
GSSGGSGGSGGSGGGS

SEQ ID NO: 571 - protein, 10 aa, synthetic construct
GSSGGSGGSG

SEQ ID NO: 572 - protein, 11 aa, synthetic construct
GSSGGSGGSGS

SEQ ID NO: 573 - protein, 8 aa, synthetic construct
GGGSSGGS

SEQ ID NO: 574 - protein, 15 aa, synthetic construct
GGGGSGGGGSGGGGS

SEQ ID NO: 575 - protein, 4 aa, synthetic construct
GGGS

SEQ ID NO: 576 - protein, 5 aa, synthetic construct
GSSGT

SEQ ID NO: 577 - protein, 4 aa, synthetic construct
GSSG

SEQ ID NO: 578 - protein, 13 aa, synthetic construct
GGGSSGGSGGSGG

SEQ ID NO: 579-580 - skipped (2 entries)

SEQ ID NO: 581 - protein, 5 aa, synthetic construct
TYAMN

SEQ ID NO: 582 - protein, 19 aa, synthetic construct
RIRSKYNNYATYYADSVKD

SEQ ID NO: 583 - protein, 14 aa, synthetic construct
HGNFGNSYVSWFAY

SEQ ID NO: 584 - protein, 14 aa, synthetic construct
RSSTGAVTTSNYAN

SEQ ID NO: 585 - protein, 7 aa, synthetic construct
GTNKRAP

SEQ ID NO: 586 - protein, 9 aa, synthetic construct
ALWYSNLWV

SEQ ID NO: 587 - protein, 125 aa, synthetic construct
EVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 588 - protein, 109 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVL

SEQ ID NO: 589 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 590 - protein, 124 aa, synthetic construct
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVS

SEQ ID NO: 591 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 592 - protein, 249 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 593 - protein, 125 aa, synthetic construct; identical to SEQ ID NO: 589

SEQ ID NO: 594 - protein, 249 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 595 - protein, 109 aa, synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVL

SEQ ID NO: 596 - protein, 249 aa, synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 597 - protein, 249 aa, synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 598 - protein, 15 aa, synthetic construct
MMYCGGNEVLCGPRV

SEQ ID NO: 599 - protein, 15 aa, synthetic construct
GYLWGCEWNCGGITT

SEQ ID NO: 600 - protein, 15 aa, synthetic construct
GYRWGCEWNCGGITT

SEQ ID NO: 601 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 602 - DNA, 1350 bp, synthetic construct
  source 1..1350: mol_type = other DNA
gaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgctctagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 603 - protein, 261 aa, synthetic construct
QGQSGQGSWCMMHHEGNVCQTVGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 604 - DNA, 783 bp, synthetic construct
  source 1..783: mol_type = other DNA
cagggacagtccggccagggctcatggtgcatgatgcaccacgaggggaatgtgtgccagaccgtgggcggcggatcctctggcggttcaatctcatcaggactgctgtctgggcggagcgataatcatggcggaggcagtgatatacagatgacacagagcccaagtagtcttagcgccagcgtgggcgatcgggtgactatcacttgccgggcttctcaggatgtcaataccgcagttgcttggtatcagcagaagcctgggaaagctccgaagctgcttatttattctgcaagtttcctgtactccggggtaccctcaagattcagcgggtctagaagtgggaccgacttcaccctcacaatcagcagcctgcagcccgaagatttcgccacctactattgtcagcagcattacacgaccccacctactttcgggcagggaaccaaggtggagataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 605 - protein, 261 aa, synthetic construct
QGQSGQGLDCVRTLYYSDCYHRGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 606 - skipped

SEQ ID NO: 607 - DNA, 783 bp, synthetic construct
  source 1..783: mol_type = other DNA
cagggacagagcggccaagggttggactgcgtgcggactctctactactccgattgttaccacaggggcggaggttcttccggcgggtccatttcctcaggtctcctcagtggccggtccgataaccacgggggcggcagcgatatccagatgactcagagcccctcatccctctccgcttccgttggcgaccgggtgactatcacttgcagggcttcacaggacgttaacacagcagtggcgtggtatcagcagaaacctggcaaggccccaaaactgctgatctatagcgcgagcttcctctactccggggtacccagcagattttcaggcagccggagcggcaccgactttacattgactatcagcagtttgcaacccgaggatttcgcaacctactactgtcagcagcattacacaaccccccccacgtttggacagggtactaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 608 - protein, 261 aa, synthetic construct
QGQSGQGICSYNWHFDTIDCNSGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 609 - DNA, 783 bp, synthetic construct
  source 1..783: mol_type = other DNA
caaggacagagcggccagggcatctgctcatacaattggcatttcgacaccattgattgtaatagtgggggtggaagttcagggggctctatctccagcggtctcctctcagggagaagcgacaaccatggcggcgggagcgacatccagatgacccagagccctagctctttgagcgccagtgtaggagatagggtcactatcacctgcagggcgtcacaagatgtaaacacggctgttgcttggtatcagcagaagcctggcaaggctcccaaactgctgatttactccgcaagttttctctattccggggtgcccagcaggttttccggatctcggtctgggaccgatttcactctcacaatctccagtcttcagcctgaggattttgccacatactactgccagcaacattacaccaccccacccacgttcggccagggcacaaaagttgagataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 610 - protein, 261 aa, synthetic construct
QGQSGQGCCYVLLDGILMPCDCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 611 - DNA, 783 bp, synthetic construct
  source 1..783: mol_type = other DNA
cagggccaaagtggccagggatgctgttacgtgctcctggatggcatcttgatgccatgcgattgtggcgggggctcttcaggtggcagcatttctagcggcctgctctctgggaggtccgacaatcatgggggtgggtcagacattcagatgacacagtccccgagcagtctctcagcctcagtgggcgacagggtgacaataacttgtagggcatctcaggatgttaacactgccgtagcatggtaccagcagaagccggggaaagctccaaagctgcttatctatagtgccagttttctgtatagcggggtgcctagccgattcagcggcagccggagcggcactgatttcaccctcaccatttcaagtttgcagccagaggatttcgccacgtactattgtcagcaacactacacaacaccaccgaccttcggacagggaacgaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 612 - protein, 261 aa, synthetic construct
QGQSGQGSCYVLLDGILMPSDCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 613 - DNA, 783 bp, synthetic construct
  source 1..783: mol_type = other DNA
cagggccagtctggccagggctcttgttacgtcttgttggacgggatcctcatgccttccgattgcggaggtggtagcagtggtggctccatttcatccggcctgctgtctggcaggtcagataaccacggcgggggctctgatatccagatgacccagtccccaagttccctgtccgccagcgtaggcgatagagtgaccatcacgtgccgggccagtcaggacgtgaacacggctgttgcatggtatcaacaaaaaccaggaaaagcccccaaattgctgatctattccgcgagtttcctttacagtggagttccctcccgattttccggaagccggtccggaactgattttactcttactatctcctcactgcaaccggaggatttcgcaacgtattattgccaacagcattataccaccccacctacattcggccagggaacaaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 614 - protein, 261 aa, synthetic construct
QGQSGQGSCYVLLDGILMPCDSGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 615 - DNA, 783 bp, synthetic construct
  source 1..783: mol_type = other DNA
cagggccagtcagggcagggaagttgctatgttctgctcgacgggatcctgatgccttgtgatagcggcggaggtagctccggcgggtccatcagttcagggctgctgtcagggcgatccgacaaccatggcggaggctctgatatccaaatgacccagagtccgtcaagcctgagcgctagcgtgggtgatcgagttaccattacatgcagggctagccaggacgtcaataccgcggtagcctggtaccaacaaaagcctggaaaagcccctaaactcctcatctattctgcgagtttcctgtacagtggcgtgccaagtagattcagcggatctcgctcaggcactgactttactctcacgatatctagtttgcagccagaggactttgcaacctattactgccagcagcattatacaaccccccctaccttcgggcaaggaactaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 616 - protein, 261 aa, synthetic construct
QGQSGQGCSYVLLDGILMPSDCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 617 - DNA, 783 bp, synthetic construct
  source 1..783: mol_type = other DNA
cagggccagtctggacagggttgttcttatgttctgctcgacggcatcctgatgccaagtgattgcggaggagggagctcaggtggatccatctcctctgggctcctttcaggtaggtcagacaaccacggcggaggctctgacattcagatgactcaatcccccagctccctgtccgccagtgttggggaccgcgtaaccatcacctgtagggcctctcaggacgtcaacaccgctgtggcttggtatcaacagaagccaggcaaagctcccaagctcttgatctactcagctagcttcctttattccggcgtccccagccgctttagcggcagccgcagtggtacagattttacattgacaatcagctcccttcagcctgaggatttcgcaacctattattgccaacagcattacaccacaccgcccacttttggccaggggactaaagtcgagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 618 - protein, 261 aa, synthetic construct
QGQSGQGCSYVLLDGILMPCDSGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 783 bp, synthetic construct
  source 1..783: mol_type = other DNA
caggggcagtcaggccaggggtgcagctacgtgctgctggatggaatccttatgccttgcgacagtggtggcggcagcagcgggggctccatcagtagtgggctgctgtccggaaggtcagataatcatggtgggggaagcgatatacagatgacacagagcccctctagcctgtccgcgagcgtcggggaccgagttacgatcacctgtagggccagtcaggacgtcaacacagcggtggcatggtaccagcagaagcccggcaaggcccccaaactcctgatttatagtgctagcttcctgtattccggggttccaagtcggttctccgggtctagaagcggcaccgacttcacgctgacaatatcatcactgcagcctgaagacttcgccacatactactgtcagcagcactacaccactcctccaaccttcgggcaggggaccaaggtcgagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 620 - protein, 261 aa, synthetic construct
QGQSGQGSECMISQWGPDCTVAGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 621 - DNA, 783 bp, synthetic construct
  source 1..783: mol_type = other DNA
cagggccagtctgggcaggggtccgagtgtatgatctcccagtggggtcccgactgtaccgttgcgggagggggcagttccggcggcagtatttccagtggattgctgtctggcagatctgataaccatggtggtggtagcgacatccagatgacccaaagcccctcaagcctgtctgcatccgtcggggatcgggtgactatcacgtgtagagcctcccaggatgtgaatacagccgtcgcctggtatcagcagaagcccgggaaggctcccaagttgttgatctattccgcctcttttttgtattccggcgtgccttcaagattcagcggttccagatcaggtacggactttaccctcacaatcagctctctgcaaccagaagacttcgcgacctattattgtcagcaacattacaccacaccgccaacctttgggcagggcacaaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 622 - protein, 261 aa, synthetic construct
QGQSGQGYCLYNKLNDTINCSDGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 623 - DNA, 783 bp, synthetic construct
  source 1..783: mol_type = other DNA
cagggccagagcggccaaggatactgcctctacaacaaactgaatgacactatcaactgttccgacggaggtggatccagcgggggatcaatcagcagcgggcttctcagtgggcggagcgacaatcacggcgggggatccgacattcagatgacacagtctccatctagcctttctgccagtgtgggtgaccgggtcacgatcacatgcagagccagccaggacgtgaataccgcagtcgcctggtaccagcagaagcccgggaaagcgccgaagcttctgatctattctgctagttttctctattcaggagtcccaagccgattctctggatctagatctggtacggactttacactcacgattagcagtctccaacccgaagacttcgctacctattattgccagcaacattatactacccctccaaccttcggacagggaaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 624 - protein, 261 aa, synthetic construct
QGQSGQGRCVMDPVTDTIDCNKGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 625 - DNA, 783 bp, synthetic construct
  source 1..783: mol_type = other DNA
cagggccaatccggtcagggacggtgtgttatggatcccgtcaccgatacaatcgactgcaataaaggaggaggctcttctgggggaagcatttctagtgggcttctgtccggacggtccgacaaccatggaggcggatccgacattcagatgactcaatctccctcatctctgagtgctagcgttggcgatcgcgtcacgatcacttgccgcgcttcacaggacgtgaataccgccgtggcgtggtaccagcagaagcctggaaaggcccccaagttgcttatatatagcgcctcctttctgtattctggtgtcccttcccgcttctctggttcccggtccggcaccgacttcacacttacaatctcctccctgcagccagaagatttcgctacttattattgtcagcaacactatacaacacctcctacttttgggcaaggaactaaagtcgaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 626 - protein, 261 aa, synthetic construct
QGQSGQGALICCSDVSGLCRWCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 783 bp, synthetic construct
  source 1..783: mol_type = other DNA
cagggccagtcagggcagggagccctgatctgctgtagcgacgtgtcagggctttgtagatggtgcgggggaggatcctctggtggatctatctctagcggactgctgagcgggcgcagcgacaaccacggcggcggaagtgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 628 - protein, 261 aa, synthetic construct
QGQSGQGALISCSDVSGLSRWCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 629 - DNA, 783 bp, synthetic construct
  source 1..783: mol_type = other DNA
caggggcaatccggtcaaggggcactgatcagttgcagcgatgtcagtggcctcagtagatggtgcggtggcggctcatctggcggcagcatctctagtgggctgctctctggtagaagcgataaccacggcggaggcagtgatatccagatgactcagtctccctccagtctctctgcttctgtgggagacagagttaccataacttgtcgagcttcccaggatgttaacacagcagtcgcatggtatcagcagaagcccggcaaagctcccaagctgctcatatacagcgcctcctttctgtactccggggtccctagcagatttagcgggagtaggtccggcaccgatttcacactcacaatctccagcctgcagcccgaggacttcgcaacatattattgtcagcagcactacactacaccgcccactttcggccaaggcacgaaagttgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 630 - protein, 261 aa, synthetic construct
QGQSGQGALISCSDVSGLCRWSGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 631 - DNA, 783 bp, synthetic construct
  source 1..783: mol_type = other DNA
cagggacagtccgggcaaggtgctctgatctcctgctcagatgttagtggtctctgtagatggagcgggggcgggagttccggaggcagcatttcttccggcctgctctctgggagatcagacaaccatggcggcggttctgatatacagatgacccagtctccatcctccctcagcgcatccgtgggtgatcgggtgactatcacttgtcgcgcctctcaggacgtgaacaccgctgtggcctggtaccagcagaaacccggcaaagctccaaaactccttatttacagcgccagcttcctctattctggggtcccttcacggttttctgggagccggtccgggaccgactttaccctcacaatcagttccctccagcctgaggacttcgctacttattattgtcaacaacactataccacaccccctacttttggccaggggacaaaggttgagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 632 - protein, 261 aa, synthetic construct
QGQSGQGALICSSDVSGLSRWCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 633 - DNA, 783 bp, synthetic construct
  source 1..783: mol_type = other DNA
cagggacagtcagggcagggagcactgatatgcagcagcgacgtctccgggctctcccggtggtgcgggggtggatccagtggagggagtatctcaagtggactcctgagcggcagatctgacaaccacggcgggggctcagatattcaaatgacccagtcacccagtagtctgtctgccagtgtgggagaccgggtgactatcacatgtcgggccagtcaggatgtgaatacggcggtggcatggtatcagcagaagccaggcaaagccccgaaactgcttatctattccgcgtctttcctgtactctggcgtgcccagtcggtttagcggctcccggtcaggaacagactttacactgaccatcagttcactgcagccagaagacttcgccacctactactgccagcagcattatacaacacccccaacgtttggccagggaaccaaggttgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 634 - protein, 261 aa, synthetic construct
QGQSGQGALICSSDVSGLCRWSGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 635 - DNA, 783 bp, synthetic construct
  source 1..783: mol_type = other DNA
caagggcagtctgggcagggtgcgttgatatgttcttctgacgtatcaggtctgtgtcggtggtccggcgggggcagctcaggtggctccattagcagtgggctgctcagcgggcgctccgataatcacgggggcggctctgacattcagatgacccaatctccctcctccctgtctgcgtctgtcggcgatagagtcaccatcacctgcagggccagccaagacgtaaacacagctgtggcatggtatcagcaaaaacccgggaaggcaccaaaactcctcatctattccgcttcatttttgtatagcggggtgccatctcggtttagtgggtcccggtccggcactgacttcactctgacaatctcaagtctccaacccgaagacttcgcaacctattattgtcagcagcattataccacacctccaacatttggccagggaactaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 636 - protein, 261 aa, synthetic construct
QGQSGQGSSCRTTGLMTTCDLDGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 637 - DNA, 783 bp, synthetic construct
  source 1..783: mol_type = other DNA
cagggccagtccggccagggtagtagctgtaggaccacaggccttatgaccacgtgtgacttggatggcggcgggtcctctgggggttccatatctagtggcctgttgtctgggcgaagcgataatcatggcggaggtagcgatatccaaatgacacagtcacctagttcactgtctgcatccgtgggcgacagggtcaccatcacttgtcgcgcttcccaggatgtcaataccgcagttgcctggtatcaacagaaacccggcaaggctccaaagctcctgatctacagcgcaagtttcctttatagtggtgtacccagccgcttttctggcagcaggagtggaactgatttcaccctgactatctccagcctgcagcctgaagattttgccacttattactgccaacagcactatacaactccacctacatttggtcagggcacgaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 638 - protein, 753 aa, synthetic construct
QGQSGQMMYCGGNEVLCGPRVGSSGGSGGSGGISSGLLSGRSDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 639 - DNA, 2259 bp, synthetic construct
  source 1..2259: mol_type = other DNA
caaggccagtctggccaaatgatgtattgcggtgggaatgaggtgttgtgcgggccgcgggttggctcgagcggtggcagcggtggctctggtggtatctcttccggactgctgtccggcagatccgacaatcacggcggcggttctcagaccgtggtcacacaggagccctcactgacagtgagccctggcgggaccgtcacactgacttgtcgcagttcaactggcgccgtgactaccagcaattacgctaactgggtccagcagaaaccaggacaggcaccacgaggactgatcggaggaactaataagagagcaccaggaacccctgcaaggttctccggatctctgctggggggaaaagccgctctgacactgagcggcgtgcagcctgaggacgaagctgagtactattgcgcactgtggtactccaacctgtgggtgtttggcgggggaactaagctgaccgtcctgggaggaggaggaagcggaggaggagggagcggaggaggaggatccgaagtgcagctggtcgagagcggaggaggactggtgcagccaggaggatccctgaagctgtcttgtgcagccagtggcttcaccttcaacacttacgcaatgaactgggtgcggcaggcacctgggaagggactggaatgggtcgcccggatcagatctaaatacaataactatgccacctactatgctgacagtgtgaaggataggttcaccatttcacgcgacgatagcaaaaacacagcttatctgcagatgaataacctgaagaccgaggatacagcagtgtactattgcgtcagacacggcaatttcgggaactcttacgtgagttggtttgcctattggggacaggggacactggtcaccgtctcctcaggaggtggtggatccgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgctctagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 640 - protein, 763 aa, synthetic construct
QGQSGQMMYCGGNEVLCGPRVGSSGGSGGSGGISSGLLSGRSDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 641 - DNA, 2289 bp, synthetic construct
  source 1..2289: mol_type = other DNA
caaggccagtctggccaaatgatgtattgcggtgggaatgaggtgttgtgcgggccgcgggttggctcgagcggtggcagcggtggctctggtggtatctcttccggactgctgtccggcagatccgacaatcacggcggcggttctcagaccgtggtcacacaggagccctcactgacagtgagccctggcgggaccgtcacactgacttgtcgcagttcaactggcgccgtgactaccagcaattacgctaactgggtccagcagaaaccaggacaggcaccacgaggactgatcggaggaactaataagagagcaccaggaacccctgcaaggttctccggatctctgctggggggaaaagccgctctgacactgagcggcgtgcagcctgaggacgaagctgagtactattgcgcactgtggtactccaacctgtgggtgtttggcgggggaactaagctgaccgtcctgggaggaggaggaagcggaggaggagggagcggaggaggaggatccgaagtacagttggttgagtcaggagggggtctcgtgcaaccgggaggcagcttgaaattgtcttgcgcagcctccggtttcaccttcaatacgtatgctatgaactgggtgcgccaagcgcctggtaaagggctcgaatgggttgcacggataagaagcaaatataacaactacgcaacttattacgctgatagcgtaaaagataggttcaccatttcacgagacgattccaaaaatacagcgtatttgcaaatgaataatctcaaaaccgaagataccgctgtatattattgtgttcggcacggaaacttcggaaactcttacgttagctggttcgcatactggggccaagggacgctcgtgacagtatcctcaggaggtggtggatctggaggtggtggatctggaggtggtggatccgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgctctagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 642 - protein, 722 aa, synthetic construct
SDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 643 - DNA, 2166 bp, synthetic construct
  source 1..2166: mol_type = other DNA
tccgacaatcacggcggcggttctcagaccgtggtcacacaggagccctcactgacagtgagccctggcgggaccgtcacactgacttgtcgcagttcaactggcgccgtgactaccagcaattacgctaactgggtccagcagaaaccaggacaggcaccacgaggactgatcggaggaactaataagagagcaccaggaacccctgcaaggttctccggatctctgctggggggaaaagccgctctgacactgagcggcgtgcagcctgaggacgaagctgagtactattgcgcactgtggtactccaacctgtgggtgtttggcgggggaactaagctgaccgtcctgggaggaggaggaagcggaggaggagggagcggaggaggaggatccgaagtacagttggttgagtcaggagggggtctcgtgcaaccgggaggcagcttgaaattgtcttgcgcagcctccggtttcaccttcaatacgtatgctatgaactgggtgcgccaagcgcctggtaaagggctcgaatgggttgcacggataagaagcaaatataacaactacgcaacttattacgctgatagcgtaaaagataggttcaccatttcacgagacgattccaaaaatacagcgtatttgcaaatgaataatctcaaaaccgaagataccgctgtatattattgtgttcggcacggaaacttcggaaactcttacgttagctggttcgcatactggggccaagggacgctcgtgacagtatcctcaggaggtggtggatctggaggtggtggatctggaggtggtggatccgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgctctagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 644 - protein, 222 aa, synthetic construct
SDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - DNA, 666 bp, synthetic construct
  source 1..666: mol_type = other DNA
agcgacaaccacggcggcggaagtgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 646 - protein, 763 aa, synthetic construct
QGQSGSGYLWGCEWNCGGITTGSSGGSGGSGGISSGLLSGRSDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 647 - DNA, 2289 bp, synthetic construct
  source 1..2289: mol_type = other DNA
caaggccagtctggttcaggttatctgtggggttgcgagtggaattgcggagggatcactacaggatctagtggtggcagcggtggctctggtggtatatcgagtggattgctgtctggcagatctgacaatcacggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggaggtggtggatcaggaggtggtggttctggaggtggtggaagtgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgcagtagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 648 - protein, 722 aa, synthetic construct
SDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 649 - DNA, 2166 bp, synthetic construct
  source 1..2166: mol_type = other DNA
tctgacaatcacggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggaggtggtggatcaggaggtggtggttctggaggtggtggaagtgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgcagtagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 650-669 - skipped (20 entries)

SEQ ID NO: 670 - protein, 758 aa, synthetic construct
QGQSGSGYLWGCEWNCGGITTGSSGGSGGSGGLSGRSDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 671 - DNA, 2274 bp, synthetic construct
  source 1..2274: mol_type = other DNA
caaggccagtctggttcaggttatctgtggggttgcgagtggaattgcggagggatcactacaggatctagtggtggcagcggtggctctggtggtctgagcggccgttccgataatcatggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggaggtggtggatcaggaggtggtggttctggaggtggtggaagtgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgcagtagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 672 - protein, 256 aa, synthetic construct
QGQSGQGALICCSDVSGLCRWCGGGSSGGSLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 673 - DNA, 768 bp, synthetic construct
  source 1..768: mol_type = other DNA
cagggccagtcagggcagggagccctgatctgctgtagcgacgtgtcagggctttgtagatggtgcgggggaggatcctctggtggatctctgagcggccgttccgataatcatggcggcggaagtgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 674 - protein, 763 aa, synthetic construct
QGQSGSGYLWGCEWNCGGITTGSSGGSGGSGGISSGLLSGRSDNIGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 675 - DNA, 2289 bp, synthetic construct
  source 1..2289: mol_type = other DNA
caaggccagtctggttcaggttatctgtggggttgcgagtggaattgcggagggatcactacaggatctagtggtggcagcggtggctctggtggtatatcgagtggattgctgtctggcagatctgacaatattggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggaggtggtggatcaggaggtggtggttctggaggtggtggaagtgaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgcagtagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 676 - protein, 262 aa, synthetic construct
QSGQLYCTEGRWYEGTCAGGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 677 - DNA, 786 bp, synthetic construct
  source 1..786: mol_type = other DNA
cagtcagggcagctttactgcacggagggtcgttggtatgagggaacttgtgctggaggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 678 - protein, 262 aa, synthetic construct
QSGQLTKCATSTSTTCDLDGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 679 - DNA, 786 bp, synthetic construct
  source 1..786: mol_type = other DNA
cagtcagggcagttgacgaaatgcgctaccagcacatccactacttgtgatcttgatggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 680 - protein, 262 aa, synthetic construct
QSGQFGCGVVLDLDHCAPWGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 681 - DNA, 786 bp, synthetic construct
  source 1..786: mol_type = other DNA
cagtcagggcagttcggttgtggtgttgtgttggaccttgaccattgtgcaccatggggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 682 - protein, 262 aa, synthetic construct
QSGQTPCWHMIMTNPECMRGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 683 - DNA, 786 bp, synthetic construct
  source 1..786: mol_type = other DNA
cagtcagggcagactccttgttggcatatgataatgactaaccctgagtgcatgaggggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 684 - protein, 262 aa, synthetic construct
QSGQYNACWFGPYDCWYRPGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 685 - DNA, 786 bp, synthetic construct
  source 1..786: mol_type = other DNA
cagtcagggcagtacaacgcttgctggtttggtccttatgactgctggtatcgacctggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 686 - protein, 262 aa, synthetic construct
QSGQFLCEPPSWGPDCTMIGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 687 - DNA, 786 bp, synthetic construct
  source 1..786: mol_type = other DNA
cagtcagggcagttcctgtgcgagcctccatcttggggaccagactgtaccatgataggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 688 - protein, 262 aa, synthetic construct
QSGQDLCSFGLWYRGTCEGGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 689 - DNA, 786 bp, synthetic construct
  source 1..786: mol_type = other DNA
cagtcagggcaggatttgtgctctttcggactctggtaccgaggtacttgcgaaggtggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 690 - protein, 262 aa, synthetic construct
QSGQTCYHDMHKDTIHCNNGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 691 - DNA, 786 bp, synthetic construct
  source 1..786: mol_type = other DNA
cagtcagggcagacttgttatcacgatatgcacaaagacacgatacactgcaataacggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 692 - protein, 262 aa, synthetic construct
QSGQLYRDMCGGGCEDWHDGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 693 - DNA, 786 bp, synthetic construct
  source 1..786: mol_type = other DNA
cagtcagggcagctttatcgagacatgtgcggtggaggttgcgaagattggcatgacggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 694 - protein, 262 aa, synthetic construct
QSGQLGSLCDPFDCPGKNLGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 695 - DNA, 786 bp, synthetic construct
  source 1..786: mol_type = other DNA
cagtcagggcagcttggaagcctttgtgatcctttcgattgccctggtaagaatctcggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 696 - protein, 262 aa, synthetic construct
QSGQRGCSFGYWGNGTCLGGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 697 - DNA, 786 bp, synthetic construct
  source 1..786: mol_type = other DNA
cagtcagggcagaggggatgcagctttggttattggggtaacggtacatgcttgggtggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 698 - protein, 262 aa, synthetic construct
QSGQRACSFGVWWKGTCEGGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 699 - DNA, 786 bp, synthetic construct
  source 1..786: mol_type = other DNA
cagtcagggcagcgtgcatgtagcttcggagtttggtggaagggaacttgcgagggaggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 700 - protein, 262 aa, synthetic construct
QSGQANVCWLGPYDCRWESGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 701 - DNA, 786 bp, synthetic construct
  source 1..786: mol_type = other DNA
cagtcagggcaggctaatgtctgctggcttggaccatatgactgccgatgggagtcaggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 702 - protein, 262 aa, synthetic construct
QSGQGMCTSGRWYGGDCTGGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - DNA, 786 bp, synthetic construct
  source 1..786: mol_type = other DNA
cagtcagggcagggtatgtgtacctctggtcgatggtatggtggtgactgcaccggtggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 704 - protein, 262 aa, synthetic construct
QSGQACTHNTMTDKIYCNNGSSGGSGGSGGSGISSGLLSGRSDNIGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 705 - DNA, 786 bp, synthetic construct
  source 1..786: mol_type = other DNA
cagtcagggcaggcttgcacgcacaacacgatgaccgacaaaatatattgtaacaacggttccagtggtggttctggaggatcaggaggaagcggtatctctagcggactgctgagcgggcgcagcgacaacattggcggatccgacatacagatgacccagagtcccagctctctttctgctagtgtcggcgacagggtgacaatcacgtgccgcgcatcccaagacgtcaacaccgccgtggcatggtaccagcaaaaaccaggcaaagcccctaaactgttgatttattccgcttcattcctgtatagtggggtaccctcccgatttagcggctcccggtccggaacagatttcactctcactataagctcacttcagccagaagatttcgcaacatactattgtcaacagcattacacaaccccgccaaccttcggccaggggaccaaagtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 706 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 707 - DNA, 1350 bp, synthetic construct
  source 1..1350: mol_type = other DNA
gaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgcagtagatgggggggcgatggattttacgcaatggactactgggggcagggaaccttggtgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 708 - protein, 234 aa, synthetic construct
GSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 709 - DNA, 702 bp, synthetic construct
  source 1..702: mol_type = other DNA
ggctcgagccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggacgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 710 - protein, 447 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 711 - DNA, 1341 bp, synthetic construct
  source 1..1341: mol_type = other DNA
gaagtgcagctggtggagtccggaggcggtctggtacaacccggcggctccctgcgcctgagttgcgccgctagtggattcaatataaaggacacctacatccattgggtaaggcaagcacctggcaagggtctggagtgggtggctcgcatctacccaaccaacggctataccaggtacgcggattcagttaaggggcggttcactatcagcgccgacaccagcaagaatacagcttacctgcagatgaatagcctgcgcgcagaggatactgcggtgtattactgcagtagatgggggggcgatggattttacgcaatggactactgggggcagggaaccttggtgaccgtgtccagcgctagcaccaagggcccatcggtcttccccctggcaccctgtagcagaagcaccagcgagtctacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcaccaagacctacacctgtaacgtggaccacaagcccagcaacaccaaggtggacaagcgggtggaatctaagtacggccctccctgccctccttgcccagcccctgaatttctggggggaccgtcagtcttcctcttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctgcccagctccatcgagaaaaccatctccaaagccaaagggcagccccgagaaccacaggtgtgcaccctgcccccatcccaggaggagatgaccaagaaccaggtcagcctgagctgcgcagtcaaaggcttctatcccagcgacatcgccgtggagtgggagagcaatgggcagccggagaacaactacaagaccacgcctcccgtgctggactccgacggctccttcttcctcgtcagcagactcaccgtggacaagagcaggtggcaggaagggaacgtcttctcatgctccgtgatgcatgaggctctgcacaaccactacacgcagaagagcctctccctgtctctgggtaaa

SEQ ID NO: 712 - protein, 235 aa, synthetic construct
GSSKYGPPCPPCPAPEFLGPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 713 - DNA, 690 bp, synthetic construct
  source 1..690: mol_type = other DNA
ggctcgagcaagtacggccctccctgccctccttgcccagcccctgaatttctggggggaccgtcagtcttcctcttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctgcccagctccatcgagaaaaccatctccaaagccaaagggcagccccgagaaccacaggtgtacaccctgcccccatgccaggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcagactcaccgtggacaagagcaggtggcaggaagggaacgtcttctcatgctccgtgatgcatgaggctctgcacaaccactacacgcagaagagcctctccctgtctctgggtaaa

SEQ ID NO: 714 - protein, 528 aa, synthetic construct
QGQSGSGYLWGCEWNCGGITTGSSGGSGGSGGISSGLLSGRSDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGSSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 715 - DNA, 1584 bp, synthetic construct
  source 1..1584: mol_type = other DNA
caaggccagtctggttcaggttatctgtggggttgcgagtggaattgcggagggatcactacaggatctagtggtggcagcggtggctctggtggtatatcgagtggattgctgtctggcagatctgacaatcacggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggctcgagcaagtacggccctccctgccctccttgcccagcccctgaatttctggggggaccgtcagtcttcctcttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctgcccagctccatcgagaaaaccatctccaaagccaaagggcagccccgagaaccacaggtgtacaccctgcccccatgccaggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcagactcaccgtggacaagagcaggtggcaggaagggaacgtcttctcatgctccgtgatgcatgaggctctgcacaaccactacacgcagaagagcctctccctgtctctgggtaaa

SEQ ID NO: 716 - protein, 533 aa, synthetic construct
QGQSGSGYLWGCEWNCGGITTGSSGGSGGSGGISSGLLSGRSDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGSSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 717 - DNA, 1599 bp, synthetic construct
  source 1..1599: mol_type = other DNA
caaggccagtctggttcaggttatctgtggggttgcgagtggaattgcggagggatcactacaggatctagtggtggcagcggtggctctggtggtatatcgagtggattgctgtctggcagatctgacaatcacggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggaggtggtggatcaggctcgagcaagtacggccctccctgccctccttgcccagcccctgaatttctggggggaccgtcagtcttcctcttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctgcccagctccatcgagaaaaccatctccaaagccaaagggcagccccgagaaccacaggtgtacaccctgcccccatgccaggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcagactcaccgtggacaagagcaggtggcaggaagggaacgtcttctcatgctccgtgatgcatgaggctctgcacaaccactacacgcagaagagcctctccctgtctctgggtaaa

SEQ ID NO: 718 - protein, 543 aa, synthetic construct
QGQSGSGYLWGCEWNCGGITTGSSGGSGGSGGISSGLLSGRSDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGGGSGGGGSGGGGSGSSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 719 - protein, 6 aa, synthetic construct
QGQSGS

SEQ ID NO: 720 - protein, 5 aa, synthetic construct
GQSGS

SEQ ID NO: 721 - protein, 4 aa, synthetic construct
QSGS

SEQ ID NO: 722 - protein, 7 aa, synthetic construct
QGQSGQG

SEQ ID NO: 723 - protein, 6 aa, synthetic construct
GQSGQG

SEQ ID NO: 724 - protein, 5 aa, synthetic construct
QSGQG

SEQ ID NO: 725 - protein, 4 aa, synthetic construct
SGQG

SEQ ID NO: 726 - protein, 6 aa, synthetic construct
QGQSGQ

SEQ ID NO: 727 - protein, 5 aa, synthetic construct
GQSGQ

SEQ ID NO: 728 - protein, 4 aa, synthetic construct
QSGQ

SEQ ID NO: 729 - protein, 5 aa, synthetic construct
QGQSG

SEQ ID NO: 730 - protein, 99 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 731 - protein, 99 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVE

SEQ ID NO: 732 - protein, 107 aa, synthetic construct [sequence omitted]

SEQ ID NO: 733 - protein, 20 aa, synthetic construct
PKSCDKTHTCPPCPAPELLG

SEQ ID NO: 734 - protein, 17 aa, synthetic construct
SKYGPPCPPCPAPEFLG

SEQ ID NO: 735 - protein, 210 aa, synthetic construct
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 736 - protein, 210 aa, synthetic construct
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 737 - protein, 210 aa, synthetic construct
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 738 - protein, 7 aa, synthetic construct
GGGGSGS

SEQ ID NO: 739 - protein, 17 aa, synthetic construct
GGGGSGGGGSGGGGSGS

SEQ ID NO: 740 - DNA, 1629 bp, synthetic construct
  source 1..1629: mol_type = other DNA
caaggccagtctggttcaggttatctgtggggttgcgagtggaattgcggagggatcactacaggatctagtggtggcagcggtggctctggtggtatatcgagtggattgctgtctggcagatctgacaatcacggcggcggatcccagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggaggtggtggatcaggaggtggtggttctggaggtggtggaagtggctcgagcaagtacggccctccctgccctccttgcccagcccctgaatttctggggggaccgtcagtcttcctcttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctgcccagctccatcgagaaaaccatctccaaagccaaagggcagccccgagaaccacaggtgtacaccctgcccccatgccaggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcagactcaccgtggacaagagcaggtggcaggaagggaacgtcttctcatgctccgtgatgcatgaggctctgcacaaccactacacgcagaagagcctctccctgtctctgggtaaa

SEQ ID NO: 741 - protein, 479 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGSSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 742 - DNA, 1437 bp, synthetic construct
  source 1..1437: mol_type = other DNA
cagacggtagtgactcaggagccatcattttctgtctctcctggaggtactgtgacactcacatgtagaagctcaactggtgcagtcaccacttcaaattacgcgaattgggtccagcagacccctgggcaggctccgagagggttgattggaggtactaacaaacgggcaccgggagtgcctgataggttttccggttctattctcggaaacaaggcggctctcacgatcacgggtgcgcaggccgacgatgaatcagactattactgcgctttgtggtactcaaacctgtgggtattcggagggggcaccaagctgacggtgttgggtggggggggctctgggggagggggaagcggaggtgggggcagcgaggttcagcttgttgaaagtggtggcggactcgtacaaccgggtggaagtcttagactctcatgtgcagcatctggatttactttttctacttatgctatgaactgggtaagacaggcaccggggaaagggctggaatgggttgcacgcattcgatctaaatacaataactatgctacatactacgccgatagtgttaaggatcgattcactatatctcgggacgacagtaagaactcactttacctgcagatgaattccttgaaaactgaggacacggccgtttattattgtgtacggcacgggaatttcggcaattcttacgtttcctggttcgcctattgggggcaaggtaccctcgtgacagtatcctcaggctcgagcaagtacggccctccctgccctccttgcccagcccctgaatttctggggggaccgtcagtcttcctcttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttcaacagcacgtaccgtgtggtcagcgtcctcaccgtcctgcaccaggactggctgaatggcaaggagtacaagtgcaaggtctccaacaaaggcctgcccagctccatcgagaaaaccatctccaaagccaaagggcagccccgagaaccacaggtgtacaccctgcccccatgccaggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcagactcaccgtggacaagagcaggtggcaggaagggaacgtcttctcatgctccgtgatgcatgaggctctgcacaaccactacacgcagaagagcctctccctgtctctgggtaaa

SEQ ID NO: 743 - protein, 4 aa, synthetic construct
QGQS